(19) United States
(12) Patent Application Publication
(10) Pub. No.: US 2026/0327246 A1
(43) Pub. Date: Oct. 22, 2026

(54) FUSED RING KRAS INHIBITORS FOR TREATING DISEASE

(71) Applicant: BLOSSOMHILL THERAPEUTICS, INC., San Diego, CA (US)

(72) Inventors: Jingrong Jean CUI, San Diego, CA (US); Eugene Yuanjin RUI, San Diego, CA (US); Evan W. ROGERS, San Diego, CA (US)

(21) Appl. No.: 19/483,410

(22) PCT Filed: May 10, 2024
(86) PCT No.: PCT/US2024/028825

§ 371 Date: Nov. 11, 2025

Related U.S. Application Data
(60) Provisional application No. 63/502,046, filed on May 12, 2023.
(60) Provisional application No. 63/469,292, filed on May 26, 2023.

(51) Int. Cl. C07D 519/00; A61K 31/519; A61K 31/5383; A61K 31/55; A61K 31/553; C07D 471/22

(52) U.S. Cl. CPC C07D 519/00; A61K 31/519; A61K 31/5383; A61K 31/55; A61K 31/553; C07D 471/22

(57) ABSTRACT

The present disclosure relates to fused ring compounds targeting KRAS, pharmaceutical compositions containing the compounds, and methods of using such compounds to treat disease, such as cancer.

### RELATED APPLICATIONS

This application claims the benefit of U.S. Provisional Application No. 63/502,046, filed May 12, 2023, and U.S. Provisional Application No. 63/469,292, filed May 26, 2023, the entire disclosures of all of which are incorporated herein by reference.

### TECHNICAL FIELD

The present disclosure relates to fused ring compounds targeting KRAS, pharmaceutical compositions containing the compounds, and methods of using such compounds to treat disease, such as cancer.

### BACKGROUND

Ras is a GTP-binding protein and regulates many important physiologic processes within a cell, such as cell cycle progression, survival, apoptosis, etc., H-Ras, K-Ras, and N-Ras are the main members of Ras superfamily, which are tightly regulated by factors that switch on/off the GTPase activity. Somatic mutations at codons 12, 13 and 61 in the RAS genes are associated with about 16% of all human cancers and KRAS is the most frequently mutated RAS isoform, accounting for 85% of all RAS-related cancers (Prior I. A. et al, A comprehensive survey of Ras mutations in cancer. Cancer Res. 2012, 72, 2457-2467), including 86-96% in pancreatic cancers, 40-50% in colorectal cancers, and 27-39% in lung adenocarcinomas (Kessler D. et al. Drugging an undruggable pocket on KRAS Proc Nat Acad Sci USA. 2019, 116(32):15823-15829). Mutated RAS is locked in the constitutively activated GTP bound state and facilitates enhanced Ras signaling in cancer cells.

Direct targeting of mutant KRAS has previously proven challenging because of its high affinity for nucleotide and the lack of tractable binding pockets for small-molecule inhibitors. Recent successful inhibition of the KRAS G12C mutant by covalent chemical modifiers sotorasib and adagrasib (Stower K, KRAS inhibitors at last, Nature Medicine 2020, 26, 1804) in KRAS G12C mutated lung cancer patients has shed lights on targeting KRAS mutants for therapeutic invention. However, inhibitors targeting KRAS mutants without covalent formation at KRAS G12C are still absent. The increased understanding of structural elements of the KRAS switch II pocket made it possible to design KRAS inhibitors selective for different mutant variants. MRTX1133 has been reported as potent and highly selective noncovalent KRAS G12D inhibitor (Wang X. et al, Identification of MRTX1133, a noncovalent, potent, and selective KRAS^{G12D} inhibitor, J. Med. Chem. 2022, 65: 3123-3133). However, intraperitoneal injection of MRTX1133 was required to achieve sufficient plasma exposure and demonstrate drug efficacy in mice. This suggests MRTX1133 may have poor bioavailability.

Therefore, there is unmet medical need to develop new noncovalent KRAS inhibitors that can selectively target KRAS mutants such as KRAS G12C, KRAS G12D, KRAS G12V, KRAS G12R, KRAS G12S, KRAS G13C, KRAS G13D with good in vivo efficacy, safety, and predicted human oral pharmacokinetic profile for treating patients with KRAS mutant cancers.

### SUMMARY

In one aspect, the disclosure relates to a compound of the formula I, or a pharmaceutically acceptable salt thereof,

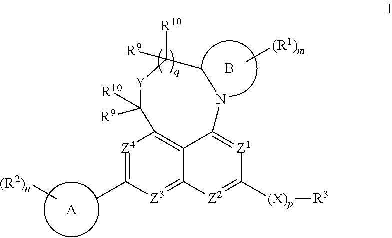

wherein R¹, R², R³, R⁹, R¹⁰, A, B, X, Y, Z¹, Z², Z³, Z⁴, m, n, p, and q are as described herein.

In another aspect, the disclosure relates to a compound of the formula II, III, IV, V, VI, VII, VIII, or IX, or a pharmaceutically acceptable salt thereof,

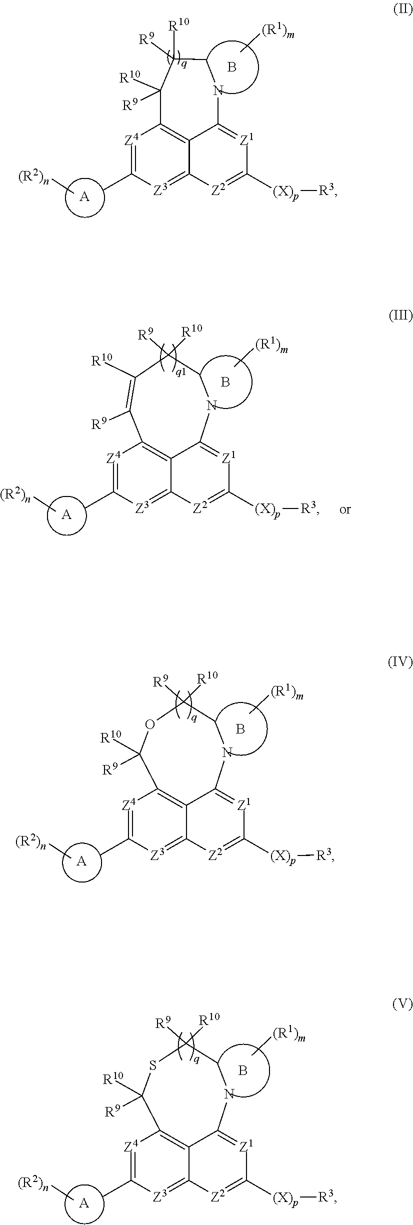

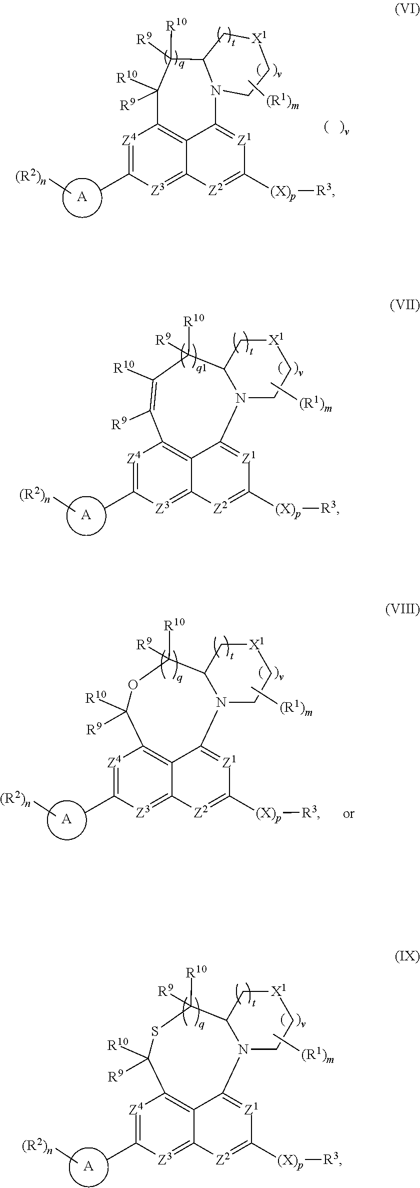

wherein R¹, R², R³, R⁹, R¹⁰, A, B, X, X¹, Z¹, Z², Z³, Z⁴, m, n, p, q, q1, t, and v are as described herein.

In another aspect, the disclosure relates to a compound of the formula X, XI, XII, XIII, XIV, XV, XVI, or XVII, or a pharmaceutically acceptable salt thereof,

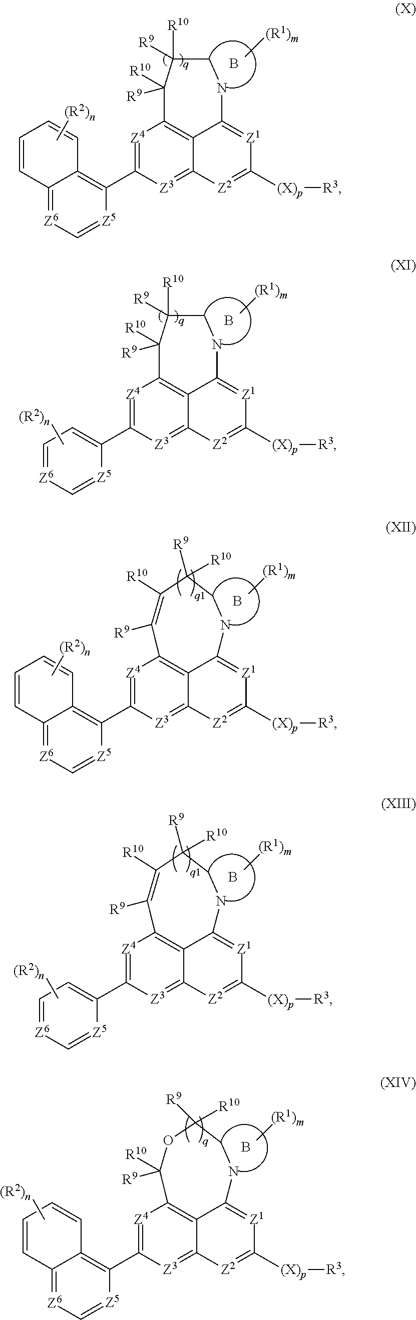

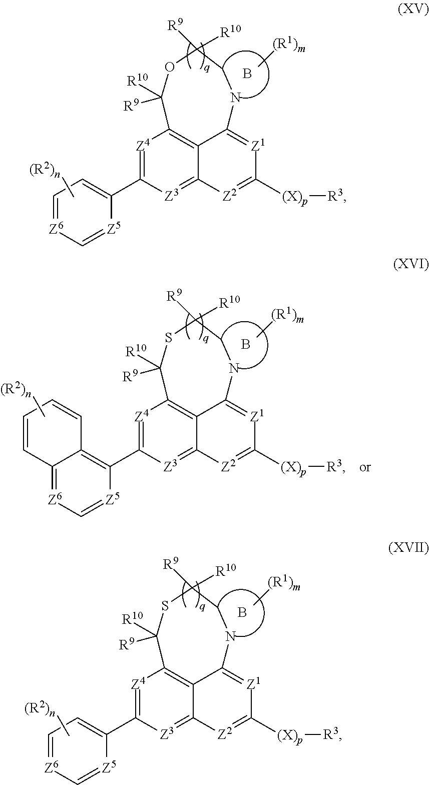

wherein R¹, R², R³, R⁹, R¹⁰, B, X, Z¹, Z², Z³, Z⁴, Z⁵, Z⁶, m, n, p, q, and q1, are as described herein.

In another aspect, the disclosure relates to a compound of the formula XVIII, XIX, XX, XXI, XXII, XXIII, XXIV, or XXV, or a pharmaceutically acceptable salt thereof,

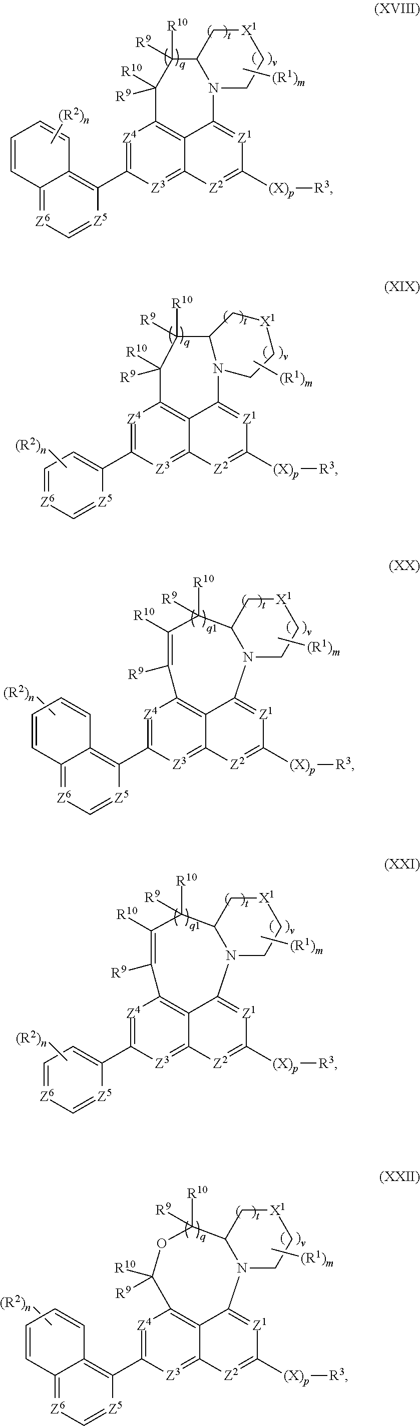

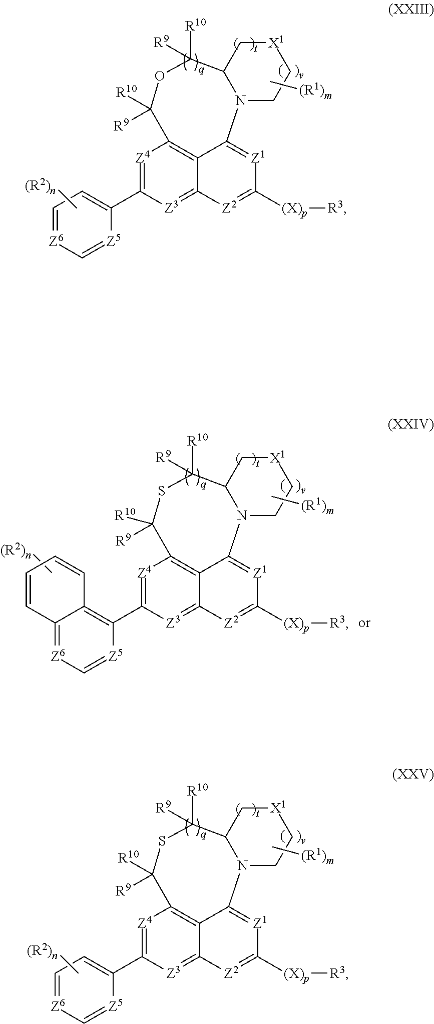

wherein R¹, R², R³, R⁹, R¹⁰, X, X¹, Z¹, Z², Z³, Z⁴, Z⁵, Z⁶, m, n, p, q, q1, t, and v are as described herein.

In another aspect, the disclosure relates to a compound of the formula XXVI, XXVII, XXVIII, XXIX, XXX, XXXI, XXXII, or XXXIII, or a pharmaceutically acceptable salt thereof,

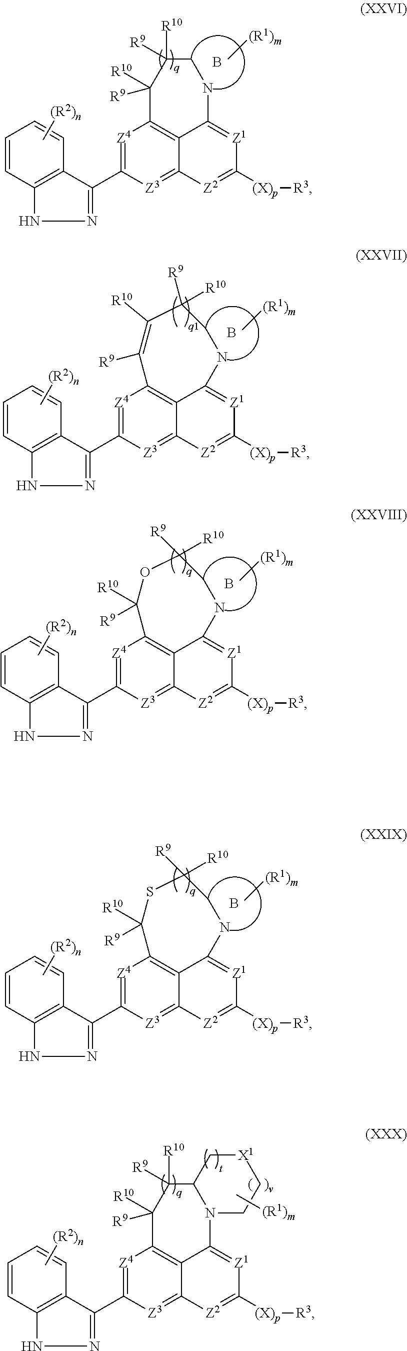

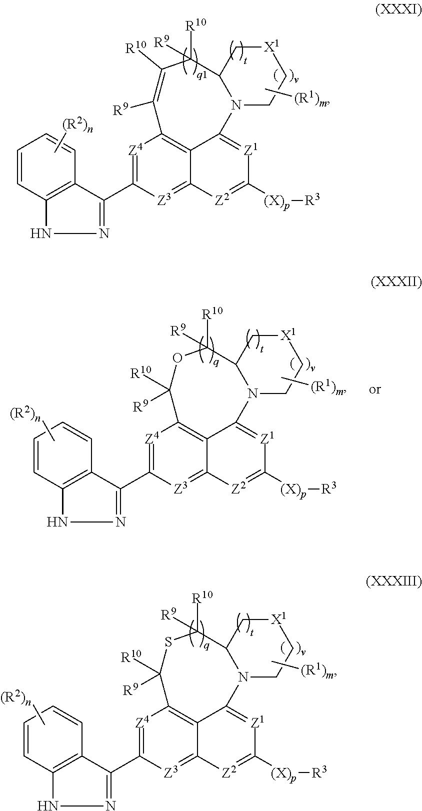

wherein R¹, R², R³, R⁹, R¹⁰, B, X, X¹, Z¹, Z², Z³, Z⁴, m, n, p, q, q1, t, and v are as described herein.

In another aspect, the disclosure relates to a compound of the formula XXXIV, XXXV, XXXVI, XXXVII, XXXVIII, XXXIX, XXXX, or XXXXI, or a pharmaceutically acceptable salt thereof,

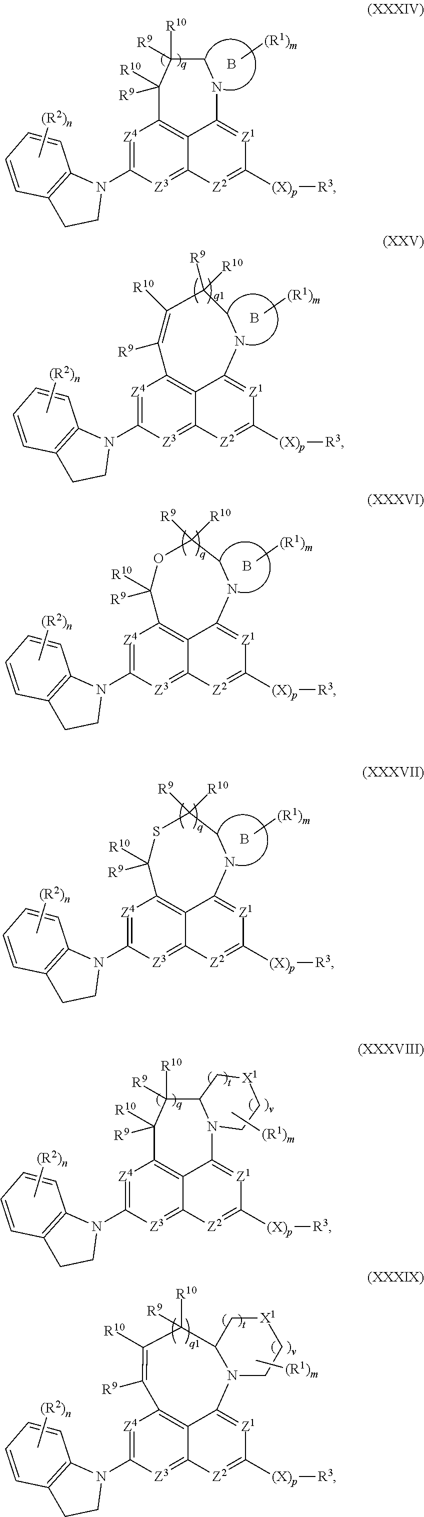

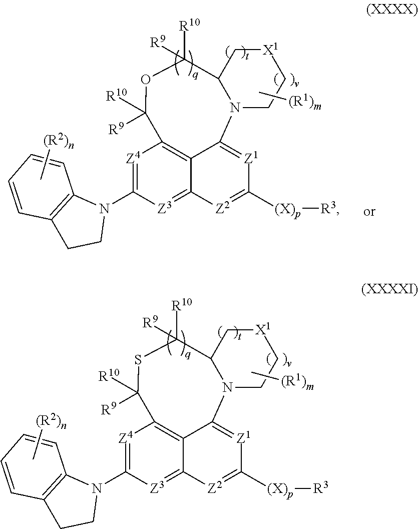

wherein R¹, R², R³, R⁹, R¹⁰, B, X, X¹, Z¹, Z², Z³, Z⁴, m, n, p, q, q1, t, and v are as described herein.

In another aspect, the disclosure relates to a compound of the formula XXXXII, XXXXIII, XXXXIV, XXXV, XXXVI, XXXXVII, XXXXVIII, or XXXXIX, or a pharmaceutically acceptable salt thereof,

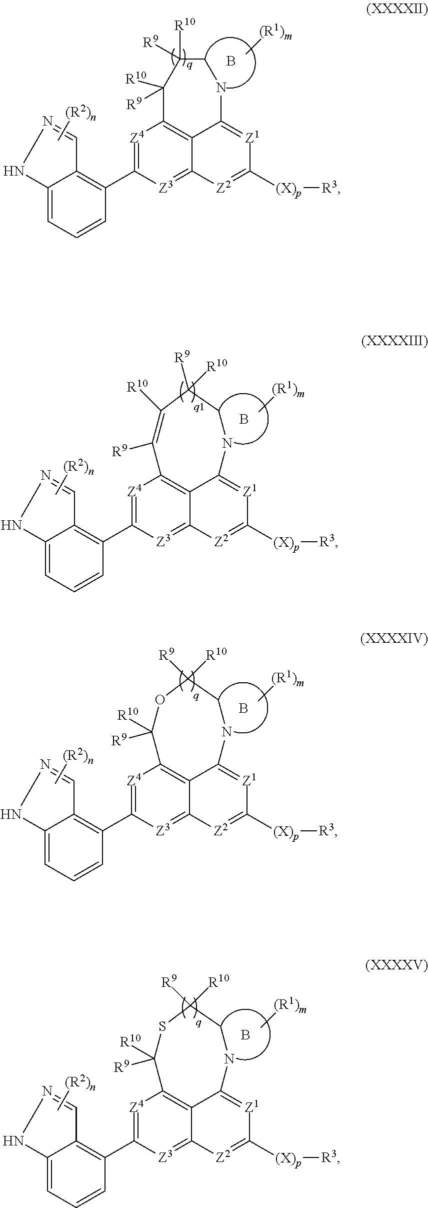

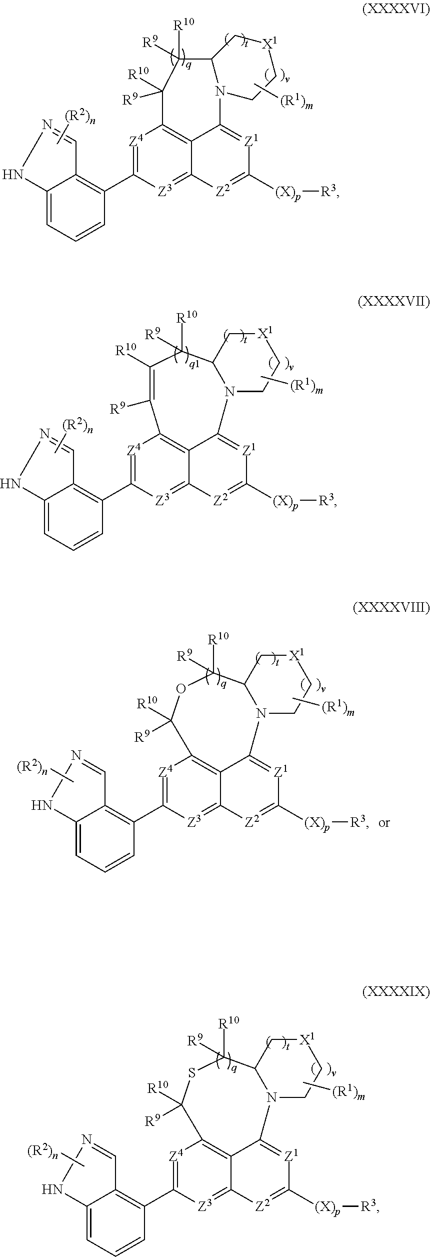

wherein R¹, R², R³, R⁹, R¹⁰, B, X, X¹, Z¹, Z², Z³, Z⁴, m, n, p, q, q1, t, and v are as described herein.

In another aspect, the disclosure relates to a compound of the formula L, LI, LII, LIII, LIV, LV, LVI, or LVII, or a pharmaceutically acceptable salt thereof,

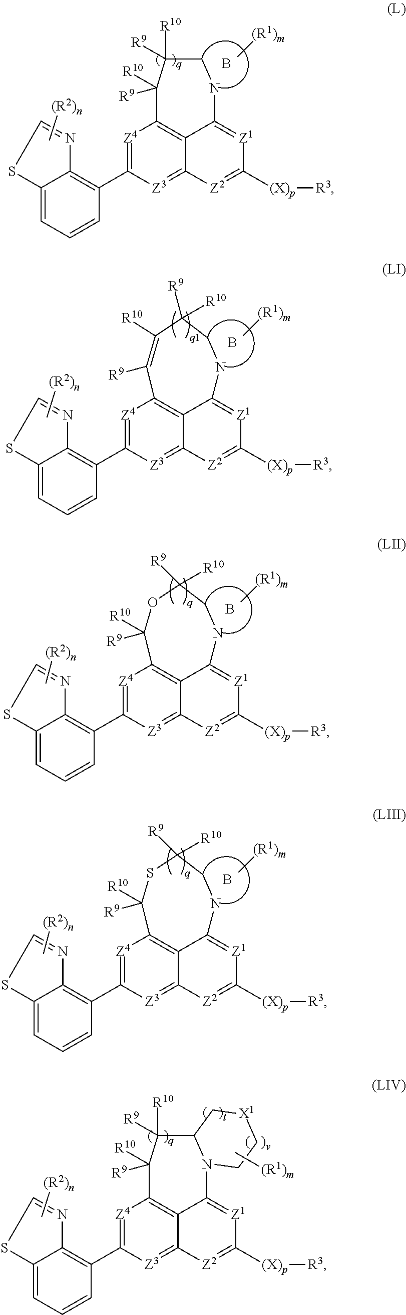

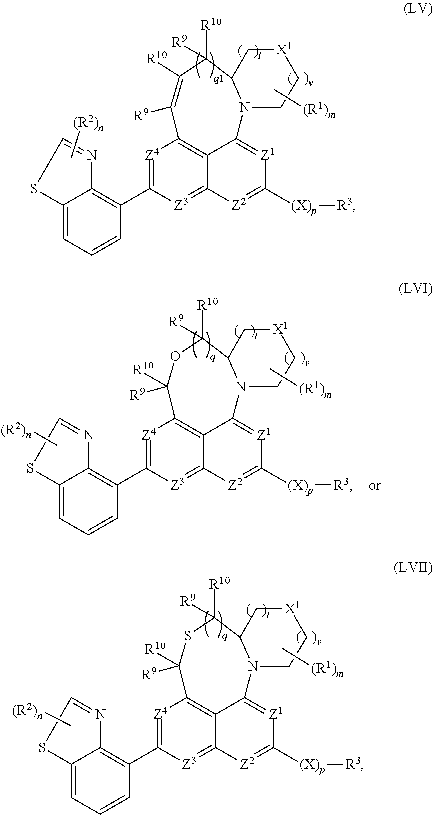

wherein R¹, R², R³, R⁹, R¹⁰, B, X, X¹, Z¹, Z², Z³, Z⁴, m, n, p, q, q1, t, and v are as described herein.

In another aspect, the disclosure relates to a compound of the formula LVIII, LVIX, LX, LXI, LXII, LXIII, LXIV, or LXV, or a pharmaceutically acceptable salt thereof,

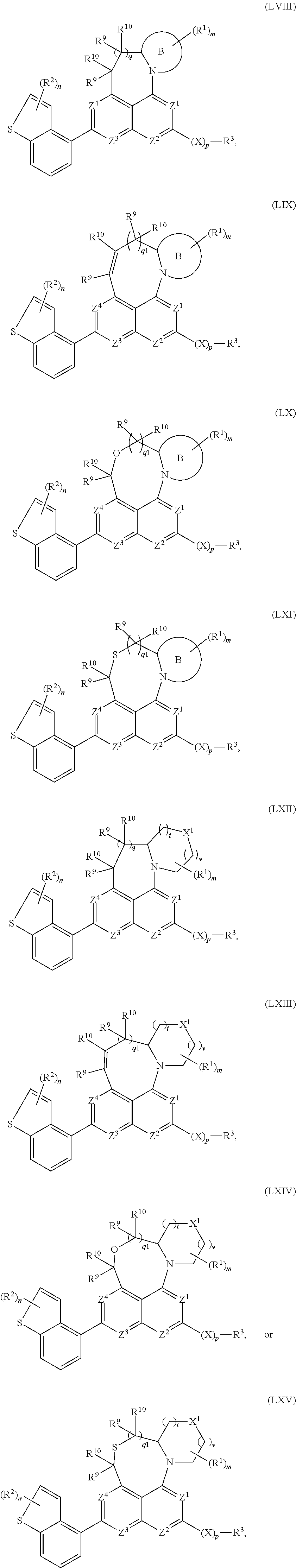

R¹, R², R³, R⁹, R¹⁰, B, X, X¹, Z¹, Z², Z³, Z⁴, m, n, p, q, q1, t, and v are as described herein.

In another aspect, the disclosure relates to a compound of the formula LXVI, LXVII, LXVIII, LXIX, LXX, LXXI, LXXII, LXXIII, LXXIV, LXXV, LXXVI, LXXVII, LXXVIII, LXXIX, LXXX, or LXXXI, or a pharmaceutically acceptable salt thereof,

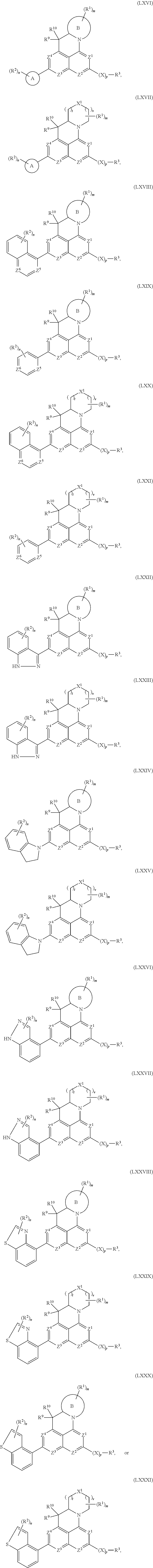

R¹, R², R³, R⁹, R¹⁰, B, X, X¹, Z¹, Z², Z³, Z⁴, m, n, p, q, q1, t, and v are as described herein.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I, or a pharmaceutically acceptable salt thereof,

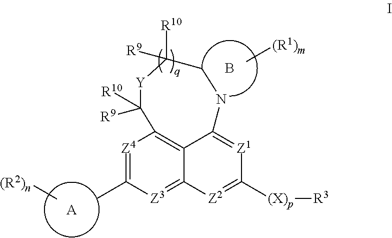

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl, a 5- to 10-membered heteroaryl, or a 4- to 10-membered heterocycloalkyl;
ring B is a 4- to 9-membered heterocycloalkyl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R¹ and an R⁹ or an R¹⁰ on an adjacent carbon to ring B, taken together with the carbon atoms to which they are attached, combine form a carbon-carbon double bond;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{b} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

2. A compound of the formula I, or a pharmaceutically acceptable salt thereof,

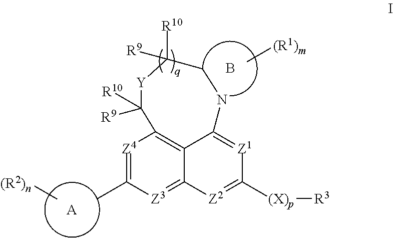

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl or 5- to 10-membered heteroaryl;
ring B is a 4- to 9-membered heterocycloalkyl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NRC(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

3. A compound of the formula I, or a pharmaceutically acceptable salt thereof,

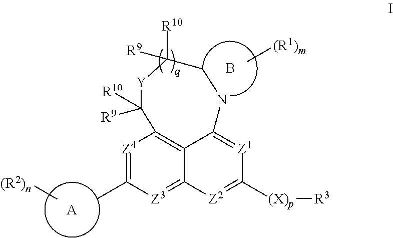

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl or 5- to 10-membered heteroaryl;
ring B is a 4- to 9-membered heterocycloalkyl ring having one nitrogen atom in the 4- to 9-membered heterocycloalkyl ring;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁O aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂₀R^{a}, —CN, or —NO₂, or R⁹ and R¹⁰, taken together with the carbon atom to which they are attached, form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

4. The compound of any one of the preceeding clauses, or a pharmaceutically acceptable salt thereof, wherein the compound is of the formula II, III, IV, V, VI, VII, VIII, or IX,

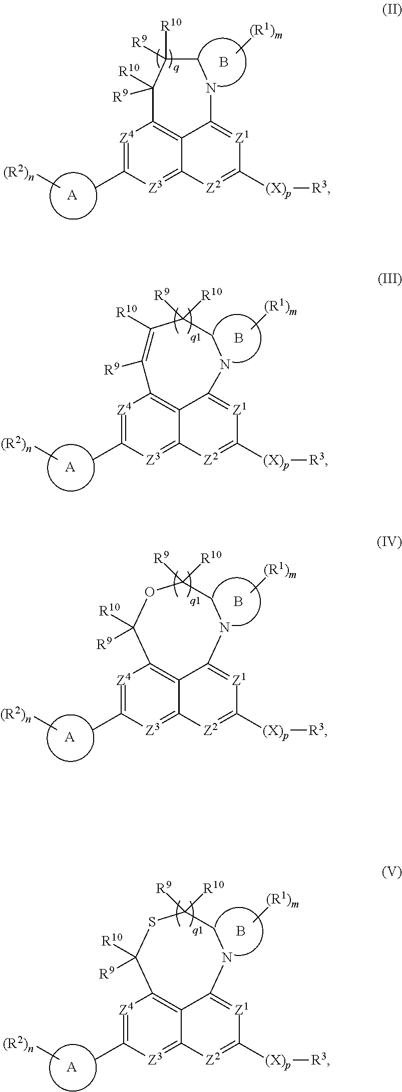

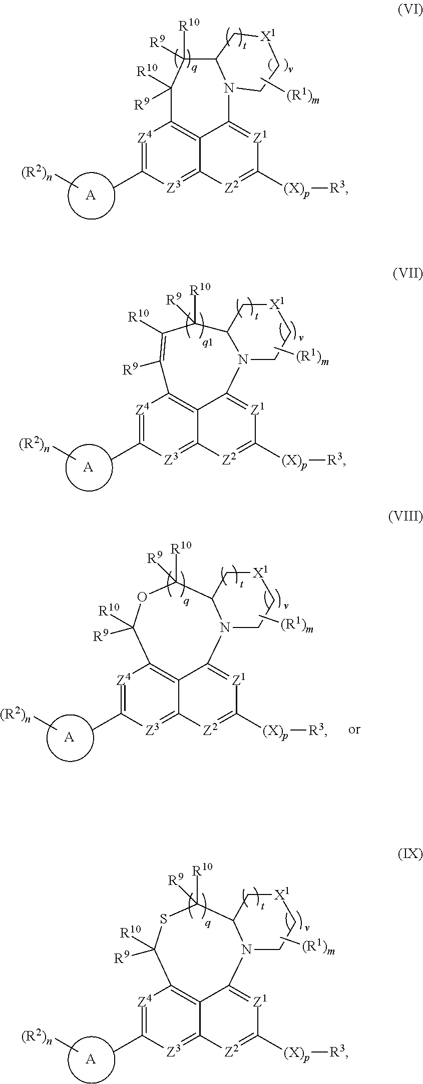

wherein
X¹ is —O—, —S—, —S(O)—, —S(O)₂—, or —C(R¹)₂—;
q1 is 0 or 1;
t is 0, 1, 2, or 3; and
v is 0, 1, or 2.

5. The compound of any one of the preceding clauses, or a pharmaceutically acceptable salt thereof, wherein the compound is of the formula X, XI, XII, XIII, XIV, XV, XVI, or XVII,

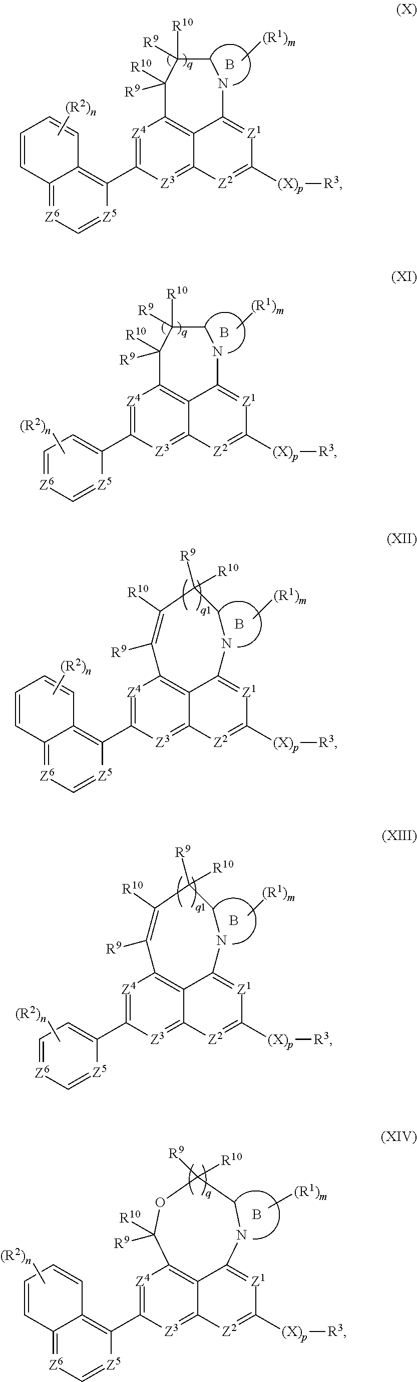

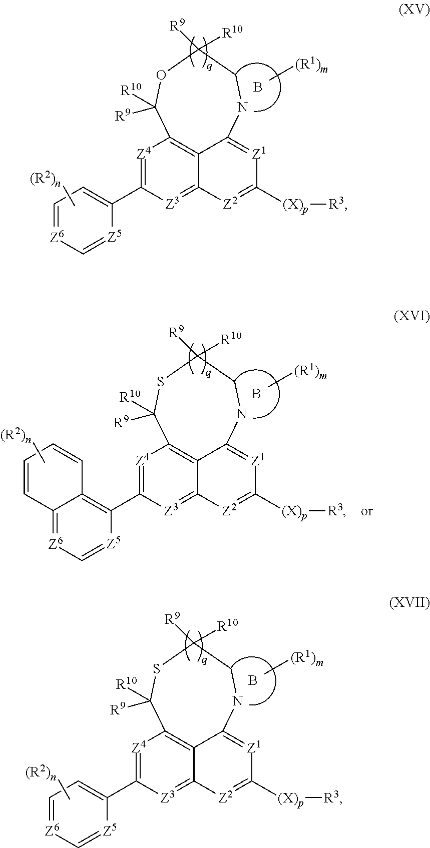

wherein
Z⁵ is N or C(R¹¹);
Z⁶ is N or C(R¹²);
each of R¹¹ and R¹², when present, is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂; and
q1 is 0 or 1.

6. The compound of any one of the preceding clauses, or a pharmaceutically acceptable salt thereof, wherein the compound is of the formula XVIII, XIX, XX, XXI, XXII, XXIII, XXIV, or XXV,

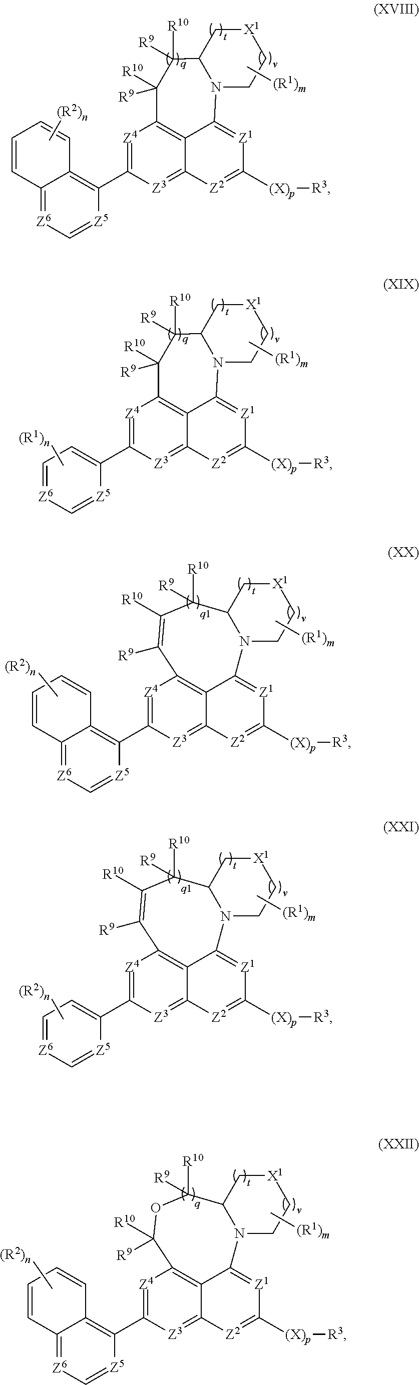

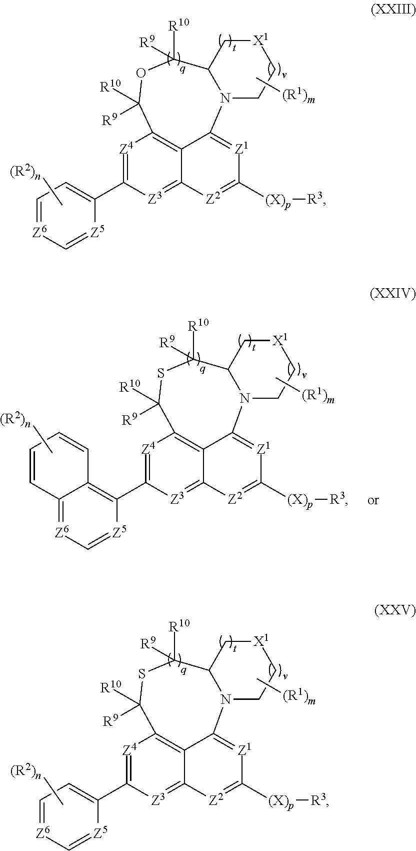

wherein
X¹ is —O—, —S—, —S(O)—, —S(O)₂—, —C(O)O—, —OC(O)—, or —C(R¹)₂—;
Z⁵ is N or C(R¹¹);
Z⁶ is N or C(R¹²);
each of R¹¹ and R¹², when present, is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR, —NR^{g}C(O)NR^{g}R^{h}, —NR^{g}S(O)R^{h}, —NR^{g}S(O)₂R, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR⁸, —CN, or —NO₂, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{C}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂₀R^{e}, —CN, or —NO₂;
q1 is 0 or 1; t is 0, 1, 2, or 3; and
v is 0, 1, or 2.

7. The compound of any one of clauses 1 to 4, or a pharmaceutically acceptable salt thereof, wherein the compound is of the formula XXVI, XXVII, XXVIII, XXIX, XXX, XXXI, XXXII, or XXXIII,

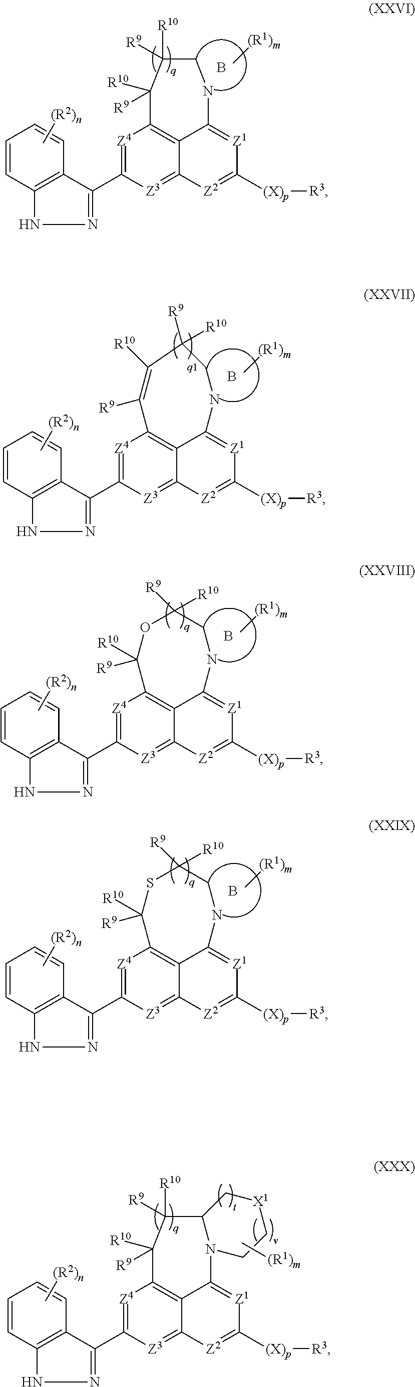

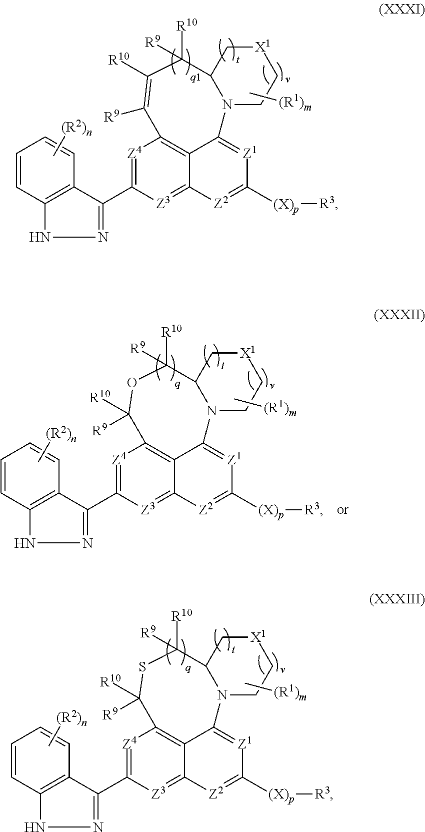

wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

8. The compound of clause 1 or 4, or a pharmaceutically acceptable salt thereof, wherein the compound is of the formula XXXIV, XXXV, XXXVI, XXXVII, XXXVIII, XXXIX, XXXX, or XXXXI,

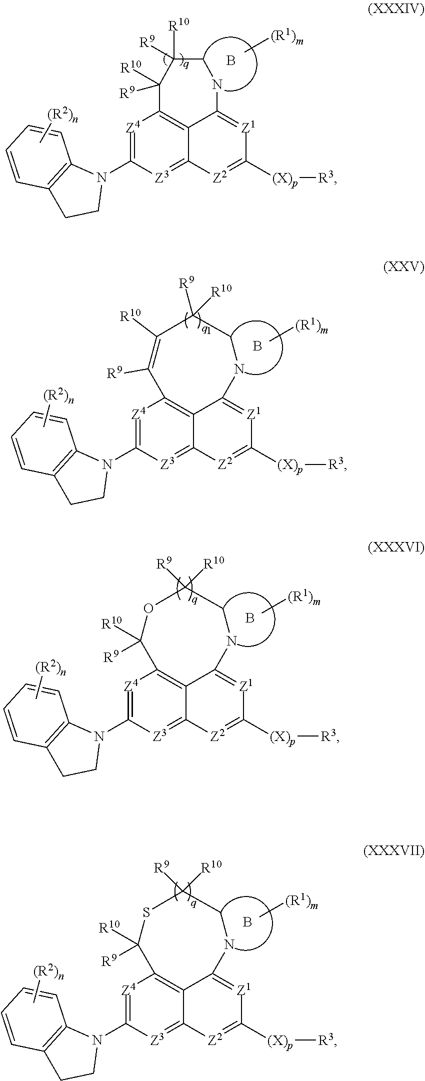

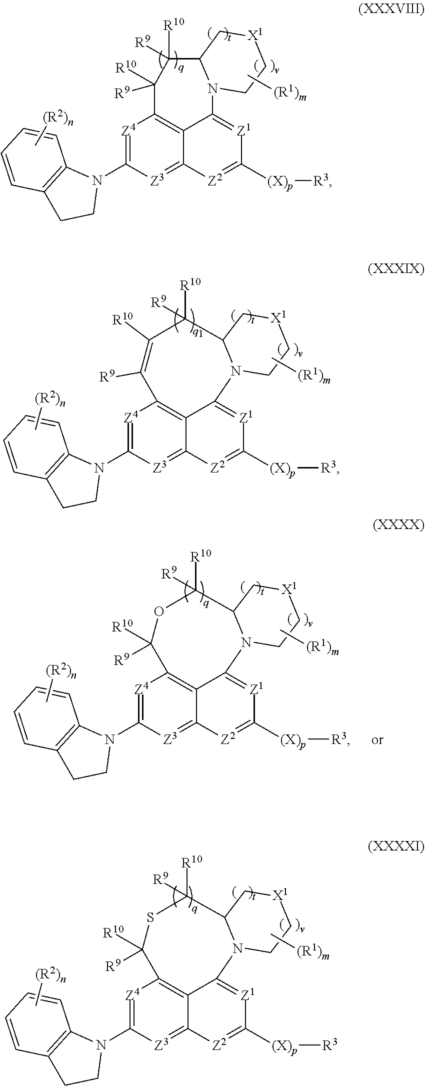

wherein
X¹ is —O—, —S(O)—, —S(O)₂—, —C(O)O—, —OC(O)—, or —C(R¹)₂—;
q1 is 0 or 1;
t is 0, 1, 2, or 3; and
v is 0, 1, or 2.

9. The compound of any one of clauses 1 to 4, or a pharmaceutically acceptable salt thereof, wherein the compound is of the formula XXXXII, XXXXIII, XXXXIV, XXXV, XXXVI, XXXXVII, XXXXVIII, or XXXXIX,

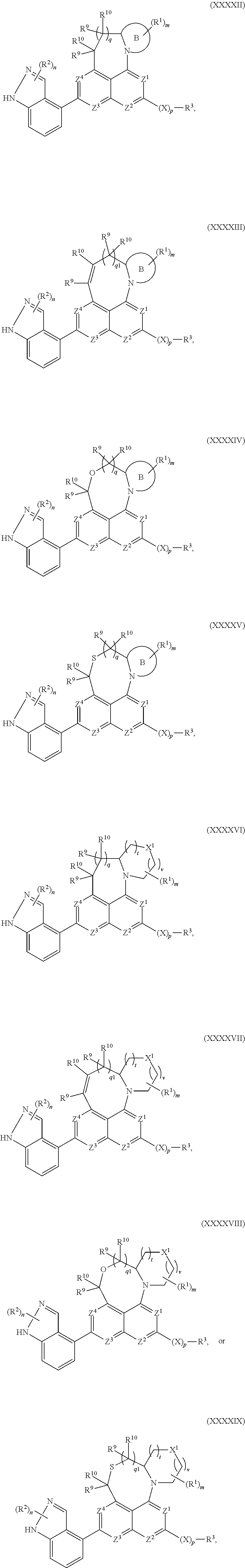

wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

10. The compound of any one of clauses I to 4, or a pharmaceutically acceptable salt thereof, wherein the compound is of the formula L, LI, LII, LIII, LIV, LV, LVI, or LVII,

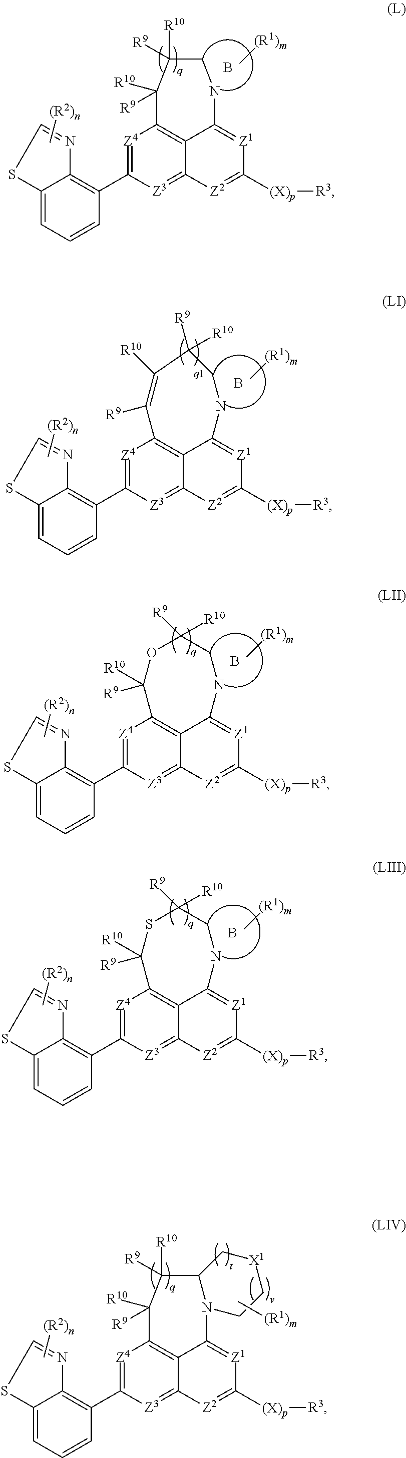

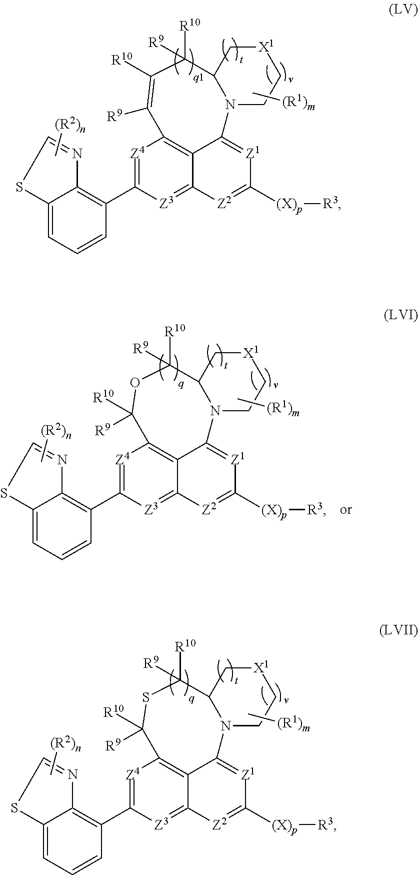

wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

11. The compound of any one of clauses 1 to 4, or a pharmaceutically acceptable salt thereof, wherein the compound is of the formula LVIII, LVIX, LX, LXI, LXII, LXIII, LXIV, or LXV,

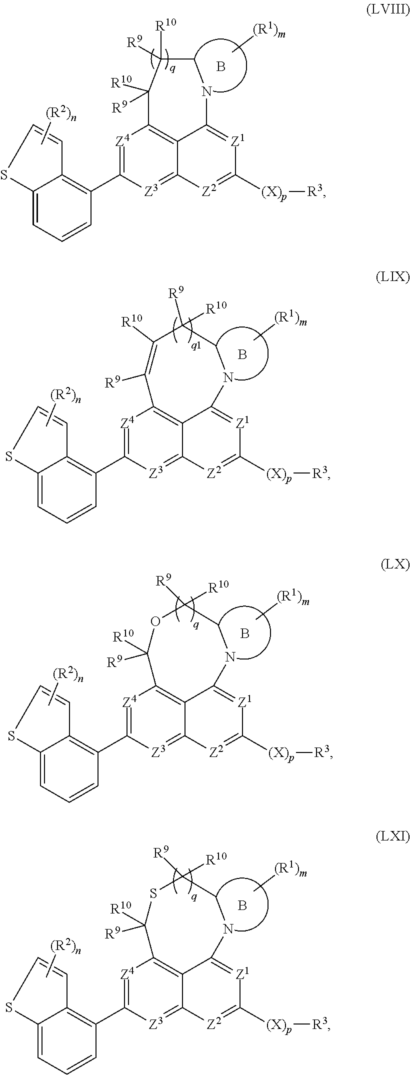

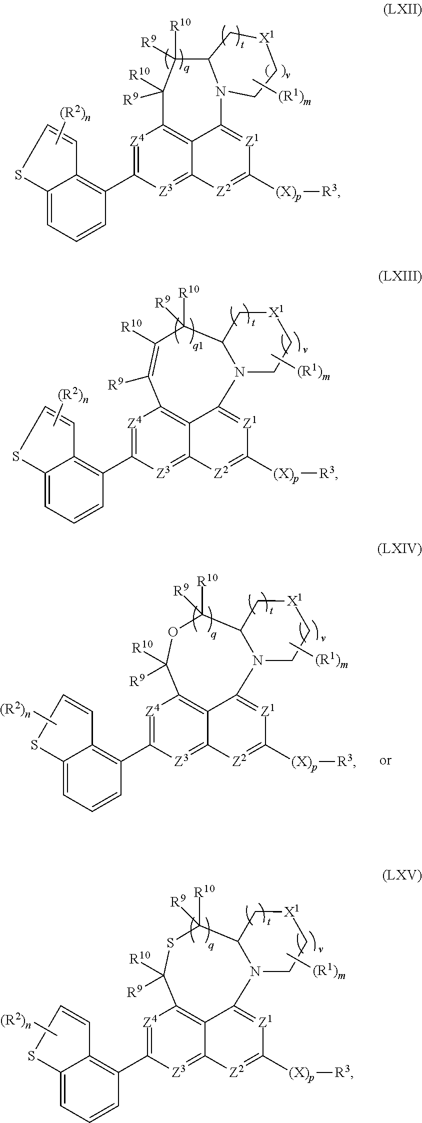

wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

12. The compound of any one of the preceding clauses, or a pharmaceutically acceptable salt thereof, wherein q, when present, is 0.

13. The compound of any one of the preceding clauses, or a pharmaceutically acceptable salt thereof, wherein q, when present, is 1.

14. The compound of any one of the preceding clauses, or a pharmaceutically acceptable salt thereof, wherein q1, when present, is 0.

15. The compound of any one of the preceding clauses, or a pharmaceutically acceptable salt thereof, wherein q1, when present, is 1.

16. The compound of any one of the preceding clauses, or a pharmaceutically acceptable salt thereof, wherein R¹ is deuterium, C₁-C₆ alkyl, C₂-C₆ alkenyl, or C₂-C₆ alkynyl, wherein each hydrogen atom in C₁-C₆ alkyl, C₂-C₆ alkenyl, or C₂-C₆ alkynyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂ or two hydrogen atoms on a single carbon atom of the 4- to 10-membered heterocycloalkyl combine to form an oxo group or an C₂-C₆ alkenyl group; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

17. The compound of any one of the preceding clauses, or a pharmaceutically acceptable salt thereof, wherein the compound comprises a core having the formula

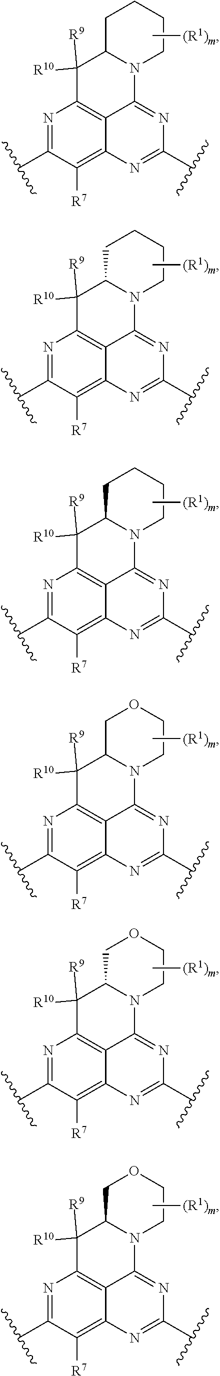

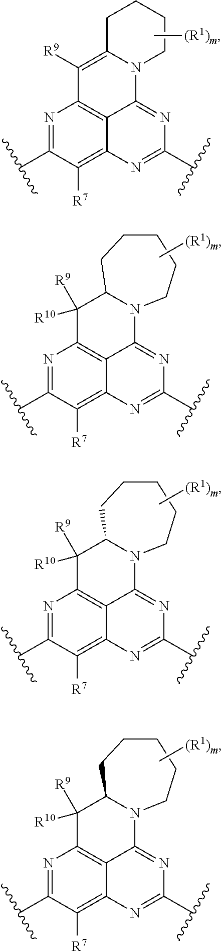

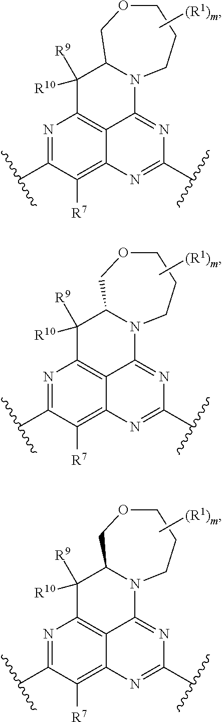

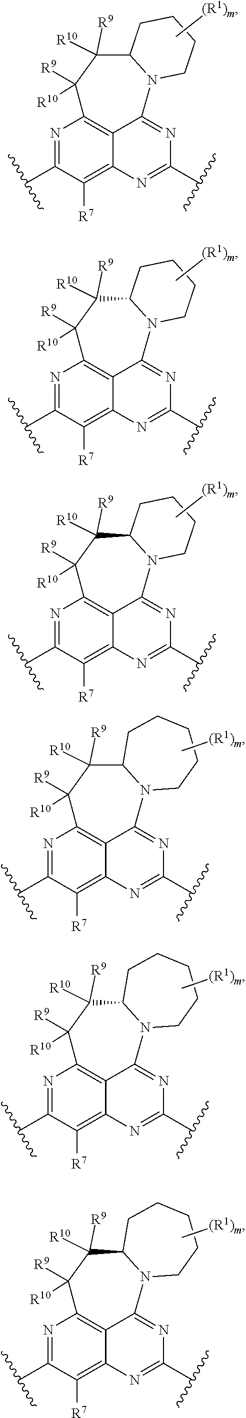

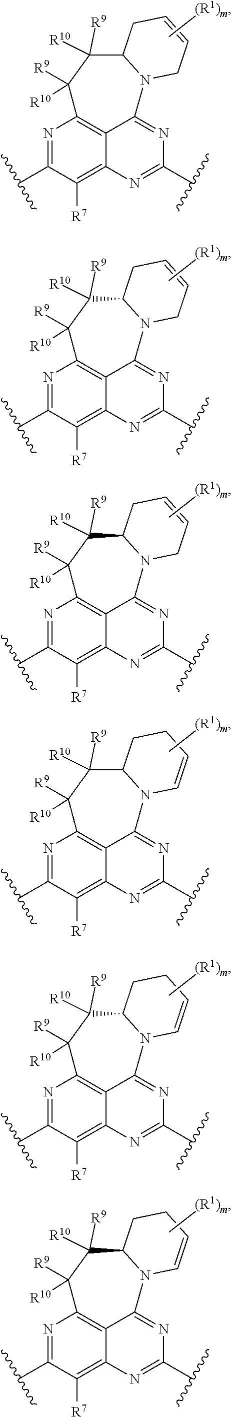

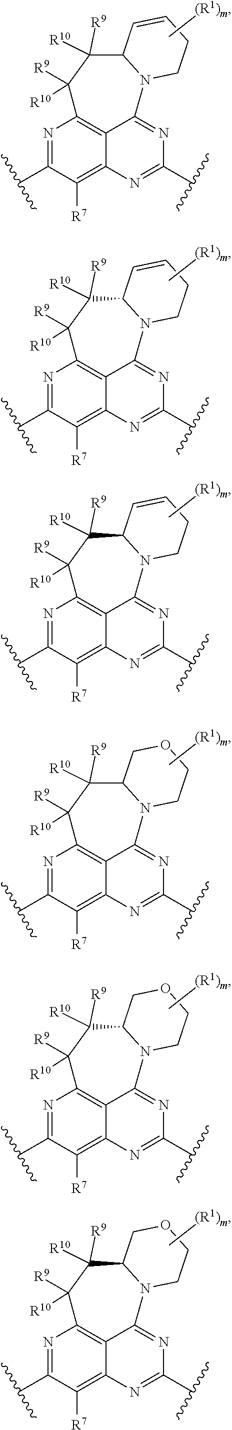

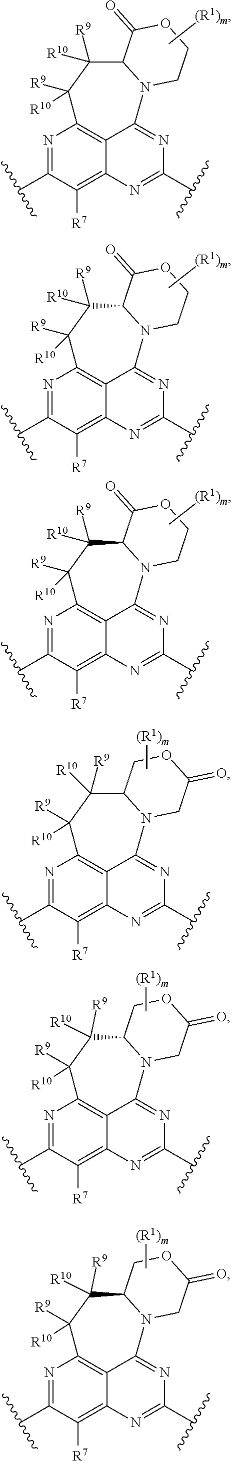

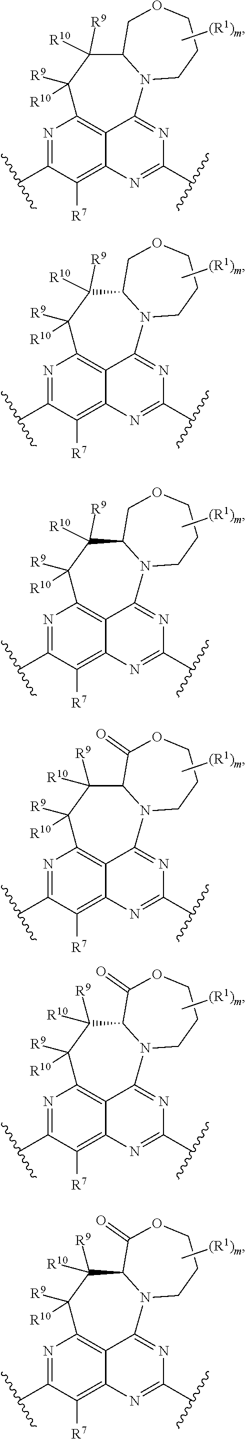

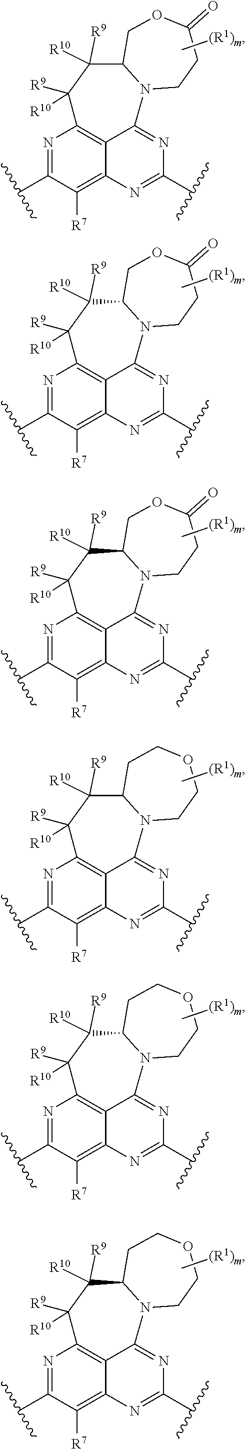

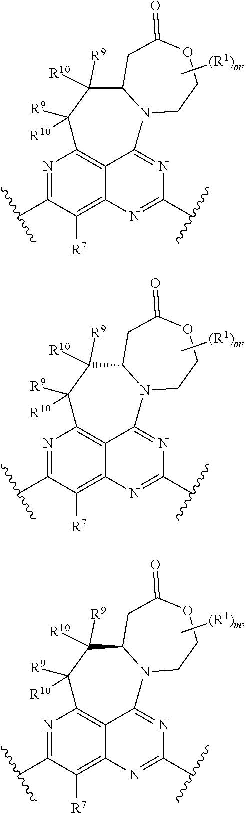

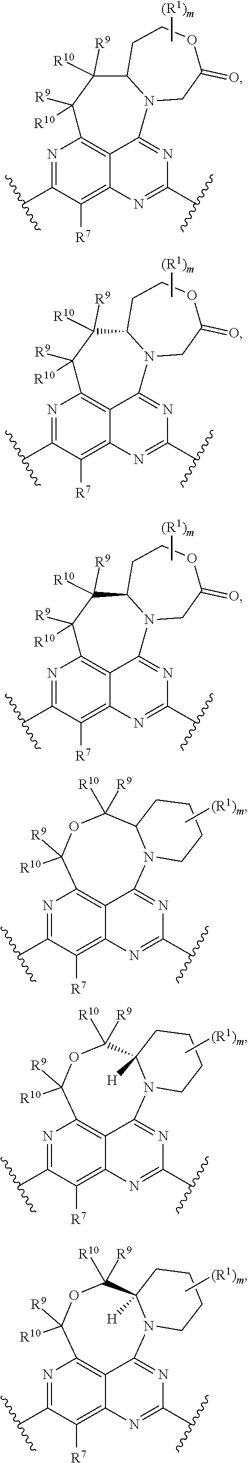

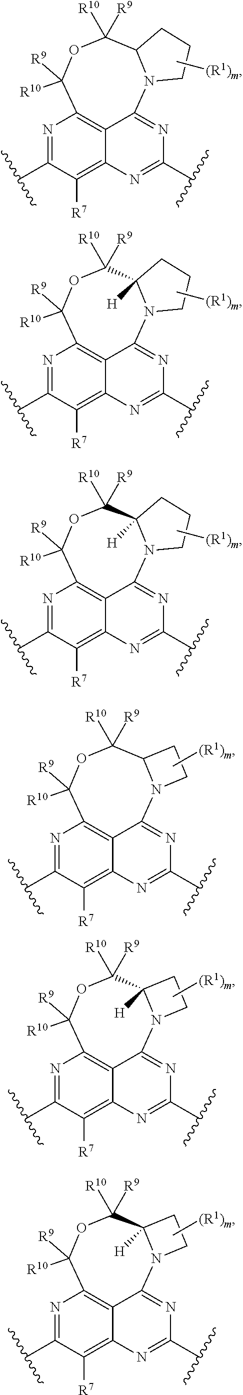

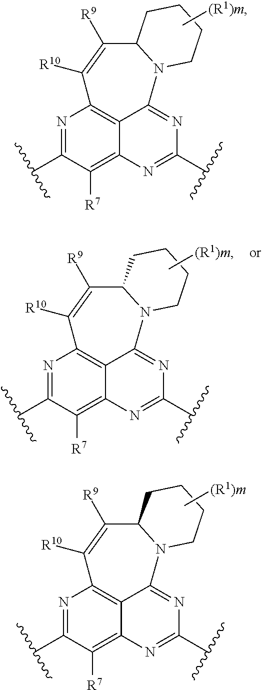

wherein each “ [symbol] ” is a point of covalent attachment to either ring A or —(X)ₚ—R³.

18. The compound of any one of the preceding clauses, or a pharmaceutically acceptable salt thereof, wherein m is 0, 1, 2, or 3.

19. The compound of any one of the preceding clauses, or a pharmaceutically acceptable salt thereof, wherein n is 0, 1, 2, or 3.

20. The compound of any one of the preceding clauses, or a pharmaceutically acceptable salt thereof, wherein Z¹ is N.

21. The compound of any one of the preceding clauses, or a pharmaceutically acceptable salt thereof, wherein Z² is N.

22. The compound of any one of the preceding clauses, or a pharmaceutically acceptable salt thereof, wherein Z³ is CR⁷, and Z⁴ is N.

23. The compound of any one of clauses 1 to 21, or a pharmaceutically acceptable salt thereof, wherein Z³ is CR⁷, and Z⁴ is CR⁸.

24. The compound of any one of clauses 1 to 21, or a pharmaceutically acceptable salt thereof, wherein Z³ is N, and Z⁴ is CR^{S}.

25. The compound of any one of clauses 1 to 21, or a pharmaceutically acceptable salt thereof, wherein Z³ is N, and Z⁴ is N.

26. The compound of any one of clauses 1 to 21, or a pharmaceutically acceptable salt thereof, wherein Z³ is N, Z⁴ is CR⁸, Z⁵ is CR¹¹, and Z⁶ is CR¹².

27. The compound of any one of clauses 1 to 21, or a pharmaceutically acceptable salt thereof, wherein Z³ is CR⁷, Z⁴ is N, Z⁵ is CR¹¹, and Z⁶ is CR¹².

28. The compound of any one of clauses 1 to 21, or a pharmaceutically acceptable salt thereof, wherein Z³ is N, Z⁴ is N, Z⁵ is CR¹¹, and Z⁵ is CR¹².

29. The compound of any one of clauses 1 to 22, or a pharmaceutically acceptable salt thereof, wherein the compound comprises a core having the formula

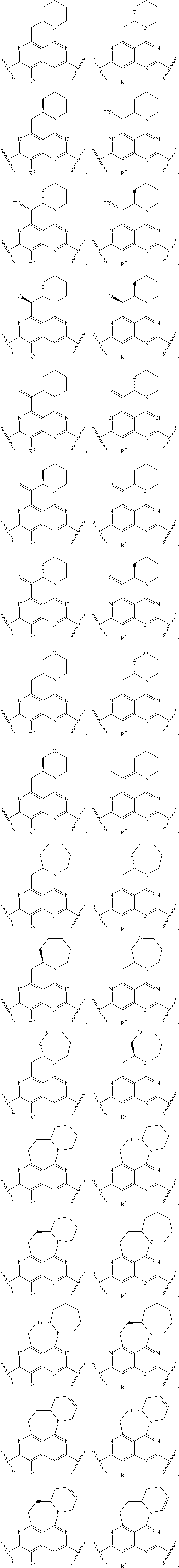

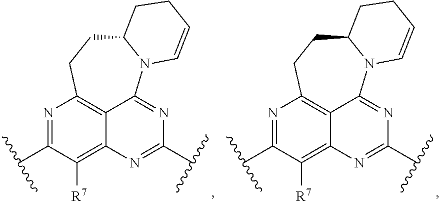

[chemical structure]

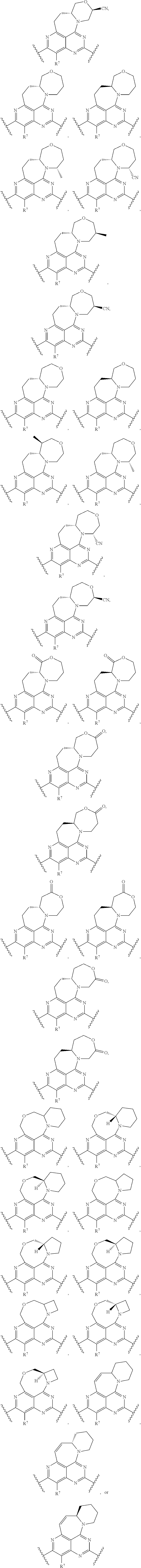

wherein each “ [symbol] ” is a point of covalent attachment to either ring A or —(X)ₚ—R³.

30. The compound of any one of the preceding clauses, or a pharmaceutically acceptable salt thereof, wherein p is 0.

31. The compound of any one of clauses 1 to 29, or a pharmaceutically acceptable salt thereof, wherein p is 1.

32. The compound of any one of clauses 1 to 29, or 31, or a pharmaceutically acceptable salt thereof, wherein X is O.

33. The compound of any one of clauses 1 to 29, or 31, or a pharmaceutically acceptable salt thereof, wherein X is —NR⁴—.

34. The compound of any one of clauses 1 to 29, or 31, or a pharmaceutically acceptable salt thereof, wherein X is —S—.

35. The compound of any one of the preceding clauses, or a pharmaceutically acceptable salt thereof, wherein R³ is —C₁-C₆ alkyl, 4- to 10-membered heterocycloalkyl, or —C₁-C₆ alkylene-(4- to 10-membered heterocycloalkyl), wherein each hydrogen atom in —C₁-C₆ alkyl, 4- to 10-membered heterocycloalkyl, or —C₁-C₆ alkylene-(4- to 10-membered heterocycloalkyl), is independently optionally substituted by deuterium, halogen, C₁-C₆ alkyl, —C₁-C₆alkylene-O—C₁-C₆alkyl, —OC₁-C₆alkylene-O—C₁-C₆ alkyl, —C₁-C₆ alkylene-O—R^{a}, C₆-C₁₀ aryl, —C₁-C₆alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

36. The compound of any one of the preceding clauses, or a pharmaceutically acceptable salt thereof, wherein R³ is —C₁-C₆ alkylene-(4- to 10-membered heterocycloalkyl), wherein each hydrogen atom in —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

37. The compound of any one of the preceding clauses, or a pharmaceutically acceptable salt thereof, wherein R³ is

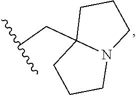 [SMILES: *CC12CCCN1CCC2]

wherein each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and “ [symbol] ” is a point of covalent attachment.

38. The compound of any one of the preceding clauses, or a pharmaceutically acceptable salt thereof, wherein R³ is

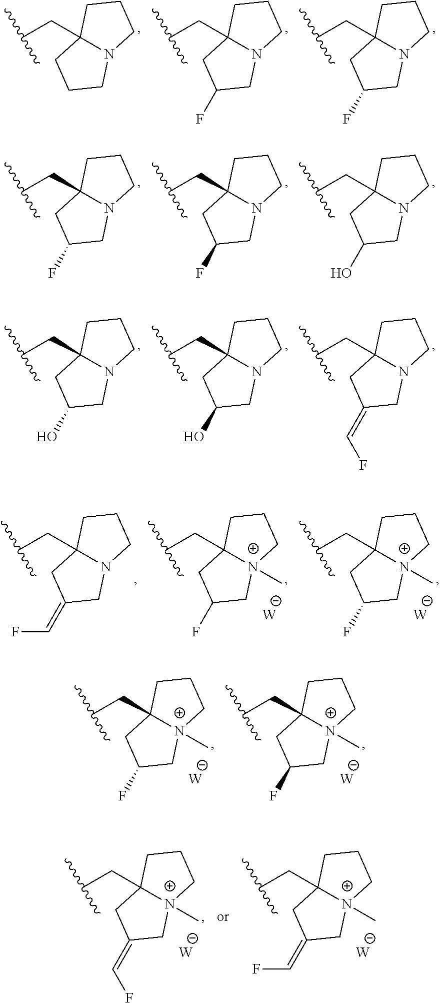

wherein W^{⊖} is an inorganic counter ion or an organic counter ion.

39. The compound of any one of clauses 1 to 35, or a pharmaceutically acceptable salt thereof, wherein R³ is 4- to 10-membered heterocycloalkyl, wherein each hydrogen atom in 4- to 10-membered heterocycloalkyl is independently optionally substituted by deuterium, halogen, C₁-C₆ alkyl, C₂-C₆ alkenyl optionally substituted by halo —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

40. The compound of any one of clauses 1 to 35, or 39, or a pharmaceutically acceptable salt thereof, wherein R³ is

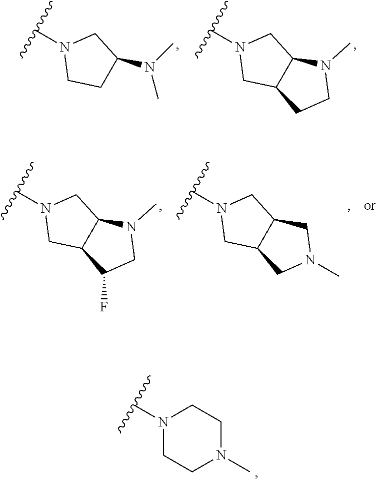

wherein “ [symbol] ” is a point of covalent attachment.

41. The compound of any one of the preceding clauses, or a pharmaceutically acceptable salt thereof, wherein each R² is independently deuterium, halogen, C₁-C₆ alkyl, C₂-C₆ alkenyl, C₂-C₆ alkynyl, —OR^{c}, —C(O)R^{c}, —NR^{c}R^{d}, or —CN, wherein each hydrogen atom in C₁-C₆ alkyl, C₂-C₆ alkenyl, and C₂-C₆ alkynyl is independently optionally substituted with a deuterium or halogen.

42. The compound of any one of the preceding clauses, or a pharmaceutically acceptable salt thereof, wherein ring A is

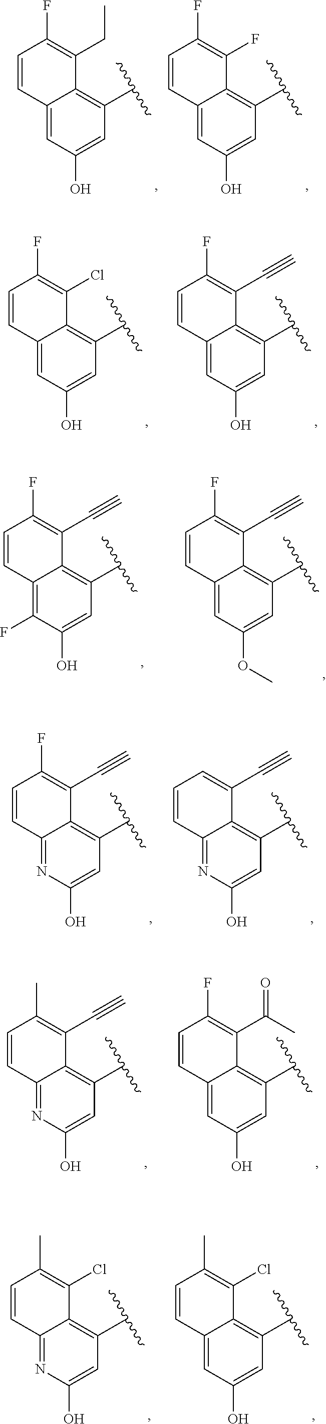

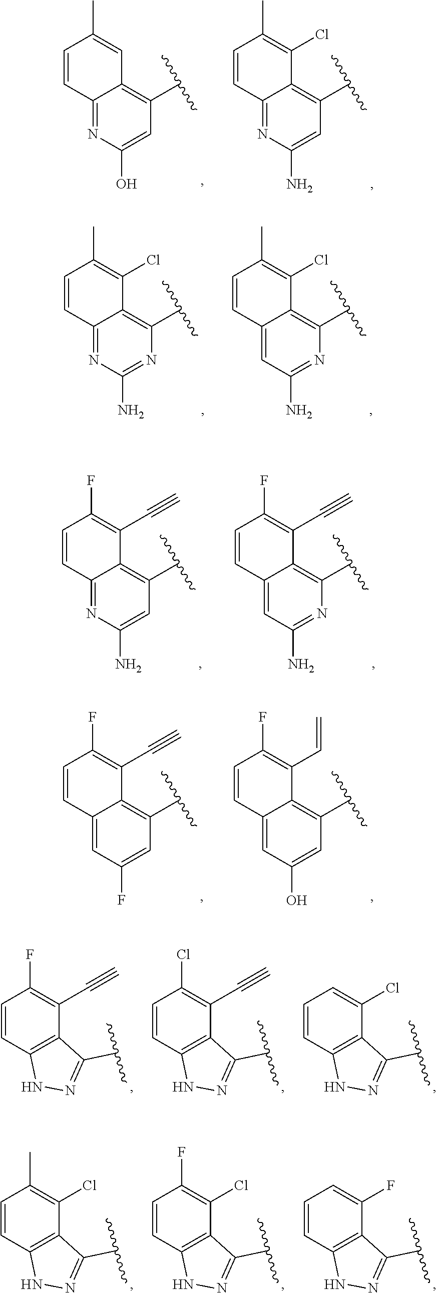

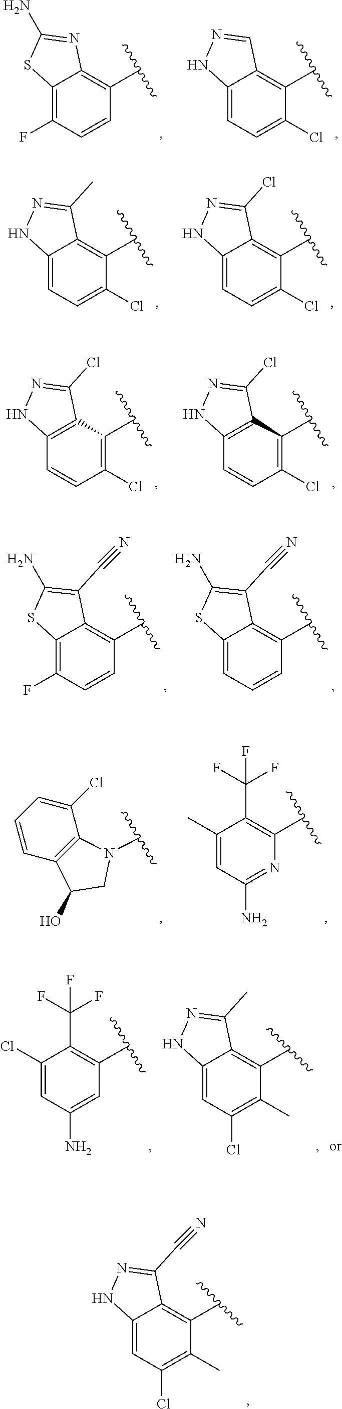

wherein “ [symbol] ” is a point of covalent attachment.

43. The compound of any one of the preceding clauses, or a pharmaceutically acceptable salt thereof, wherein R⁴, when present, is H or methyl.

44. The compound of any one of the preceding clauses, or a pharmaceutically acceptable salt thereof, wherein R⁵, when present, is H or deuterium.

45. The compound of any one of the preceding clauses, or a pharmaceutically acceptable salt thereof, wherein R⁶, when present, is H or deuterium.

46. The compound of any one of the preceding clauses, or a pharmaceutically acceptable salt thereof, wherein R⁷, when present, is H, deuterium, or F.

47. The compound of any one of the preceding clauses, or a pharmaceutically acceptable salt thereof, wherein R⁸, when present, is H or deuterium.

48. The compound of any one of the preceding clauses, or a pharmaceutically acceptable salt thereof, wherein Z⁵, when present, is N.

49. The compound of any one of the preceding clauses, or a pharmaceutically acceptable salt thereof, wherein Z⁶, when present, is N.

50. The compound of any one of clauses 1 to 42, or a pharmaceutically acceptable salt thereof, wherein Z⁵, when present, is CR¹¹.

51. The compound of clause 50, or a pharmaceutically acceptable salt thereof, wherein R¹¹, when present, is H or deuterium.

52. The compound of clause 50 or 51, or a pharmaceutically acceptable salt thereof, wherein Z⁶, when present, is N.

53. The compound of any one of clauses 1 to 48, or a pharmaceutically acceptable salt thereof, wherein Z⁶, when present, is CR¹².

54. The compound of clause 53, or a pharmaceutically acceptable salt thereof, wherein R¹², when present, is H or deuterium.

55. The compound of clause 1, selected from the group consisting of

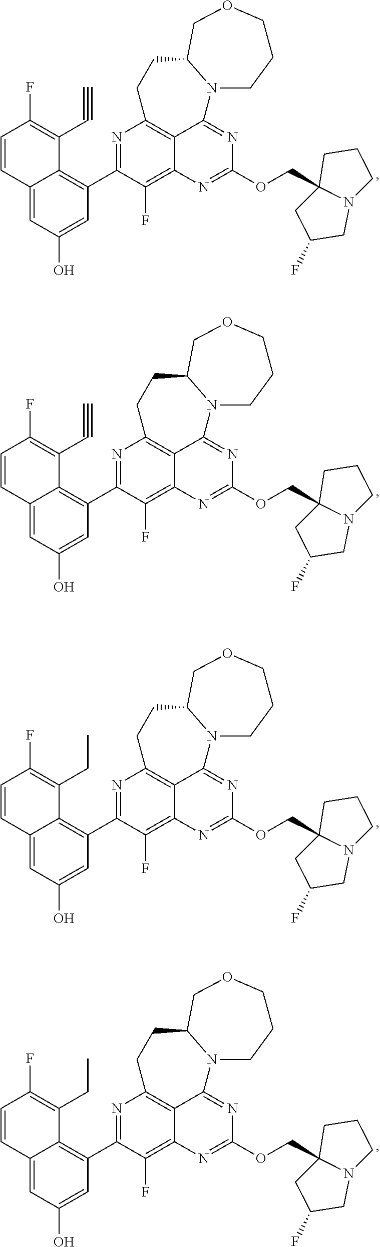 [SMILES: C#Cc1c(F)ccc2cc(O)cc(-c3nc4c5c(nc(OC[C@@]67CCCN6C[C@H](F)C7)nc5c3F)N3CCCOC[C@H]3CC4)c12 ; C#Cc1c(F)ccc2cc(O)cc(-c3nc4c5c(nc(OC[C@@]67CCCN6C[C@H](F)C7)nc5c3F)N3CCCOC[C@@H]3CC4)c12 ; CCc1c(F)ccc2cc(O)cc(-c3nc4c5c(nc(OC[C@@]67CCCN6C[C@H](F)C7)nc5c3F)N3CCCOC[C@H]3CC4)c12 ; CCc1c(F)ccc2cc(O)cc(-c3nc4c5c(nc(OC[C@@]67CCCN6C[C@H](F)C7)nc5c3F)N3CCCOC[C@@H]3CC4)c12]

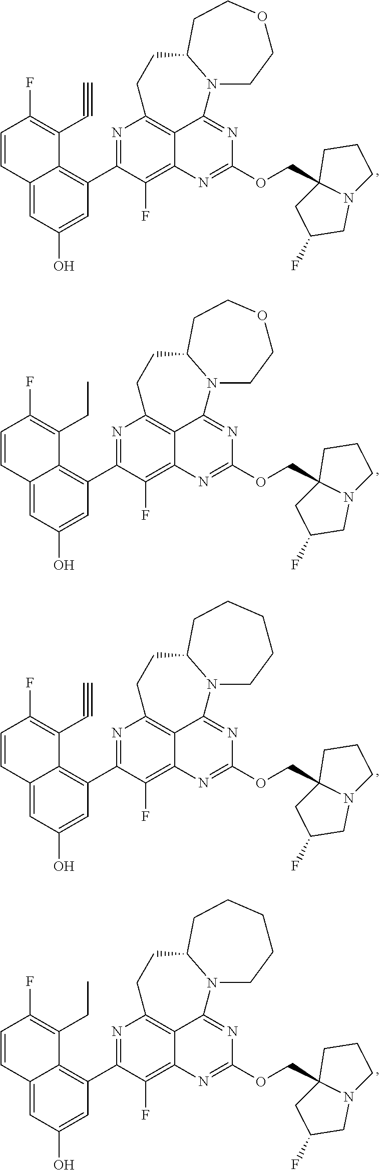 [SMILES: C#Cc1c(F)ccc2cc(O)cc(-c3nc4c5c(nc(OC[C@@]67CCCN6C[C@H](F)C7)nc5c3F)N3CCOCC[C@H]3CC4)c12 ; CCc1c(F)ccc2cc(O)cc(-c3nc4c5c(nc(OC[C@@]67CCCN6C[C@H](F)C7)nc5c3F)N3CCOCC[C@H]3CC4)c12 ; C#Cc1c(F)ccc2cc(O)cc(-c3nc4c5c(nc(OC[C@@]67CCCN6C[C@H](F)C7)nc5c3F)N3CCCCC[C@H]3CC4)c12 ; CCc1c(F)ccc2cc(O)cc(-c3nc4c5c(nc(OC[C@@]67CCCN6C[C@H](F)C7)nc5c3F)N3CCCCC[C@H]3CC4)c12]

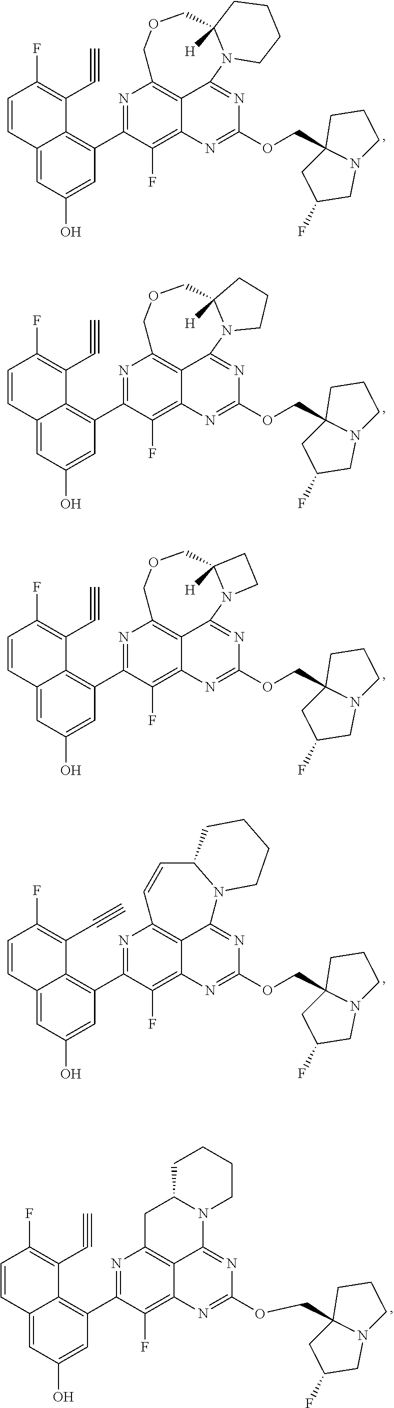

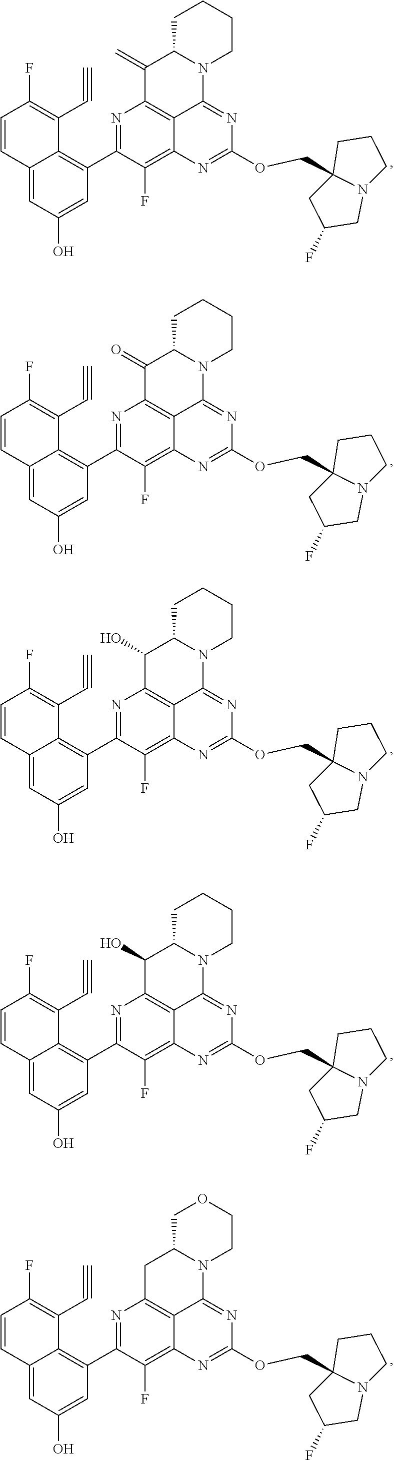 [SMILES: C#Cc1c(F)ccc2cc(O)cc(-c3nc4c5c(nc(OC[C@@]67CCCN6C[C@H](F)C7)nc5c3F)N3CCCC[C@H]3C4=C)c12 ; C#Cc1c(F)ccc2cc(O)cc(-c3nc4c5c(nc(OC[C@@]67CCCN6C[C@H](F)C7)nc5c3F)N3CCCC[C@H]3C4=O)c12 ; C#Cc1c(F)ccc2cc(O)cc(-c3nc4c5c(nc(OC[C@@]67CCCN6C[C@H](F)C7)nc5c3F)N3CCCC[C@H]3[C@H]4O)c12 ; C#Cc1c(F)ccc2cc(O)cc(-c3nc4c5c(nc(OC[C@@]67CCCN6C[C@H](F)C7)nc5c3F)N3CCCC[C@H]3[C@@H]4O)c12 ; C#Cc1c(F)ccc2cc(O)cc(-c3nc4c5c(nc(OC[C@@]67CCCN6C[C@H](F)C7)nc5c3F)N3CCOC[C@H]3C4)c12]

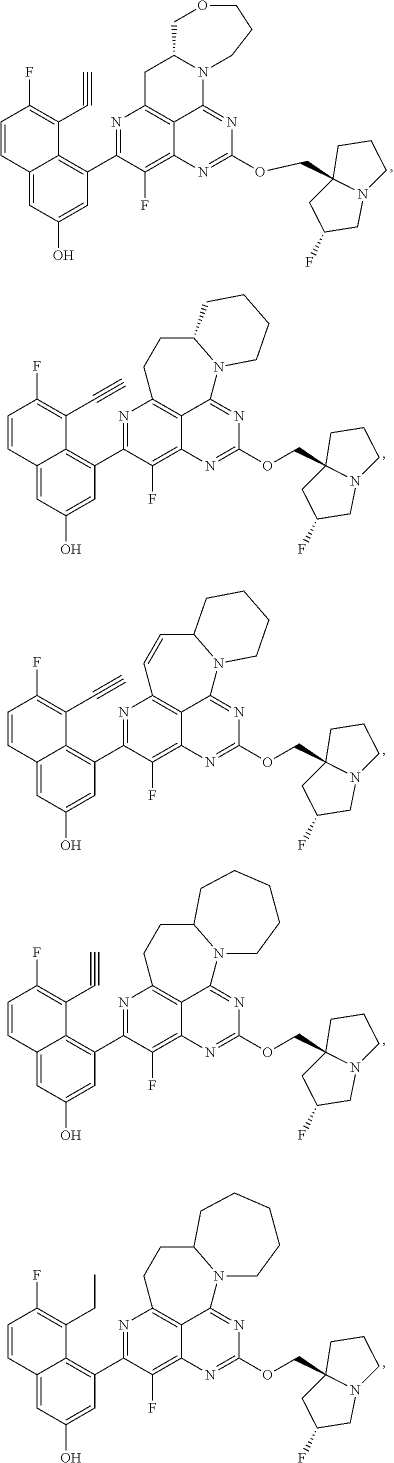 [SMILES: C#Cc1c(F)ccc2cc(O)cc(-c3nc4c5c(nc(OC[C@@]67CCCN6C[C@H](F)C7)nc5c3F)N3CCCOC[C@H]3C4)c12 ; C#Cc1c(F)ccc2cc(O)cc(-c3nc4c5c(nc(OC[C@@]67CCCN6C[C@H](F)C7)nc5c3F)N3CCCC[C@H]3CC4)c12 ; C#Cc1c(F)ccc2cc(O)cc(-c3nc4c5c(nc(OC[C@@]67CCCN6C[C@H](F)C7)nc5c3F)N3CCCCC3C=C4)c12 ; C#Cc1c(F)ccc2cc(O)cc(-c3nc4c5c(nc(OC[C@@]67CCCN6C[C@H](F)C7)nc5c3F)N3CCCCCC3CC4)c12 ; CCc1c(F)ccc2cc(O)cc(-c3nc4c5c(nc(OC[C@@]67CCCN6C[C@H](F)C7)nc5c3F)N3CCCCCC3CC4)c12]

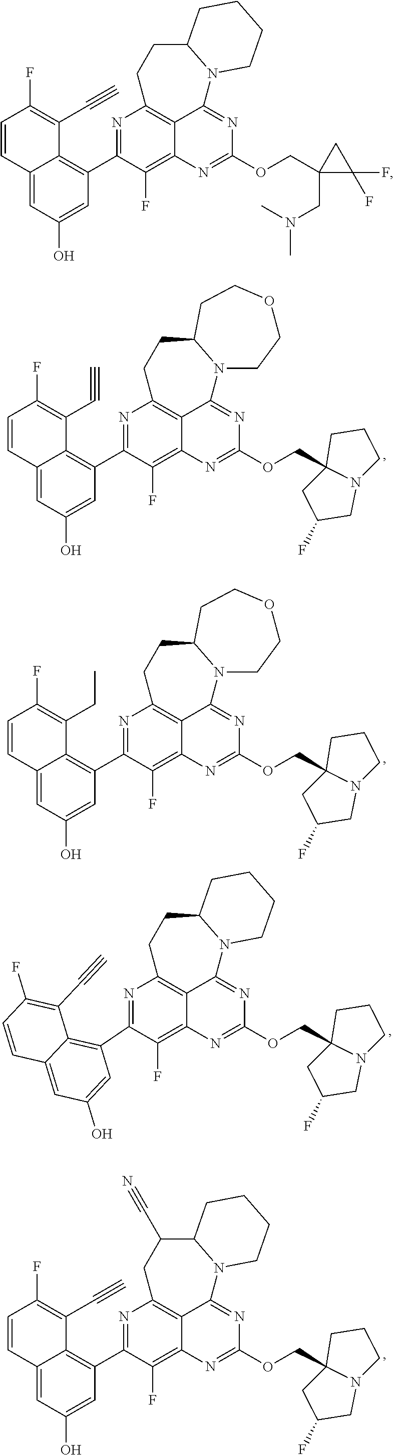 [SMILES: C#Cc1c(F)ccc2cc(O)cc(-c3nc4c5c(nc(OCC6(CN(C)C)CC6(F)F)nc5c3F)N3CCCCC3CC4)c12 ; C#Cc1c(F)ccc2cc(O)cc(-c3nc4c5c(nc(OC[C@@]67CCCN6C[C@H](F)C7)nc5c3F)N3CCOCC[C@@H]3CC4)c12 ; CCc1c(F)ccc2cc(O)cc(-c3nc4c5c(nc(OC[C@@]67CCCN6C[C@H](F)C7)nc5c3F)N3CCOCC[C@@H]3CC4)c12 ; C#Cc1c(F)ccc2cc(O)cc(-c3nc4c5c(nc(OC[C@@]67CCCN6C[C@H](F)C7)nc5c3F)N3CCCC[C@@H]3CC4)c12 ; C#Cc1c(F)ccc2cc(O)cc(-c3nc4c5c(nc(OC[C@@]67CCCN6C[C@H](F)C7)nc5c3F)N3CCCCC3C(C#N)C4)c12]

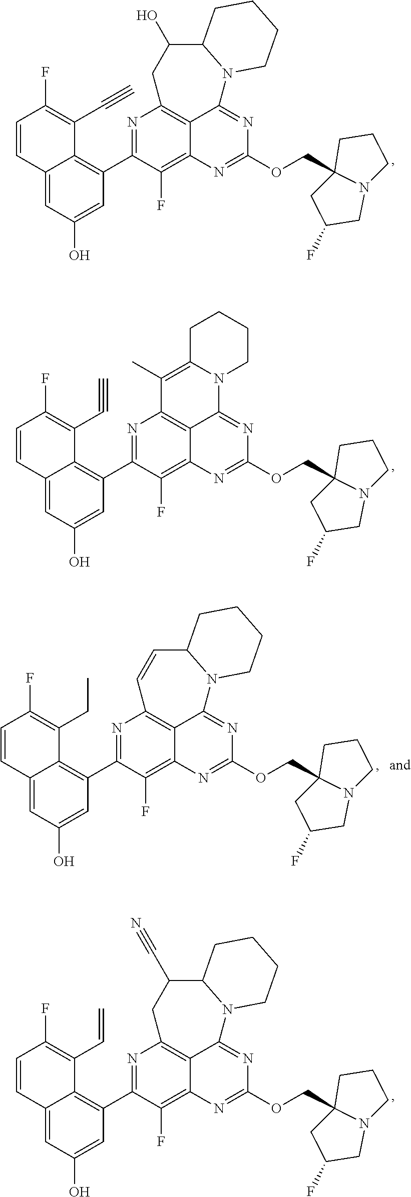 [SMILES: C#Cc1c(F)ccc2cc(O)cc(-c3nc4c5c(nc(OC[C@@]67CCCN6C[C@H](F)C7)nc5c3F)N3CCCCC3C(O)C4)c12 ; C#Cc1c(F)ccc2cc(O)cc(-c3nc4c5c(nc(OC[C@@]67CCCN6C[C@H](F)C7)nc5c3F)N3CCCCC3=C4C)c12 ; CCc1c(F)ccc2cc(O)cc(-c3nc4c5c(nc(OC[C@@]67CCCN6C[C@H](F)C7)nc5c3F)N3CCCCC3C=C4)c12 ; C=Cc1c(F)ccc2cc(O)cc(-c3nc4c5c(nc(OC[C@@]67CCCN6C[C@H](F)C7)nc5c3F)N3CCCCC3C(C#N)C4)c12]

or a pharmaceutically acceptable salt thereof.

56. A pharmaceutical composition comprising at least one compound of any one of clauses 1 to 55, or a pharmaceutically acceptable salt thereof, and optionally one or more pharmaceutically acceptable excipients.

57. A method of treating disease, such as cancer, comprising administering to a subject in need of such treatment an effective amount of a compound of any one of clauses 1 to 55, or a pharmaceutically acceptable salt thereof.

58. A compound of any one of clauses 1 to 55, or a pharmaceutically acceptable salt thereof, for use in a method of treating cancer in a subject.

59. A compound of any one of clauses 1 to 55, or a pharmaceutically acceptable salt thereof, for treating cancer in a subject.

60. Use of a compound of any one of clauses 1 to 55, or a pharmaceutically acceptable salt thereof, in the manufacture of a medicament for treating cancer in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or “A

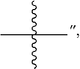

where each of “-*”, “-**”, and

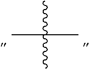

represents a bond to A and the point of covalent bond attachment to B. Alternatively, in some embodiments, the portion of A-B defined by the group or chemical structure B can be represented by “*-B”, “**-B”, or

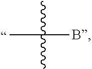

where each of “-*”, “-**”, and

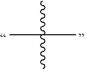

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “alkynyl” refers to a straight- or branched-chain monovalent hydrocarbon group having one or more triple bonds. In some embodiments, it can be advantageous to limit the number of atoms in an “alkynyl” to a specific range of atoms, such as C₂-C₂₀ alkynyl, C₂-C₁₂ alkynyl, or C₂-C₆ alkynyl. Examples of alkynyl groups include acetylenyl (—C≡CH) and propargyl (—CH₂C≡CH), but-3-yn-1,4-diyl (—C≡C—CH₂CH₂—), and the like. It will be appreciated that an alkynyl group can be unsubstituted or substituted as described herein. An alkynyl group can be substituted with any of the substituents in the various embodiments described herein, including one or more of such substituents.

The term “cycloalkyl” refers to a saturated or partially saturated, monocyclic or polycyclic mono-valent carbocycle. In some embodiments, it can be advantageous to limit the number of atoms in a “cycloalkyl” to a specific range of atoms, such as having 3 to 12 ring atoms. Polycyclic carbocycles include fused, bridged, and spiro polycyclic systems. Illustrative examples of cycloalkyl groups include monovalent radicals of the following entities:

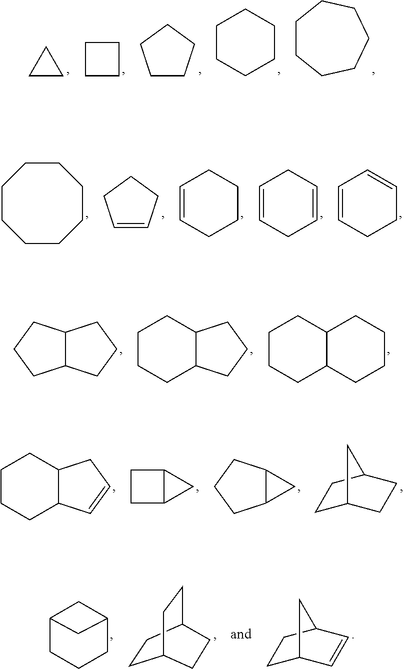

In particular, a cyclopropyl moiety can be depicted by the structural formula

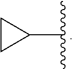 [SMILES: *C1CC1]

It will be appreciated that a cycloalkyl group can be unsubstituted or substituted as described herein. A cycloalkyl group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F.

In certain embodiments, alkenyl may be substituted. For example, the alkenyl may be substituted with halo (e.g., fluoro). Illustratively, the alkenyl substituted with halo may be a ═CHF.

The term “aryl” refers to a monovalent all-carbon monocyclic or fused-ring polycyclic group having a completely conjugated pi-electron system. In some embodiments, it can be advantageous to limit the number of atoms in an “aryl” to a specific range of atoms, such as mono-valent all-carbon monocyclic or fused-ring polycyclic groups of 6 to 14 carbon atoms (C₆-C₁₄ aryl), or monovalent all-carbon monocyclic or fused-ring polycyclic groups of 6 to 10 carbon atoms (C₆-C₁₀ aryl). Examples, without limitation, of aryl groups are phenyl, naphthalenyl and anthracenyl. It will be appreciated that an aryl group can be unsubstituted or substituted as described herein. An aryl group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. In some embodiments, it can be advantageous to limit the number of atoms in a “heterocycloalkyl” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), 5 to 7 ring atoms (5- to 7-membered), or 4 to 10 ring atoms (4- to 10-membered). In some embodiments, it can be advantageous to limit the number and type of ring heteroatoms in “heterocycloalkyl” or to a specific range or type of heteroatoms, such as 1 to 5 ring heteroatoms selected from nitrogen, oxygen, and sulfur. Examples, without limitations, of mono-cyclic heterocycloalkyl groups include tetrahydrofuran, pyrrolidine, and morpholine. Polycyclic ring systems include fused, bridged, and spiro systems. In some embodiments, it can be advantageous to limit the number of atoms in a bicyclic “heterocycloalkyl” to a specific range of ring atoms, such as from 5 to 10 ring atoms (5- to 10-membered), or 6 to 10 ring atoms (6- to 10-membered). The ring structure may optionally contain an oxo group or an imino group on a carbon ring member or up to two oxo groups on sulfur ring members. Examples, without limitations, of fused bicyclic, bridged bicyclic, and spiro bicyclic heterocycloalkyl groups include pyrrolizinyl, 2,5-diazabicyclo[2.2.2]octanyl, 1-oxaspiro[4.5]decanyl, and hexahydro-1H-pyrrolizinyl. Illustrative examples of heterocycloalkyl groups include monovalent radicals of the following entities:

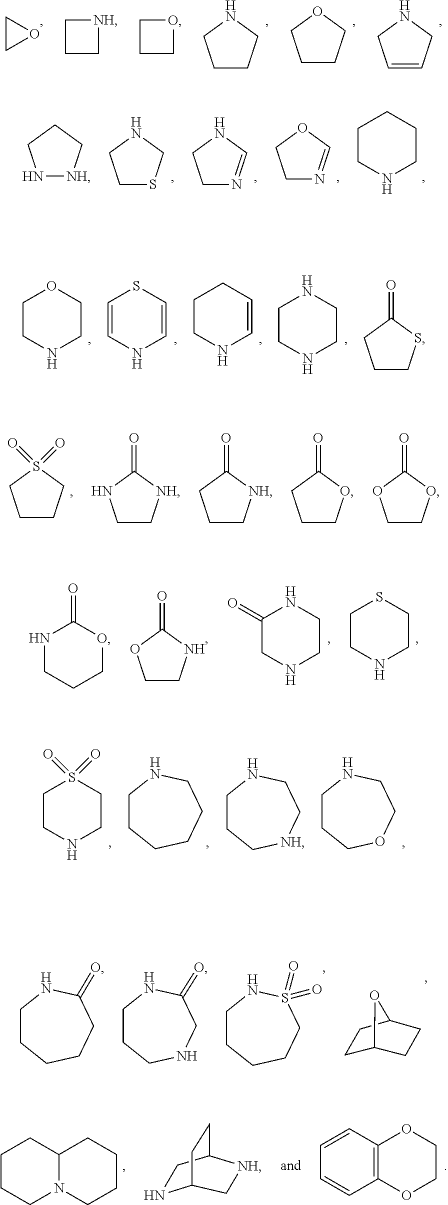

In particular, a hexahydro-1H-pyrrolizinyl moiety can be depicted by the structural formula

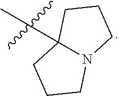 [SMILES: *C12CCCN1CCC2]

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2, 5-dihydro-1H-pyrrole, pyrazolidine, thiazolidine, 4,5-dihydro-1H-imidazole, dihydrothiophen-2(3H)-one, tetrahydrothiophene 1,1-dioxide, imidazolidin-2-one, pyrrolidin-2-one, dihydrofuran-2(3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

It will be appreciated that a heterocycloalkyl group can be unsubstituted or substituted as described herein. A heterocycloalkyl group can be substituted with any of the substituents in the various embodiments described herein, including one or more of such substituents.

A heterocycloalkyl group can be combined with another group, such as an alkenyl group, to provide a moiety that may be unsubstituted or substituted with one or more groups, such as one of more halogen atoms. Illustratively, a hexahydro-1H-pyrrolizinyl moiety may be combined with an alkenyl group to provide an exocyclic alkenyl group on the hexahydro-1H-pyrrolizinyl, which alkenyl group may be further substituted by halogen. For example, a haloalkenyl substituted hexahydro-1H-pyrrolizinyl moiety can be depicted by the structural formula

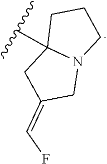 [SMILES: *C12CCCN1C/C(=C\F)C2]

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a divalent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl. In some instances, a 5- to 10-membered heteroaryl can be a monocyclic ring or fused bicyclic rings having 5- to 10-ring atoms wherein at least one ring atom is a heteroatom, such as N, O, or S. The ring structure may optionally contain an oxo group or an imino group on a carbon ring member or up to two oxo groups on sulfur ring members. Illustrative examples of 5- to 10-membered heteroaryl groups include monovalent radicals of the following entities, while examples of 5- to 10-membered heteroarylene groups include divalent radicals of the following entities, in the form of properly bonded moieties:

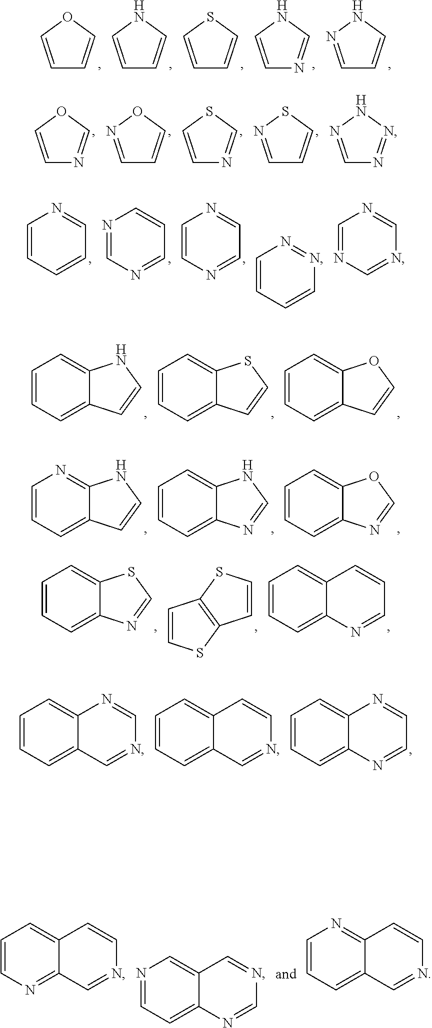

In some embodiments, a “monocyclic” heteroaryl can be an aromatic five- or six-membered heterocycle. A five-membered heteroaryl can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. A “bicyclic heteroaryl” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, indazole, 1H-benzo[d]imidazole, benzo[d]oxazole, and benzo[d]thiazole.

In particular, an isoquinolin-3(2H)-onyl moiety can be depicted by the structural formula

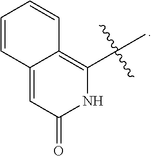 [SMILES: *c1[nH]c(=O)cc2ccccc12]

In particular, a divalent pyridopyrimidine moiety can be depicted by the structural formula

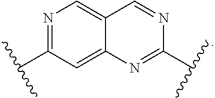 [SMILES: *c1cc2nc(*)ncc2cn1]

It will be appreciated that a heteroaryl group can be unsubstituted or substituted as described herein. A heteroaryl group can be substituted with any of the substituents in the various embodiments described herein, including one or more of such substituents.

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Certain chemical entities of Formula (I)-(LXXXI) may be depicted in two or more tautomeric forms. Any and all alternative tautomers are included within the scope of these formulas, and no inference should be made as to whether the chemical entity exists as the tautomeric form in which it is drawn. It will be understood that the chemical entities described herein, and their constituent rings A, B, etc. can exist in different tautomeric forms. It will be readily appreciated by one of skill in the art that because of rapid interconversion, tautomers can generally be considered to be the same chemical compound. Examples of tautomers include but are not limited to enol-keto tautomers amine-imine tautomers and the like.

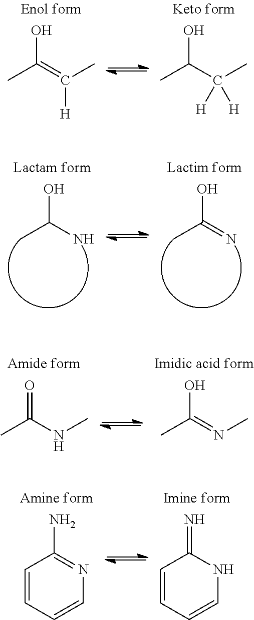

In particular, a ring option of isoquinolin-3(2H)-oneylene can exist as the following tautomers

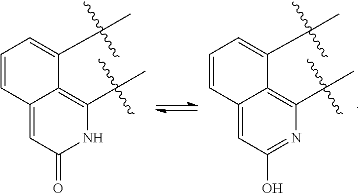 [SMILES: *c1cccc2cc(=O)[nH]c(*)c12 ; *c1cccc2cc(O)nc(*)c12]

The nomenclature “(ATOM)ᵢ-(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

The disclosure also includes pharmaceutically acceptable salts of the compounds represented by Formula (I)-(LXXXI), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound (e.g., a salt or a pharmaceutically acceptable salt) or can substituted to a salt for further manufacture, formulation, or administration reasons. As illustrated herein, certain compounds include a “W^{⊖},” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)-(LXXXI) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)-(LXXXI), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)-(LXXXI). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs,” ed. H. Bundgaard, Elsevier, 1985.

The present disclosure also relates to pharmaceutically active metabolites of compounds of Formula (I)-(LXXXI), and uses of such metabolites in the methods of the disclosure. A “pharmaceutically active metabolite” means a pharmacologically active product of metabolism in the body of a compound of Formula (I)-(LXXXI) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

As used herein, the term “KRAS inhibitor” includes, but is not limited to, a compound that is capable of inhibiting the protein encoded by the KRAS gene, called K-Ras, that is involved in the RAS/MAPK signaling pathway. The terms KRAS gene, K-Ras, and RAS/MAPK signaling pathway will be known and understood by one of skill in the art. It will be appreciated that KRAS mutations occur in approximately one in seven of all human metastatic cancers, and that those mutations can occur in a variety of locations in the KRAS gene coding sequence. KRAS mutations primarily occur in KRAS codons 12 and 13, and also occur in codons 18, 61, 117, and 146 at low frequencies and have distinct effects on tumor cell signaling based on the codon and missense mutation. Examples of KRAS mutations include, but are not limited to KRAS G12C, KRAS G12D, KRAS G12V, KRAS G12R, KRAS G12S, KRAS G13C, KRAS G13D, KRAS A18D, KRAS Q61H, KRAS K117N, and the like. It will be understood by a person having ordinary skill in the art that reference to a inhibiting of KRAS mutations, such as KRAS G12D refers to inhibiting the protein encoded by the KRAS G12D gene, having a coding sequence (e.g. a guanine to adenine substitution, at position 35 on codon 12 of the KRAS coding sequence) that produces a K-Ras G12D protein, where a glycine at position 12 of the protein sequence is replaced by am aspartic acid.

### Representative Embodiments

In some embodiments, the disclosure relates to a compound of the formula I, or a pharmaceutically acceptable salt thereof,

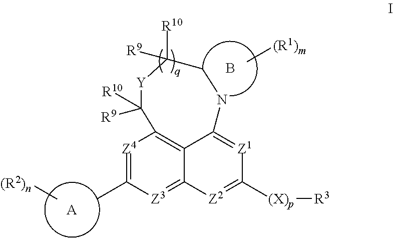

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl, a 5- to 10-membered heteroaryl, or a 4- to 10-membered heterocycloalkyl;
ring B is a 4- to 9-membered heterocycloalkyl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R¹ and an R⁹ or an R¹⁰ on an adjacent carbon to ring B, taken together with the carbon atoms to which they are attached, combine form a carbon-carbon double bond;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)ORC, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR⁶C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NRS(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

In some embodiments, the disclosure relates to a compound of the formula I, or a pharmaceutically acceptable salt thereof,

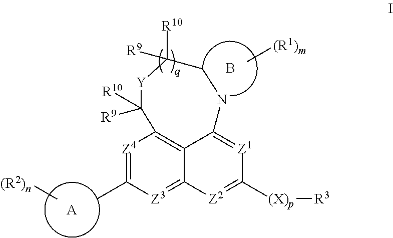

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl or 5- to 10-membered heteroaryl;
ring B is a 4- to 9-membered heterocycloalkyl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NRS(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{e}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

In some embodiments, the disclosure relates to a compound of the formula I, or a pharmaceutically acceptable salt thereof,

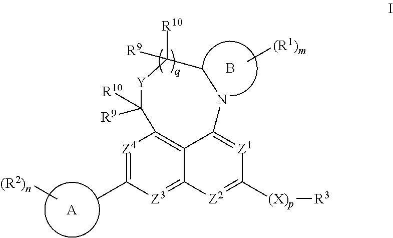

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl or 5- to 10-membered heteroaryl;
ring B is a 4- to 9-membered heterocycloalkyl ring having one nitrogen atom in the 4- to 9-membered heterocycloalkyl ring;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{e})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂RC, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁O aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂₀R^{a}, —CN, or —NO₂, or R⁹ and R¹⁰, taken together with the carbon atom to which they are attached, form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

In some embodiments, the disclosure relates to a compound of the formula I, or a pharmaceutically acceptable salt thereof,

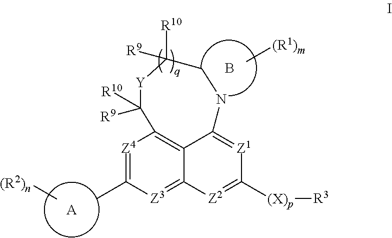

wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl, 5- to 10-membered heteroaryl, or

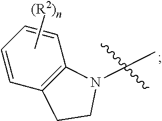

ring B is a 4- to 9-membered heterocycloalkyl;
each R³ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{e}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆—Co aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

In some embodiments, the disclosure relates to a compound of the formula II, 111, IV, V, VI, VII, VIII, or IX, or a pharmaceutically acceptable salt thereof,

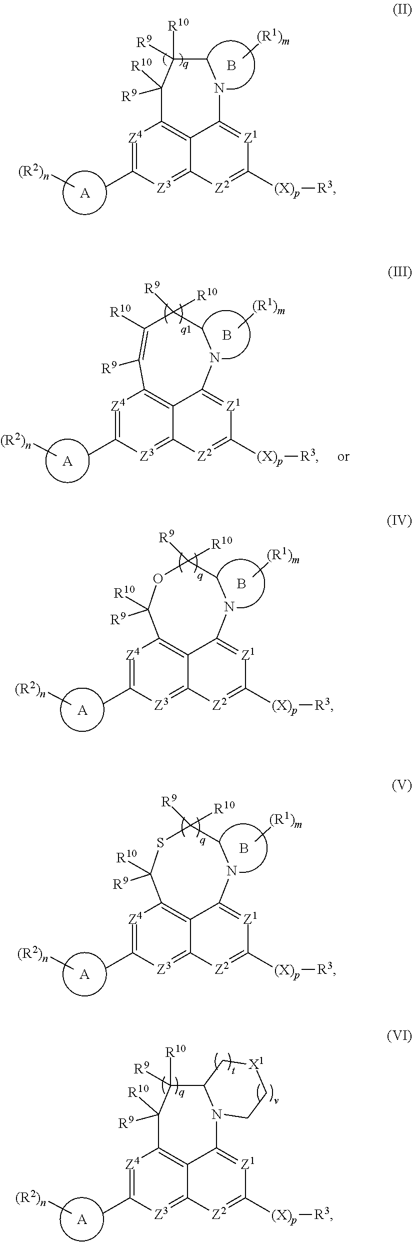

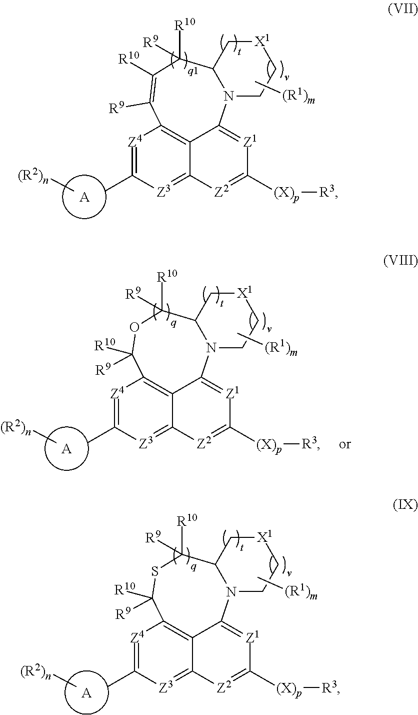

wherein R¹, R², R³, R⁹, R¹⁰, A, B, X, X¹, Y, Z¹, Z², Z³, Z⁴, m, n, p, q, q1, t, and v are as described herein.

In some embodiments, the disclosure relates to a compound of the formula X, XI, XII, XIII, XIV, XV, XVI, or XVII, or a pharmaceutically acceptable salt thereof,

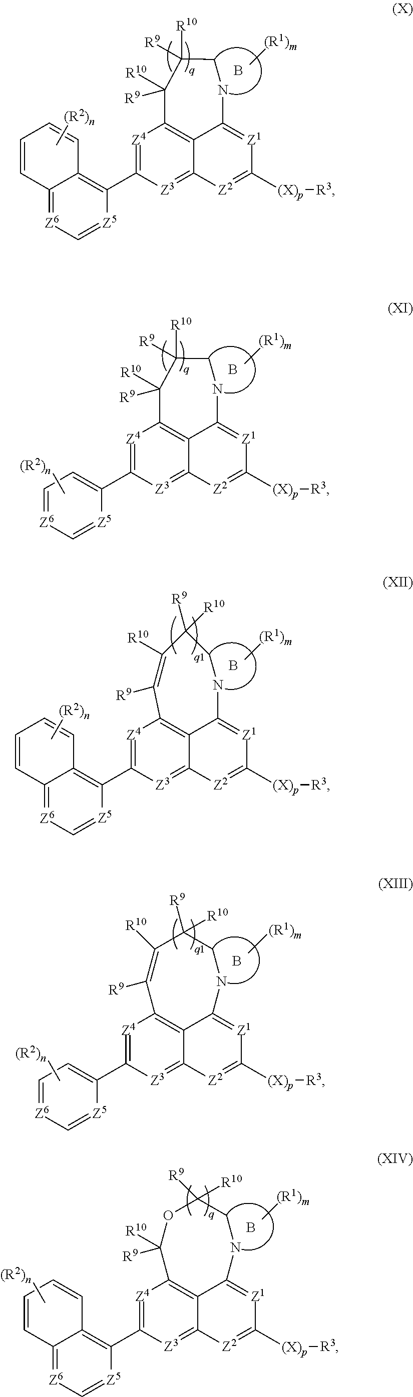

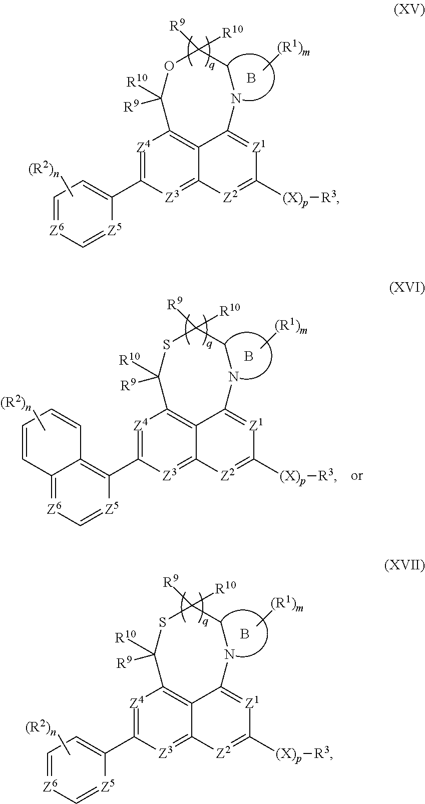

wherein R¹, R², R³, R⁹, R¹⁰, B, X, Z¹, Z², Z³, Z⁴, Z⁵, Z⁶, m, n, p, q, and q1, are as described herein.

In some embodiments, the disclosure relates to a compound of the formula XVIII, XIX,

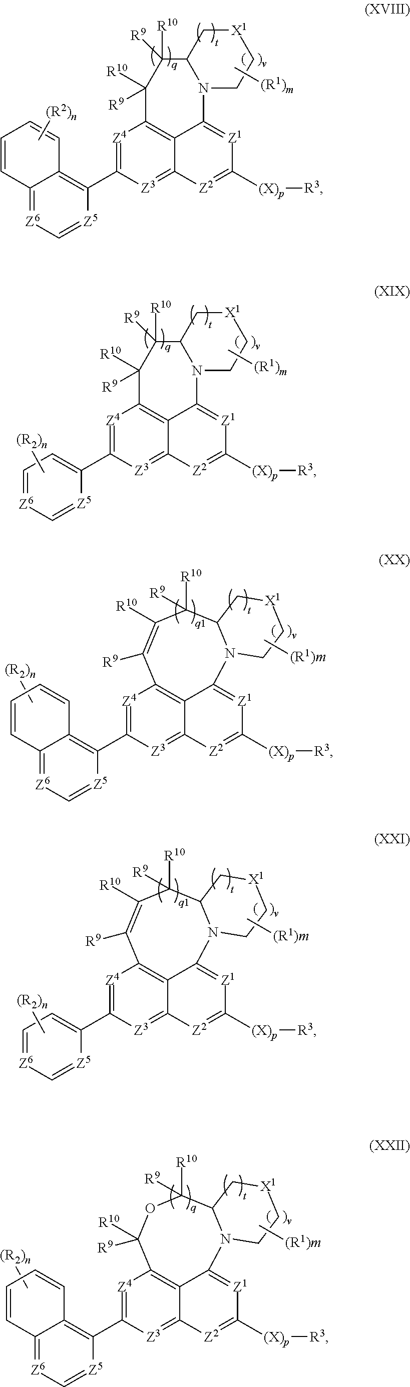

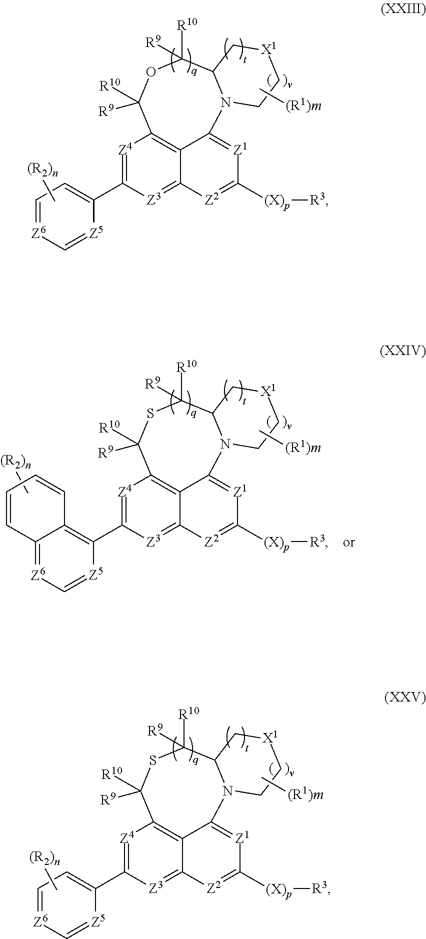

wherein R¹, R², R³, R⁹, R¹⁰, X, X¹, Z¹, Z², Z³, Z⁴, Z⁵, Z⁶, m, n, p, q, q1, t, and v are as described herein.

In some embodiments, the disclosure relates to a compound of the formula XXVI, XXVII, XXVIII, XXIX, XXX, XXXI, XXXII, or XXXIII, or a pharmaceutically acceptable salt thereof,

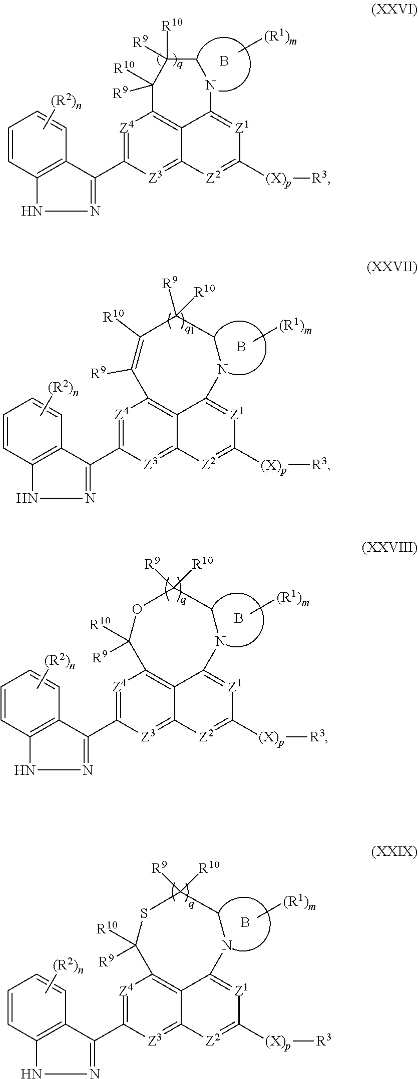

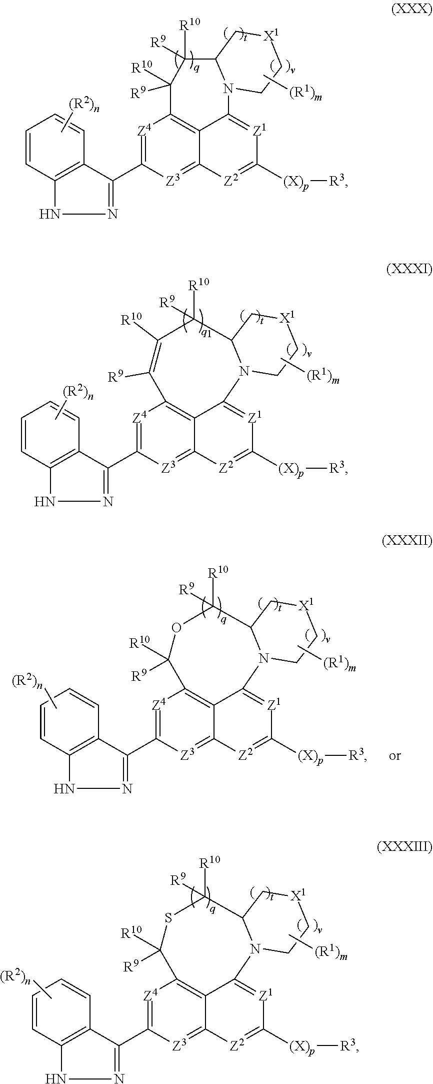

wherein R³, R², R³, R⁹, R¹⁰, B, X, X¹, Z¹, Z², Z³, Z⁴, m, n, p, q, q1, t, and v are as described herein.

In some embodiments, the disclosure relates to a compound of the formula XXXIV, XXXV, XXXVI, XXXVII, XXXVIII, XXXIX, XXXX, or XXXXI, or a pharmaceutically acceptable salt thereof,

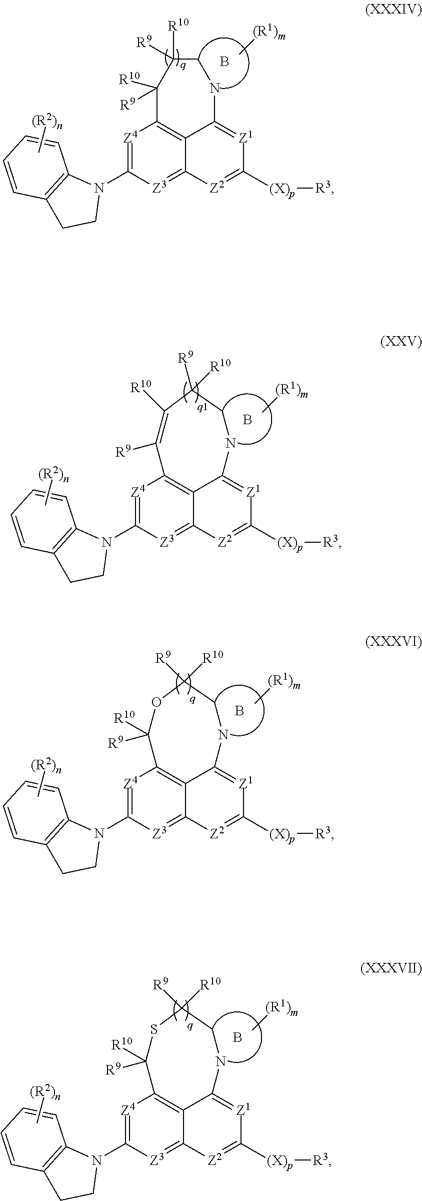

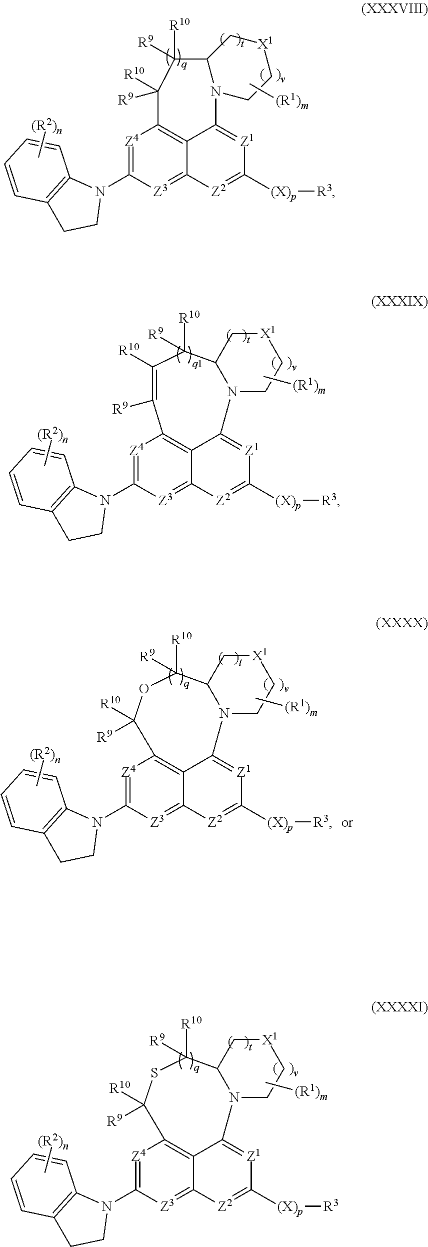

wherein R¹, R², R³, R⁹, R¹⁰, B, X, X¹, Z¹, Z², Z³, Z⁴, m, n, p, q, q1, t, and v are as described herein.

In some embodiments, the disclosure relates to a compound of the formula XXXXII, XXXXIII, XXXXIV, XXXV, XXXVI, XXXXVII, XXXXVIII, or XXXXIX, or a pharmaceutically acceptable salt thereof,

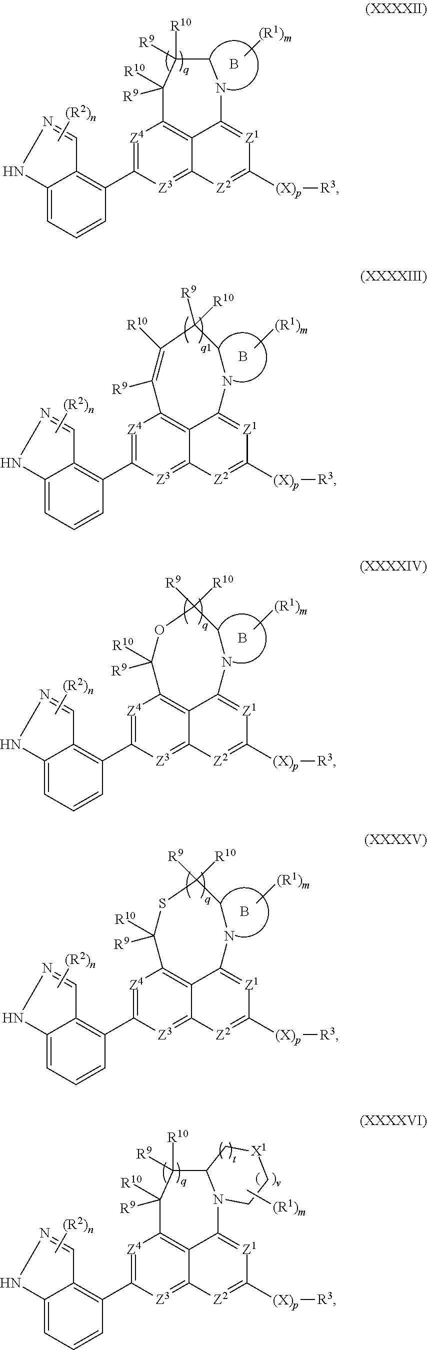

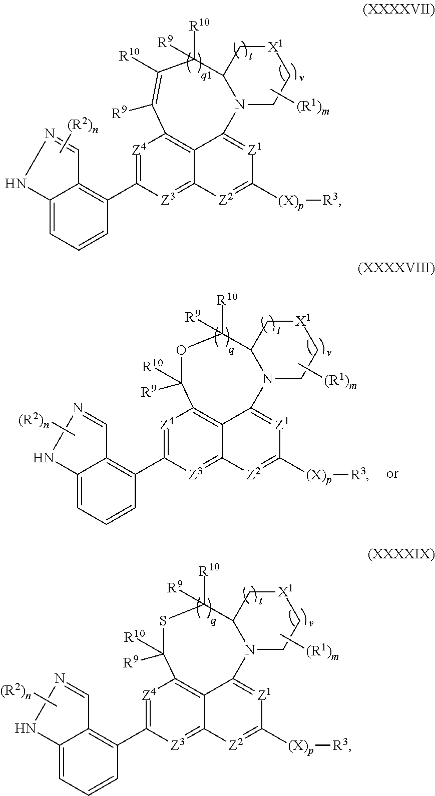

wherein R¹, R², R³, R⁹, R¹⁰, B, X, X¹, Z¹, Z², Z³, Z⁴, m, n, p, q, q1, t, and v are as described herein.

In some embodiments, the disclosure relates to a compound of the formula L, LI, LII, LIII, LIV, LV, LVI, or LVII, or a pharmaceutically acceptable salt thereof,

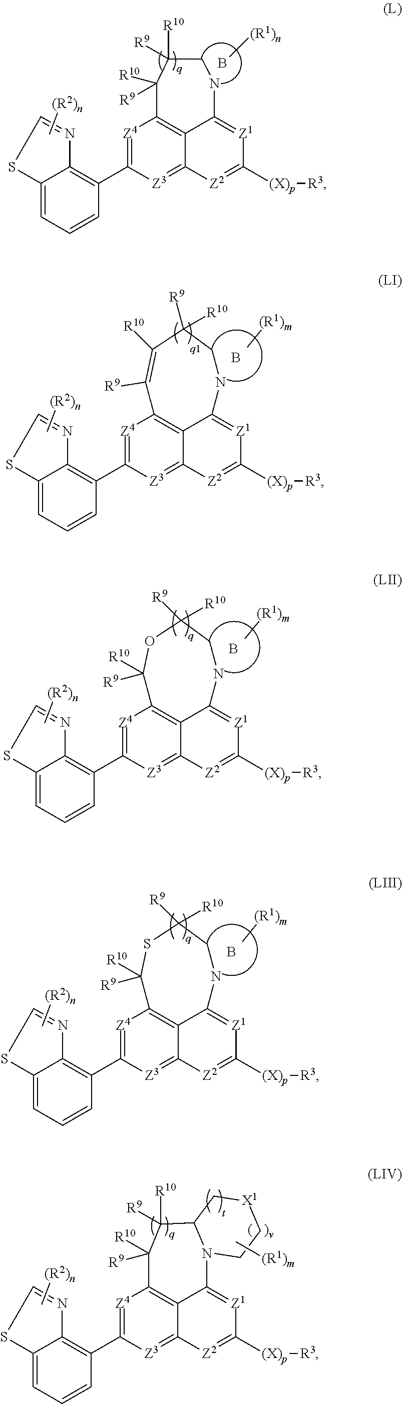

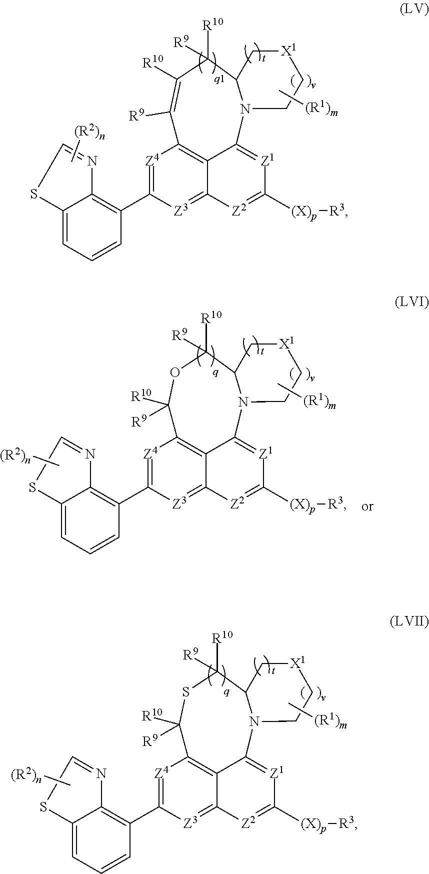

wherein R¹, R², R³, R⁹, R¹⁰, B, X, X¹, Z¹, Z², Z³, Z⁴, m, n, p, q, q1, t, and v are as described herein.

In some embodiments, the disclosure relates to a compound of the formula LVIII, LVIX, LX, LXI, LXII, LXIII, LXIV, or LXV, or a pharmaceutically acceptable salt thereof,

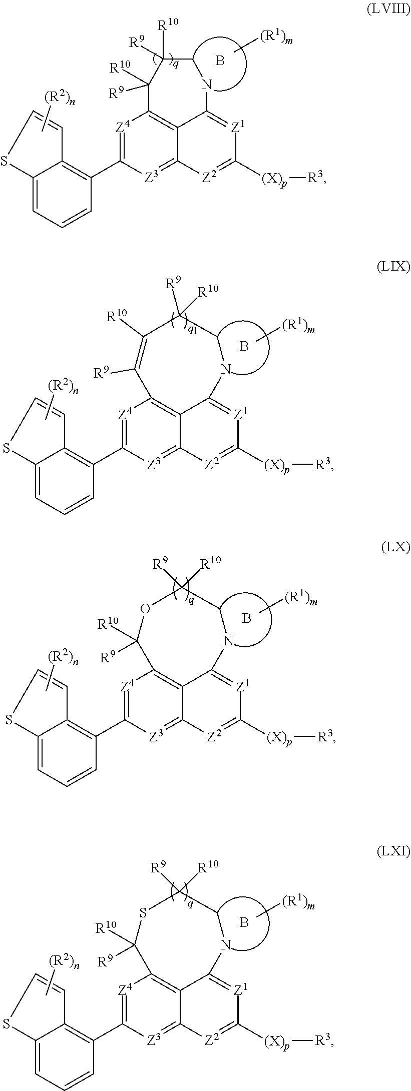

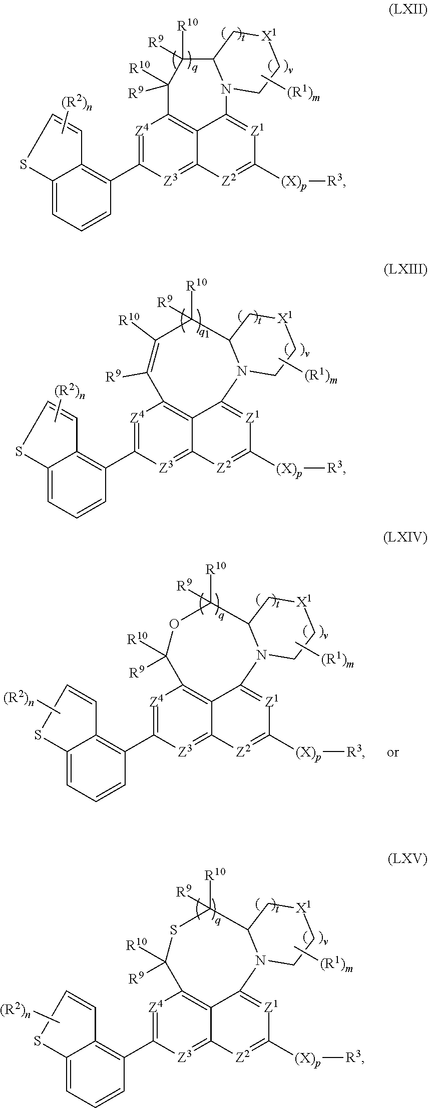

R¹, R², R³, R⁹, R¹⁰, B, X, X¹, Z¹, Z², Z³, Z⁴, m, n, p, q, q1, t, and v are as described herein.

In some embodiments, the disclosure relates to a compound of the formula LXVI, LXVII, LXVIII, LXIX, LXX, LXXI, LXXII, LXXIII, LXXIV, LXXV, LXXVI, LXXVII, LXXVIII, LXXIX, LXXX, or LXXXI, or a pharmaceutically acceptable salt thereof,

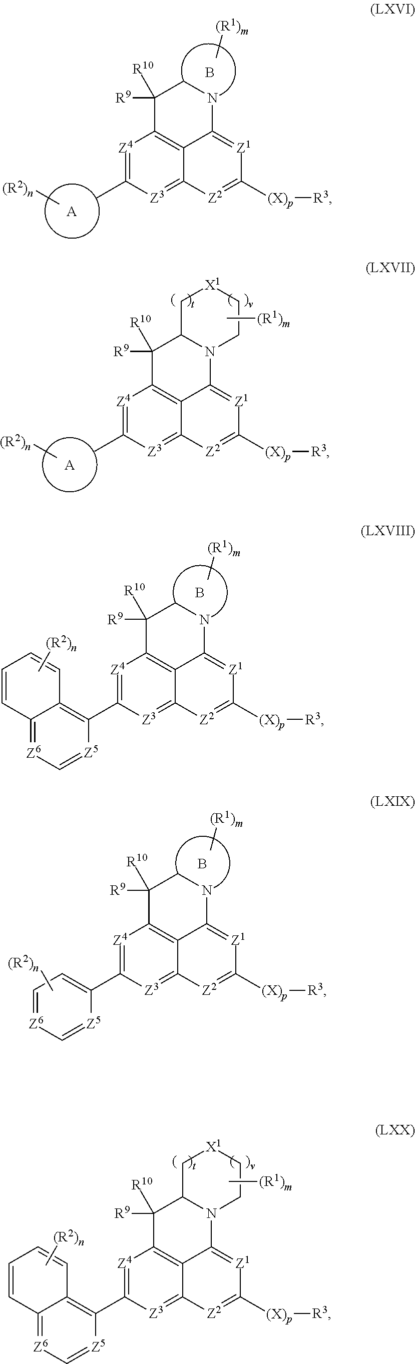

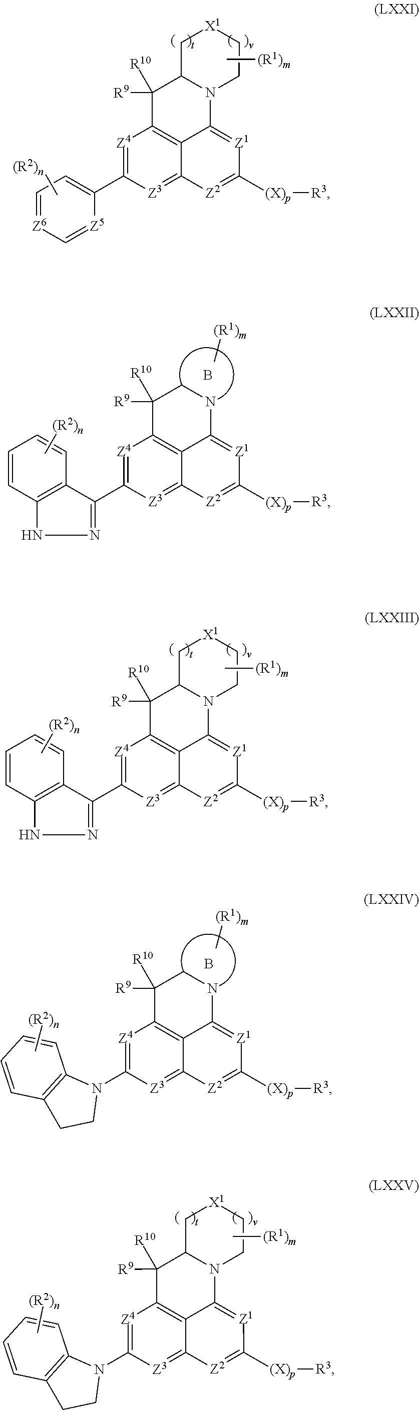

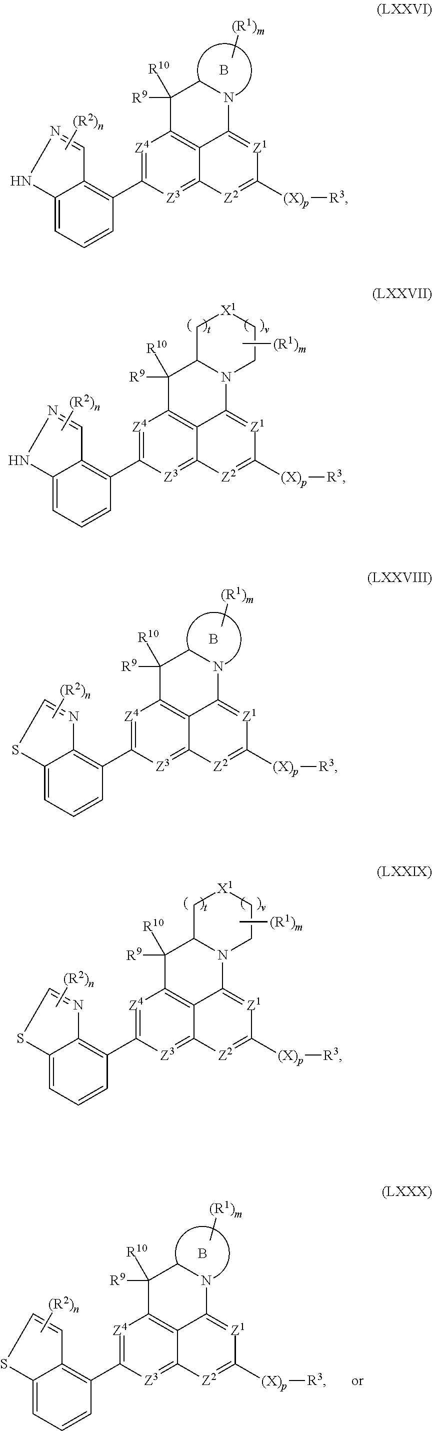

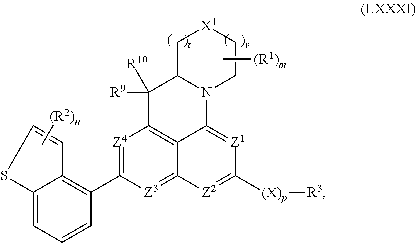

R¹, R², R³, R⁹, R¹⁰, B, X, X¹, Z¹, Z², Z³, Z⁴, m, n, p, q, q1, t, and v are as described herein.

In some embodiments, ring A is a C₆-C₁₀ aryl or 5- to 10-membered heteroaryl.

In some embodiments, ring B is a 4- to 9-membered heterocycloalkyl. In some embodiments, ring B is a 4- to 7-membered heterocycloalkyl. In some embodiments, ring B is a 4- to 9-membered heterocycloalkyl ring having one nitrogen atom in the 4- to 9-membered heterocycloalkyl ring. In some embodiments, ring B is a 4- to 7-membered heterocycloalkyl ring having one nitrogen atom in the 4- to 9-membered heterocycloalkyl ring. For example, in some embodiments ring B is a fused 4- to 9-membered heterocycloalkyl that forms a core of the compound of the formula I, the compound comprises a core having the formula

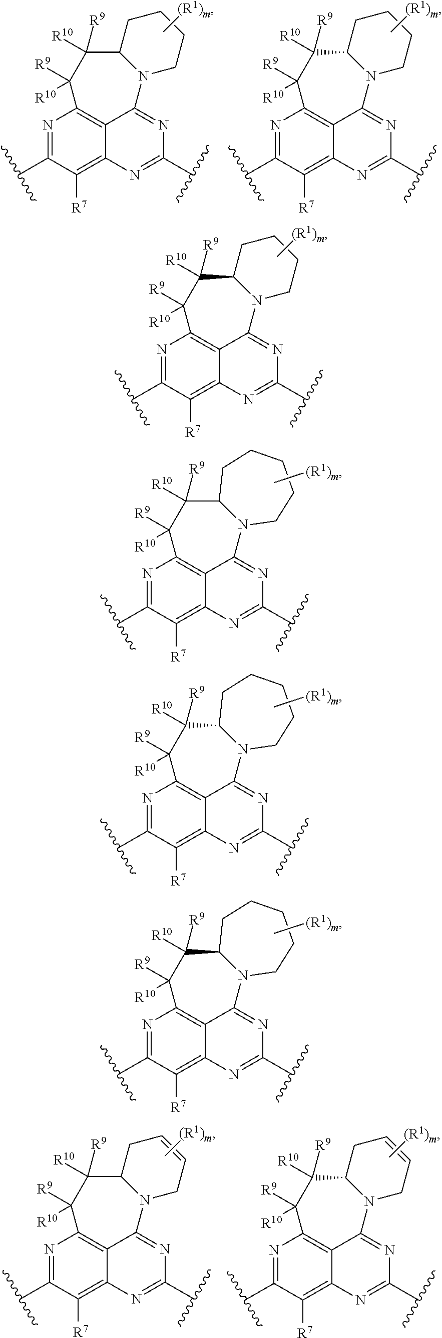

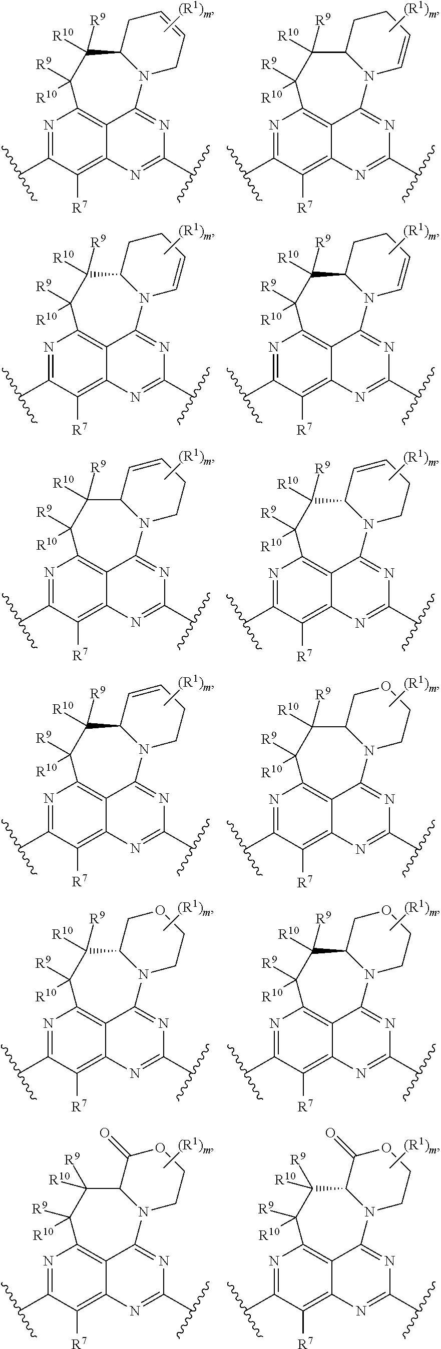

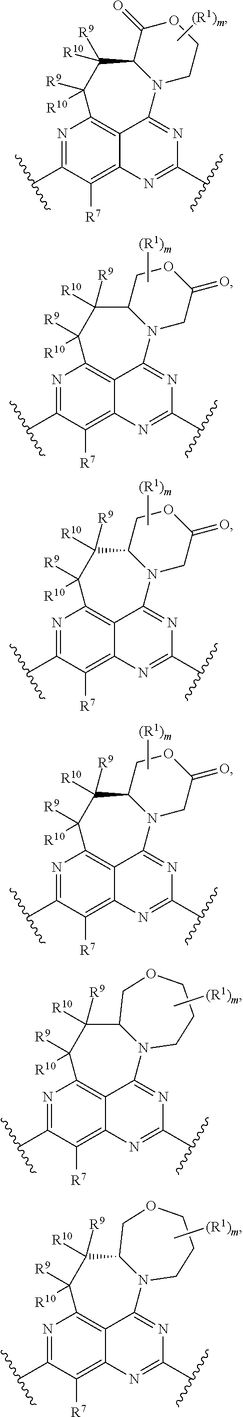

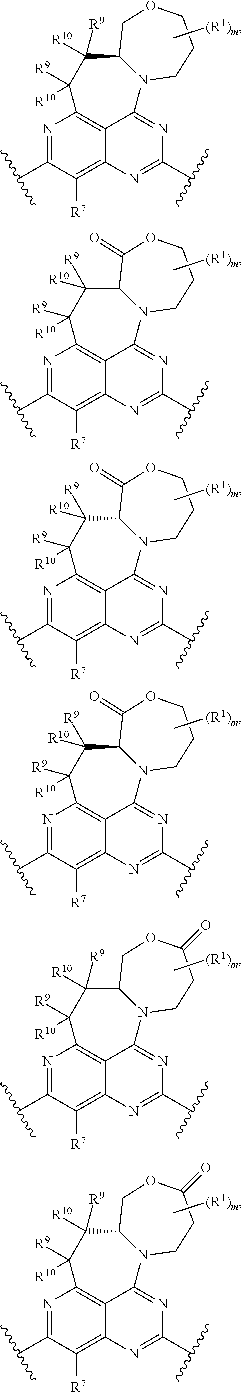

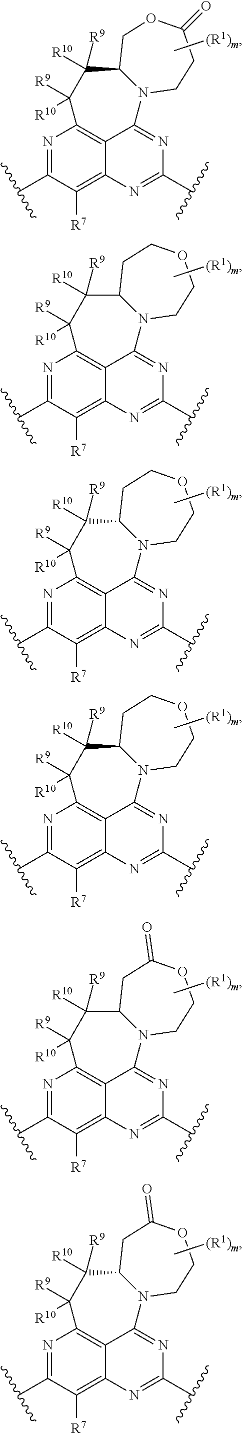

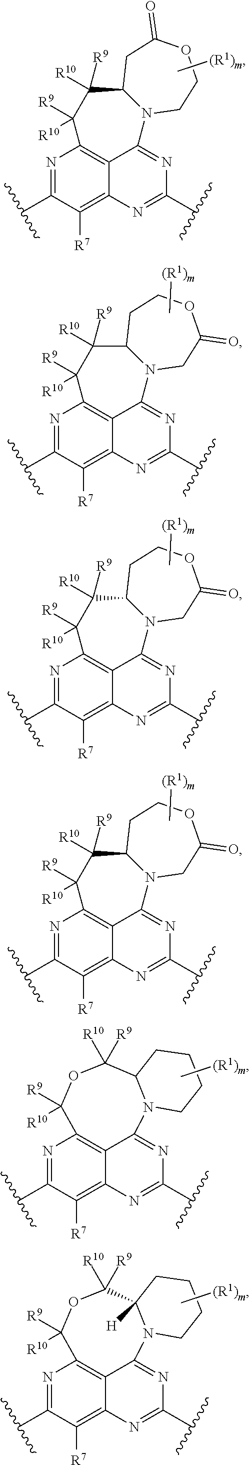

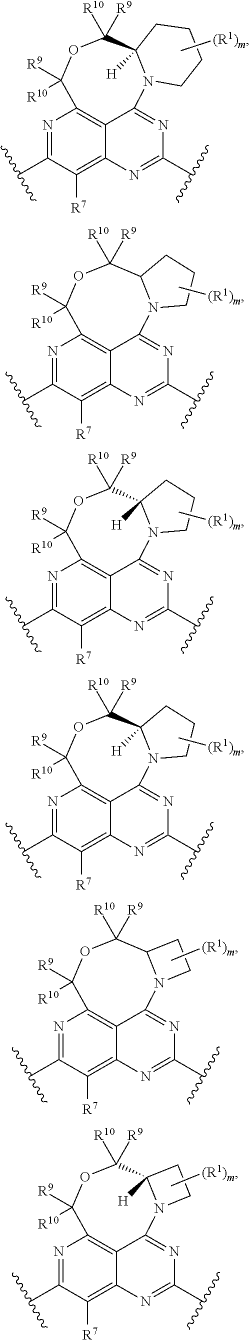

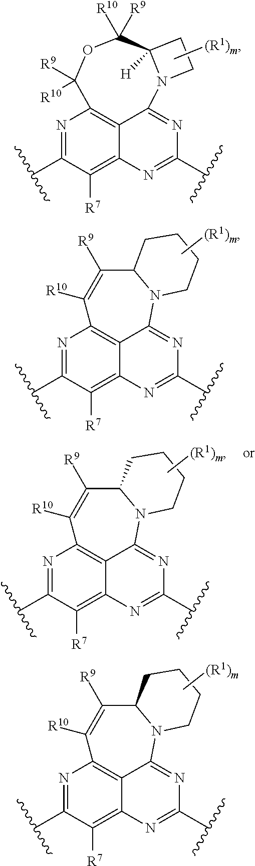

wherein each “ [symbol] ” is a point of covalent attachment to either ring A or —(X)ₚ—R³.

In some embodiments, the compound comprises a core having the formula

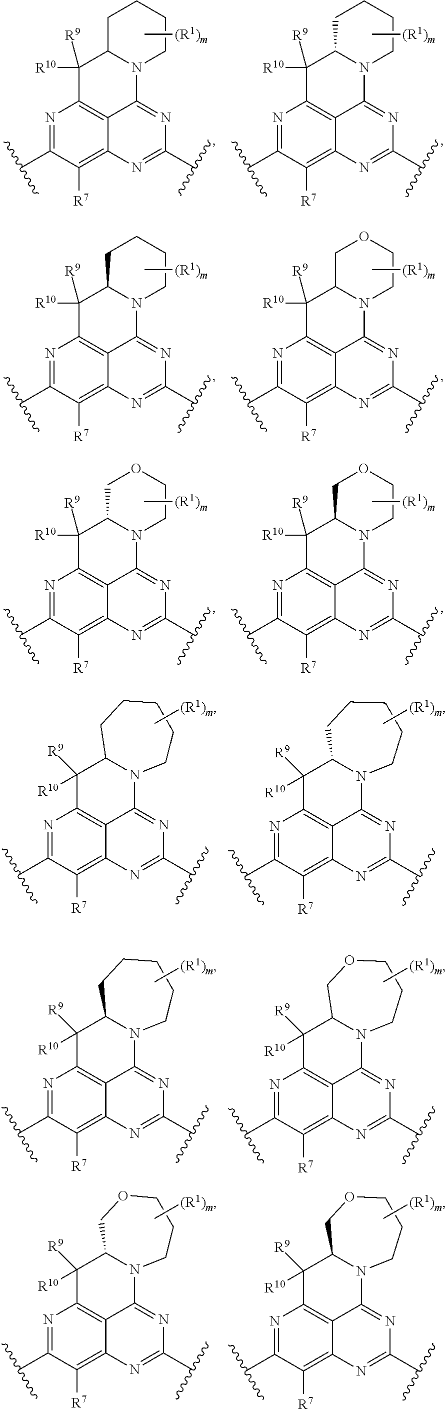

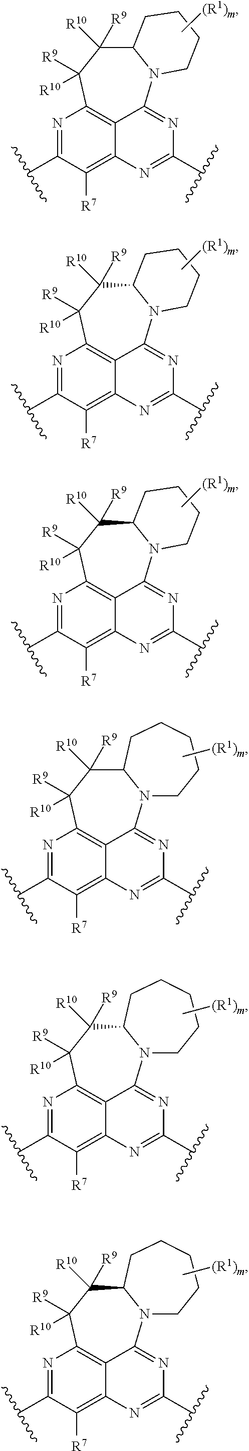

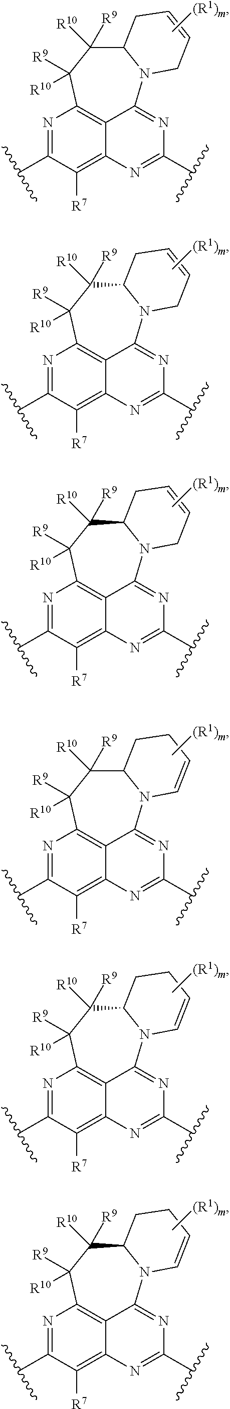

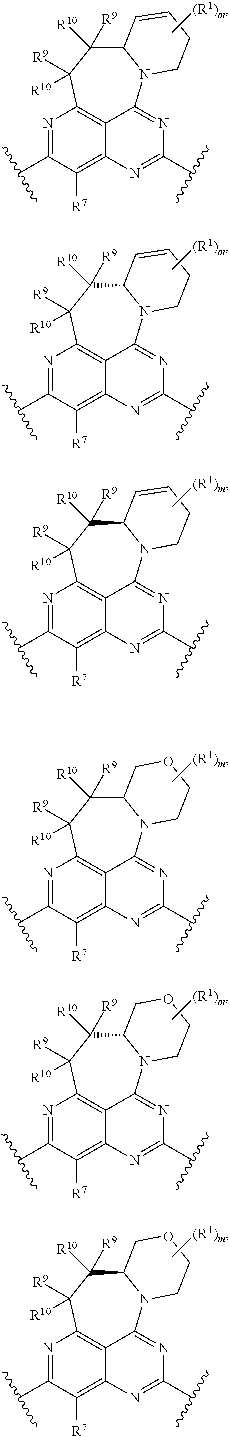

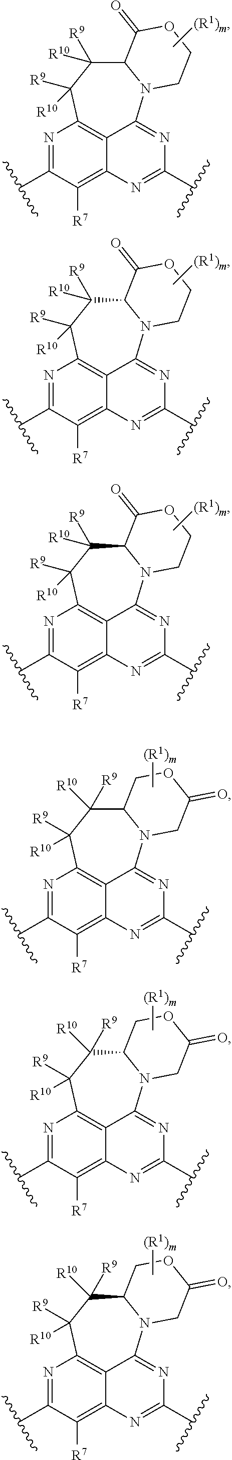

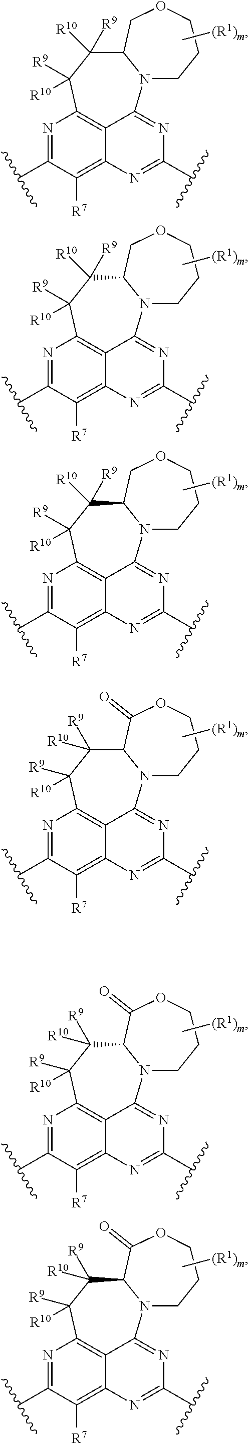

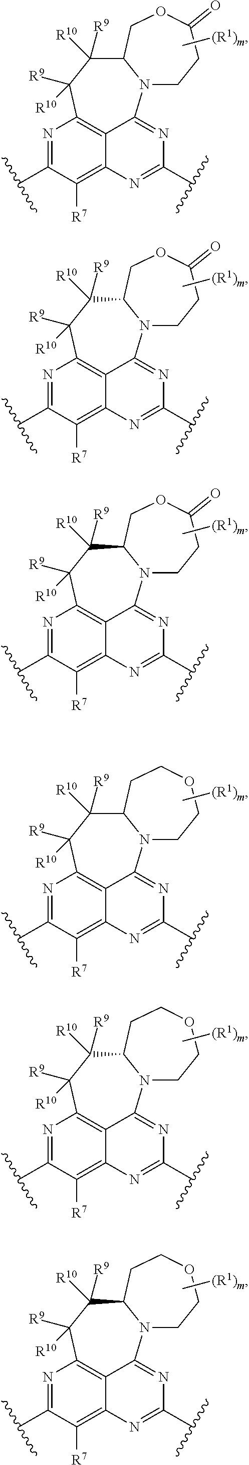

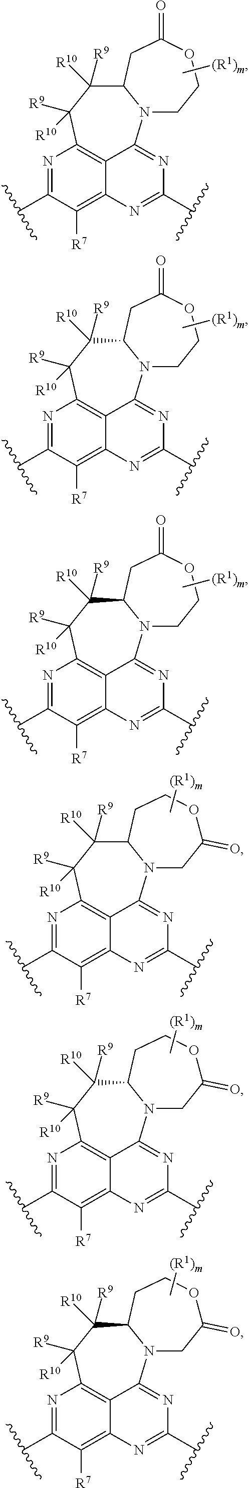

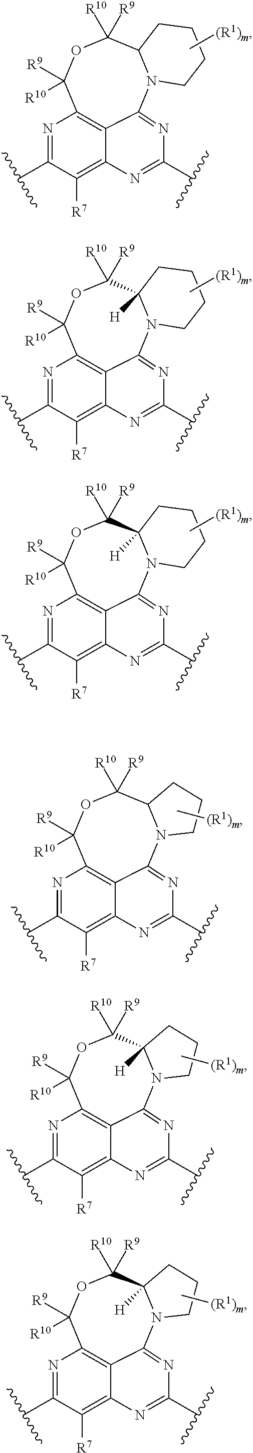

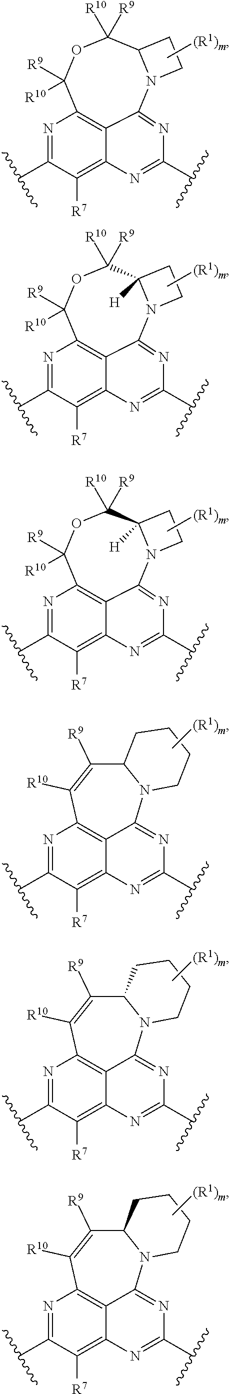

wherein each “ [symbol] ” is a point of covalent attachment to either ring A or —(X)ₚ—R³.

In some embodiments, the compound comprises a core having the formula

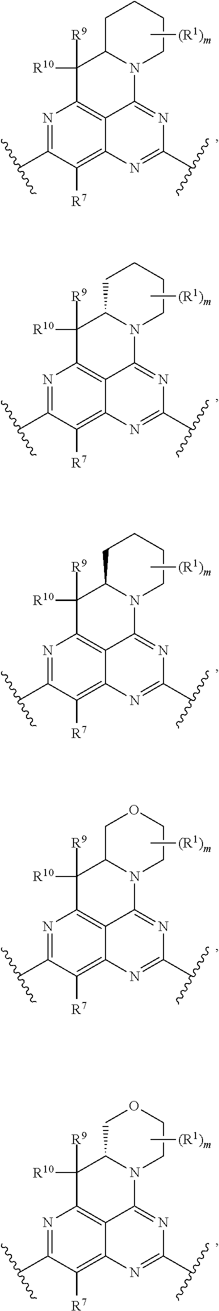

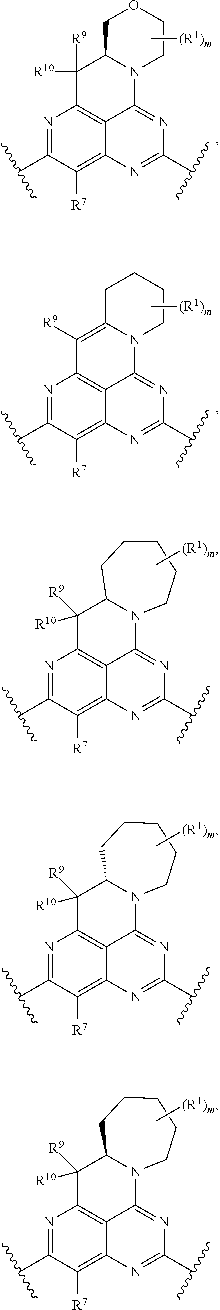

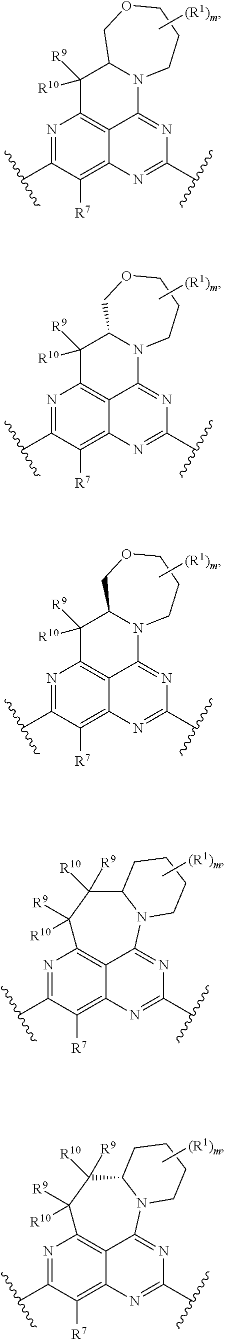

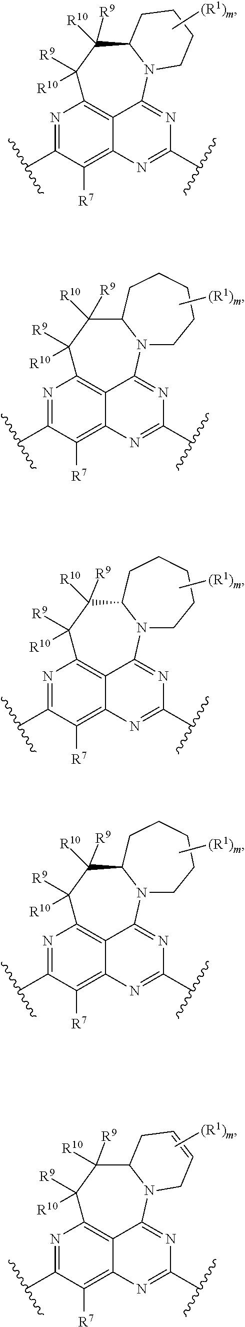

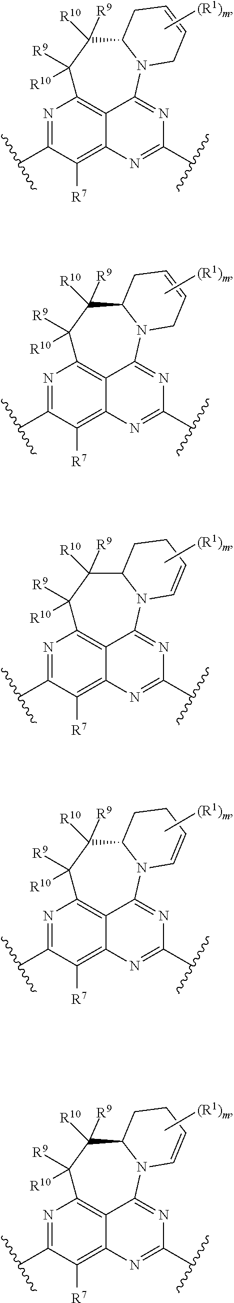

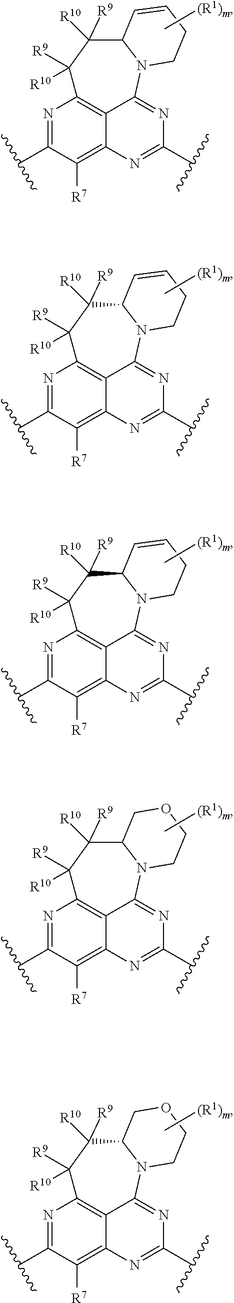

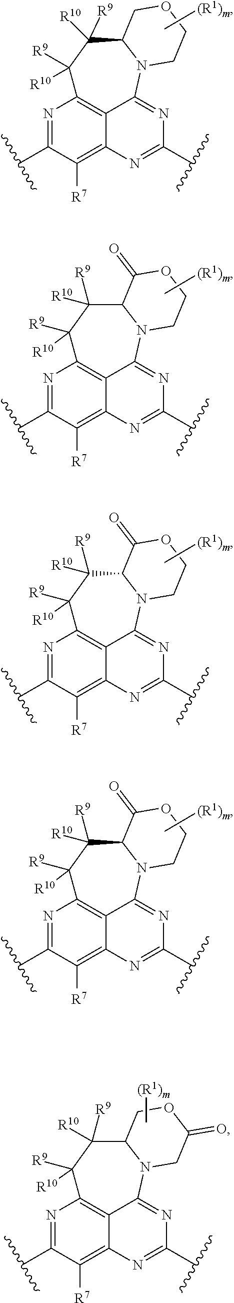

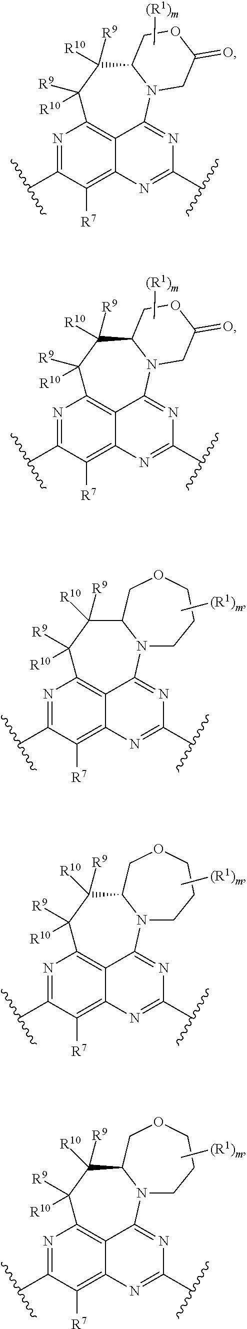

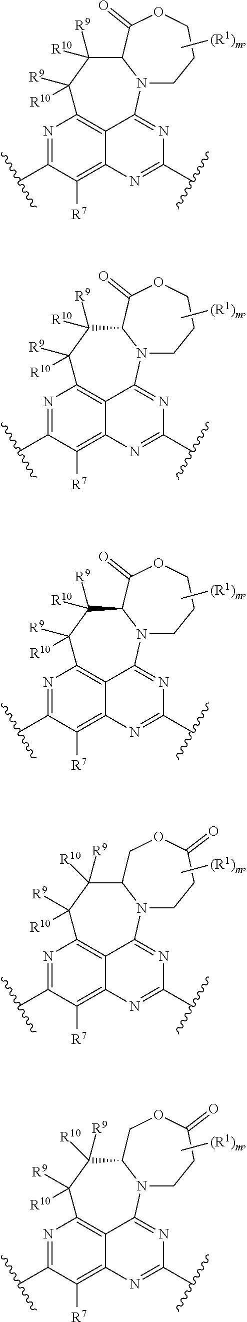

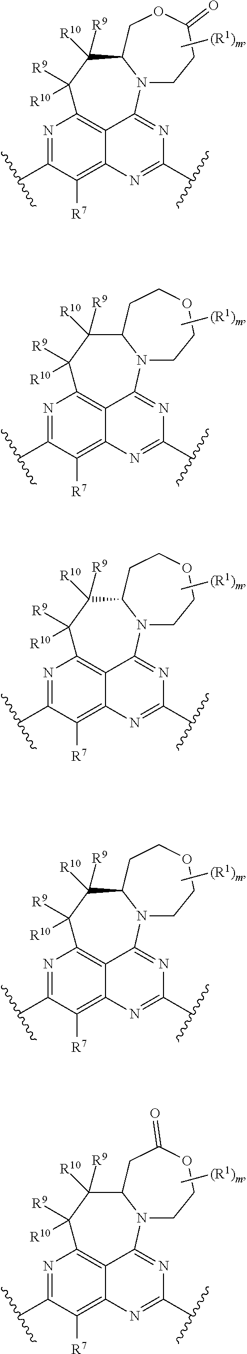

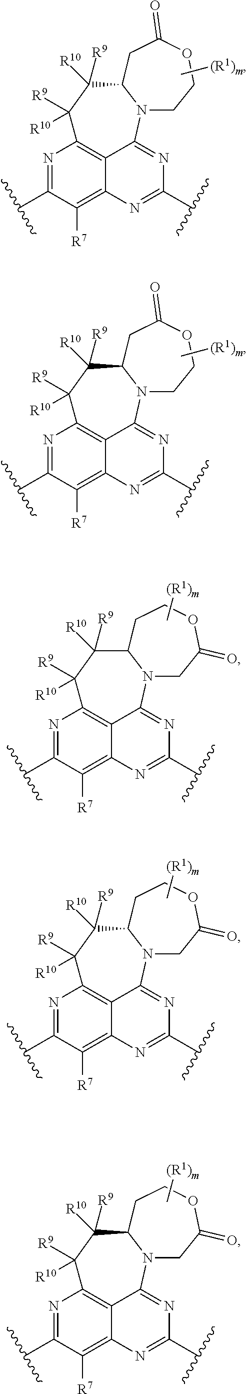

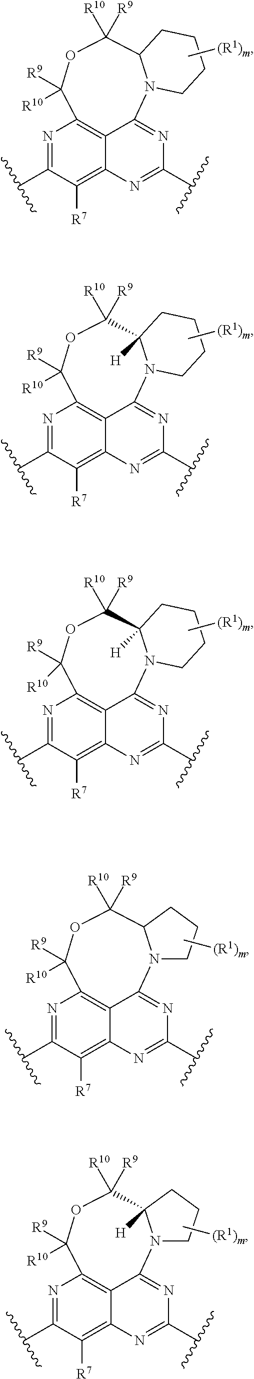

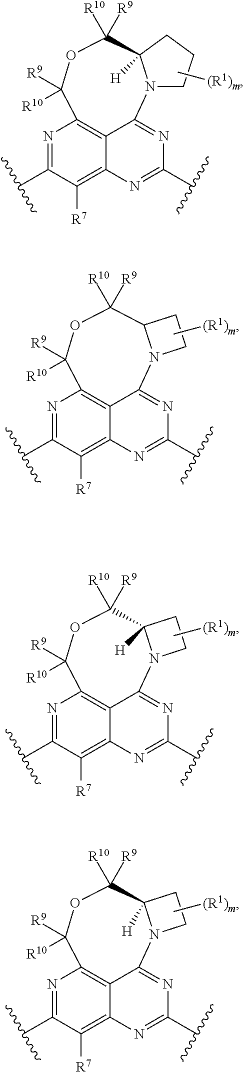

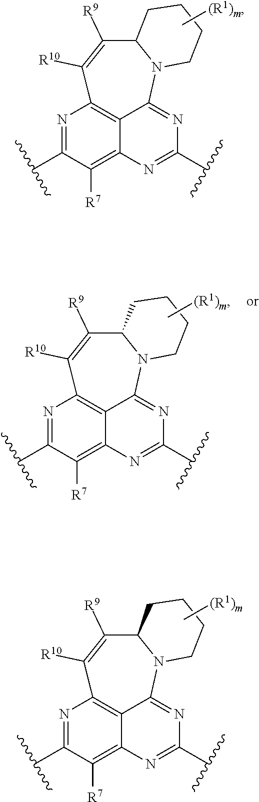

wherein each “ [symbol] ” is a point of covalent attachment to either ring A or —(X)ₚ—R³.

In some embodiments, the compound comprises a core having the formula

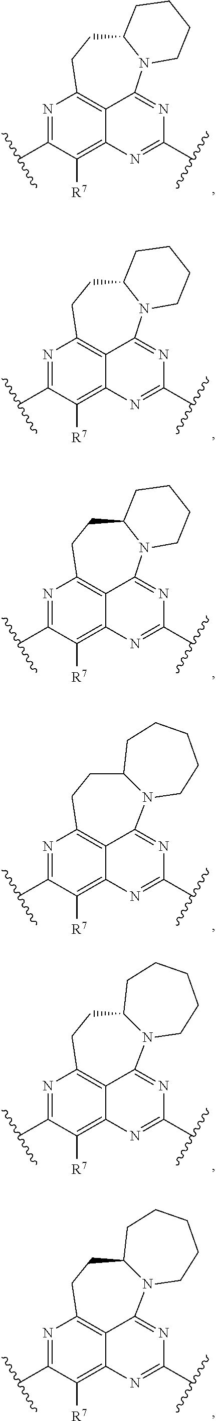

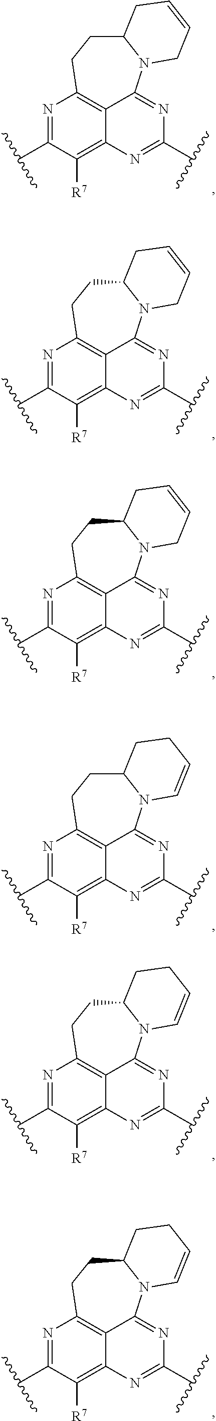

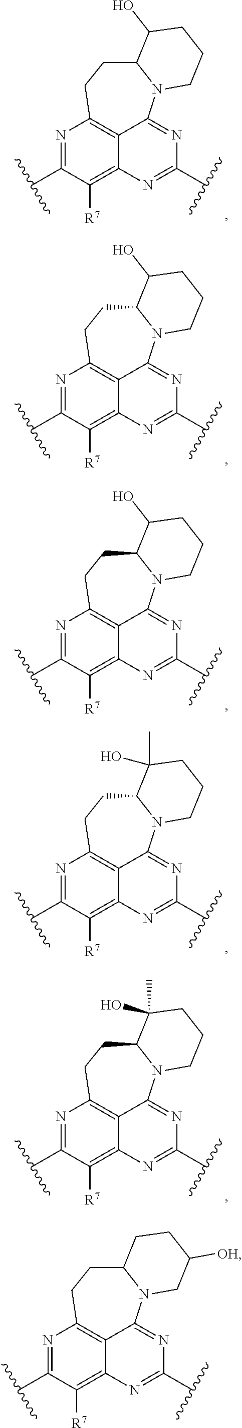

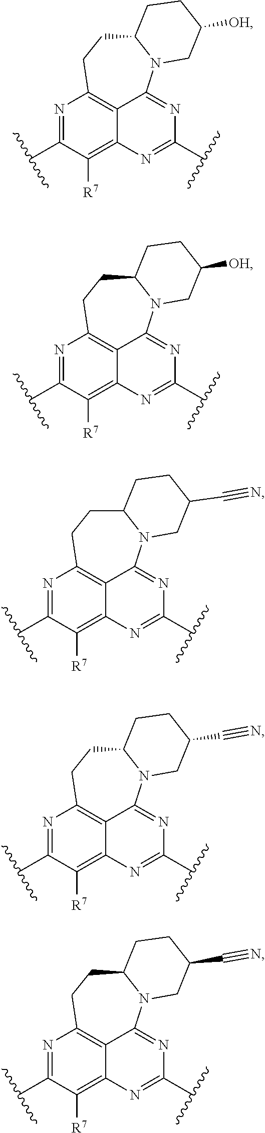

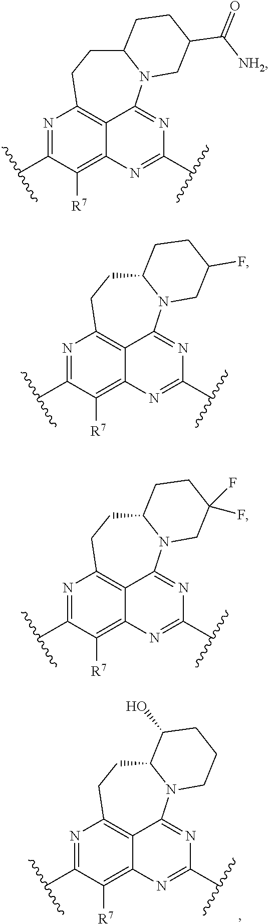

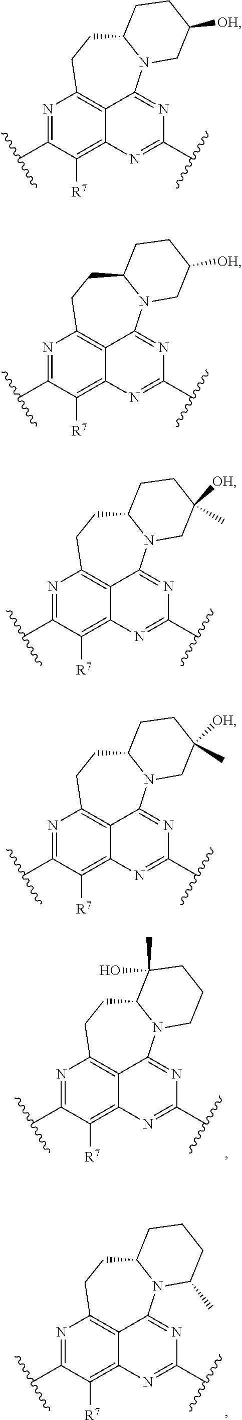

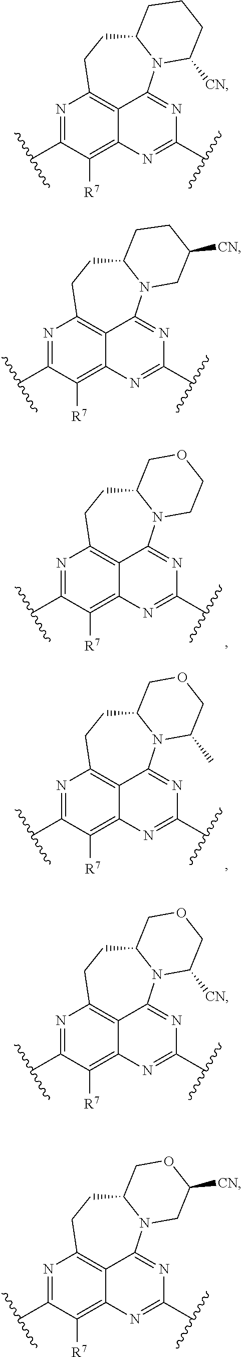

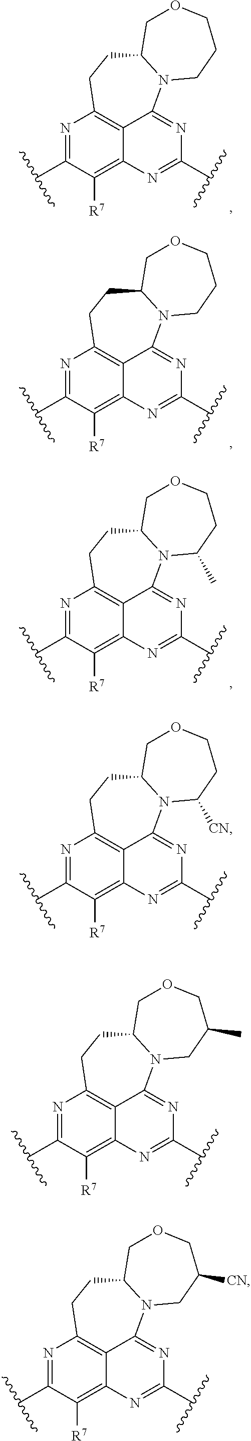

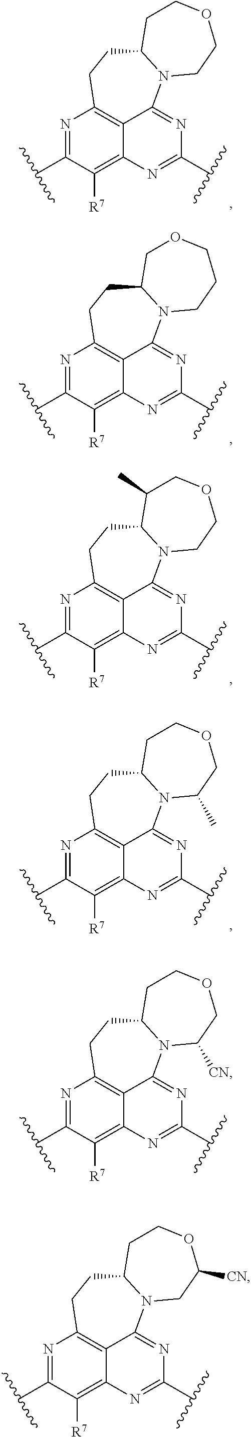

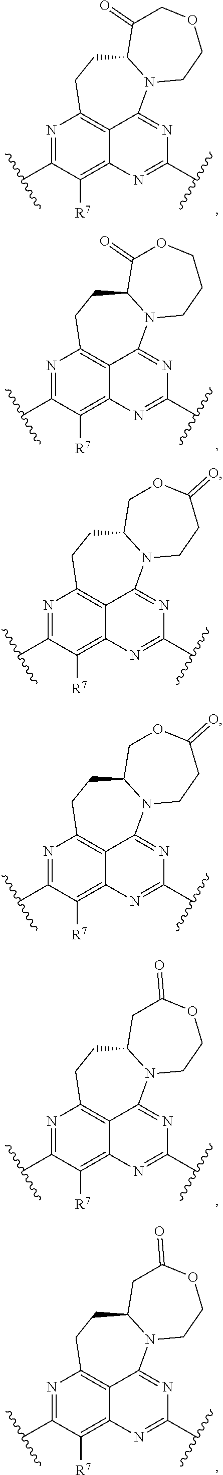

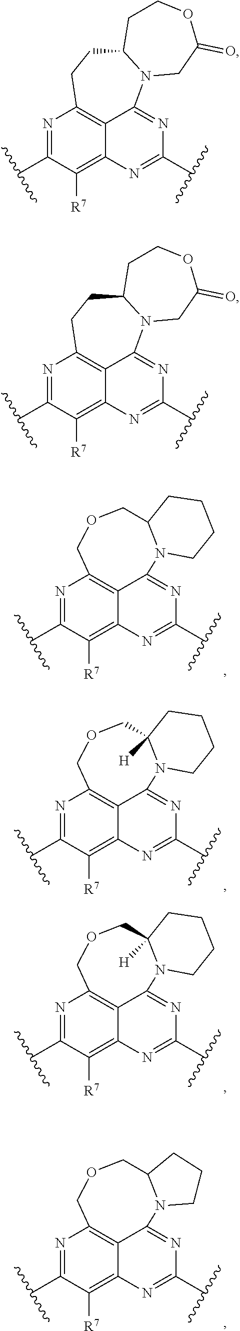

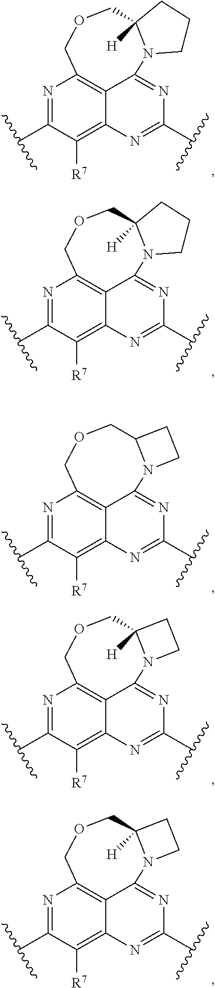

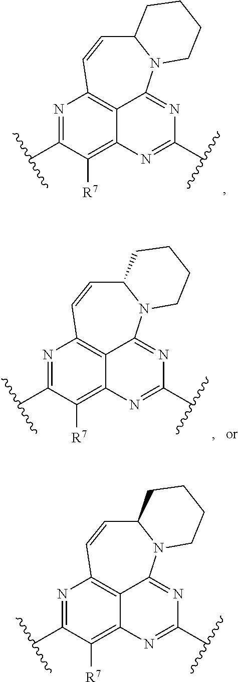

wherein each “ [symbol] ” is a point of covalent attachment to either ring A or —(X)ₚ—R³.

In some embodiments, the compound comprises a core having the formula

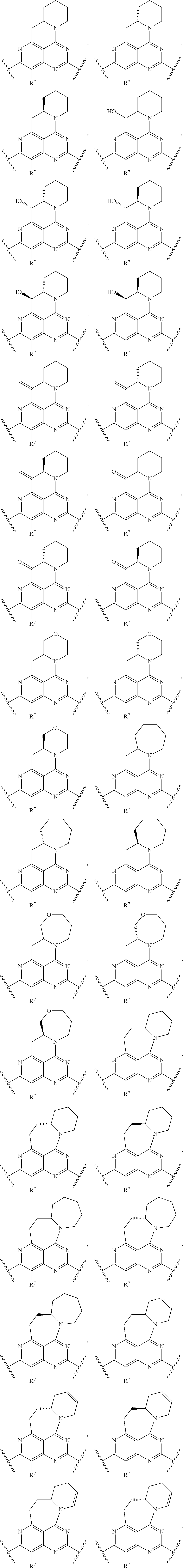

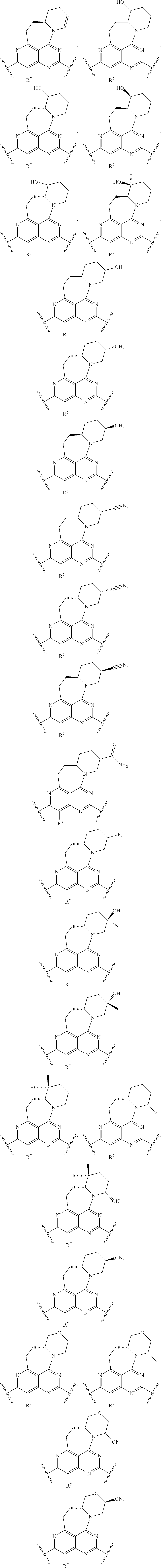

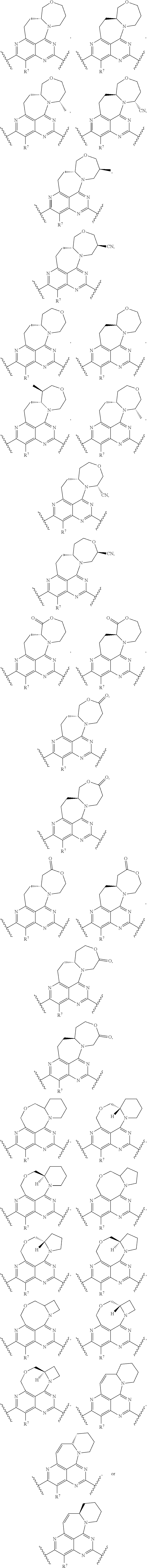

wherein each “ [symbol] ” is a point of covalent attachment to either ring A or —(X)ₚ—R³.

In some embodiments, the compound comprises a core having the formula

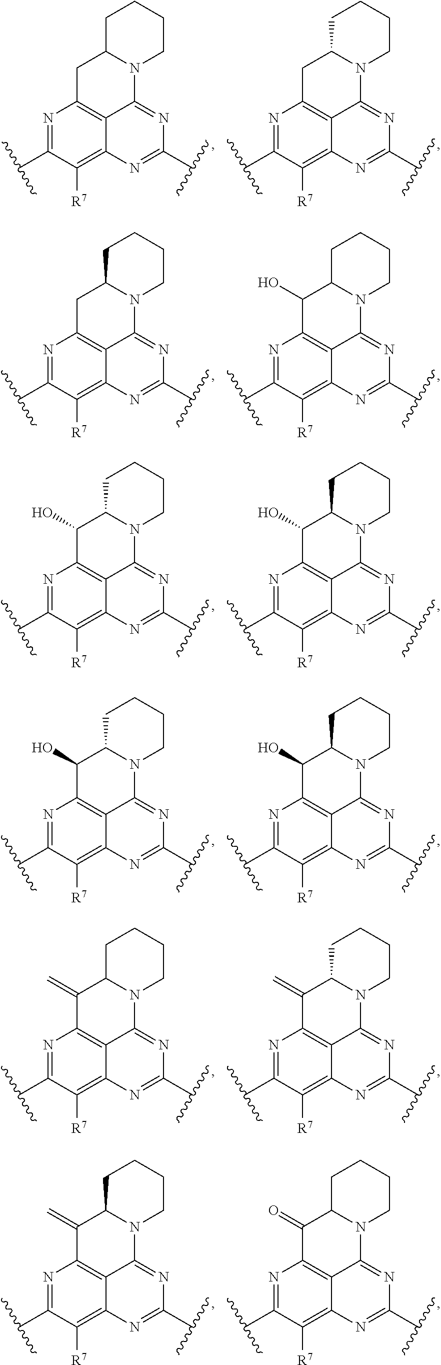

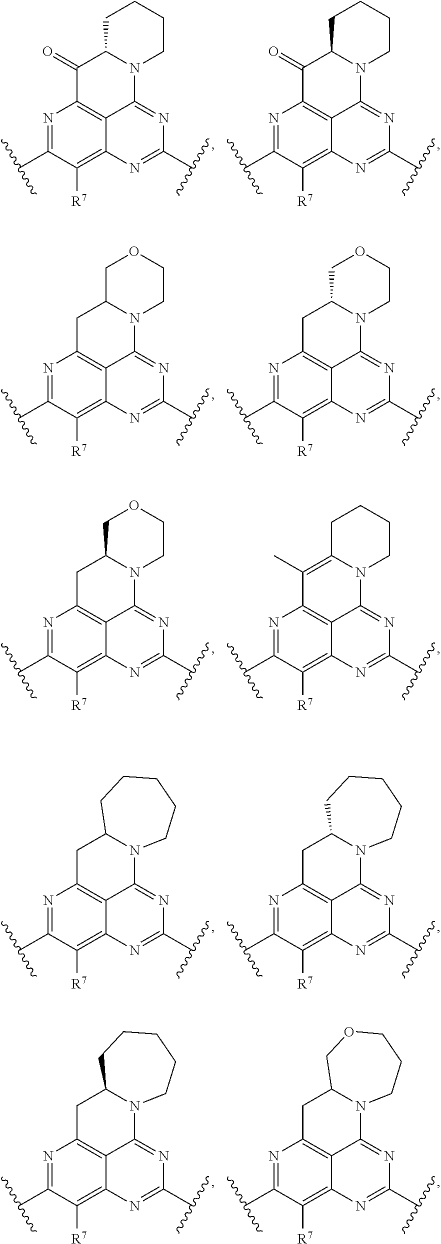

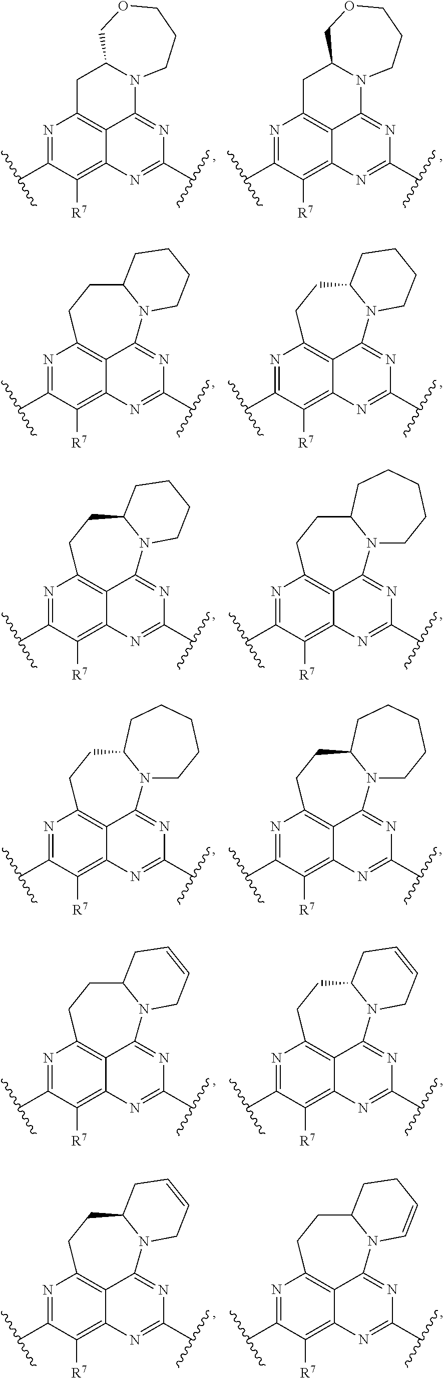

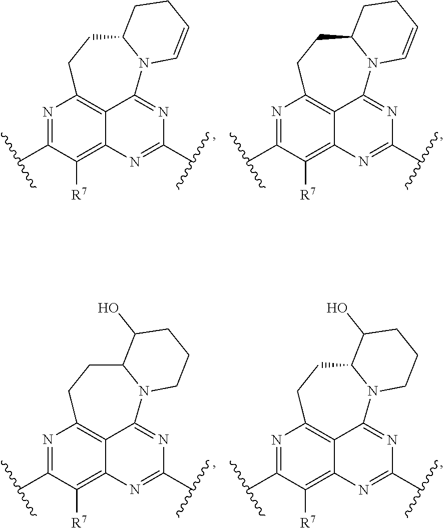

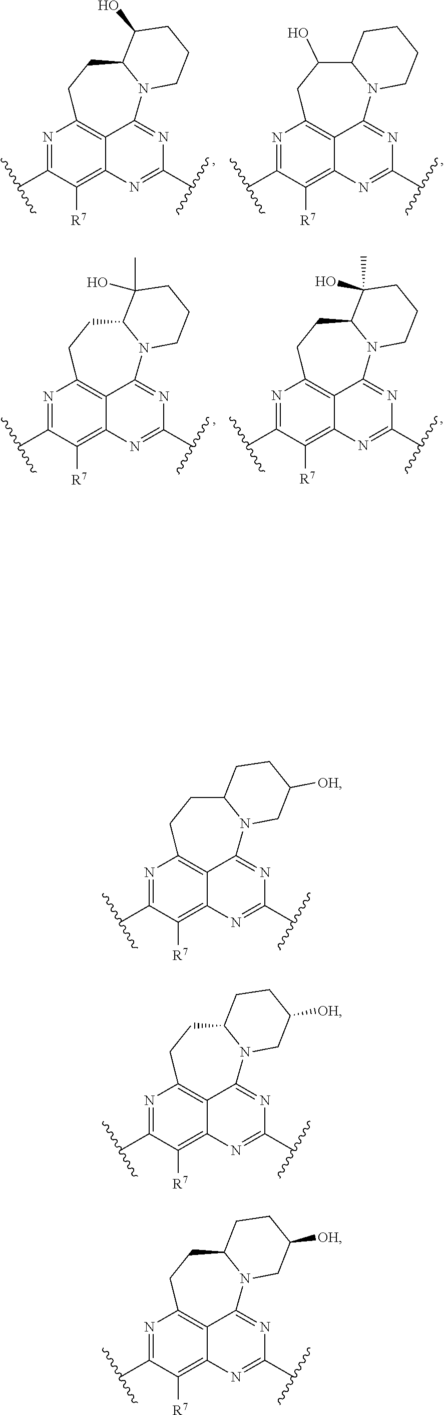

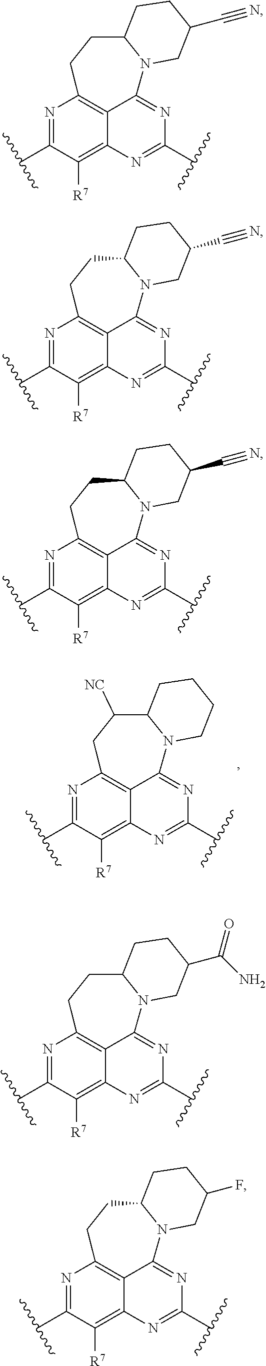

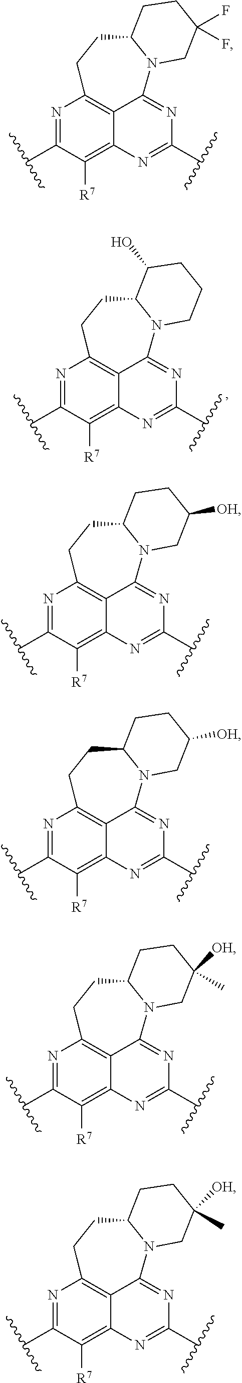

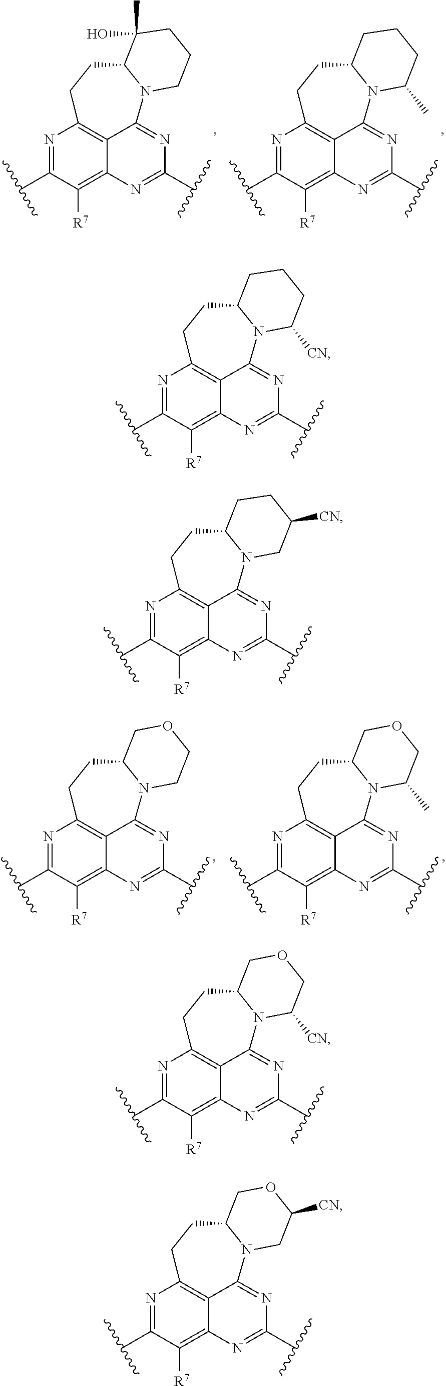

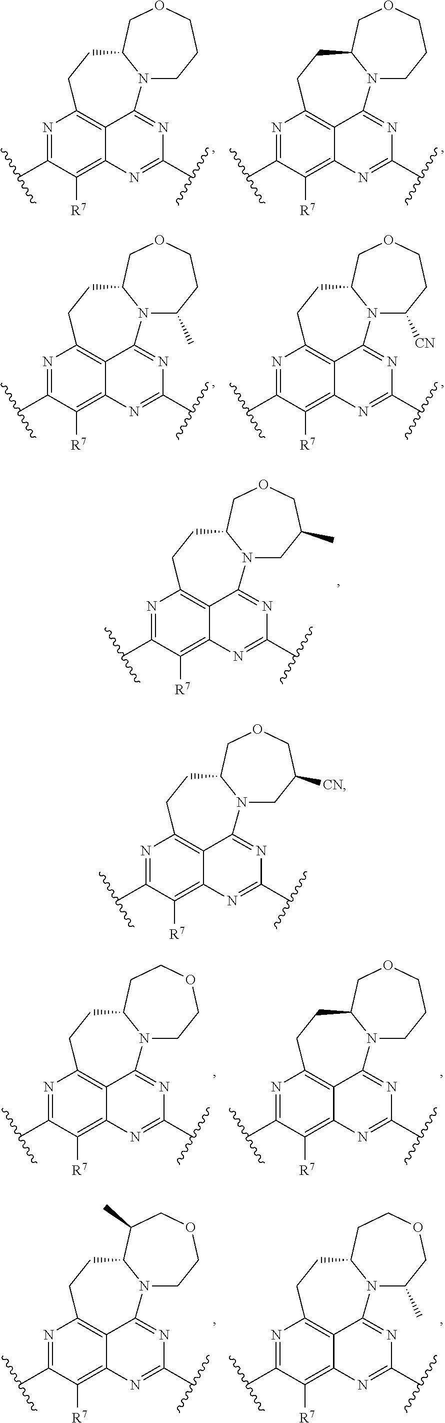

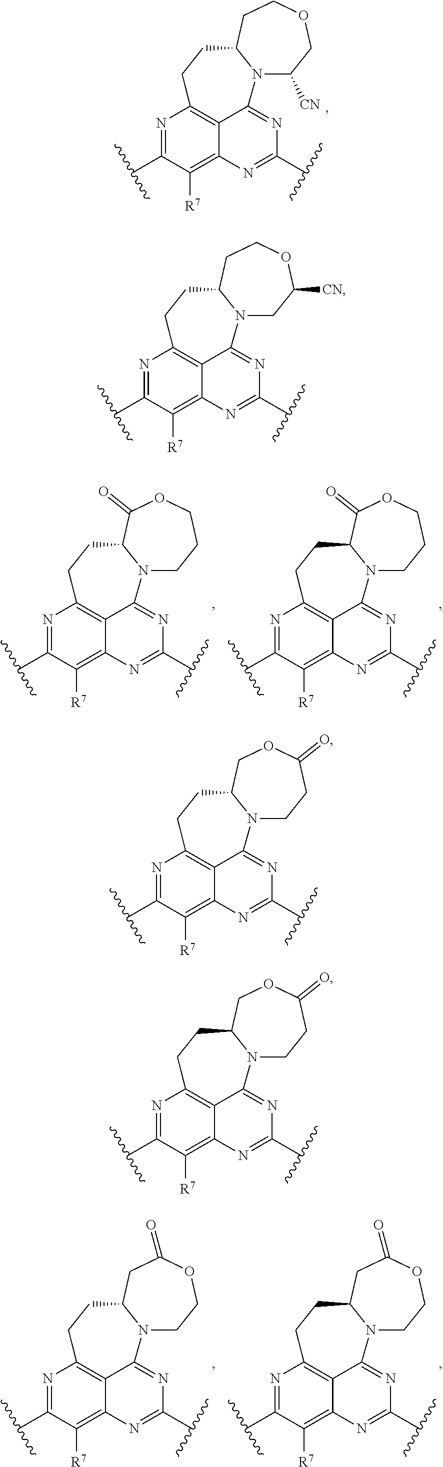

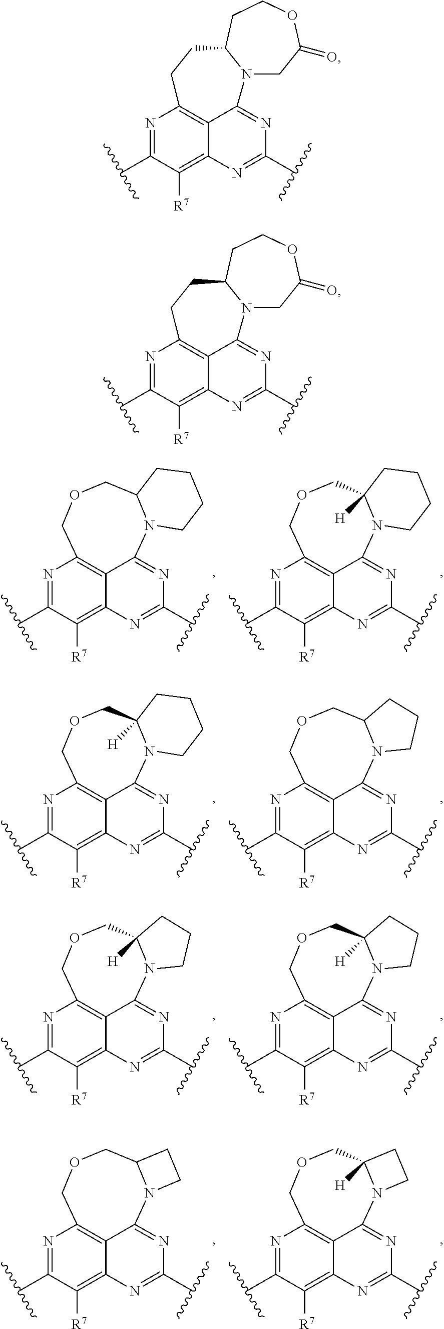

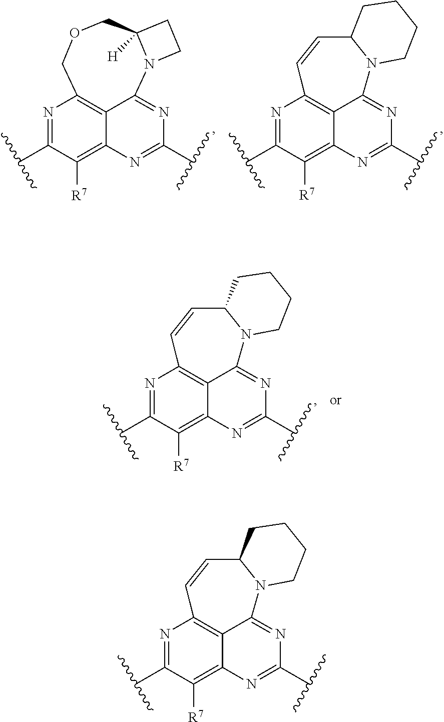

wherein each “ [symbol] ” is a point of covalent attachment to either ring A or —(X)ₚ—R³.

In some embodiments, ring A is a C₆-C₁₀ aryl or a 5- to 10-membered heteroaryl. In some embodiments, ring A is a C₆-C₁₀ aryl, a 5- to 10-membered heteroaryl, or a 4- to 10-membered heterocycloalkyl. In some embodiments, ring A is a C₆-C₁₀ aryl, a 5- to 10-membered heteroaryl, or

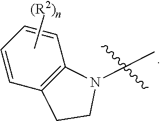

In some embodiments, ring A is a C₆-C₁₀ aryl. In some embodiments, ring A is a 5- to 10-membered heteroaryl. In some embodiments, ring A is

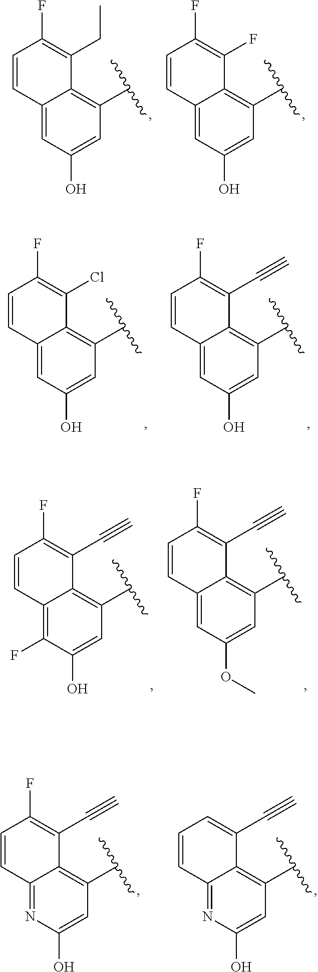

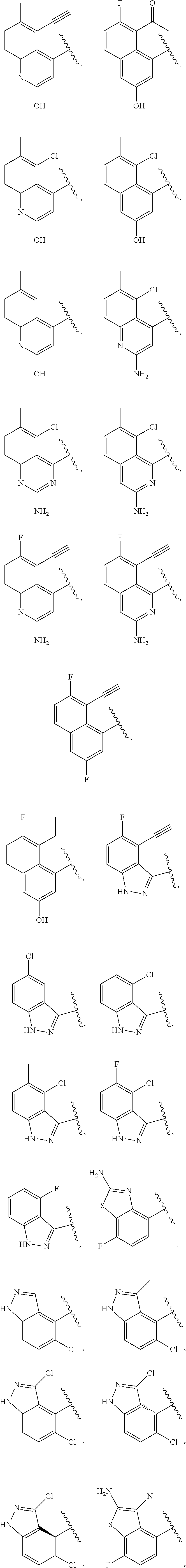

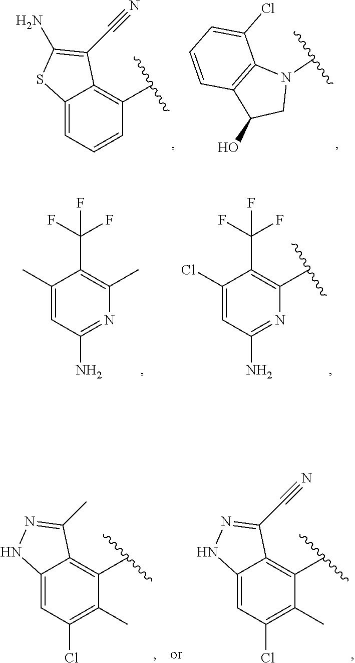

wherein “ [symbol] ” is a point of covalent attachment.

In some embodiments, ring A is

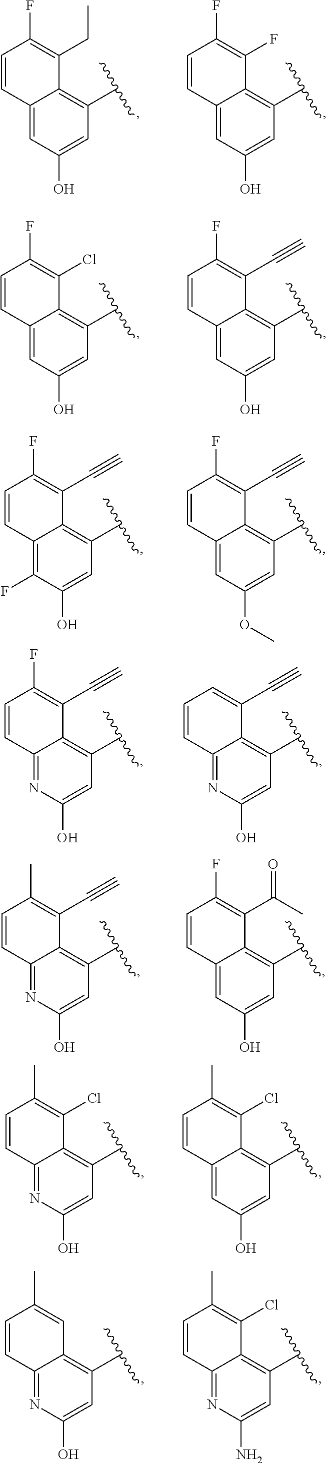

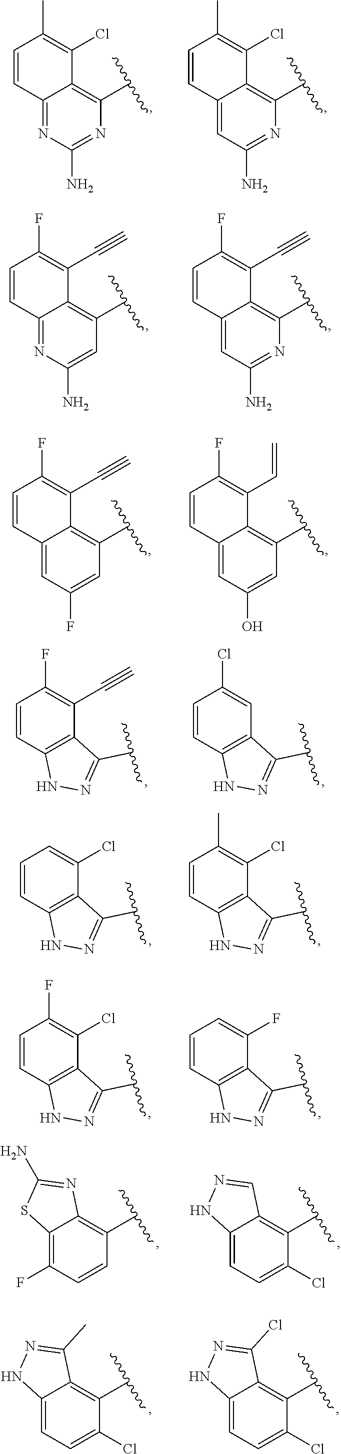

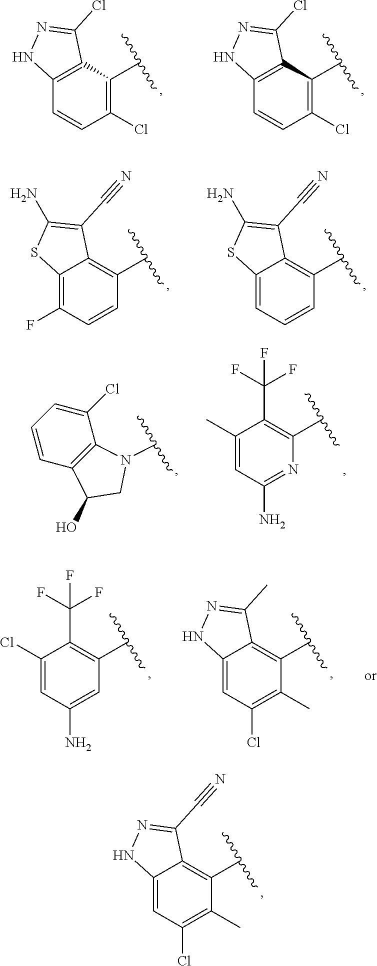

wherein “ [symbol] ” is a point of covalent attachment.

In some embodiments, X is a —O—, —S—, or —NR⁴—. In some embodiments, X is —O—. In some embodiments, X is —NR⁴—. In some embodiments X is —S—.

In some embodiments, Y is bond, —O—, —S—, —S(O)—, or —S(O)₂—.

In some embodiments, Z¹ is N or C(R′). In some embodiments, Z¹ is N.

In some embodiments, Z² is N or C(R⁶). In some embodiments, Z² is N.

In some embodiments, Z³ is N or C(R⁷). In some embodiments, Z³ is CR⁷.

In some embodiments, Z⁴ is N or C(R⁸). In some embodiments, Z⁴ is N.

In some embodiments, Z³ is CR⁷ and Z⁴ is N. In some embodiments, Z³ is CR⁷, and Z⁴ is CR⁸. In some embodiments, Z³ is N, and Z⁴ is CR⁸. In some embodiments, Z³ is N, and Z⁴ is N. In some embodiments, Z³ is N, Z⁴ is CR⁸, Z⁵ is CR¹, and Z⁶ is CR¹². In some embodiments, Z³ is CR⁷, Z⁴ is N, Z⁵ is CR¹¹, and Z⁶ is CR¹². In some embodiments, Z³ is N, Z⁴ is N, Z⁵ is CR¹¹, and Z⁶ is CR¹².

In some embodiments, at least two of Z³-Z⁴ are N, for example,

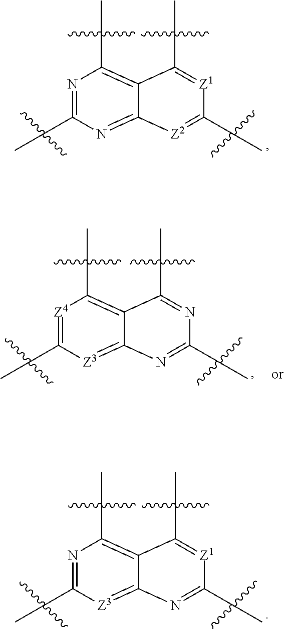

In some embodiments, three of Z¹-Z⁴ are N, for example,

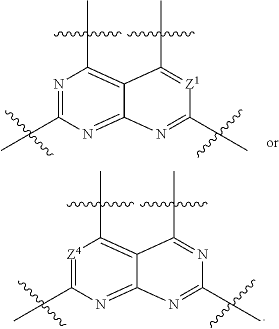

In some embodiments, at least Z⁴ is N, for example,

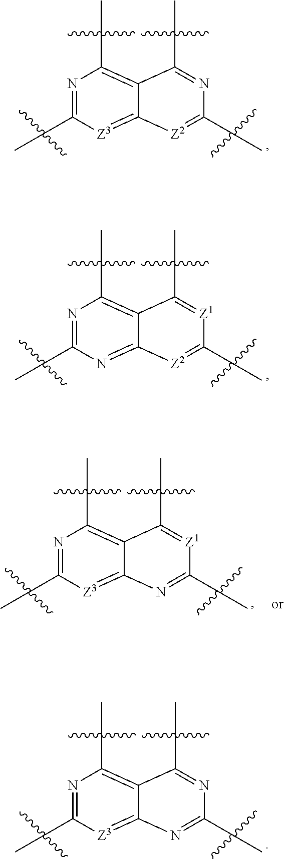

In some embodiments, each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each R¹ is independently deuterium, halogen, C₁-C₆ alkyl, C₂-C₆alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{c}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R¹ and an R⁹ or an R¹⁰ on an adjacent carbon to ring B, taken together with the carbon atoms to which they are attached, combine form a carbon-carbon double bond.

In some embodiments, each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{e}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NRC(O)OR^{f}, —NR^{e}C(O)NR^{e}R^{f}, —NRS(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, each R² is independently deuterium, halogen, C₁-C₆ alkyl, C₂-C₆ alkenyl, C₂-C₆ alkynyl, —OR^{c}, —C(O)R^{c}, —NR^{c}R^{d}, or —CN, wherein each hydrogen atom in C₁-C₆ alkyl, C₂-C₆ alkenyl, and C₂-C₆ alkynyl is independently optionally substituted with a deuterium or halogen. In some embodiments, each R² is independently halogen, C₁-C₆ alkyl, C₂-C₆ alkenyl, C₂-C₆ alkynyl, or —OR^{c}. In some embodiments, each R² is independently halogen, C₁-C₆ alkyl, C₂-C₆ alkynyl, or —OR^{c}. In some embodiments, R² is fluoro. In some embodiments, R² is ethyl. In some embodiments, R² is ethynyl. In some embodiments, R² is —OH.

In some embodiments, R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R³ is —C₁-C₆ alkyl, 4- to 10-membered heterocycloalkyl, or —C₁-C₆ alkylene-(4- to 10-membered heterocycloalkyl), wherein each hydrogen atom in —C₁-C₆ alkyl, 4- to 10-membered heterocycloalkyl, or —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-OR^{a}, C₆-C₁₀ aryl, —C₁-C₆alkylene-(C₆-C₁₀ aryl), haloalkyl, C₃-C₆cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e′}, —CN, or —NO₂. In some embodiments, R³ is —C₁-C₆ alkylene-(4- to 10-membered heterocycloalkyl), wherein each hydrogen atom in —C₁-C₆ alkylene-(4- to 10-membered heterocycloalkyl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R³ is -methylene-(4- to 10-membered heterocycloalkyl), wherein each hydrogen atom in -methylene-(4- to 10-membered heterocycloalkyl) is independently optionally substituted by halogen. In some embodiments, R³ is

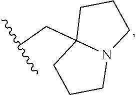 [SMILES: *CC12CCCN1CCC2]

wherein each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e′}, —CN, or —NO₂, and “ [symbol] ” is a point of covalent attachment. In some embodiments R³ is

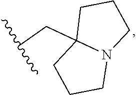 [SMILES: *CC12CCCN1CCC2]

wherein each hydrogen atom is independently optionally substituted by halogen, wherein “ [symbol] ” is a point of covalent attachment. In some embodiments, R³ is

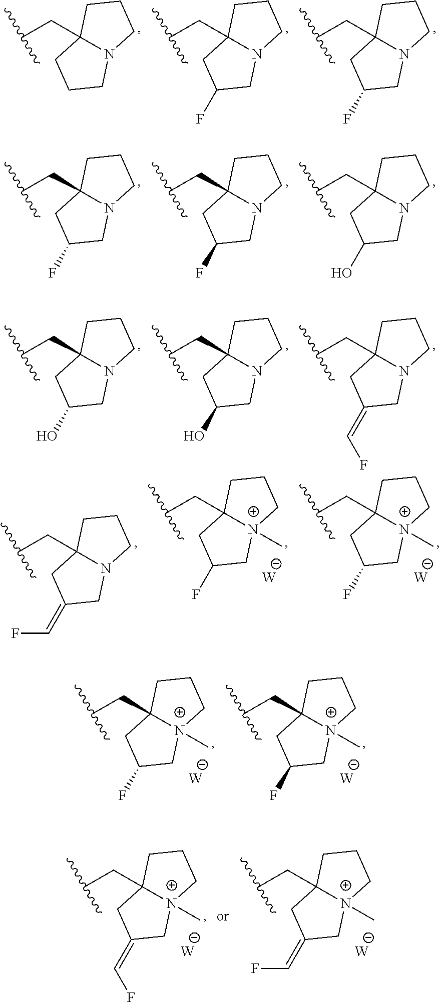

wherein W^{⊖} is an inorganic counter ion or an organic counter ion.

In some embodiments, R³ is 4- to 10-membered heterocycloalkyl, wherein each hydrogen atom in 4- to 10-membered heterocycloalkyl is independently optionally substituted by deuterium, halogen, C₁-C₆alkyl, —C₁-C₆ alkylene-O—C₁-C₆alkyl, —OC₁-C₆alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e′}, —CN, or —NO₂. In some embodiments, R³ is

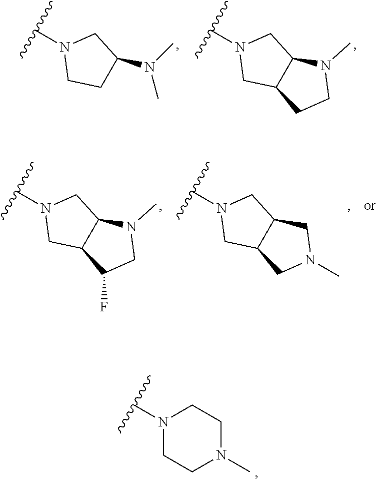

wherein “ [symbol] ” is a point of covalent attachment.

In some embodiments, R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; In some embodiments, R⁴ is H or C₁-C₆ alkyl. In some embodiments, R⁴ is H or methyl. In some embodiments, R⁴ is H.

In some embodiments, each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂. In some embodiments, R⁵ is H or deuterium. In some embodiments, R⁶ is H or deuterium. In some embodiments, R⁷ is H, deuterium, or F. In some embodiments, R⁸ is H or deuterium.

In some embodiments, each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or R⁹ and R¹⁰, taken together with the carbon atom to which they are attached, form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰. In some embodiments, R⁹ is H, deuterium, or methyl. In some embodiments, R^{1O} is H, deuterium, or methyl. In some embodiments, R⁹ and R¹⁰ are H or deuterium. In some embodiments, an R⁹ and an R¹⁰ on adjacent carbon atoms, taken together with the carbon atoms to which they are attached, combine form a carbon-carbon double bond substituted by an R⁹ and an R¹⁰.

In some embodiments, each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or CJ-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl.

In some embodiments, m is 0, 1, 2, 3, 4, 5, 6, or 7. In some embodiments, m is 1, 2, 3, or 4. In some embodiments, m is 0, 1, 2, or 3. In some embodiments, m is 0, 1, or 2. In some embodiments, m is 0 or 1. In some embodiments, m is 0. In some embodiments, m is 1. In some embodiments, m is 2. In some embodiments, m is 3. In some embodiments, m is 4.

In some embodiments, n is 0, 1, 2, 3, 4, 5, 6, or 7. In some embodiments, n is 0, 1, 2, or 3. In some embodiments, n is 1, 2, 3, or 4. In some embodiments, n is 1, 2, or 3. In some embodiments, n is 0. In some embodiments, n is 1. In some embodiments, n is 2. In some embodiments, n is 3. In some embodiments, n is 4.

In some embodiments, p is 0 or 1. In some embodiments, p is 0. In some embodiments, p is 1.

In some embodiments, q is 0, 1, or 2. In some embodiments, q is 0. In some embodiments, q is 1. In some embodiments, q is 2.

In some embodiments, q1 is 0 or 1. In some embodiments, q1 is 0. In some embodiments, q1 is 1.

In some embodiments, t is 0, 1, 2 or 3. In some embodiments, t is 0, 1, or 2. In some embodiments, t is 0 or 1. In some embodiments, t is 1 or 2. In some embodiments, t is 0. In some embodiments, t is 1. In some embodiments, t is 2.

In some embodiments, v is 0, 1, or 2. In some embodiments, v is 0. In some embodiments, v is 1. In some embodiments, v is 2. In some embodiments, v is 1 or 2. In some embodiments, v is 0 or 1.

In some embodiments, X¹ is —O—, —S—, —S(O)—, —S(O)₂—, —NR¹³—, —C(O)NR¹³—, —NR¹³C(O)—, —C(O)O—, —OC(O)—, or —C(R¹)₂—. In some embodiments, X¹ is —O—, —S—, —S(O)—, —S(O)₂—, —NR¹³—, —C(O)NR¹³—, —NR¹³C(O)—, —C(O)O—, —OC(O)—, or —C(R¹)₂—, q, when present is 0, and q1, when present is 0. In some embodiments, X¹ is —O—, —S—, —S(O)—, —S(O)₂—, —C(O)O—, —OC(O)—, or —C(R¹)₂—. In some embodiments, X¹ is —O—, —S—, —S(O)—, —S(O)₂—, or —C(R¹)₂—.

In some embodiments, R¹¹ is H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e′}, —CN, or —NO₂; or two hydrogen atoms on a single carbon atom of the 4- to 10-membered heterocycloalkyl combine to form an oxo group or a C₂-C₆ alkenyl group. In some embodiments, R¹¹ is H, deuterium, or methyl.

In some embodiments, Z⁵ is N or C(R¹¹). In some embodiments, Z⁵ is N. In some embodiments, Z⁵ is CR¹¹.

In some embodiments, Z⁶ is N or C(R¹²). In some embodiments, Z⁶ is N. In some embodiments, Z⁶ is CR¹².

In some embodiments, each of R¹¹ and R¹² is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-Clo aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂. In some embodiments, R¹¹ is H or deuterium. In some embodiments, R¹² is H or deuterium.

In some embodiments, R¹³ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, the compound is selected from the group consisting of

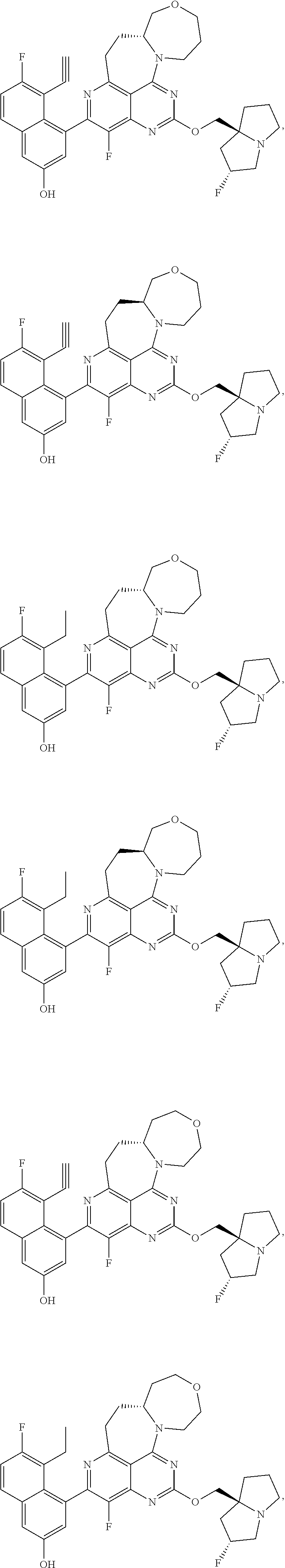

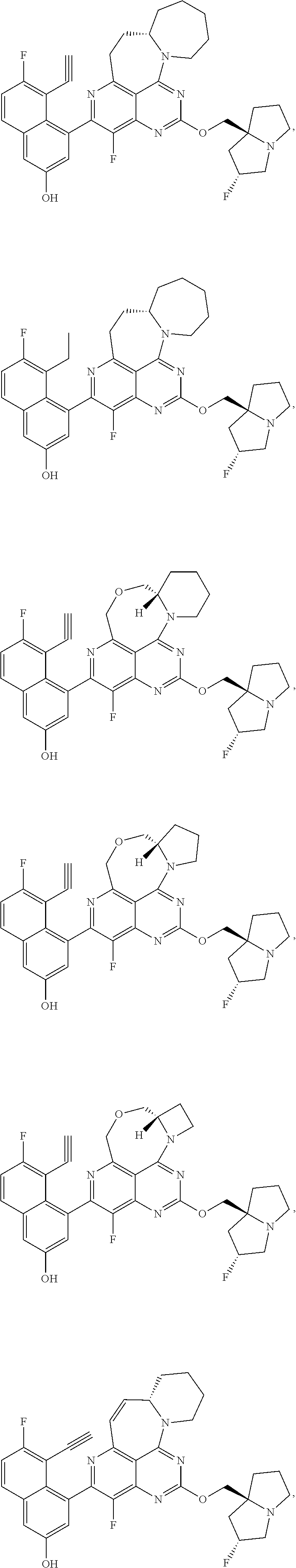

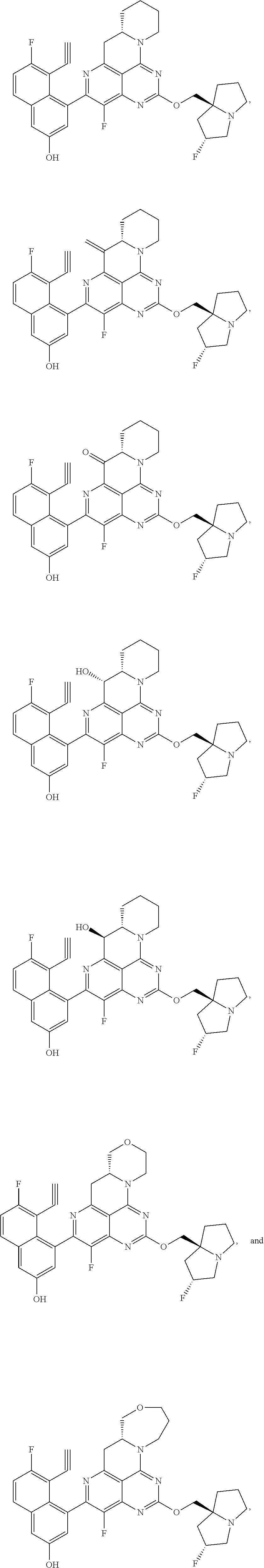

or a pharmaceutically acceptable salt thereof.

The following represent illustrative embodiments of compounds of Formula (I)-(LXXXI):

| Ex | Structure | Name |
|---|---|---|
| 1 | 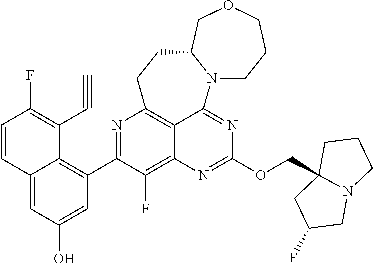 [SMILES: C#Cc1c(F)ccc2cc(O)cc(-c3nc4c5c(nc(OC[C@@]67CCCN6C[C@H](F)C7)nc5c3F)N3CCCOC[C@H]3CC4)c12] | 5-ethynyl-6-fluoro-4-[(8aR)-4-fluoro-2- {[(2R,7aS)-2-fluorotetrahydro-1H- pyrrolizin-7a(5H)-yl]methoxy}- 7,8,8a,9,12,13-hexahydro-11H-10-oxa- 1,3,6,13a-tetraazanaphtho[1,8-ab]heptalen- 5-yl]naphthalen-2-ol |
| 2 | 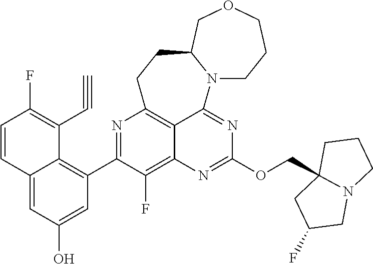 [SMILES: C#Cc1c(F)ccc2cc(O)cc(-c3nc4c5c(nc(OC[C@@]67CCCN6C[C@H](F)C7)nc5c3F)N3CCCOC[C@@H]3CC4)c12] | 5-ethynyl-6-fluoro-4-[(8aS)-4-fluoro-2- {[(2R,7aS)-2-fluorotetrahydro-1H- pyrrolizin-7a(5H)-yl]methoxy}- 7,8,8a,9,12,13-hexahydro-11H-10-oxa- 1,3,6,13a-tetraazanaphtho[1,8-ab]heptalen- 5-yl]naphthalen-2-ol |
| 3 | 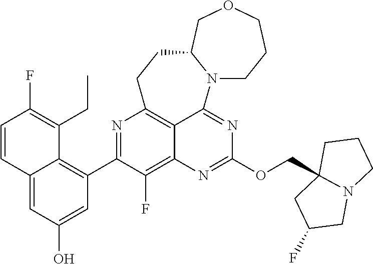 [SMILES: CCc1c(F)ccc2cc(O)cc(-c3nc4c5c(nc(OC[C@@]67CCCN6C[C@H](F)C7)nc5c3F)N3CCCOC[C@H]3CC4)c12] | 5-ethyl-6-fluoro-4-[(8aR)-4-fluoro-2- {[(2R,7aS)-2-fluorotetrahydro-1H- pyrrolizin-7a(5H)-yl]methoxy}- 7,8,8a,9,12,13-hexahydro-11H-10-oxa- 1,3,6,13a-tetraazanaphtho[1,8-ab]heptalen- 5-yl]naphthalen-2-ol |
| 4 | 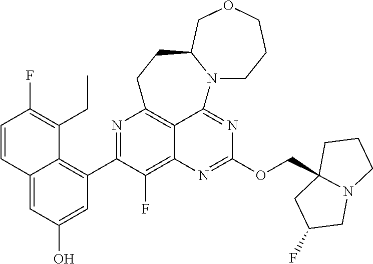 [SMILES: CCc1c(F)ccc2cc(O)cc(-c3nc4c5c(nc(OC[C@@]67CCCN6C[C@H](F)C7)nc5c3F)N3CCCOC[C@@H]3CC4)c12] | 5-ethyl-6-fluoro-4-[(8aS)-4-fluoro-2- {[(2R,7aS)-2-fluorotetrahydro-1H- pyrrolizin-7a(5H)-yl]methoxy}- 7,8,8a,9,12,13-hexahydro-11H-10-oxa- 1,3,6,13a-tetraazanaphtho[1,8-ab]heptalen- 5-yl]naphthalen-2-ol |
| 5 | 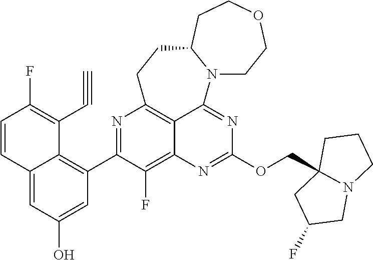 [SMILES: C#Cc1c(F)ccc2cc(O)cc(-c3nc4c5c(nc(OC[C@@]67CCCN6C[C@H](F)C7)nc5c3F)N3CCOCC[C@H]3CC4)c12] | 5-ethynyl-6-fluoro-4-[(8aR)-4-fluoro-2- {[(2R,7aS)-2-fluorotetrahydro-1H- pyrrolizin-7a(5H)-yl]methoxy}- 8,8a,9,10,12,13-hexahydro-7H-11-oxa- 1,3,6,13a-tetraazanaphtho[1,8-ab]heptalen- 5-yl]naphthalen-2-ol |
| 6 | 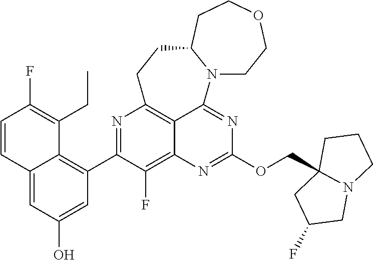 [SMILES: CCc1c(F)ccc2cc(O)cc(-c3nc4c5c(nc(OC[C@@]67CCCN6C[C@H](F)C7)nc5c3F)N3CCOCC[C@H]3CC4)c12] | 5-ethyl-6-fluoro-4-[(8aR)-4-fluoro-2- {[(2R,7aS)-2-fluorotetrahydro-1H- pyrrolizin-7a(5H)-yl]methoxy}- 8,8a,9,10,12,13-hexahydro-7H-11-oxa- 1,3,6,13a-tetraazanaphtho[1,8-ab]heptalen- 5-yl]naphthalen-2-ol |
| 7 | 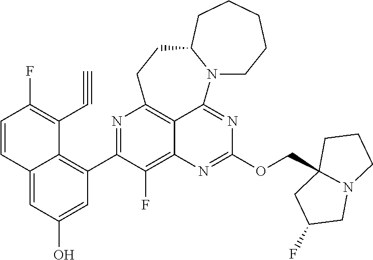 [SMILES: C#Cc1c(F)ccc2cc(O)cc(-c3nc4c5c(nc(OC[C@@]67CCCN6C[C@H](F)C7)nc5c3F)N3CCCCC[C@H]3CC4)c12] | 5-ethynyl-6-fluoro-4-[(8aS)-4-fluoro-2- {[(2R,7aS)-2-fluorotetrahydro-1H- pyrrolizin-7a(5H)-yl]methoxy}- 7,8,8a,9,10,11,12,13-octahydro-1,3,6,13a- tetraazanaphtho[1,8-ab]heptalen-5- yl]naphthalen-2-ol |
| 8 | 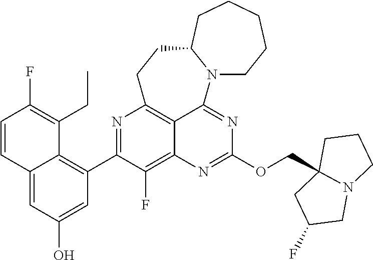 [SMILES: CCc1c(F)ccc2cc(O)cc(-c3nc4c5c(nc(OC[C@@]67CCCN6C[C@H](F)C7)nc5c3F)N3CCCCC[C@H]3CC4)c12] | 5-ethyl-6-fluoro-4-[(8aS)-4-fluoro-2- {[(2R,7aS)-2-fluorotetrahydro-1H- pyrrolizin-7a(5H)-yl]methoxy}- 7,8,8a,9,10,11,12,13-octahydro-1,3,6,13a- tetraazanaphtho[1,8-ab]heptalen-5- yl]naphthalen-2-ol |
| 9 | 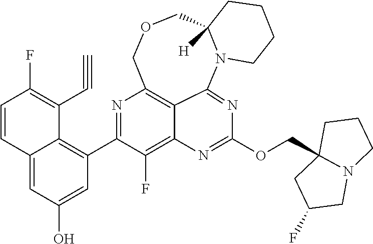 | 5-ethynyl-6-fluoro-4-[(6aS)-1-fluoro-12- {[(2R,7aS)-2-fluorotetrahydro-1H- pyrrolizin-7a(5H)-yl]methoxy}- 6,6a,7,8,9,10-hexahydro-4H-5-oxa- 3,10a,11,13- tetraazabenzo[4,5]cycloocta[1,2,3- de]naphthalen-2-yl]naphthalen-2-ol |
| 10 | 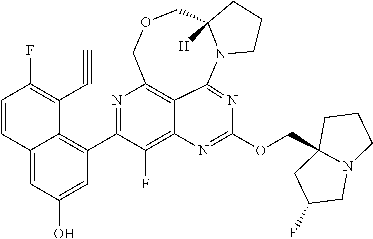 | 5-ethynyl-6-fluoro-4-[(6aS)-1-fluoro-11- {[(2R,7aS)-2-fluorotetrahydro-1H- pyrrolizin-7a(5H)-yl]methoxy}-6a,7,8,9- tetrahydro-4H,6H-5-oxa-3,9a,10,12- tetraazacyclopenta[4,5]cycloocta[1,2,3- de]naphthalen-2-yl]naphthalen-2-ol |
| 11 | 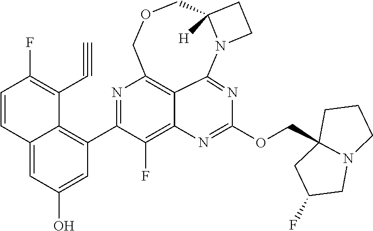 | 5-ethynyl-6-fluoro-4-[(6aS)-1-fluoro-10- {[(2R,7aS)-2-fluorotetrahydro-1H- pyrrolizin-7a(5H)-yl]methoxy}-6,6a,7,8- tetrahydro-4H-5-oxa-3,8a,9,11- tetraazacyclobuta[4,5]cycloocta[1,2,3- de]naphthalen-2-yl]naphthalen-2-ol |
| 12 | 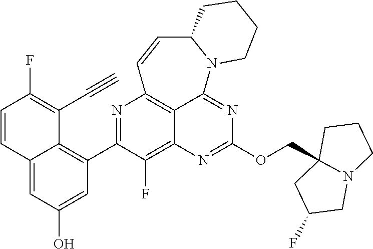 [SMILES: C#Cc1c(F)ccc2cc(O)cc(-c3nc4c5c(nc(OC[C@@]67CCCN6C[C@H](F)C7)nc5c3F)N3CCCC[C@H]3C=C4)c12] | 5-ethynyl-6-fluoro-4-((S)-1-fluoro-11- (((2R,7aS)-2-fluorotetrahydro-1H- pyrrolizin-7a(5H)-yl)methoxy)-5a,6,8,9- tetrahydro-7H-3,9a,10,12- tetraazabenzo[4,5]cyclohepta[1,2,3- de]naphthalen-2-yl)naphthalen-2-ol |
| 13 | 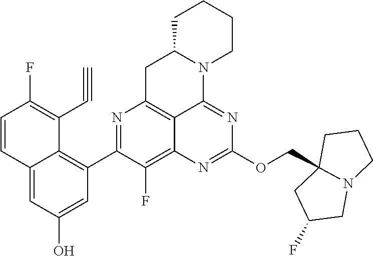 [SMILES: C#Cc1c(F)ccc2cc(O)cc(-c3nc4c5c(nc(OC[C@@]67CCCN6C[C@H](F)C7)nc5c3F)N3CCCC[C@H]3C4)c12] | 5-ethynyl-6-fluoro-4-((S)-4-fluoro-2- (((2R,7aS)-2-fluorotetrahydro-1H- pyrrolizin-7a(5H)-yl)methoxy)- 7,7a,8,9,10,11-hexahydropyrido[1,2- g]pyrimido[4,5,6-de][1,6]naphthyridin-5- yl)naphthalen-2-ol |
| 14 | 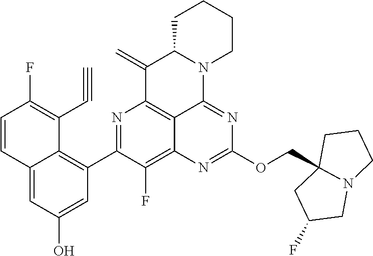 [SMILES: C#Cc1c(F)ccc2cc(O)cc(-c3nc4c5c(nc(OC[C@@]67CCCN6C[C@H](F)C7)nc5c3F)N3CCCC[C@H]3C4=C)c12] | 5-ethynyl-6-fluoro-4-((S)-4-fluoro-2- (((2R,7aS)-2-fluorotetrahydro-1H- pyrrolizin-7a(5H)-yl)methoxy)-7- methylene-7,7a,8,9,10,11- hexahydropyrido[1,2-g]pyrimido[4,5,6- de][1,6]naphthyridin-5-yl)naphthalen-2- ol |
| 15 | 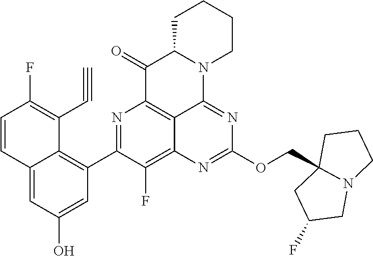 [SMILES: C#Cc1c(F)ccc2cc(O)cc(-c3nc4c5c(nc(OC[C@@]67CCCN6C[C@H](F)C7)nc5c3F)N3CCCC[C@H]3C4=O)c12] | (S)-5-(8-ethynyl-7-fluoro-3- hydroxynaphthalen-1-yl)-4-fluoro-2- (((2R,7aS)-2-fluorotetrahydro-1H- pyrrolizin-7a(5H)-yl)methoxy)-8,9,10,11- tetrahydropyrido[1,2-g]pyrimido[4,5,6- de][1,6]naphthyridin-7(7aH)-one |
| 16 | 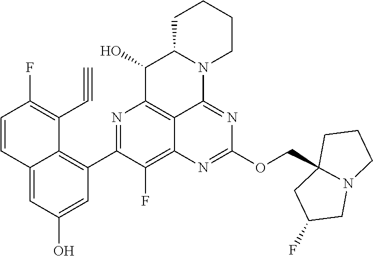 [SMILES: C#Cc1c(F)ccc2cc(O)cc(-c3nc4c5c(nc(OC[C@@]67CCCN6C[C@H](F)C7)nc5c3F)N3CCCC[C@H]3[C@H]4O)c12] | (7R,7aS)-5-(8-ethynyl-7-fluoro-3- hydroxynaphthalen-1-yl)-4-fluoro-2- (((2R,7aS)-2-fluorotetrahydro-1H- pyrrolizin-7a(5H)-yl)methoxy)- 7,7a,8,9,10,11-hexahydropyrido[1,2- g]pyrimido[4,5,6-de][1,6]naphthyridin-7- ol |
| 17 | 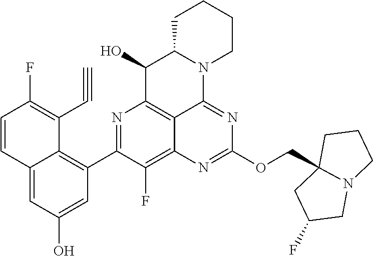 [SMILES: C#Cc1c(F)ccc2cc(O)cc(-c3nc4c5c(nc(OC[C@@]67CCCN6C[C@H](F)C7)nc5c3F)N3CCCC[C@H]3[C@@H]4O)c12] | (7S,7aS)-5-(8-ethynyl-7-fluoro-3- hydroxynaphthalen-1-yl)-4-fluoro-2- (((2R,7aS)-2-fluorotetrahydro-1H- pyrrolizin-7a(5H)-yl)methoxy)- 7,7a,8,9,10,11-hexahydropyrido[1,2- g]pyrimido[4,5,6-de][1,6]naphthyridin- 7-ol |
| 18 | 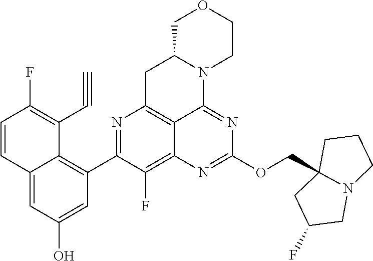 [SMILES: C#Cc1c(F)ccc2cc(O)cc(-c3nc4c5c(nc(OC[C@@]67CCCN6C[C@H](F)C7)nc5c3F)N3CCOC[C@H]3C4)c12] | 5-ethynyl-6-fluoro-4-((R)-4-fluoro-2- (((2R,7aS)-2-fluorotetrahydro-1H- pyrrolizin-7a(5H)-yl)methoxy)-7a,8,10,11- tetrahydro-7H-[1,4]oxazino[4,3- g]pyrimido[4,5,6-de][1,6]naphthyridin- 5-yl)naphthalen-2-ol |
| 19 | 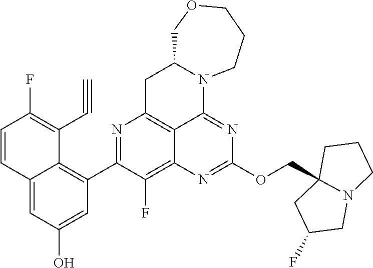 [SMILES: C#Cc1c(F)ccc2cc(O)cc(-c3nc4c5c(nc(OC[C@@]67CCCN6C[C@H](F)C7)nc5c3F)N3CCCOC[C@H]3C4)c12] | 5-ethynyl-6-fluoro-4-((R)-4-fluoro-2- (((2R,7aS)-2-fluorotetrahydro-1H- pyrrolizin-7a(5H)-yl)methoxy)-7a,8,11, 12-tetrahydro-7H,10H-[1,4]oxazepino[4,3- g]pyrimido[4,5,6-de][1,6]naphthyridin- 5-yl)naphthalen-2-ol |
| 20 | 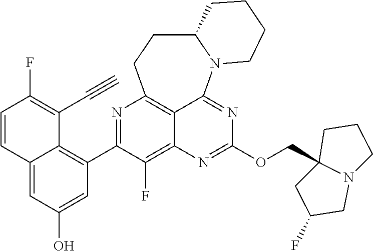 [SMILES: C#Cc1c(F)ccc2cc(O)cc(-c3nc4c5c(nc(OC[C@@]67CCCN6C[C@H](F)C7)nc5c3F)N3CCCC[C@H]3CC4)c12] | 5-ethynyl-6-fluoro-4-[(8aS)-4-fluoro-2- {[(2R,7aS)-2-fluorotetrahydro-1H- pyrrolizin-7a(5H)-yl]methoxy}- 8,8a,9,10,11,12-hexahydro-7H-1,3,6,12a- tetraazabenzo[4,5]cyclohepta[1,2,3- de]naphthalen-5-yl]naphthalen-2-ol |
| 21 | 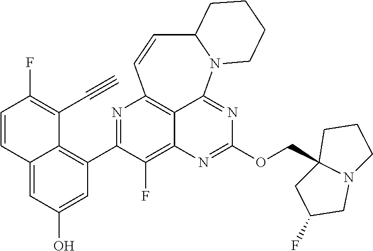 [SMILES: C#Cc1c(F)ccc2cc(O)cc(-c3nc4c5c(nc(OC[C@@]67CCCN6C[C@H](F)C7)nc5c3F)N3CCCCC3C=C4)c12] | 5-ethynyl-6-fluoro-4-(4-fluoro-2- {[(2R,7aS)-2-fluorotetrahydro-1H- pyrrolizin-7a(5H)-yl]methoxy}-9,10,11, 12-tetrahydro-8aH-1,3,6,12a- tetraazabenzo[4,5]cyclohepta[1,2,3- de]naphthalen-5-yl)naphthalen-2-ol |
| 22 | 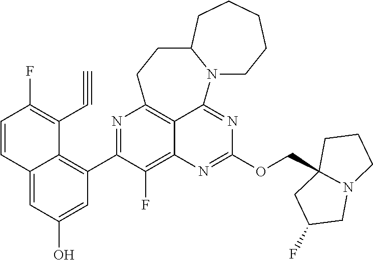 [SMILES: C#Cc1c(F)ccc2cc(O)cc(-c3nc4c5c(nc(OC[C@@]67CCCN6C[C@H](F)C7)nc5c3F)N3CCCCCC3CC4)c12] | 5-ethynyl-6-fluoro-4-(4-fluoro-2- {[(2R,7aS)-2-fluorotetrahydro-1H- pyrrolizin-7a(5H)-yl]methoxy}- 7,8,8a,9,10,11,12,13-octahydro-1,3,6,13a- tetraazanaphtho[1,8-ab]heptalen-5- yl)naphthalen-2-ol |
| 23 | 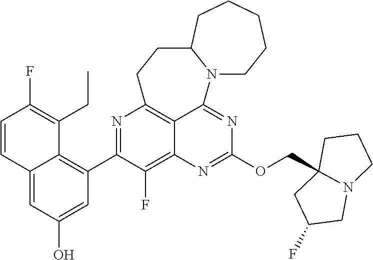 [SMILES: CCc1c(F)ccc2cc(O)cc(-c3nc4c5c(nc(OC[C@@]67CCCN6C[C@H](F)C7)nc5c3F)N3CCCCCC3CC4)c12] | 5-ethyl-6-fluoro-4-(4-fluoro-2-{[(2R,7aS)- 2-fluorotetrahydro-1H-pyrrolizin-7a(5H)- yl]methoxy}-7,8,8a,9,10,11,12,13- octahydro-1,3,6,13a-tetraazanaphtho[1,8- ab]heptalen-5-yl)naphthalen-2-ol |
| 24 | 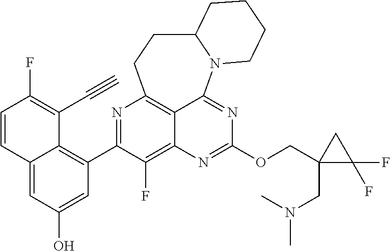 [SMILES: C#Cc1c(F)ccc2cc(O)cc(-c3nc4c5c(nc(OCC6(CN(C)C)CC6(F)F)nc5c3F)N3CCCCC3CC4)c12] | 4-[2-({1-[(dimethylamino)methyl]-2,2- difluorocyclopropyl}methoxy)-4-fluoro- 8,8a,9,10,11,12-hexahydro-7H-1,3,6,12a- tetraazabenzo[4,5]cyclohepta[1,2,3- de]naphthalen-5-yl]-5-ethynyl-6- fluoronaphthalen-2-ol |
| 25 | 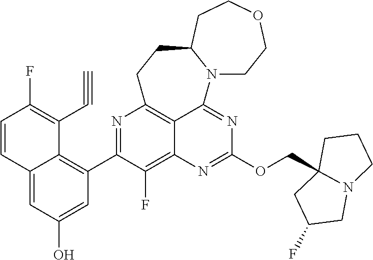 [SMILES: C#Cc1c(F)ccc2cc(O)cc(-c3nc4c5c(nc(OC[C@@]67CCCN6C[C@H](F)C7)nc5c3F)N3CCOCC[C@@H]3CC4)c12] | 5-ethynyl-6-fluoro-4-[(8aS)-4-fluoro-2- {[(2R,7aS)-2-fluorotetrahydro-1H- pyrrolizin-7a(5H)-yl]methoxy}- 8,8a,9,10,12,13-hexahydro-7H-11-oxa 1,3,6,13a-tetraazanaphtho[1,8-ab]heptalen- 5-yl]naphthalen-2-ol |
| 26 | 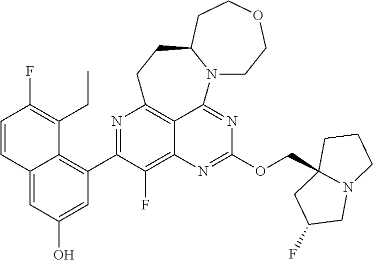 [SMILES: CCc1c(F)ccc2cc(O)cc(-c3nc4c5c(nc(OC[C@@]67CCCN6C[C@H](F)C7)nc5c3F)N3CCOCC[C@@H]3CC4)c12] | 5-ethyl-6-fluoro-4-[(8aS)-4-fluoro-2- {[(2R,7aS)-2-fluorotetrahydro-1H- pyrrolizin-7a(5H)-yl]methoxy}- 8,8a,9,10,12,13-hexahydro-7H-11-oxa- 1,3,6,13a-tetraazanaphtho[1,8-ab]heptalen- 5-yl]naphthalen-2-ol |
| 27 | 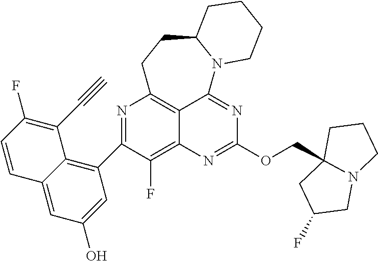 [SMILES: C#Cc1c(F)ccc2cc(O)cc(-c3nc4c5c(nc(OC[C@@]67CCCN6C[C@H](F)C7)nc5c3F)N3CCCC[C@@H]3CC4)c12] | 5-ethynyl-6-fluoro-4-[(8aR)-4-fluoro-2- {[(2R,7aS)-2-fluorotetrahydro-1H- pyrrolizin-7a(5H)-yl]methoxy}- 8,8a,9,10,11,12-hexahydro-7H-1,3,6,12a- tetraazabenzo[4,5]cyclohepta[1,2,3- de]naphthalen-5-yl]naphthalen-2-ol |
| 28 | 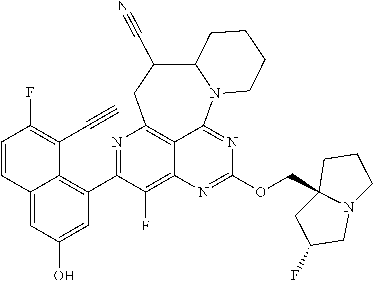 [SMILES: C#Cc1c(F)ccc2cc(O)cc(-c3nc4c5c(nc(OC[C@@]67CCCN6C[C@H](F)C7)nc5c3F)N3CCCCC3C(C#N)C4)c12] | 5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7H-1,3,6,12a- tetraazabenzo[4,5]cyclohepta[1,2,3- de]naphthalene-8-carbonitrile |
| 29 | 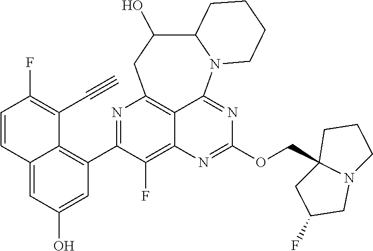 [SMILES: C#Cc1c(F)ccc2cc(O)cc(-c3nc4c5c(nc(OC[C@@]67CCCN6C[C@H](F)C7)nc5c3F)N3CCCCC3C(O)C4)c12] | 5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7H-1,3,6,12a- tetraazabenzo[4,5]cyclohepta[1,2,3- de]naphthalen-8-ol |
| 30 | 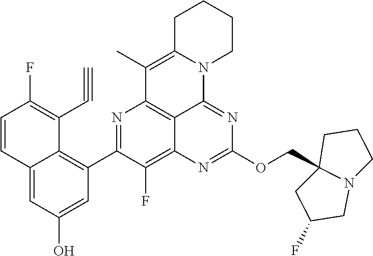 [SMILES: C#Cc1c(F)ccc2cc(O)cc(-c3nc4c5c(nc(OC[C@@]67CCCN6C[C@H](F)C7)nc5c3F)N3CCCCC3=C4C)c12] | 5-ethynyl-6-fluoro-4-(4-fluoro-2- {[(2R,7aS)-2-fluorotetrahydro-1H- pyrrolizin-7a(5H)-yl]methoxy}-7-methyl- 8,9,10,11-tetrahydropyrido[1,2- g]pyrimido[4,5,6-de][1,6]naphthyridin-5- yl)naphthalen-2-ol |
| 31 | 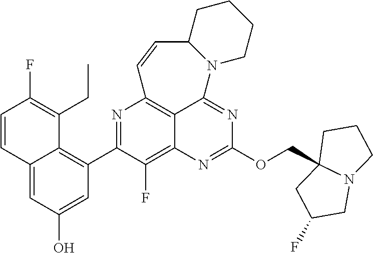 [SMILES: CCc1c(F)ccc2cc(O)cc(-c3nc4c5c(nc(OC[C@@]67CCCN6C[C@H](F)C7)nc5c3F)N3CCCCC3C=C4)c12] | 5-ethyl-6-fluoro-4-(4-fluoro-2-{[(2R,7aS)- 2-fluorotetrahydro-1H-pyrrolizin-7a(5H)- yl]methoxy}-9,10,11,12-tetrahydro-8aH- 1,3,6,12a- tetraazabenzo[4,5]cyclohepta[1,2,3- de]naphthalen-5-yl)naphthalen-2-ol |
| 32 | 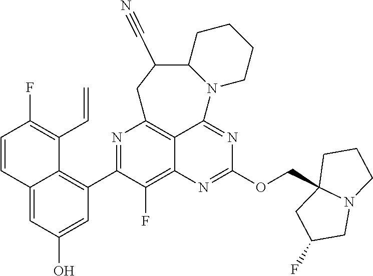 [SMILES: C=Cc1c(F)ccc2cc(O)cc(-c3nc4c5c(nc(OC[C@@]67CCCN6C[C@H](F)C7)nc5c3F)N3CCCCC3C(C#N)C4)c12] | 5-(8-ethenyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7H-1,3,6,12a- tetraazabenzo[4,5]cyclohepta[1,2,3- de]naphthalene-8-carbonitrile |

and pharmaceutically acceptable salts thereof.

Those skilled in the art will recognize that the species listed or illustrated herein are not exhaustive, and that additional species within the scope of these defined terms may also be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cutaneous melanoma, head and neck squamous cell carcinoma, pediatric glioma CML, prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma. Pain includes, for example, pain from any source or etiology, including cancer pain, pain from chemotherapeutic treatment, nerve pain, pain from injury, or other sources. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the disclosure specifically target Ras, in particular K-Ras. Thus, these compounds and pharmaceutical compositions can be used to prevent, reverse, slow, or inhibit the activity of one or more KRAS mutations, such as KRAS G12C, KRAS G12D, KRAS G12V, KRAS G12R, KRAS G12S, KRAS G13C, KRAS G13D, KRAS A18D, KRAS Q61H, KRAS K117N, and the like. In preferred embodiments, methods of treating a target cancer are described.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to a mutation of KRAS, such as KRAS G12C, KRAS G12D, KRAS G12V, KRAS G12R, KRAS G12S, KRAS G13C, KRAS G13D, KRAS A18D, KRAS Q61H, KRAS K117N, and the like.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g., crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the disclosure may additional comprise one or more of such active agents, and methods of treatment may additionally comprise administering an effective amount of one or more of such active agents.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)-(LXXXI).

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc or EA | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| R_{f} | retardation factor |
| N | normal |
| J | coupling constant |
| DMSO-d₆ | deuterated dimethyl sulfoxide |
| n-BuOH | n-butanol |
| TMSCl | trimethylsilyl chloride |
| TIPS | triisopropylsilyl |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos or Ts | toluenesulfonyl |
| DMAP | 4-(dimethylamino)pyridine |
| mM | millimolar |
| uM or μM | micromolar |
| nM | nanomolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| SEM | [2-(Trimethylsilyl)ethoxy]methyl acetal |
| Hex | hexanes |
| Pd(dppf)Cl₂ | [1,1′-Bis(diphenylphosphino)ferro- |
| | cene]dichloropalladium(II) |
| NiCl₂•dtbbpy | [4,4′-Bis(1,1-dimethylethyl)-2,2′-bipyridine] |
| | nickel (II) dichloride |
| MeCN (ACN) | Acetonitrile |
| Pd₂(dba)₃ | Tris(dibenzylideneacetone)dipalladium(0) |
| Pd(OAc)₂ | palladium(II) acetate |
| Pd(dtbpf)Cl₂ | [1,1′-Bis(di-tert-butylphosphino)ferro- |
| | cene]dichloropalladium(II) |
| cataCXium A Pd G3 | Mesylate[(di(1-adamantyl)-n-butylphosphine)-2-(2′- |
| | amino-1,1′-biphenyl)]palladium(II) |
| Xphos Pd G4 | (SP-4-3)-[Dicyclohexyl[2′,4′,6′-tris(1- |
| | methylethyl)[1,1′-biphenyl]-2- |
| | yl]phosphine](methanesulfonato-κO)[2′- |
| | (methylamino-κN)[1,1′-biphenyl]-2-yl- |
| | κC]palladium |
| Hunig's Base or | N,N-diisopropylethylamine |
| DIEA or DIPEA | |
| TBAF | Tetra-n-butyl ammonium fluoride |
| TBAC | Tetra-n-butylammonium chloride |
| PPh₃ | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine]palladium(II) |
| LiHMDS | Lithium bis(trimethylsilyl)amide |
| mCPBA | Meta-Chloroperoxy benzoic acid |
| AcOH | Acetic Acid |
| DMAc or DMA | N,N-Dimethylacetamide |
| BPD or B₂pin₂ | 4,4,5,5-tetramethyl-2-(4,4,5,5-tetramethyl-1,3,2- |
| | dioxaborolan-2-yl)-1,3,2-dioxaborolane |
| MTBE | Methyl tert-Butyl Ether |

The proposed targets can be prepared via conventional chemical means.

The proposed targets can be prepared via conventional chemical means or following the general scheme as shown below.

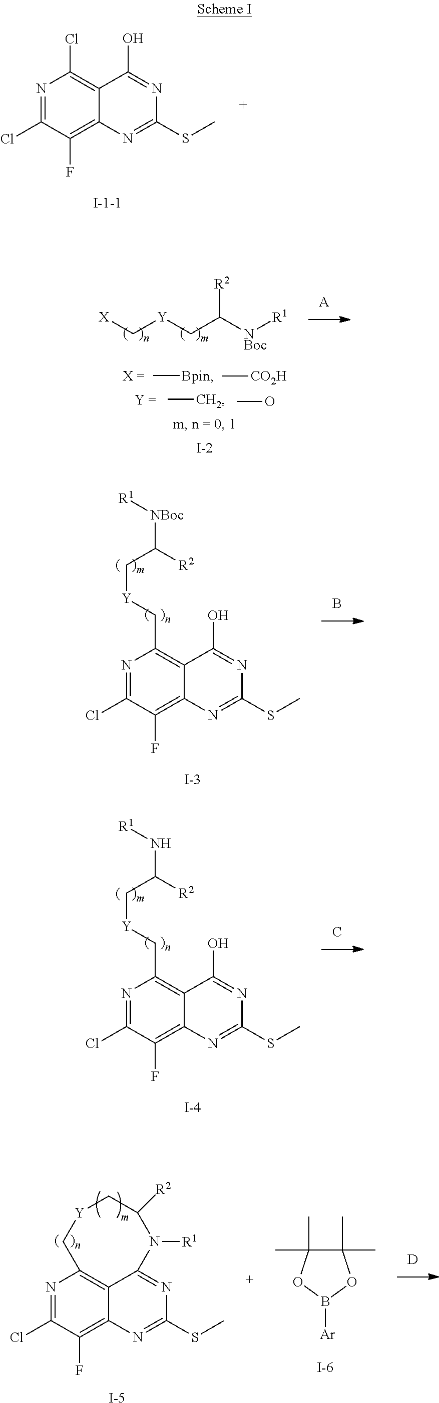

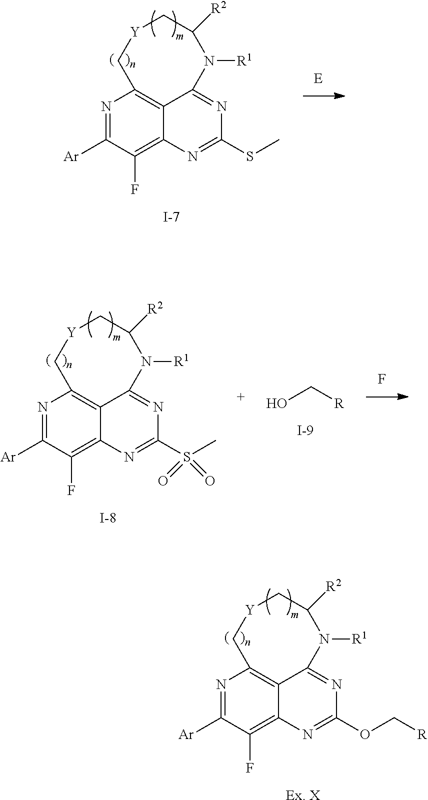

The general scheme I is used to prepare the products with general structure Ex. X. The bicyclic aryl core I-1-1 and a variety of amines I-2 are either commercially available or prepared from commercially available materials. A: under photochemical conditions (such as 10 W blue LED lamp) in the presence of an Ir(III) catalyst, a Ni(II) co-catalyst and a base such as morpholine in DMF at room temperature, compound I-1-1 and amine I-2 are converted to a product I-3. B: under acidic conditions such as HCl in dioxane at room temperature, compound I-3 is converted to product 1-4. C: in the presence of phosphorus oxychloride and a base such as diisopropylethylamine in THE at room temperature, compound I-4 is converted to product I-5. D: under palladium-catalyzed Suzuki coupling condition, compound I-5 and a boronic ester 1-6 are converted to product 1-7. E: in the presence of an oxidizing agent such as m-CPBA in DCM at room temperature, compound I-7 is converted to product 1-8. F: under basic conditions such as potassium tert-butoxide in toluene at reduced temperature such as 0° C., compound I-8 and an alcohol 1-9 are converted to a product which is deprotected if required to provide the final product Ex. X.

### Intermediate Synthesis

INTERMEDIATE TABLE 1

| Intermediate # | Structure | Notes |
|---|---|---|
| I-1-1 | 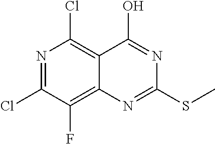 [SMILES: CSc1nc(O)c2c(Cl)nc(Cl)c(F)c2n1] | Commercially available |
| I-1-2 | 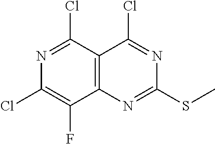 [SMILES: CSc1nc(Cl)c2c(Cl)nc(Cl)c(F)c2n1] | Commercially available |
| I-1-3 | 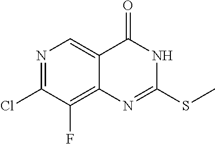 | Commercially available |

### Preparation of tert-butyl 3-(2-(4,4,5,5-tetramethyl-1,3,2-dioxaborolan-2-yl)ethyl)-1,4-oxazepane-4-carboxylate (I-2-1)

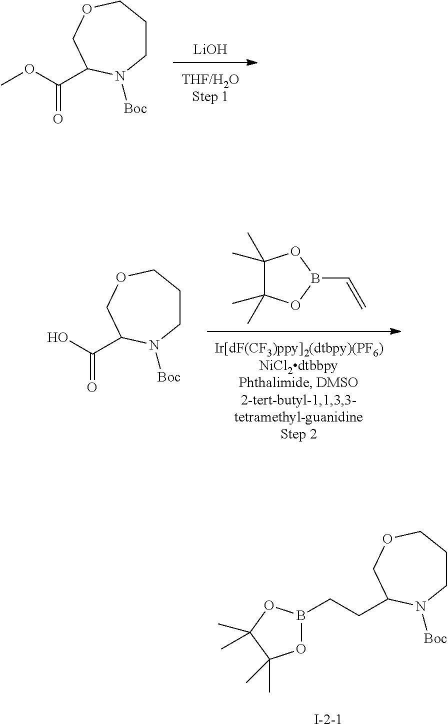 [SMILES: COC(=O)C1COCCCN1C(=O)OC(C)(C)C ; "LiOH" ; CC(C)(C)OC(=O)N1CCCOCC1C(=O)O ; C=CB1OC(C)(C)C(C)(C)O1 ; CC(C)(C)OC(=O)N1CCCOCC1CCB1OC(C)(C)C(C)(C)O1]

Step 1. To a solution of 4-(tert-butyl) 3-methyl 1,4-oxazepane-3,4-dicarboxylate (1.00 g, 3.86 mmol, 1 eq) in THE (2 mL) and H₂O (1 mL) was added LiOH·H₂O (178 mg, 4.24 mmol, 1.1 eq). The mixture was stirred at 25° C. for 0.5 h. On completion, the mixture was concentrated to give a residue. The residue was purified by column chromatography (SiO₂, DCM:MeOH=20:1) to give 4-(tert-butoxycarbonyl)-1,4-oxazepane-3-carboxylic acid (940 mg, 3.83 mmol, 99% yield) as yellow oil.

Step 2. To a 15 mL vial equipped with a stir bar was added 4-(tert-butoxycarbonyl)-1,4-oxazepane-3-carboxylic acid (940 mg, 3.83 mmol, 1 eq), 4,4,5,5-tetramethyl-2-vinyl-1,3,2-dioxaborolane (590 mg, 3.83 mmol, 0.65 mL, 1 eq), Ir[dF(CF₃)ppy]₂(dtbpy)(PF₆) (43.0 mg, 0.038 mmol, 0.01 eq), NiCl₂·dtbbpy (7.63 mg, 0.019 mmol, 0.005 eq), 2-tert-butyl-1,1,3,3-tetramethyl-guanidine (984 mg, 5.75 mmol, 1.2 mL, 1.5 eq), Phthalimide (563 mg, 3.83 mmol, 1 eq) in DMSO (10 mL). The reaction was stirred under air and irradiated with a 10 W blue LED lamp (3 cm away) with cooling water to keep the reaction temperature at 25° C. for 2 h. On completion, the reaction mixture was diluted with water (30 mL) and extracted with EA (20 mL×3). The combined organic layers were dried over anhydrous sodium sulfate, filtered, and concentrated to give a residue. The residue was purified by column chromatography (SiO₂, Petroleum ether/Ethyl acetate=100/1 to 50/1) to give tert-butyl 3-(2-(4,4,5,5-tetramethyl-1,3,2-dioxaborolan-2-yl)ethyl)-1,4-oxazepane-4-carboxylate (400 mg, 1.13 mmol, 29% yield) as a white solid.

### Preparation of tert-butyl 5-(2-(4,4,5,5-tetramethyl-1,3,2-dioxaborolan-2-yl)ethyl)-1,4-oxazepane-4-carboxylate (I-2-2)

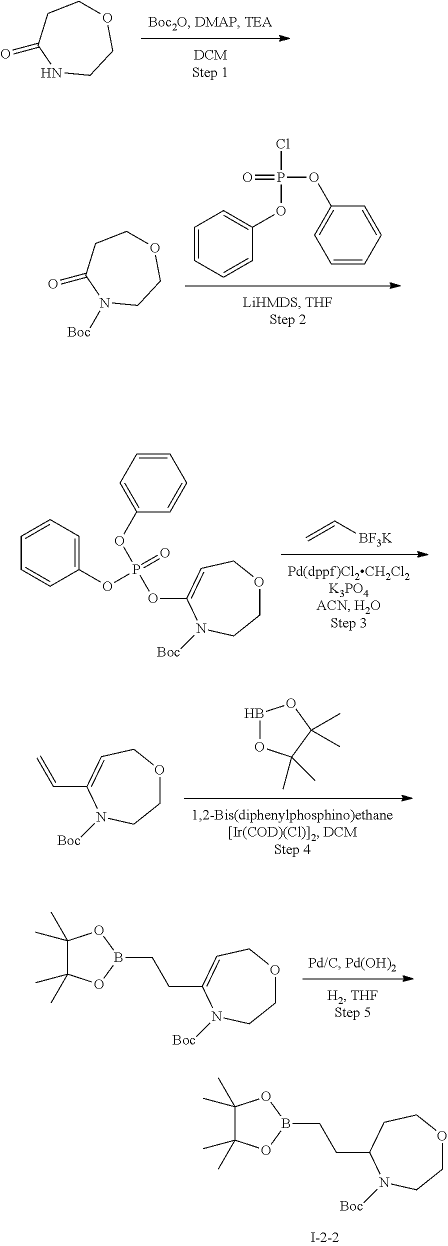

Step 1. To a solution of 1,4-oxazepan-5-one (10.0 g, 86.8 mmol, 1 eq) in DCM (150 mL) was added TEA (13.1 g, 130 mmol, 1.5 eq), DMAP (1.06 g, 8.69 mmol, 0.1 eq) and Boc₂O (24.6 g, 112 mmol, 1.3 eq). The mixture was stirred at 25° C. for 12 hours. On completion, the reaction mixture was partitioned between ethyl acetate (200 mL×3) and water (200 mL), and the combined organic phase was dried over anhydrous sodium sulfate, filtered, and concentrated to give tert-butyl 5-oxo-1,4-oxazepane-4-carboxylate (14.2 g, 65.9 mmol, 75% yield) as a yellow solid. ¹H NMR (400 MHz, DMSO-d₆) δ=3.92-3.85 (m, 2H), 3.75-3.64 (m, 4H), 2.84-2.73 (m, 2H), 1.45 (s, 9H).

Step 2. A mixture of tert-butyl 5-oxo-1,4-oxazepane-4-carboxylate (14.2 g, 65.9 mmol, 1 eq) in THE (200 mL) was degassed and purged with N₂ thrice, followed by addition of LiHMDS (1 M, 65.9 mL, 1 eq) at −70° C. for 0.5 hours. To the reaction mixture was then added [chloro(phenoxy)phosphoryl]oxybenzene (17.7 g, 65.9 mmol, 1 eq) at −70° C., and then the mixture was stirred at −70° C. for 1.5 hours under N₂ atmosphere. On completion, the reaction mixture was partitioned between ethyl acetate (200 mL×3) and water (200 mL), and the combined organic phase was dried over anhydrous sodium sulfate, filtered, and concentrated to give a residue. The residue was purified by column chromatography (SiO₂, Petroleum ether/Ethyl acetate=1:0 to 0:1) to give tert-butyl 5-((diphenoxyphosphoryl)oxy)-2,3-dihydro-1,4-oxazepine-4(7H)-carboxylate (23.0 g, 51.4 mmol, 77% yield) as yellow oil. ¹H NMR (400 MHz, CDCl₃) δ=7.47-7.16 (m, 10H), 5.52 (dt, J=2.8, 6.4 Hz, 1H), 4.09 (dd, J=1.2, 6.4 Hz, 2H), 3.77 (t, J=4.4 Hz, 2H), 3.66 (s, 2H), 1.45 (s, 9H).

Step 3. A mixture of tert-butyl 5-((diphenoxyphosphoryl)oxy)-2,3-dihydro-1,4-oxazepine-4(7H)-carboxylate (13.0 g, 29.0 mmol, 1 eq), Potassium vinyl trifluoroborate (4.67 g, 34.8 mmol, 1.2 eq), K₃PO₄ (18.0 g, 87.1 mmol, 3 eq), Pd(dppf)Cl₂·CH₂Cl₂ (2.37 g, 2.91 mmol, 0.1 eq) in ACN (200 mL) and H₂O (50 mL) was degassed and purged with N₂ thrice, and then the mixture was stirred at 75° C. for 4 hours under N₂ atmosphere. On completion, the reaction mixture was partitioned between ethyl acetate (150 mL×3) and water (150 mL), and the combined organic phase was dried over anhydrous sodium sulfate, filtered, and concentrated to give a residue. The residue was purified by column chromatography (SiO₂, Petroleum ether/Ethyl acetate=1:0 to 0:1) to give tert-butyl 5-vinyl-2,3-dihydro-1,4-oxazepine-4(7H)-carboxylate (4.60 g, 20.4 mmol, 70% yield) as white oil. ¹H NMR (400 MHz, CDCl₃) δ=6.17 (dd, J=10.4, 16.8 Hz, 1H), 5.66 (t, J=5.6 Hz, 1H), 5.32-4.96 (m, 2H), 4.05 (d, J=6.0 Hz, 2H), 3.74 (t, J=4.4 Hz, 2H), 3.45 (s, 2H), 1.51-1.27 (m, 9H).

Step 4. A mixture of tert-butyl 5-vinyl-3,7-dihydro-2H-1,4-oxazepine-4-carboxylate (4.60 g, 20.4 mmol, 1 eq), 1,2-Bis(diphenylphosphino)ethane (2.85 g, 7.15 mmol, 0.35 eq), [Ir(COD)(Cl)]₂ (2.06 g, 3.06 mmol, 0.15 eq) in DCM (60 mL) was degassed and purged with N₂ thrice, and then the mixture was stirred at 25° C. for 0.5 hours under N₂ atmosphere. Then 4,4,5,5-tetramethyl-1,3,2-dioxaborolane (3.92 g, 30.6 mmol, 1.5 eq) was added to the mixture at 0° C., and the mixture was stirred at 25° C. for 0.5 hours. On completion, the mixture was filtered and concentrated to give a residue. The residue was purified by column chromatography (SiO₂, Petroleum ether/Ethyl acetate=1:0 to 0:1) to give tert-butyl 5-(2-(4,4,5,5-tetramethyl-1,3,2-dioxaborolan-2-yl)ethyl)-2,3-dihydro-1,4-oxazepine-4(7H)-carboxylate (900 mg, 2.55 mmol, 12% yield) as yellow oil. ¹H NMR (400 MHz, CDCl₃) δ=5.39 (s, 1H), 3.95 (d, J=5.6 Hz, 2H), 3.71-3.63 (m, 2H), 3.45 (s, 2H), 2.39 (s, 2H), 1.40 (s, 9H), 1.17 (s, 12H), 0.83 (t, J=8.0 Hz, 2H).

Step 5. A mixture of tert-butyl 5-[2-(4,4,5,5-tetramethyl-1,3,2-dioxaborolan-2-yl)ethyl]-3,7-dihydro-2H-1,4-oxazepine-4-carboxylate (860 mg, 2.43 mmol, 1 eq), Pd/C (1.50 g, 1.41 mmol, 10% purity) and Pd(OH)₂ (1.20 g, 0.854 mmol, 10% purity) in THE (5 mL) was degassed and purged with H₂ thrice, and then the mixture was stirred at 25° C. for 2 hours under H₂ atmosphere. On completion, the mixture was filtered and concentrated to give a residue. The residue was purified by column chromatography (SiO₂, Petroleum ether/Ethyl acetate=1:0 to 0:1) to give tert-butyl 5-(2-(4,4,5,5-tetramethyl-1,3,2-dioxaborolan-2-yl)ethyl)-1,4-oxazepane-4-carboxylate (430 mg, 1.21 mmol, 49% yield) as yellow oil.

### Intermediate Method I-2A

### Preparation of (S)-2-((1-(tert-butoxycarbonyl)piperidin-2-yl)methoxy)acetic acid (I-2-3)

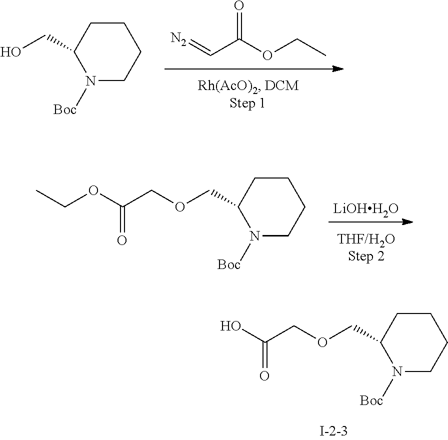

Step 1. A mixture of tert-butyl (S)-2-(hydroxymethyl)piperidine-1-carboxylate (8.00 g, 37.2 mmol, 1 eq), diacetoxyrhodium (493 mg, 2.23 mmol, 0.06 eq) in DCM (70 mL) was degassed and purged with N₂ for 3 times, ethyl 2-diazoacetate (5.09 g, 44.6 mmol, 4.69 mL, 1.2 eq) in DCM (10 mL) was added at 0° C. under N₂. The mixture was stirred at 25° C. for 16 h under N₂ atmosphere. On completion, the reaction mixture was concentrated under reduced pressure to remove solvent. The residue was purified by column chromatography (SiO₂, Petroleum ether/Ethyl acetate=1/0 to 0/1) to give tert-butyl (S)-2-((2-ethoxy-2-oxoethoxy)methyl)piperidine-1-carboxylate (5.60 g, 18.6 mmol, 50% yield) as yellow oil. ¹H NMR (400 MHz, CDCl₃) δ=4.47-4.31 (m, 1H), 4.30-4.16 (m, 2H), 4.13-4.05 (m, 2H), 4.03-3.92 (m, 1H), 3.69-3.55 (m, 2H), 2.86-2.69 (m, 1H), 1.92-1.71 (m, 2H), 1.65-1.50 (m, 4H), 1.41 (d, J=3.2 Hz, 9H), 1.31-1.23 (m, 3H).

Step 2. To a solution of tert-butyl (S)-2-((2-ethoxy-2-oxoethoxy)methyl)piperidine-1-carboxylate (2.00 g, 6.64 mmol, 1 eq) in THE (20 mL) and H₂O (4 mL) was added LiOH·H₂O (835 mg, 19.9 mmol, 3 eq). The mixture was stirred at 25° C. for 2 hr. On completion, the reaction mixture was concentrated under reduced pressure to remove solvent. The residue was purified by column chromatography (SiO₂, Petroleum ether/Ethyl acetate=I/O to 0/1) to give (S)-2-((1-(tert-butoxycarbonyl)piperidin-2-yl)methoxy)acetic acid (1.34 g, 4.90 mmol, 74% yield) as yellow oil.

### Intermediate Method I-2B

### Preparation of tert-butyl 2-(2-(4,4,5,5-tetramethyl-1,3,2-dioxaborolan-2-yl)ethyl)azepane-1-carboxylate (I-2-8)

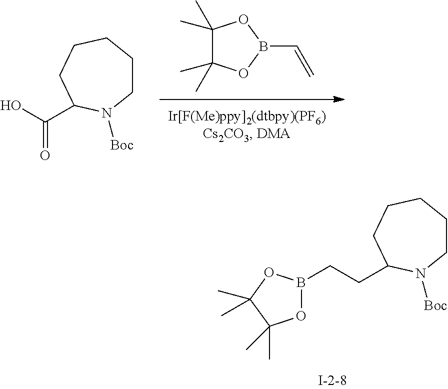 [SMILES: CC(C)(C)OC(=O)N1CCCCCC1C(=O)O ; C=CB1OC(C)(C)C(C)(C)O1 ; CC(C)(C)OC(=O)N1CCCCCC1CCB1OC(C)(C)C(C)(C)O1]

To a 40 mL vial equipped with a stir bar was added 1-(tert-butoxycarbonyl)azepane-2-carboxylic acid (4.50 g, 18.5 mmol, 1 eq), 4,4,5,5-tetramethyl-2-vinyl-1,3,2-dioxaborolane (4.27 g, 27.7 mmol, 4.71 mL, 1.5 eq), Ir[F(Me)ppy]₂(dtbpy)(PF₆) (362 mg, 0.370 mmol, 0.02 eq), and Cs₂CO₃ (6.03 g, 18.5 mmol, 1 eq) in DMA (24 mL). The vial was sealed and placed under nitrogen. The reaction was stirred and irradiated with a blue 4×50 W LED lamp (3 cm away) with cooling water to keep the reaction temperature at 25° C. for 14 hours. On completion, the reaction mixture diluted with H₂O (200 mL) and extracted with EA (50 mL×3), and the combined organic phase was dried over anhydrous sodium sulfate, filtered, and concentrated under reduced pressure to give a residue. The residue was purified by column chromatography (SiO₂, Petroleum ether/Ethyl acetate=1:0 to 10:1) to give compound tert-butyl 2-[2-(4,4,5,5-tetramethyl-1,3,2-dioxaborolan-2-yl)ethyl]azepane-1-carboxylate (1.22 g, 2.76 mmol, 15% yield, 80% purity) as an orange liquid.

### Preparation of tert-butyl 2-(2-(4,4,5,5-tetramethyl-1,3,2-dioxaborolan-2-yl)ethyl)piperidine-1-carboxylate (I-2-12)

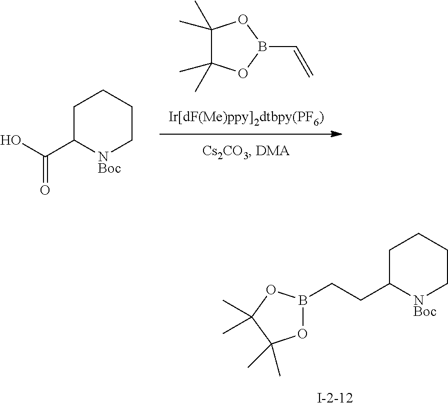 [SMILES: CC(C)(C)OC(=O)N1CCCCC1C(=O)O ; C=CB1OC(C)(C)C(C)(C)O1 ; CC(C)(C)OC(=O)N1CCCCC1CCB1OC(C)(C)C(C)(C)O1]

To a 40-mL vial equipped with a stir bar was added 1-(tert-butoxycarbonyl)piperidine-2-carboxylic acid (5.00 g, 21.8 mmol, 1 eq), 4,4,5,5-tetramethyl-2-vinyl-1,3,2-dioxaborolane (4.37 g, 28.4 mmol, 4.81 mL, 1.3 eq), Cs₂CO₃ (7.11 g, 21.8 mmol, 1 eq), Ir[4dF(Me)ppy]₂dtbpy(PF₆) (442 mg, 0.436 mmol, 0.02 eq) in DMA (50 mL). The vial was sealed and placed under nitrogen was added. The reaction was stirred and irradiated with a 4×50 W [1455 nm] blue LED lamp (3 cm away) with cooling water to keep the reaction temperature at 25° C. for 14 h. On completion, the reaction mixture was concentrated under reduced pressure to give a residue. The residue was purified by flash silica gel chromatography (0-100% Ethyl acetate/Petroleum) to give tert-butyl 2-(2-(4,4,5,5-tetramethyl-1,3,2-dioxaborolan-2-yl)ethyl)piperidine-d-carboxylate (1.20 g, 3.18 mmol, 15% yield, 90% purity) as a yellow oil.

INTERMEDIATE TABLE 2

| # | Method | Structure | SM | ¹H NMR |
|---|---|---|---|---|
| I-2-1 | | 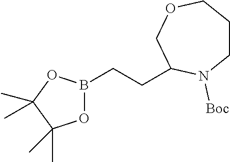 [SMILES: CC(C)(C)OC(=O)N1CCCOCC1CCB1OC(C)(C)C(C)(C)O1] | 4-(tert-butyl)-3- methyl 1,4- oxazepane-3,4- dicarboxylate | (400 MHz, DMSO-d₆) δ = 4.09-3.98 (m, 1H), 3.91-3.79 (m, 2H), 3.60 (s, 1H), 3.40- 3.36 (m, 1H), 3.27-3.17 (m, 1H), 3.02- 2.93 (m, 1H), 1.74-1.64 (m, 1H), 1.57- 1.48 (m, 1H), 1.40 (s, 9H), 1.35-1.30 (m, 2H), 1.17 (s, 12H), 0.68-0.52 (m, 2H) |
| I-2-2 | | 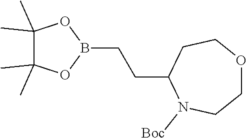 [SMILES: CC(C)(C)OC(=O)N1CCOCCC1CCB1OC(C)(C)C(C)(C)O1] | 1,4-oxazepan-5- one | (400 MHz, CDCl₃) δ = 4.23-3.99 (m, 1H), 3.87-3.80 (m, 1H), 3.78-3.56 (m, 3H), 3.48-3.08 (m, 3H), 2.96-2.81 (m, 1H), 2.17-1.99 (m, 1H), 1.47 (s, 1H), 1.39 (d, J = 6.0 Hz, 9H), 1.17 (s, 12H), 0.81 (t, J = 7.2 Hz, 2H) |
| I-2-3 | I-2A | 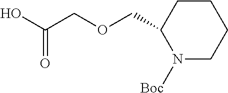 [SMILES: CC(C)(C)OC(=O)N1CCCC[C@H]1COCC(=O)O] | (S)-2- (hydroxymethyl) piperidine-1- carboxylate | (400 MHz, DMSO-d₆) δ = 13.18-11.89 (m, 1H), 4.29-4.18 (m, 1H), 4.06-3.97 (m, 2H), 3.88-3.75 (m, 1H), 3.63-3.50 (m, 2H), 2.83-2.68 (m, 1H), 1.75-1.65 (m, 1H), 1.62-1.42 (m, 5H), 1.38 (s, 9H) |
| I-2-4 | I-2A | 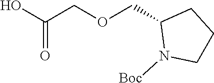 [SMILES: CC(C)(C)OC(=O)N1CCC[C@H]1COCC(=O)O] | tert-butyl (S)-2- (hydroxymethyl) pyrrolidine-1- carboxylate | (400 MHz, DMSO-d₆) δ = 12.58 (s, 1H), 4.02- 3.96 (m, 2H), 3.78 (s, 1H), 3.57-3.49 (m, 1H), 3.33 (t, J = 8.4 Hz, 1H), 3.23-3.16 (m, 2H), 1.92-1.68 (m, 4H), 1.39 (s, 9H) |
| I-2-5 | | 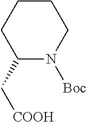 | | Commerically Available |
| I-2-6 | | 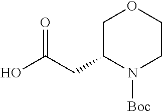 [SMILES: CC(C)(C)OC(=O)N1CCOC[C@H]1CC(=O)O] | | Commerically Available |
| I-2-7 | | 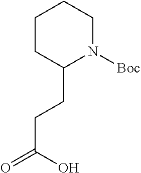 [SMILES: CC(C)(C)OC(=O)N1CCCCC1CCC(=O)O] | | Commercially Available |
| I-2-8 | I-2B | 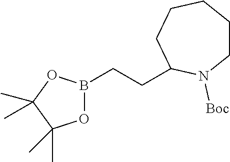 [SMILES: CC(C)(C)OC(=O)N1CCCCCC1CCB1OC(C)(C)C(C)(C)O1] | 1-(tert- butoxycarbonyl) azepane-2- carboxylic acid | (400 MHz, DMSO-d₆) δ = 3.90-3.81 (m, 1H), 3.77-3.65 (m, 1H), 3.47 (d, J = 16.8 Hz, 1H), 2.94 (s, 1H), 2.78 (s, 1H), 2.66 (t, J = 12.8 Hz, 1H), 2.05-1.86 (m, 1H), 1.75- 1.57 (m, 2H), 1.39 (s, 9H), 1.24-0.96 (m, 17H), 0.68-0.48 (m, 1H) |
| I-2-12 | | 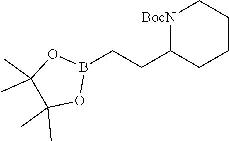 [SMILES: CC(C)(C)OC(=O)N1CCCCC1CCB1OC(C)(C)C(C)(C)O1] | -(tert- butoxycarbonyl) piperidine-2- carboxylic acid | (400 MHz, CDCl₃) δ = 4.13 (d, J = 2.8 Hz, 1H), 4.05-3.89 (m, 1H), 2.86-2.69 (m, 1H), 1.89-1.69 (m, 1H), 1.57 (s, 7H), 1.47 (s, 9H), 1.27 (s, 12H), 0.73 (t, J = 8.4 Hz, 2H) |
| I-2-13 | | 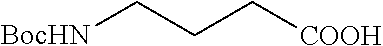 [SMILES: CC(C)(C)OC(=O)NCCCC(=O)O] | | Commerically Available |

INTERMEDIATE TABLE 3

| Intermediate # | Structure | Notes |
|---|---|---|
| I-6-1 | 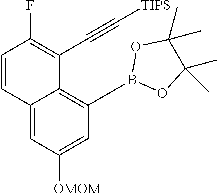 | Commercially Available |
| I-6-2 | 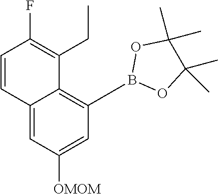 | Commercially Available |
| I-9-1 | 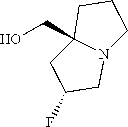 [SMILES: OC[C@@]12CCCN1C[C@H](F)C2] | Commercially Available |
| I-9-2 | 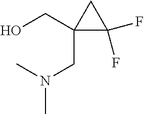 | Commercially Available |

### General Method A

### Preparation of 5-ethynyl-6-fluoro-4-[(8aR)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (Ex. 1) and 5-ethynyl-6-fluoro-4-[(8aS)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (Ex. 2)

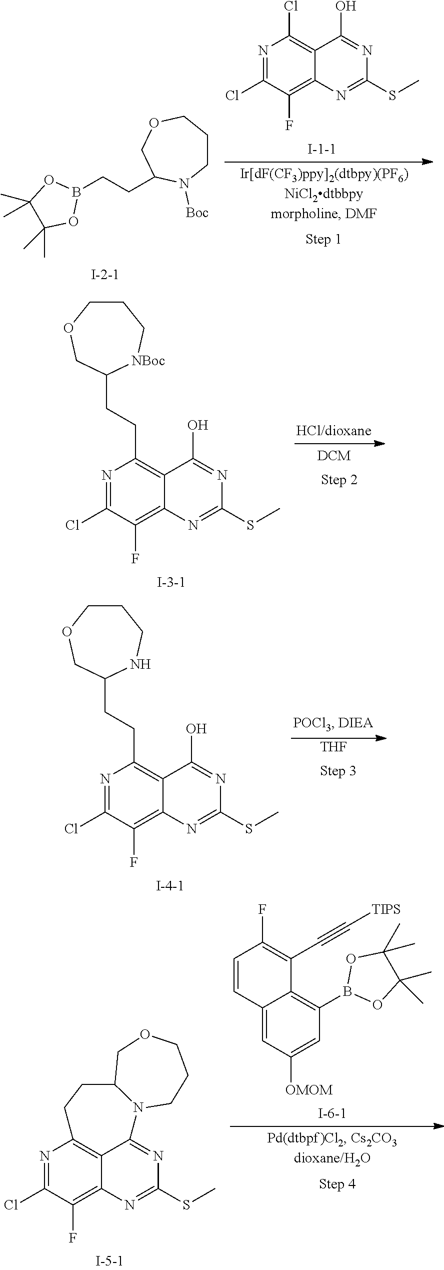

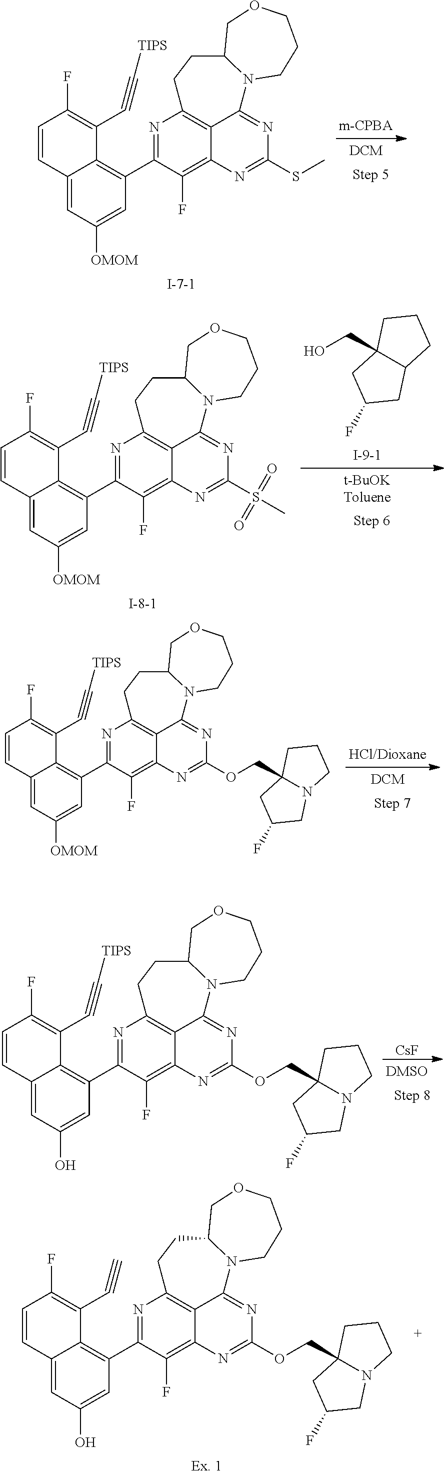

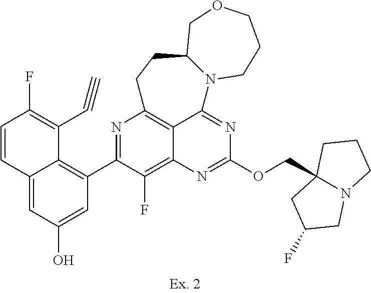 [SMILES: C#Cc1c(F)ccc2cc(O)cc(-c3nc4c5c(nc(OC[C@@]67CCCN6C[C@H](F)C7)nc5c3F)N3CCCOC[C@@H]3CC4)c12]

Step 1. To a 15-mL vial equipped with a stir bar was added tert-butyl 3-(2-(4,4,5,5-tetramethyl-1,3,2-dioxaborolan-2-yl)ethyl)-1,4-oxazepane-4-carboxylate (I-2-1, 480 mg, 1.35 mmol, 2 eq), 5,7-dichloro-8-fluoro-2-(methylthio)pyrido[4,3-d]pyrimidin-4-ol (189 mg, 0.675 mmol, 1 eq), Ir[dF(CF₃)ppy]₂(dtbpy)(PF₆) (7.58 mg, 0.0068 mmol, 0.01 eq), NiCl₂·dtbbpy (13.4 mg, 0.034 mmol, 0.05 eq), morpholine (88.2 mg, 1.01 mmol, 0.09 mL, 1.5 eq) in DMF (2 mL). The reaction was stirred under air and irradiated with a 10 W blue LED lamp (3 cm away), with cooling water to keep the reaction temperature at 25° C. for 2 h. On completion, the mixture was filtered and the filtrate was purified by prep-HPLC to give tert-butyl 3-(2-(7-chloro-8-fluoro-4-hydroxy-2-(methylthio)pyrido[4,3-d]pyrimidin-5-yl)ethyl)-1,4-oxazepane-4-carboxylate (105 mg, 0.206 mmol, 30% yield) as a white solid. ¹H NMR (400 MHz, DMSO-d₆) δ=13.12 (s, 1H), 4.21-4.09 (m, 1H), 4.08-3.89 (m, 2H), 3.89-3.78 (m, 2H), 3.76-3.54 (m, 2H), 3.28-3.23 (m, 2H), 2.59 (s, 3H), 1.69-1.65 (m, 2H), 1.60-1.51 (m, 2H), 1.34 (s, 9H); LCMS: (M+1:473.0).

Step 2. To a solution of tert-butyl 3-(2-(7-chloro-8-fluoro-4-hydroxy-2-(methylthio)pyrido[4,3-d]pyrimidin-5-yl)ethyl)-1,4-oxazepane-4-carboxylate (80.0 mg, 0.169 mmol, 1 eq) in DCM (0.5 mL) was added HCl/dioxane (2 M, 1 mL, 11.8 eq). The mixture was stirred at 25° C. for 0.5 h. On completion, the reaction mixture was concentrated under reduced pressure to give 5-(2-(1,4-oxazepan-3-yl)ethyl)-7-chloro-8-fluoro-2-(methylthio)pyrido[4,3-d]pyrimidin-4-ol (63.0 mg, 0.168 mmol, 99% yield) as a white solid. LCMS: (M+1:373.0).

Step 3. To a solution of 5-(2-(1,4-oxazepan-3-yl)ethyl)-7-chloro-8-fluoro-2-(methylthio)pyrido[4,3-cd]pyrimidin-4-ol (63.0 mg, 0.168 mmol, 1 eq) in THE (1 mL) was added POCl₃ (0.506 mmol, 0.048 mL, 3 eq) and DIEA (1.52 mmol, 0.264 mL, 9 eq). The mixture was stirred at 25° C. for I h. On completion, the reaction mixture was quenched with H₂O (0.1 mL) at 25° C., and then concentrated under reduced pressure to give a residue. The residue was purified by prep-TLC (SiO₂, PE:EA=0:1) to give 2-chloro-1-fluoro-12-(methylthio)-4,5,5a,6,9,10-hexahydro-8H-7-oxa-3,10a,11,13-tetraazanaphtho[1,8-ab]heptalene (50.0 mg, 0.140 mmol, 83% yield) as a white solid. LCMS: (M+1: 354.9).

Step 4. A mixture of ((2-fluoro-6-(methoxymethoxy)-8-(4,4,5,5-tetramethyl-1,3,2-dioxaborolan-2-yl)naphthalen-1-yl)ethynyl)triisopropylsilane (91.3 mg, 0.183 mmol, 1.3 eq), 2-chloro-1-fluoro-12-(methylthio)-4,5,5a,6,9,10-hexahydro-8H-7-oxa-3,10a,11,13-tetraazanaphtho[1,8-ab]heptalene (50.0 mg, 0.140 mmol, 1 eq), Cs₂CO₃ (91.8 mg, 0.281 mmol, 2 eq), Pd(dtbpf)Cl₂ (9.18 mg, 0.014 mmol, 0.1 eq) in dioxane (1 mL) and H₂O (0.2 mL) was degassed and purged with N₂ for 3 times, and then the mixture was stirred at 80° C. for 2 h under N₂ atmosphere. On completion, the reaction mixture was concentrated under reduced pressure to give a residue. The residue was purified by prep-TLC (SiO₂, PE:EA=2:1) to give 1-fluoro-2-(7-fluoro-3-(methoxymethoxy)-8-((triisopropylsilyl)ethynyl)naphthalen-1-yl)-12-(methylthio)-4,5,5a,6,9,10-hexahydro-8H-7-oxa-3,10a,11,13-tetraazanaphtho[1,8-ab]heptalene (80.0 mg, 0.106 mmol, 76% yield) as a white solid. ¹H NMR (400 MHz, DMSO-d₆) δ=8.12-8.05 (m, 1H), 7.74-7.70 (m, 1H), 7.59-7.51 (m, 1H), 7.47-7.22 (m, 1H), 5.40-5.31 (m, 2H), 4.45-4.28 (m, 1H), 3.92-3.79 (m, 4H), 3.68-3.61 (m, 1H), 3.61-3.52 (m, 2H), 3.43 (s, 3H), 3.12-3.04 (m, 1H), 3.03-2.92 (m, 2H), 2.29-2.07 (m, 3H), 2.04-1.79 (m, 3H), 0.85-0.77 (m, 18H); LCMS: (M+1:705.3).

Step 5. To a solution of 1-fluoro-2-(7-fluoro-3-(methoxymethoxy)-8-((triisopropylsilyl)ethynyl)naphthalen-1-yl)-12-(methylthio)-4,5,5a,6,9,10-hexahydro-8H-7-oxa-3,10a,11,13-tetraazanaphtho[1,8-ab]heptalene (80.0 mg, 0.113 mmol, 1 eq) in DCM (1 mL) was added m-CPBA (57.6 mg, 0.283 mmol, 85% purity, 2.5 eq). The mixture was stirred at 25° C. for 1 h. On completion, the reaction mixture was quenched with sat. Na₂S₂O₃ (1.5 mL) at 0° C., and then diluted with sat. NaHCO₃ (5 mL) and extracted with DCM (10 mL×3). The combined organic layers were dried over anhydrous Na₂SO₄, filtered and concentrated under reduced pressure to give 1-fluoro-2-(7-fluoro-3-(methoxymethoxy)-8-((triisopropylsilyl)ethynyl)naphthalen-1-yl)-12-(methylsulfonyl)-4,5,5a,6,9,10-hexahydro-8H-7-oxa-3,10a,11,13-tetraazanaphtho[1,8-ab]heptalene (83.0 mg, 0.112 mmol, 99% yield) as a white solid. LCMS: (M+1:737.3).

Step 6. To a solution of ((2R,7aS)-2-fluorotetrahydro-1H-pyrrolizin-7a(5H)-yl)methanol (53.7 mg, 0.337 mmol, 3 eq) in toluene (2 mL) was added t-BuOK (37.9 mg, 0.337 mmol, 3 eq) at 0° C. for 30 minutes, and then, 1-fluoro-2-(7-fluoro-3-(methoxymethoxy)-8-((triisopropylsilyl)ethynyl)naphthalen-1-yl)-12-(methylsulfonyl)-4,5,5a,6,9,10-hexahydro-8H-7-oxa-3,10a,11,13-tetraazanaphtho[1,8-ab]heptalene (83.0 mg, 0.112 mmol, 1 eq) was added. The mixture was stirred at 0° C. for 1 h. On completion, the reaction mixture was diluted with water (10 mL) and extracted with EA (5 mL×3). The combined organic layers were washed with brine (5 mL×2), dried over anhydrous sodium sulfate, filtered, and concentrated to give a residue. The residue was purified by prep-TLC (SiO₂, DCM:MeOH=10:1) to give 1-fluoro-2-(7-fluoro-3-(methoxymethoxy)-8-((triisopropylsilyl)ethynyl)naphthalen-1-yl)-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (30.0 mg, 0.033.0 mmol, 30% yield) as a white solid. LCMS: (M+1:816.4).

Step 7. To a solution of 1-fluoro-2-(7-fluoro-3-(methoxymethoxy)-8-((triisopropylsilyl)ethynyl)naphthalen-1-yl)-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (30.0 mg, 0.037 mmol, 1 eq) in DCM (0.5 mL) was added HCl/dioxane (2 M, 0.5 mL, 27 eq). The mixture was stirred at 25° C. for 1 h. On completion, the mixture was concentrated in vacuum to give 6-fluoro-4-(1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-ol (28.0 mg, 0.036 mmol, 98% yield) as a white solid. LCMS: (M+1:772.3).

Step 8. To a solution of 6-fluoro-4-(1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalen-2-yl)-5-((triisopropylsilyl)ethynyl)naphthalen-2-ol (22.0 mg, 0.028 mmol, 1 eq) in DMSO (2 mL) was added CsF (25.9 mg, 0.170 mmol, 6 eq). The mixture was stirred at 25° C. for 16 h. On completion, the mixture was filtered, and the filtrate was purified by prep-HPLC followed by SFC separation to furnish two crude products, which were further purified by preparative HPLC to afford 5-ethynyl-6-fluoro-4-[(8aR)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (2.57 mg, 0.0039 mmol, 19.9% yield, FA salt) as a yellow solid (Ex. 1) and 5-ethynyl-6-fluoro-4-[(8aS)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (3.14 mg, 0.0048 mmol, 24.4% yield, FA salt) as a yellow solid (Ex. 2). The analytical data for Ex. 1 and Ex. 2 can be found in the table below.

### Preparation of 5-ethyl-6-fluoro-4-[(8aR)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (Ex. 3) and 5-ethyl-6-fluoro-4-[(8aS)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (Ex. 4)

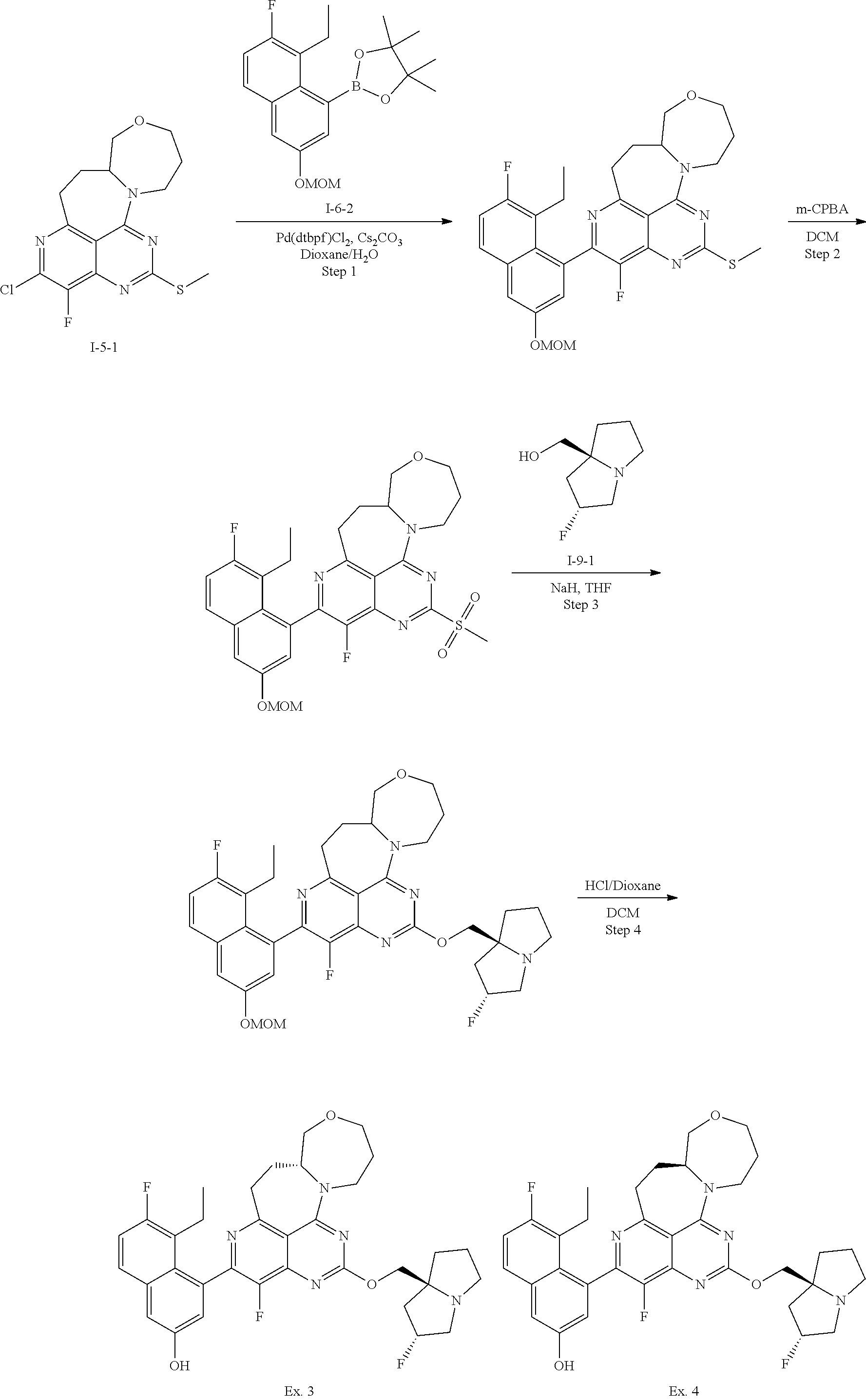

Step 1. A mixture of 2-chloro-1-fluoro-12-(methylthio)-4,5,5a,6,9,10-hexahydro-8H-7-oxa-3,10a,11,13-tetraazanaphtho[1,8-ab]heptalene, which was prepared following the method described in General Method A, (160 mg, 0.451 mmol, 1 eq), 2-(8-ethyl-7-fluoro-3-(methoxymethoxy)naphthalen-1-yl)-4,4,5,5-tetramethyl-1,3,2-dioxaborolane (211 mg, 0.586 mmol, 1.3 eq), Cs₂CO₃ (441 mg, 1.35 mmol, 3 eq), Pd(dtbpf)Cl₂ (29.4 mg, 0.045 mmol, 0.1 eq) in dioxane (2 mL) and H₂O (0.4 mL) was degassed and purged with N₂ thrice, and then the mixture was stirred at 80° C. for 1 h under N₂ atmosphere. On completion, the mixture was concentrated to give a residue. The residue was purified by column chromatography (SiO₂, PE/THF=1:0 to 2:1) to give 2-(8-ethyl-7-fluoro-3-(methoxymethoxy)naphthalen-1-yl)-1-fluoro-12-(methylthio)-4,5,5a,6,9,10-hexahydro-8H-7-oxa-3,10a,11,13-tetraazanaphtho[1,8-ab]heptalene (190 mg, 0.344 mmol, 76% yield) as a white solid. LCMS: (M+1:553.2).

Step 2. To a solution of 2-(8-ethyl-7-fluoro-3-(methoxymethoxy)naphthalen-1-yl)-1-fluoro-12-(methylthio)-4,5,5a,6,9,10-hexahydro-8H-7-oxa-3,10a,11,13-tetraazanaphtho[1,8-ab]heptalene (170 mg, 0.308 mmol, 1 eq) in DCM (2 mL) was added m-CPBA (156 mg, 0.769 mmol, 85% purity, 2.5 eq) at 0° C. The mixture was stirred at 25° C. for 1 h. The mixture was quenched with sat. Na₂SO₃ (5 mL) and extracted with DCM (5 mL×3), and the combined organic phase was dried over anhydrous sodium sulfate, filtered, and concentrated to give a residue. The residue was purified by column chromatography (SiO₂, PE/THF=1:0 to 1:1) to give 2-(8-ethyl-7-fluoro-3-(methoxymethoxy)naphthalen-1-yl)-1-fluoro-12-(methylsulfonyl)-4,5,5a,6,9,10-hexahydro-8H-7-oxa-3,10a,11,13-tetraazanaphtho[1,8-ab]heptalene (136 mg, 0.233 mmol, 76% yield) as a white solid. LCMS: (M+1:585.2).

Step 3. To a mixture of ((2R,7aS)-2-fluorotetrahydro-1H-pyrrolizin-7a(5H)-yl)methanol (34.5 mg, 0.216 mmol, 1.1 eq) in THF (2 mL) was added NaH (11.8 mg, 0.295 mmol, 60% purity, 1.5 eq), and the mixture was stirred at 0° C. for 0.5 h, after which 2-(8-ethyl-7-fluoro-3-(methoxymethoxy)naphthalen-1-yl)-1-fluoro-12-(methylsulfonyl)-4,5,5a,6,9,10-hexahydro-8H-7-oxa-3,10a,11,13-tetraazanaphtho[1,8-ab]heptalene (115 mg, 0.197 mmol, 1 eq) in THF (2 mL) was added. The resulting mixture was stirred at 25° C. for 1 h. On completion, the mixture was quenched with sat. NH₄Cl (5 mL) and extracted with ethyl acetate (5 mL×3). The combined organic phase was dried over anhydrous sodium sulfate, filtered, and concentrated to give a residue. The residue was purified by column chromatography (SiO₂, PE/THF=1:0 to 1:3) to give 2-(8-ethyl-7-fluoro-3-(methoxymethoxy)naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (90.0 mg, 0.136 mmol, 69% yield) as a colorless oil. LCMS: (M+1:664.3).

Step 4. To a solution of 2-(8-ethyl-7-fluoro-3-(methoxymethoxy)naphthalen-1-yl)-1-fluoro-12-(((2R,7aS)-2-fluorotetrahydro-1H-pyrrolizin-7a(5H)-yl)methoxy)-4,5,5a,6,9,10-hexahydro-8H-7-oxa-3,10a,11,13-tetraazanaphtho[1,8-ab]heptalene (80.0 mg, 0.120 mmol, 1 eq) in DCM (1 mL) was added HCl/Dioxane (2 M, 0.301 mL, 5 eq). The mixture was stirred at 25° C. for 1 h. On completion, the mixture was concentrated to give a residue. The residue was purified by prep-HPLC to give a mixture of diastereomers (50 mg, 0.081 mmol, 66.9% yield) as a white solid. The mixture was separated by SFC to give two crude products, which were further purified by preparative HPLC to give 5-ethyl-6-fluoro-4-[(8aR)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (3.95 mg, 0.0064 mmol, 6.58% yield) as a white solid (Ex. 3) and 5-ethyl-6-fluoro-4-[(8aS)-4-fluoro-2-{[(2R,7aS)-2-fluorotetrahydro-1H-pyrrolizin-7a(5H)-yl]methoxy}-7,8,8a,9,12,13-hexahydro-11H-10-oxa-1,3,6,13a-tetraazanaphtho[1,8-ab]heptalen-5-yl]naphthalen-2-ol (5.79 mg, 0.0093 mmol, 9.65% yield) as a white solid (Ex. 4). The analytical data for Ex. 3 and Ex. 4 can be found in the table below.

### Preparation of 5-ethynyl-6-fluoro-4-[(8aR)-4-fluoro-2-{[(2R,7aS)-2-fluorotetrahydro-1H-pyrrolizin-7a(5H)-yl]methoxy}-8,8a,9,10,12,13-hexahydro-7H-11-oxa-1,3,6,13a-tetraazanaphtho[1,8-ab]heptalen-5-yl]naphthalen-2-ol (Ex. 5) and 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,12,13-hexahydro-7H-11-oxa-1,3,6,13a-tetraazanaphtho[1,8-ab]heptalen-5-yl]naphthalen-2-ol (Ex. 25)

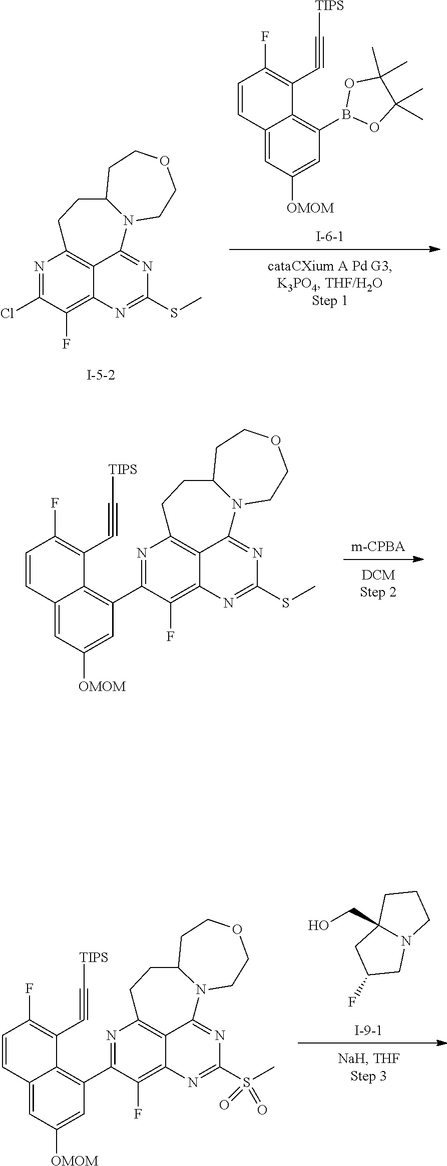

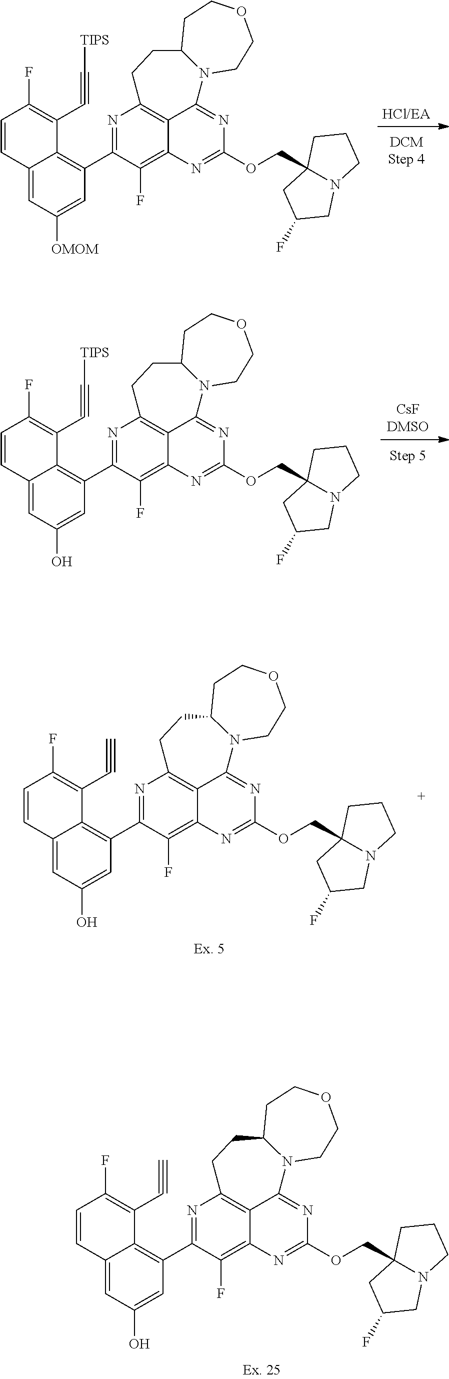

I-5-2 was prepared using intermediate I-2-2, following the procedure described in steps 1-3 for General Method A.

Step 1. A mixture of 2-chloro-1-fluoro-12-(methylthio)-5,5a,6,7,9,10-hexahydro-4H-8-oxa-3,10a,11,13-tetraazanaphtho[1,8-ab]heptalene (I-5-5, 54.0 mg, 0.152 mmol, 1 eq), ((2-fluoro-6-(methoxymethoxy)-8-(4,4,5,5-tetramethyl-1,3,2-dioxaborolan-2-yl)naphthalen-1-yl)ethynyl)triisopropylsilane (85.8 mg, 0.167 mmol, 1.1 eq), cataCXium A Pd G3 (11.0 mg, 0.015 mmol, 0.1 eq), K₃PO₄ (96.9 mg, 0.456 mmol, 3 eq) in THF (3 mL) and H₂O (0.3 mL) was degassed and purged with N₂ thrice, and then the mixture was stirred at 80° C. for 2 hours under N₂ atmosphere. On completion, the mixture was filtered and concentrated to give a residue. The residue was purified by column chromatography (SiO₂, Petroleum ether/Ethyl acetate=1:0 to 0:1) to give 1-fluoro-2-(7-fluoro-3-(methoxymethoxy)-8-((triisopropylsilyl)ethynyl)naphthalen-1-yl)-12-(methylthio)-5,5a,6,7,9,10-hexahydro-4H-8-oxa-3,10a,11,13-tetraazanaphtho[1,8-ab]heptalene (94.0 mg, 0.133 mmol, 87% yield) as a yellow solid. LCMS: (M+1: 705.3)

Step 2. To a solution of 1-fluoro-2-(7-fluoro-3-(methoxymethoxy)-8-((triisopropylsilyl)ethynyl)naphthalen-1-yl)-12-(methylthio)-5,5a,6,7,9,10-hexahydro-4H-8-oxa-3,10a,11,13-tetraazanaphtho[1,8-ab]heptalene (94.0 mg, 0.133 mmol, 1 eq) in DCM (3 mL) was added m-CPBA (115 mg, 0.400 mmol, 60% purity, 3 eq). The mixture was stirred at 25° C. for 1 hour. On completion, the mixture was quenched with saturated solution of Na₂SO₃ (10 mL) and extracted with ethyl acetate (10 mL×3), and the combined organic phase was dried over anhydrous sodium sulfate, filtered and concentrated to give 1-fluoro-2-(7-fluoro-3-(methoxymethoxy)-8-((triisopropylsilyl)ethynyl)naphthalen-1-yl)-12-(methylsulfonyl)-5,5a,6,7,9,10-hexahydro-4H-8-oxa-3,10a,11,13-tetraazanaphtho[1,8-ab]heptalene (98.0 mg, 0.132 mmol, 99% yield) as a yellow solid. LCMS: (M+1: 737.3).

Step 3. To a solution of ((2R,7aS)-2-fluorotetrahydro-1H-pyrrolizin-7a(5H)-yl)methanol (63.5 mg, 0.398 mmol, 3 eq) in THE (3 mL) was added NaH (47.8 mg, 1.20 mmol, 60% purity, 9 eq) and 1-fluoro-2-(7-fluoro-3-(methoxymethoxy)-8-((triisopropylsilyl)ethynyl)naphthalen-1-yl)-12-(methylsulfonyl)-5,5a,6,7,9,10-hexahydro-4H-8-oxa-3,10a,11,13-tetraazanaphtho[1,8-ab]heptalene (98.0 mg, 0.133 mmol, 1 eq.). The mixture was stirred at 0° C. for 1 hour. On completion, the mixture was quenched with water (1 mL) and extracted with ethyl acetate (5 mL×3), and the combined organic phase was dried over anhydrous sodium sulfate, filtered, and concentrated to give a residue. The residue was purified by column chromatography (SiO₂, Petroleum ether/Ethyl acetate=1:0 to 0:1) to give 1-fluoro-2-(7-fluoro-3-(methoxymethoxy)-8-((triisopropylsilyl)ethynyl)naphthalen-1-yl)-12-(((2R,7aS)-2-fluorotetrahydro-1H-pyrrolizin-7a(5H)-yl)methoxy)-5,5a,6,7,9,10-hexahydro-4H-8-oxa-3,10a,11,13-tetraazanaphtho[1,8-ab]heptalene (105 mg, 0.128 mmol, 96% yield) as yellow oil. LCMS: (M+1: 816.4).

Step 4. To a solution of 1-fluoro-2-(7-fluoro-3-(methoxymethoxy)-8-((triisopropylsilyl)ethynyl)naphthalen-1-yl)-12-(((2R,7aS)-2-fluorotetrahydro-1H-pyrrolizin-7a(5H)-yl)methoxy)-5,5a,6,7,9,10-hexahydro-4H-8-oxa-3,10a,11,13-tetraazanaphtho[1,8-ab]heptalene (105 mg, 0.128 mmol, 1 eq) in DCM (3 mL) was added HCl/EtOAc (2 M, 0.5 mL). The mixture was stirred at 25° C. for 1 hour. On completion, the mixture was filtered and concentrated to give 6-fluoro-4-(1-fluoro-12-(((2R,7aS)-2-fluorotetrahydro-1H-pyrrolizin-7a(5H)-yl)methoxy)-5,5a,6,7,9,10-hexahydro-4H-8-oxa-3,10a,11,13-tetraazanaphtho[1,8-ab]heptalen-2-yl)-5-((triisopropylsilyl)ethynyl)naphthalen-2-ol (99.0 mg, 0.128 mmol, 99% yield) as a yellow solid.

Step 5. To a solution of 6-fluoro-4-(1-fluoro-12-(((2R,7aS)-2-fluorotetrahydro-1H-pyrrolizin-7a(5H)-yl)methoxy)-5,5a,6,7,9,10-hexahydro-4H-8-oxa-3,10a,11,13-tetraazanaphtho[1,8-ab]heptalen-2-yl)-5-((triisopropylsilyl)ethynyl)naphthalen-2-ol (99.0 mg, 0.128 mmol, 1 eq) in DMSO (1 mL) was added CsF (97.4 mg, 0.641 mmol, 5 eq). The mixture was stirred at 25° C. for 1 hour. On completion, the mixture was filtered and concentrated to give a residue. The residue was purified by prep-HPLC to give 5-ethynyl-6-fluoro-4-(4-fluoro-2-{[(2R,7aS)-2-fluorotetrahydro-1H-pyrrolizin-7a(5H)-yl]methoxy}-8,8a,9,10,12,13-hexahydro-7H-11-oxa-1,3,6,13a-tetraazanaphtho[1,8-ab]heptalen-5-yl)naphthalen-2-ol (40.0 mg, 0.059 mmol, 46.20% yield, FA salt) as a white solid. This mixture of diastereomers was purified by SFC to give 5-ethynyl-6-fluoro-4-[(8aR)-4-fluoro-2-{[(2R,7aS)-2-fluorotetrahydro-1H-pyrrolizin-7a(5H)-yl]methoxy}-8,8a,9,10,12,13-hexahydro-7H-11-oxa-1,3,6,13a-tetraazanaphtho[1,8-ab]heptalen-5-yl]naphthalen-2-ol (15.4 mg, 0.0246 mmol, 43.34% yield) as a white solid (Ex. 5) and 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,12,13-hexahydro-7H-11-oxa-1,3,6,13a-tetraazanaphtho[1,8-ab]heptalen-5-yl]naphthalen-2-ol (14.9 mg, 0.0235 mmol, 41.49% yield) as a yellow solid (Ex. 25). The analytical data for Ex. 5 and Ex. 25 can be found in the table below.

Ex. 6 and Ex. 26 were prepared following the method used for preparation of Ex. 5, using intermediate I-6-2 in Step 1.

Ex. 9 was prepared following General Method A, using intermediate I-2-3 in Step 1.

Ex. 10 was prepared following General Method A, using intermediate I-2-4 in Step 1, and using XPhos Pd G4 as the catalyst in Step 4.

Ex. 13 was prepared following General Method A, using intermediate I-2-5 in Step 1.

Ex. 18 was prepared following General Method A, using intermediate I-2-6 in Step 1, and using Ad₂nBuP-Pd-G3/K₃PO₄/Dioxane in Step 4.

Ex. 20 and Ex. 27 were prepared following General Method A, using intermediate I-2-7 in Step 1, and using Ad₂nBuP-Pd-G3/K₃PO₄/THF in Step 4.

### General Method B

### Preparation of 5-ethynyl-6-fluoro-4-(4-fluoro-2-{[(2R,7aS)-2-fluorotetrahydro-1H-pyrrolizin-7a(5H)-yl]methoxy}-9,10,11,12-tetrahydro-8aH-1,3,6,12a-tetraazabenzo[4,5]cyclohepta[1,2,3-de]naphthalen-5-yl)naphthalen-2-ol (Ex. 21)

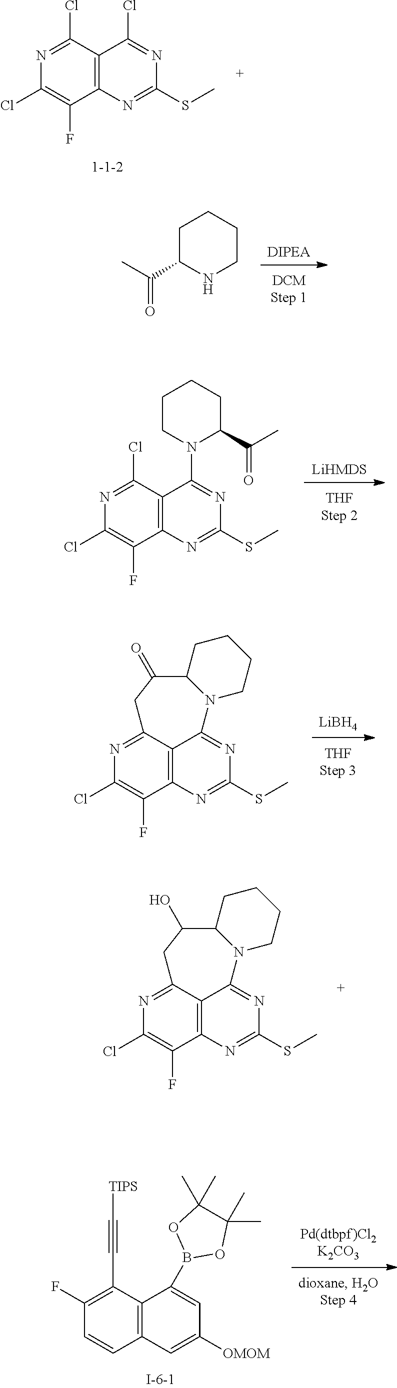

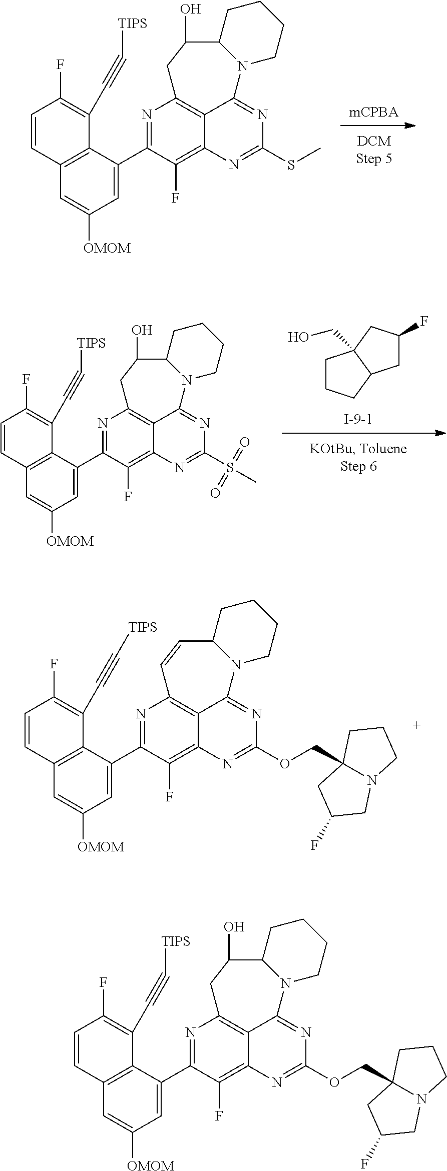

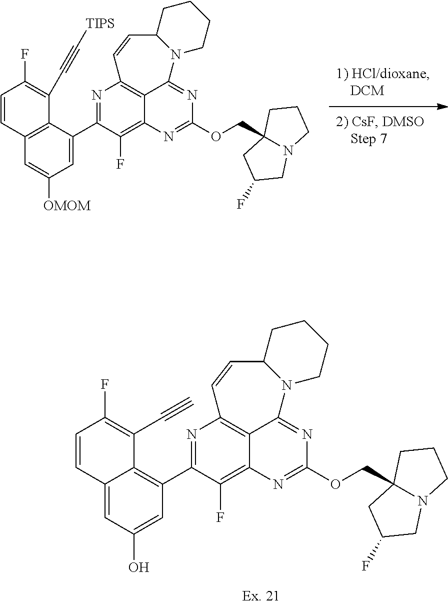

Step 1. To a solution of 4,5,7-trichloro-8-fluoro-2-(methylthio)pyrido[4,3-d]pyrimidine (2.27 g, 7.60 mmol) and 1-(piperidin-2-yl)ethan-1-one (1.49 g, 9.12 mmol, HCl salt) in DCM (33.4 mL) at 0° C. was added DIPEA (3.44 g, 26.6 mmol, 4.63 mL) slowly. The mixture was slowly warmed to ambient temperature over 1.5 h, followed by stirring for an additional 2.5 h. The reaction was then quenched with saturated NaHCO₃ solution (50 mL). The mixture was extracted with DCM (3×50 mL) and combined extracts were dried with Na₂SO₄ and then concentrated under reduced pressure. Flash chromatography (0-20% ethyl acetate in hexane) provided 1-(1-(5,7-dichloro-8-fluoro-2-(methylthio)pyrido[4,3-d]pyrimidin-4-yl)piperidin-2-yl)ethan-1-one (2.60 g, 6.67 mmol, 88% yield). LCMS: m/z 389.1 (M+1).

Step 2. To a solution of LiHMDS (1 M, 12.4 mL) in THE (55.5 mL) at −23° C. was added 1-(1-(5,7-dichloro-8-fluoro-2-(methylthio)pyrido[4,3-d]pyrimidin-4-yl)piperidin-2-yl)ethan-1-one (1.85 g, 4.75 mmol) in THE (10 mL) dropwise by a syringe pump over 100 min. The reaction was stirred at −23° C. for an additional 20 min, then quenched by addition of saturated solution of NH₄Cl (25 mL). The reaction mixture was warmed to ambient temperature and extracted with ethyl acetate (3×20 mL). The combined extracts were dried with brine (25 mL), then Na₂SO₄ and concentrated under reduced pressure. Flash chromatography, (10-20% ethyl acetate in hexane) provided 2-chloro-1-fluoro-11-(methylthio)-6,7,8,9-tetrahydro-4H-3,9a,10,12-tetraazabenzo[4,5]cyclohepta[1,2,3-de]naphthalen-5(5aH)-one (564 mg, 1.60 mmol, 34% yield) as an orange solid. LCMS: m/z 353.1 (M+1).

Step 3. To a solution of 2-chloro-1-fluoro-11-(methylthio)-6,7,8,9-tetrahydro-4H-3,9a,10,12-tetraazabenzo[4,5]cyclohepta[1,2,3-de]naphthalen-5(5aH)-one (564 mg, 1.60 mmol) in THE (8.86 mL) at 0° C. was added LiBH₄ (2 M, 1.80 mL). The mixture was stirred at 0° C. for 45 min, then quenched by addition of saturated NH₄Cl solution (25 mL). The mixture was warmed to ambient temperature then extracted with ethyl acetate (3×15 mL), and the combined extracts were dried with Na₂SO₄ and concentrated under reduced pressure. The solids were triturated with DCM and the supernatant was purified by flash chromatography, (15-25% ethyl acetate in hexane) to provide 2-chloro-1-fluoro-11-(methylthio)-5,5a,6,7,8,9-hexahydro-4H-3,9a,10,12-tetraazabenzo[4,5]cyclohepta[1,2,3-de]naphthalen-5-ol (490.6 mg, 1.38 mmol, 86% yield) (mixture of cis and trans isomers) as a white crystalline solid. LCMS: m/z 355.2 (M+1); ¹H NMR (499 MHz, DMSO-d6) δ ppm 5.65 (d, J=4.38 Hz, 1H) 5.42 (d, J=5.48 Hz, 1H) 4.32 (td, J=8.76, 5.48 Hz, 1H) 3.99-4.18 (m, 2H) 3.85-3.93 (m, 1H) 3.65 (ddd, J=12.87, 8.49, 3.83 Hz, 1H) 3.47 (dt, J=9.99, 4.04 Hz, 1H) 3.17-3.29 (m, 2H) 3.08 (dd, J=14.24, 4.93 Hz, 1H) 2.99-3.06 (m, 1H) 2.54 (s, 3H) 2.53 (s, 1H) 1.87-1.96 (m, 1H) 1.72-1.87 (m, 4H) 1.49-1.72 (m, 3H) 1.42 (ddt, J=12.59, 8.35, 4.31, 4.31 Hz, 1H).

Step 4. To an argon degassed solution of 2-chloro-1-fluoro-11-(methylthio)-5,5a,6,7,8,9-hexahydro-4H-3,9a,10,12-tetraazabenzo[4,5]cyclohepta[1,2,3-de]naphthalen-5-ol (88.42 mg, 0.249 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (159.7 mg, 0.311 mmol), and K₂CO₃ (206.6 mg, 1.50 mmol) in dioxane (1.03 mL) and water (0.212 mL) was added Pd(dtbpf)Cl₂ (16 mg, 0.025 mmol). The mixture was heated to 105° C. for 15 hr. The reaction was worked up by cooling and diluting with DCM (15 mL) and water (15 mL). The mixture was extracted with DCM (3×15 mL). The combined extracts were dried with Na₂SO₄ and then concentrated under reduced pressure. The crude material was purified by flash chromatography, (0-50% ethyl acetate in dichloromethane) to give 1-fluoro-2-(7-fluoro-3-(methoxymethoxy)-8-((triisopropylsilyl)ethynyl)naphthalen-1-yl)-11-(methylthio)-5,5a,6,7,8,9-hexahydro-4H-3,9a,10,12-tetraazabenzo[4,5]cyclohepta[1,2,3-de]naphthalen-5-ol (46.48 mg, 0.066 mmol, 26% yield). LCMS: m/z 705.1 (M+1).

Step 5. To a solution of 1-fluoro-2-(7-fluoro-3-(methoxymethoxy)-8-((triisopropylsilyl)ethynyl)naphthalen-1-yl)-11-(methylthio)-5,5a,6,7,8,9-hexahydro-4H-3,9a,10,12-tetraazabenzo[4,5]cyclohepta[1,2,3-de]naphthalen-5-ol (46.48 mg, 0.066 mmol) in DCM (0.5 mL) at 0° C. was added m-CPBA (57 mg, 0.33 mmol). The reaction mixture was stirred at 0° C. for 5 h, then purified by flash chromatography (automated system, 12 g Silica, 0-100% ethyl acetate in DCM) to give 1-fluoro-2-(7-fluoro-3-(methoxymethoxy)-8-((triisopropylsilyl)ethynyl)naphthalen-1-yl)-11-(methylsulfonyl)-5,5a,6,7,8,9-hexahydro-4H-3,9a,10,12-tetraazabenzo[4,5]cyclohepta[1,2,3-de]naphthalen-5-ol (32.30 mg, 0.044 mmol, 66% yield) as a light yellow glassy solid. LCMS: m/z 737.4 (M+1).

Step 6. To a solution of ((2R,7aS)-2-fluorotetrahydro-1H-pyrrolizin-7a(5H)-yl)methanol (28 mg, 0.175 mmol) in toluene (0.4 mL) at 0° C. was added potassium tert-butoxide (1 M, 0.18 mL). The mixture was stirred at 0° C. for 10 min, then a solution of 1-fluoro-2-(7-fluoro-3-(methoxymethoxy)-8-((triisopropylsilyl)ethynyl)naphthalen-1-yl)-11-(methylsulfonyl)-5,5a,6,7,8,9-hexahydro-4H-3,9a,10,12-tetraazabenzo[4,5]cyclohepta[1,2,3-de]naphthalen-5-ol (32.30 mg, 0.044 mmol) in toluene (0.25 mL) was added. The mixture was stirred at 0° C. for 1.5 h. The mixture was then quenched by addition of saturated solution of NH₄Cl (5 mL). The mixture was extracted with ethyl acetate (3×5 mL). The combined extracts were dried with Na₂SO₄ and then concentrated under reduced pressure. The material was purified by flash chromatography (loaded with DCM+TEA (0.1 mL), (1.25-10% methanol in dichloromethane) to give 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5a,6,8,9-tetrahydro-7H-3,9a,10,12-tetraazabenzo[4,5]cyclohepta[1,2,3-de]naphthalene (3.35 mg, 0.004 mmol, 9.6% yield) as a glassy solid; LCMS: m/z 798.5 (M+1) and 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-ol (12.42 mg, 0.015 mmol, 35% yield) as a white solid. LCMS: m/z 816.5 (M+1).

Step 7. To a solution of 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5a,6,8,9-tetrahydro-7H-3,9a,10,12-tetraazabenzo[4,5]cyclohepta[1,2,3-de]naphthalene (3.35 mg, 0.004 mmol) in DCM (1 mL) at 0° C. was added HCl (4 M, 0.2 mL) in dioxane. The mixture was stirred at 0° C. for 45 min. The mixture was concentrated under reduced pressure to give 6-fluoro-4-(1-fluoro-11-(((2R,7aS)-2-fluorotetrahydro-1H-pyrrolizin-7a(5H)-yl)methoxy)-5a,6,8,9-tetrahydro-7H-3,9a,10,12-tetraazabenzo[4,5]cyclohepta[1,2,3-de]naphthalen-2-yl)-5-((triisopropylsilyl)ethynyl)naphthalen-2-ol (3.16 mg, 0.004 mmol, assumed quantitative). LCMS: m/z 754.6 (M+1).

Then, to a solution of 6-fluoro-4-(1-fluoro-11-(((2R,7aS)-2-fluorotetrahydro-1H-pyrrolizin-7a(5H)-yl)methoxy)-5a,6,8,9-tetrahydro-7H-3,9a,10,12-tetraazabenzo[4,5]cyclohepta[1,2,3-de]naphthalen-2-yl)-5-((triisopropylsilyl)ethynyl)naphthalen-2-ol (3.16 mg, 0.004 mmol) in DMSO (0.5 mL) was added CsF (9.6 mg, 0.063 mmol). The mixture was stirred at ambient temperature for 1 h. The mixture was then diluted with ethyl acetate (15 mL), filtered through syringe filter, washed with 10% brine (5 mL), and then concentrated under reduced pressure. Purification by HPLC gave 5-ethynyl-6-fluoro-4-(4-fluoro-2-{[(2R,7aS)-2-fluorotetrahydro-1H-pyrrolizin-7a(5H)-yl]methoxy}-9,10,11,12-tetrahydro-8aH-1,3,6,12a-tetraazabenzo[4,5]cyclohepta[1,2,3-de]naphthalen-5-yl)naphthalen-2-ol (1.72 mg, 0.0024 mmol, 57% yield) as a TFA salt (Ex. 21). The analytical data for Ex. 21 can be found in the table below.

Ex. 22 was prepared following General Method A, using intermediate I-2-8 in Step 1.

### Preparation of 5-ethyl-6-fluoro-4-(4-fluoro-2-{[(2R,7aS)-2-fluorotetrahydro-1H-pyrrolizin-7a(5H)-yl]methoxy}-7,8,8a,9,10,11,12,13-octahydro-1,3,6,13a-tetraazanaphtho[1,8-ab]heptalen-5-yl)naphthalen-2-ol (Ex. 23)

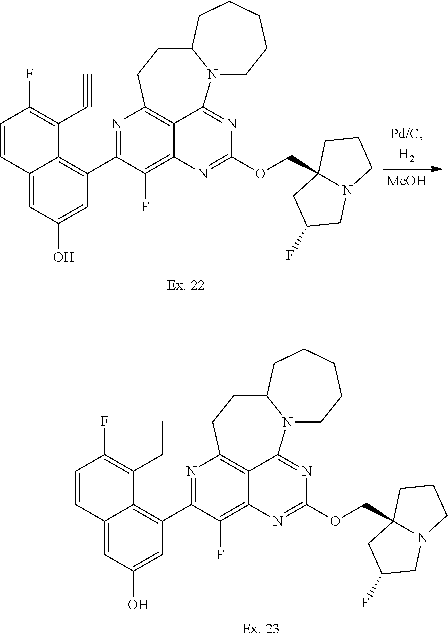 [SMILES: "MeOH" ; C#Cc1c(F)ccc2cc(O)cc(-c3nc4c5c(nc(OC[C@@]67CCCN6C[C@H](F)C7)nc5c3F)N3CCCCCC3CC4)c12 ; CCc1c(F)ccc2cc(O)cc(-c3nc4c5c(nc(OC[C@@]67CCCN6C[C@H](F)C7)nc5c3F)N3CCCCCC3CC4)c12]

Pd/C (26.0 mg, 0.024 mmol, 10% purity, 0.5 eq) was moistened with MeOH (1 mL) under Argon. Then, a solution of 5-ethynyl-6-fluoro-4-(4-fluoro-2-{[(2R,7aS)-2-fluorotetrahydro-1H-pyrrolizin-7a(5H)-yl]methoxy}-7,8,8a,9,10,11,12,13-octahydro-1,3,6,13a-tetraazanaphtho[1,8-ab]heptalen-5-yl)naphthalen-2-ol (30.0 mg, 0.049 mmol, 1 eq) in MeOH (1 mL) was added. The suspension was degassed under vacuum and purged with H₂ (15 Psi) 3 times. The mixture was stirred under H₂ at 25° C. for 1 hour under H₂ (15 Psi). On completion, the reaction solution is filtered to remove the Pd/C. The residue was purified by prep-HPLC purification min) to give 5-ethyl-6-fluoro-4-(4-fluoro-2-{[(2R,7aS)-2-fluorotetrahydro-1H-pyrrolizin-7a(5H)-yl]methoxy}-7,8,8a,9,10,11,12,13-octahydro-1,3,6,13a-tetraazanaphtho[1,8-ab]heptalen-5-yl)naphthalen-2-ol (9.62 mg, 0.015 mmol, 30.85% yield) as an off-white solid. Analytical data for Ex. 23 can be found in the table below.

Ex. 24 was prepared following General Method A, using intermediate I-2-7 in Step 1, and using intermediate I-9-2 in Step 6.

### Preparation of 5-(8-ethynyl-7-fluoro-3-hydroxynaphthalen-I-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalene-8-carbonitrile (Ex. 28) and 5-(8-ethenyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalene-8-carbonitrile (Ex. 32)

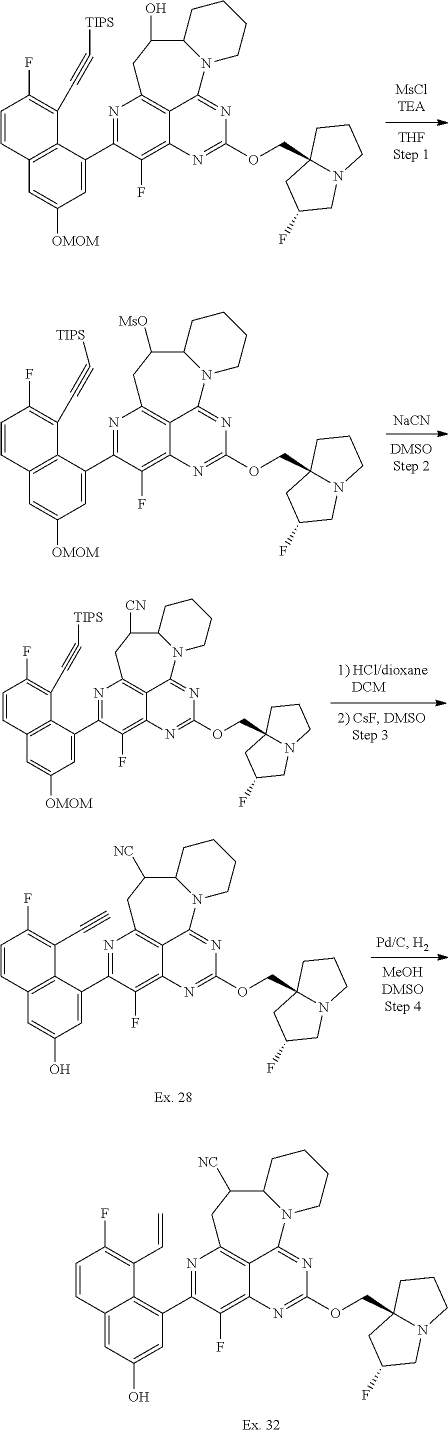

Step 1. To a solution of 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-ol, the preparation of which was described in Ex. 21 (37.19 mg, 0.045 mmol) and triethylamine (46.2 mg, 0.456 mmol, 0.064 mL) in THF (0.5 mL) at 0° C. was added methanesulfonyl chloride (26 mg, 0.228 mmol, 0.018 mL) dropwise. The reaction mixture was stirred for 15 min then quenched with water (3 mL). The mixture extracted with DCM (3×5 mL). Combined extracts were dried with Na₂SO₄ and concentrated under reduced pressure. The material was purified by flash chromatography (loaded with DCM+TEA (0.1 mL), (0-10% methanol in dichloromethane) to give 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-yl methanesulfonate (35.19 mg, 0.039 mmol, 86% yield) as a white solid. LCMS: m/z 894.6 (M+1).

Step 2. To a solution of 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-yl methanesulfonate (35.19 mg, 0.039 mmol) in DMSO (0.5 mL) was added NaCN (96 mg, 1.97 mmol). The mixture was heated to 40° C. and stirred for 50 h. The reaction was diluted with ethyl acetate (30 mL) and washed with water (20 mL) and 30% brine (20 mL). The organic layer was dried with Na₂SO₄ and concentrated under reduced pressure. Flash chromatography (loaded with 0.1 mL TEA), (0-10% methanol in dichloromethane) gave 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalene-5-carbonitrile (30.15 mg, 0.036 mmol, 92% yield, contaminated with elimination product). LCMS: m/z 825.6 (M+1).

Step 3. To a solution of 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalene-5-carbonitrile (30.15 mg, 0.036 mmol) in DCM (2 mL) at 0° C. was added HCl (4 M, 0.5 mL) in dioxane. The mixture was stirred at 0° C. for 1.5 h. The mixture was concentrated under reduced pressure to give 1-fluoro-2-(7-fluoro-3-hydroxy-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalene-5-carbonitrile (28.54 mg, 0.036 mmol, assumed quantitative). LCMS: m/z 781.4 (M+1).

Then, to a solution of 1-fluoro-2-(7-fluoro-3-hydroxy-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalene-5-carbonitrile (28.54 mg, 0.036 mmol) in DMSO (1 mL) was added CsF (139 mg, 0.915 mmol). The mixture was stirred at ambient temperature for 10 min. The mixture was diluted with ethyl acetate (25 mL), filtered, washed with 10% brine (15 mL), 50% brine (15 mL), dried with Na₂SO₄, then concentrated under reduced pressure. Purification by HPLC gave 5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalene-8-carbonitrile (10.82 mg, 0.003 mmol, 7.7% yield) as a TFA salt (Ex. 28). The analytical data for Ex. 28 can be found in the table below.

Step 4. To a solution of 5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalene-8-carbonitrile (10 mg, 0.0013 mmol) as TFA salt in methanol (4 mL) and DMSO (1.5 mL) was added Pd/C (7.2 mg, 0.007 mmol, 10% purity). The mixture was purged of air and placed under H₂ (1 atm). The mixture was stirred for 2 h, then filtered and concentrated under reduced pressure. HPLC chromatography gave 5-(8-ethenyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalene-8-carbonitrile (3.40 mg, 0.004 mmol, 34% yield, TFA salt) as a light yellow solid (Ex. 32). The analytical data for Ex. 32 can be found in the table below.

### Preparation of 5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalen-8-ol (Ex. 29)

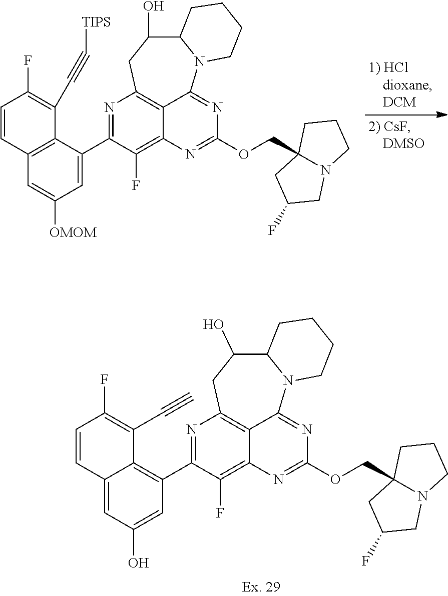

Step 1. To a solution of 1-fluoro-2-(7-fluoro-3-(methoxymethoxy)-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-ol, the preparation of which was described in Ex. 21 (12.42 mg, 0.015 mmol) in DCM (1 mL) at 0° C. was added HCl (4 M, 0.250 mL) in dioxane. The mixture was stirred at 0° C. for 1.25 h. Then, the mixture was concentrated under reduced pressure to give 1-fluoro-2-(7-fluoro-3-hydroxy-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-ol (11.75 mg, 0.015 mmol, assumed quantitative). LCMS: m/z 772.5 (M+1).

Then, to a solution of 1-fluoro-2-(7-fluoro-3-hydroxy-8-((triisopropylsilyl)ethynyl)naphthalen-1-yl)-11-(((2R,7aS)-2-fluorotetrahydro-1H-pyrrolizin-7a(5H)-yl)methoxy)-5,5a,6,7,8,9-hexahydro-4H-3,9a,10,12-tetraazabenzo[4,5]cyclohepta[1,2,3-de]naphthalen-5-ol (11.75 mg, 0.015 mmol) in DMSO (0.5 mL) was added CsF (35 mg, 0.228 mmol). The mixture was stirred at ambient temperature for 10 min. The mixture was diluted with ethyl acetate (15 mL) filtered through syringe filter, washed with 10% brine (5 mL), dried with Na₂SO₄ then concentrated under reduced pressure. Purification by HPLC gave 5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalen-8-ol (9.48 mg, 0.013 mmol, 85% yield) as a TFA salt (Ex. 29). Analytical data for Ex. 29 can be found in the table below.

### Preparation of 5-ethynyl-6-fluoro-4-(4-fluoro-2-{[(2R,7aS)-2-fluorotetrahydro-1H-pyrrolizin-7a(5H)-yl]methoxy}-7-methyl-8,9,10,11-tetrahydropyrido[1,2-g]pyrimido[4,5,6-de][1,6]naphthyridin-5-yl)naphthalen-2-ol (Ex. 30)

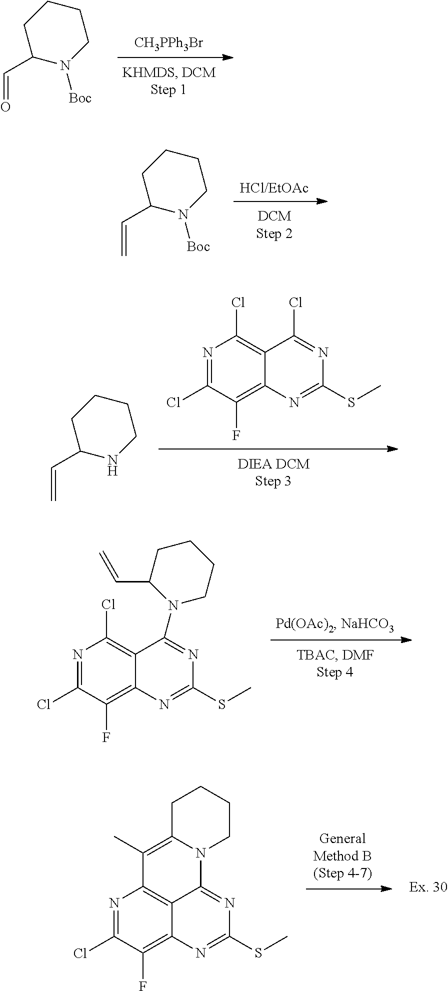

Step 1. To a solution of methyl(triphenyl)phosphonium bromide (5.02 g, 14.0 mmol, 3 eq) in THF (10 mL) was added KHMDS (1 M, 14.0 mL, 3 eq) at 0° C. The mixture was stirred at 25° C. for 0.5 h, then a solution of tert-butyl 2-formylpiperidine-1-carboxylate (1.00 g, 4.69 mmol, 1 eq) in THE (5 mL) was added at −78° C., and the mixture was stirred at 25° C. for 1.5 h. On completion, the mixture was quenched with water (50 mL) and extracted with ethyl acetate (40 mL×2). The combined organic phase was dried over anhydrous sodium sulfate, filtered, and concentrated to give a residue. The residue was purified by silica gel column chromatography (Petroleum ether/Ethyl acetate=1:0 to 3:1) to give tert-butyl 2-vinylpiperidine-1-carboxylate (780 mg, 3.69 mmol, 79% yield) as yellow oil. ¹H NMR (400 MHz, CDCl₃) δ=5.87-5.68 (m, 1H), 5.24-5.12 (m, 1H), 5.07-4.99 (m, 1H), 4.88-4.67 (m, 1H), 4.05-3.88 (m, 1H), 2.92-2.75 (m, 1H), 1.81-1.55 (m, 6H), 1.46 (s, 9H).

Step 2. To a solution of tert-butyl 2-vinylpiperidine-1-carboxylate (500 mg, 2.37 mmol, 1 eq) in DCM (5 mL) was added HCl/EtOAc (4 M, 1 mL, 1.69 eq). The mixture was stirred at 25° C. for 1 hr. On completion, the mixture was concentrated to give 2-vinylpiperidine (260 mg, crude) as yellow oil.

Step 3. To a solution of 2-vinylpiperidine (250 mg, 2.25 mmol, 1 eq), 4,5,7-trichloro-8-fluoro-2-methylsulfanyl-pyrido[4,3-d]pyrimidine (671 mg, 2.25 mmol, 1 eq) in DCM (4 mL) was added DIEA (871 mg, 6.75 mmol, 1.17 mL, 3 eq). The mixture was stirred at 25° C. for 1 h. The mixture was diluted with water (50 mL) and extracted with ethyl acetate (40 mL×3). The combined organic phase was washed with water (50 mL×2) and dried over Na₂SO₄, filtered, and the filtrate was concentrated to give a residue. The residue was purified by column chromatography (SiO₂, Petroleum ether/Ethyl acetate=1:0 to 80:20) to give 5,7-dichloro-8-fluoro-2-(methylthio)-4-(2-vinylpiperidin-1-yl)pyrido[4,3-d]pyrimidine (550 mg, 1.47 mmol, 66% yield) as yellow oil. LCMS: (M+1:373.1).

Step 4. A mixture of 5,7-dichloro-8-fluoro-2-(methylthio)-4-(2-vinylpiperidin-1-yl)pyrido[4,3-d]pyrimidine (400 mg, 1.07 mmol, 1 eq), TBAC (297 mg, 1.07 mmol, 1 eq), Pd(OAc)₂ (48.1 mg, 0.214 mmol, 0.2 eq), NaHCO₃ (225 mg, 2.68 mmol, 2.5 eq) in DMF (4 mL) was degassed and purged with N₂ 3 times, and then the mixture was stirred at 80° C. for 2 h under N₂ atmosphere. On completion, the mixture was diluted with water (50 mL) and extracted with ethyl acetate (30 mL×3). The combined organic phase was washed with water (50 mL×2) and dried over Na₂SO₄, filtered and the filtrate was concentrated to give a residue. The residue was purified by column chromatography (SiO₂, Petroleum ether/Ethyl acetate=1:0 to 5:1) to give 5-chloro-4-fluoro-7-methyl-2-(methylthio)-8,9,10,11-tetrahydropyrido[1,2-g]pyrimido[4,5,6-de][1,6]naphthyridine (100 mg, 0.296 mmol, 28% yield) as black solid. LCMS: (M+1:337.0).

Ex. 30 was synthesized using the above intermediate (5-chloro-4-fluoro-7-methyl-2-(methylthio)-8,9,10,11-tetrahydropyrido[1,2-g]pyrimido[4,5,6-de][1,6]naphthyridine) following General Method B, Steps 4 to 7.

### Preparation of 5-ethyl-6-fluoro-4-(4-fluoro-2-{[(2R,7aS)-2-fluorotetrahydro-1H-pyrrolizin-7a(5H)-yl]methoxy}-9,10,11,12-tetrahydro-8aH-1,3,6,12a-tetraazabenzo[4,5]cyclohepta[1,2,3-de]naphthalen-5-yl)naphthalen-2-ol (Ex. 31)

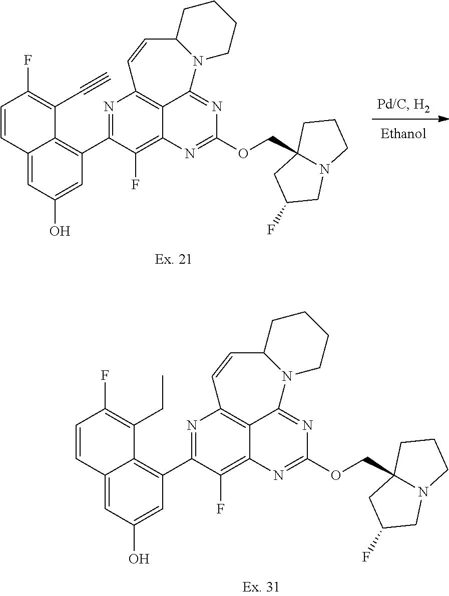 [SMILES: C#Cc1c(F)ccc2cc(O)cc(-c3nc4c5c(nc(OC[C@@]67CCCN6C[C@H](F)C7)nc5c3F)N3CCCCC3C=C4)c12 ; "Pd/C, H2" ; CCc1c(F)ccc2cc(O)cc(-c3nc4c5c(nc(OC[C@@]67CCCN6C[C@H](F)C7)nc5c3F)N3CCCCC3C=C4)c12]

To a solution of 5-ethynyl-6-fluoro-4-(4-fluoro-2-{[(2R,7aS)-2-fluorotetrahydro-1H-pyrrolizin-7a(5H)-yl]methoxy}-9,10,11,12-tetrahydro-8aH-1,3,6,12a-tetraazabenzo[4,5]cyclohepta[1,2,3-de]naphthalen-5-yl)naphthalen-2-ol (Ex. 21, 1.90 mg, 0.0027 mmol) as TFA salt in ethanol (1.5 mL) was added Pd/C (2.8 mg, 0.0028 mmol, 10% purity). The mixture was purged of air and placed under H₂ (1 atm). The mixture was stirred for 2 h, then filtered and concentrated under reduced pressure. HPLC chromatography gave 5-ethyl-6-fluoro-4-(4-fluoro-2-{[(2R,7aS)-2-fluorotetrahydro-1H-pyrrolizin-7a(5H)-yl]methoxy}-9,10,11,12-tetrahydro-8aH-1,3,6,12a-tetraazabenzo[4,5]cyclohepta[1,2,3-de]naphthalen-5-yl)naphthalen-2-ol (0.79 mg, 0.001 mmol, 39% yield, TFA salt) as a light yellow solid (Ex. 31). Analytical data for Ex. 31 can be found in the table below.

The following examples were made following General Method A or B as indicated in the Table:

| | | | MS m/z | |
|---|---|---|---|---|
| Ex. # | Structure | General Method | [M + H]⁺ | ¹H NMR (400 MHz, DMSO-d₆) δ ppm |
| 1 | 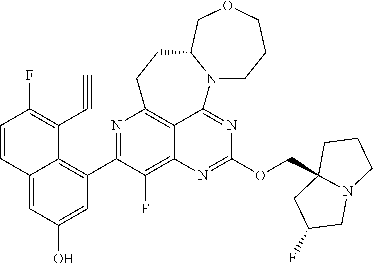 [SMILES: C#Cc1c(F)ccc2cc(O)cc(-c3nc4c5c(nc(OC[C@@]67CCCN6C[C@H](F)C7)nc5c3F)N3CCCOC[C@H]3CC4)c12] | A | 616.3 | δ = 10.53-9.83 (m, 1H), 7.99-7.92 (m, 1H), 7.45 (t, J = 8.8 Hz, 1H), 7.37 (d, J = 2.4 Hz, 1H), 7.23-7.10 (m, 1H), 5.37- 5.17 (m, 1H), 4.20-4.00 (m, 4H), 3.99- 3.92 (m, 1H), 3.87-3.78 (m, 2H), 3.75- 3.64 (m, 2H), 3.63-3.54 (m, 1H), 3.09 (d, J = 6.0 Hz, 2H), 3.06-2.98 (m, 2H), 2.96-2.81 (m, 2H), 2.28-1.97 (m, 6H), 1.93-1.75 (m, 4H). |
| 2 | 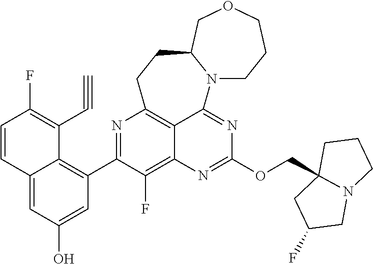 [SMILES: C#Cc1c(F)ccc2cc(O)cc(-c3nc4c5c(nc(OC[C@@]67CCCN6C[C@H](F)C7)nc5c3F)N3CCCOC[C@@H]3CC4)c12] | A | 616.3 | ¹H NMR (400 MHz, DMSO-d₆) δ = 10.65- 9.77 (m, 1H), 8.03-7.90 (m, 1H), 7.45 (t, J = 8.8 Hz, 1H), 7.37 (s, 1H), 7.22-7.10 (m, 1H), 5.40-5.15 (m, 1H), 4.20-4.12 (m, 2H), 4.11-4.06 (m, 1H), 4.06-4.00 (m, 1H), 3.99-3.92 (m, 1H), 3.86-3.77 (m, 2H), 3.75-3.65 (m, 2H), 3.64-3.55 (m, 1H), 3.09 (d, J = 9.2 Hz, 2H), 3.06- 2.99 (m, 2H), 2.95-2.81 (m, 2H), 2.25- 1.96 (m, 6H), 1.93-1.75 (m, 4H). |
| 3 | 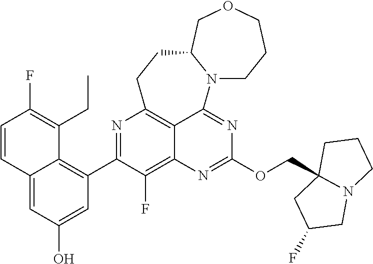 [SMILES: CCc1c(F)ccc2cc(O)cc(-c3nc4c5c(nc(OC[C@@]67CCCN6C[C@H](F)C7)nc5c3F)N3CCCOC[C@H]3CC4)c12] | A | 620.2 | ¹H NMR (400 MHz, MeOD-d₄) δ = 7.72- 7.61 (m, 1H), 7.34-7.18 (m, 2H), 7.15- 6.98 (m, 1H), 5.42-5.17 (m, 1H), 4.65- 4.45 (m, 2H), 4.41-4.18 (m, 2H), 4.12- 3.85 (m, 4H), 3.84-3.62 (m, 2H), 3.21 (d, J = 19.2 Hz, 3H), 3.08-2.97 (m, 2H), 2.55-2.07 (m, 8H), 2.07-1.81 (m, 4H), 0.93-0.76 (m, 3H). |
| 4 | 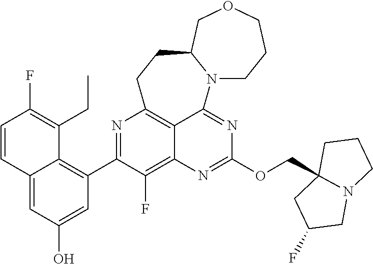 [SMILES: CCc1c(F)ccc2cc(O)cc(-c3nc4c5c(nc(OC[C@@]67CCCN6C[C@H](F)C7)nc5c3F)N3CCCOC[C@@H]3CC4)c12] | A | 620.2 | ¹H NMR (400 MHz, MeOD-d₄) δ = 7.72- 7.61 (m, 1H), 7.34-7.18 (m, 2H), 7.15- 6.98 (m, 1H), 5.42-5.17 (m, 1H), 4.65- 4.45 (m, 2H), 4.41-4.18 (m, 2H), 4.12- 3.85 (m, 4H), 3.84-3.62 (m, 2H), 3.21 (d, J = 19.2 Hz, 3H), 3.08-2.97 (m, 2H), 2.55-2.07 (m, 8H), 2.07-1.81 (m, 4H), 0.93-0.76 (m, 3H). |
| 5 | 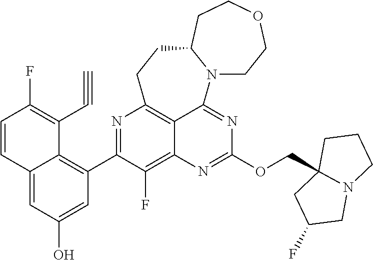 [SMILES: C#Cc1c(F)ccc2cc(O)cc(-c3nc4c5c(nc(OC[C@@]67CCCN6C[C@H](F)C7)nc5c3F)N3CCOCC[C@H]3CC4)c12] | A | 616.2 | δ = 10.29-10.03 (m, 1H), 8.01-7.93 (m, 1H), 7.46 (t, J = 8.8 Hz, 1H), 7.38 (s, 1H), 7.22 (d, J = 1.6 Hz, 1H), 5.40-5.17 (m, 1H), 4.47-4.29 (m, 1H), 4.08-4.02 (m, 2H), 3.97-3.82 (m, 2H), 3.79-3.57 (m, 4H), 3.09 (d, J = 5.2 Hz, 2H), 3.06- 3.00 (m, 2H), 2.88-2.80 (m, 2H), 2.68 (s, 1H), 2.34 (s, 2H), 2.11 (d, J = 5.6 Hz, 2H), 2.06-2.00 (m, 2H), 1.82-1.74 (m, 2H), 1.24 (s, 2H). |
| 6 | 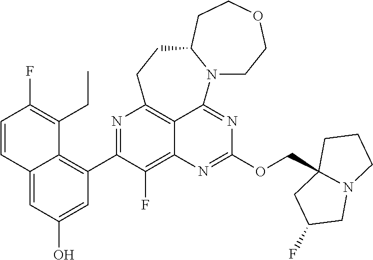 [SMILES: CCc1c(F)ccc2cc(O)cc(-c3nc4c5c(nc(OC[C@@]67CCCN6C[C@H](F)C7)nc5c3F)N3CCOCC[C@H]3CC4)c12] | A | 620.4 | δ = 9.92 (dd, J = 2.0, 3.6 Hz, 1H), 7.85- 7.69 (m, 1H), 7.42-7.29 (m, 2H), 7.12- 6.90 (m, 1H), 5.42-5.19 (m, 1H), 4.50- 4.34 (m, 1H), 4.15-3.99 (m, 3H), 3.97- 3.85 (m, 1H), 3.81-3.55 (m, 3H), 3.18- 2.98 (m, 4H), 2.95-2.78 (m, 2H), 2.39- 2.19 (m, 5H), 2.16-2.10 (m, 2H), 2.04 (d, J = 11.6 Hz, 3H), 1.92-1.72 (m, 3H), 0.86-0.74 (m, 3H). |
| 9 | 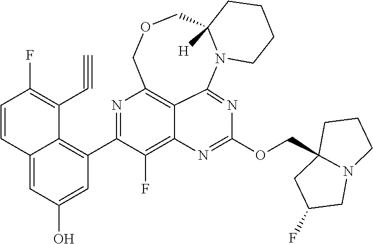 | A | 616.2 | 10.38-9.92 (m, 1H), 8.00-7.92 (m, 1H), 7.49-7.41 (m, 1H), 7.40-7.34 (m, 1H), 7.29-7.04 (m, 1H), 5.39-5.19 (m, 1H), 5.18-5.07 (m, 1H), 4.92-4.70 (m, 2H), 4.40-4.31 (m, 1H), 4.29-4.18 (m, 1H), 4.13-3.94 (m, 3H), 3.75-3.64 (m, 1H), 3.10 (br d, J = 9.8 Hz, 2H), 3.03 (br s, 1H), 2.88-2.79 (m, 1H), 2.18-2.10 (m, 1H), 2.08-2.04 (m, 1H), 2.03-1.97 (m, 1H), 1.92-1.72 (m, 5H), 1.70-1.41 (m, 5H). |
| 10 | 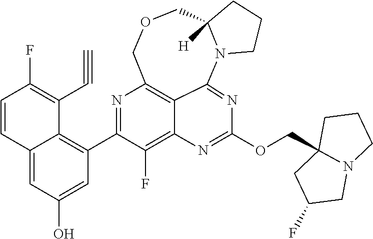 | A | 602.2 | 10.18-10.08 (m, 1H), 8.00-7.93 (m, 1H), 7.50-7.41 (m, 1H), 7.40-7.35 (m, 1H), 7.31-7.00 (m, 1H), 5.41-5.19 (m, 1H), 5.05-4.93 (m, 1H), 4.76 (d, J = 17.6 Hz, 1H), 4.42-4.16 (m, 1H), 4.15- 3.98 (m, 3H), 3.89-3.82 (m, 1H), 3.81- 3.69 (m, 3H), 3.15-3.05 (m, 2H), 3.02 (s, 1H), 2.88-2.78 (m, 1H), 2.13-1.94 (m, 6H), 1.88-1.71 (m, 4H). |
| 13 | 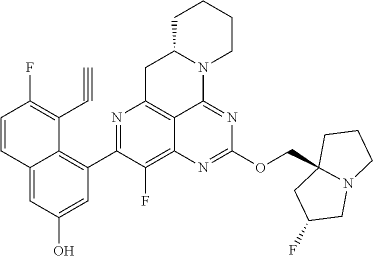 [SMILES: C#Cc1c(F)ccc2cc(O)cc(-c3nc4c5c(nc(OC[C@@]67CCCN6C[C@H](F)C7)nc5c3F)N3CCCC[C@H]3C4)c12] | A | 586.1 | δ = 7.90-7.82 (m, 1H), 7.41-7.28 (m, 2H), 7.26-7.11 (m, 1H), 5.50-5.26 (m, 1H), 4.50-4.29 (m, 2H), 4.01-3.86 (m, 1H), 3.52-3.36 (m, 6H), 3.19-3.09 (m, 1H), 3.08-2.94 (m, 2H), 2.51-2.27 (m, 2H), 2.27-2.17 (m, 1H), 2.14-2.03 (m, 2H), 2.02-1.82 (m, 4H), 1.81-1.60 (m, 3H). |
| 18 | 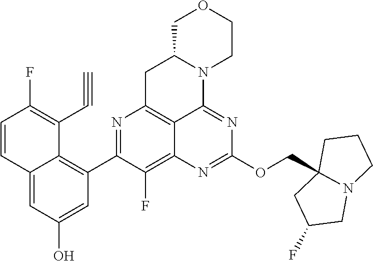 [SMILES: C#Cc1c(F)ccc2cc(O)cc(-c3nc4c5c(nc(OC[C@@]67CCCN6C[C@H](F)C7)nc5c3F)N3CCOC[C@H]3C4)c12] | A | 588.2 | δ = 7.88-7.82 (m, 1H), 7.36-7.29 (m, 2H), 7.23-7.17 (m, 1H), 5.42-5.21 (m, 1H), 4.66-4.54 (m, 2H), 4.37- 4.25 (m, 2H), 4.23-4.12 (m, 2H), 4.08-3.95 (m, 1H), 3.82-3.69 (m, 1H), 3.65-3.53 (m, 1H), 3.26-3.20 (m, 4H), 3.09-2.90 (m, 2H), 2.41-2.32 (m, 1H), 2.31-2.19 (m, 2H), 2.19-2.08 (m, 1H), 2.07-1.95 (m, 2H), 1.95-1.83 (m, 1H) |
| 20 | 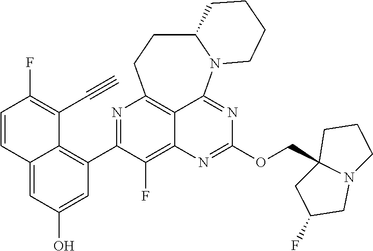 [SMILES: C#Cc1c(F)ccc2cc(O)cc(-c3nc4c5c(nc(OC[C@@]67CCCN6C[C@H](F)C7)nc5c3F)N3CCCC[C@H]3CC4)c12] | A | 600.1 | δ = 10.34-10.00 (m, 1H), 7.96 (dd, J = 6.0, 9.2 Hz, 1H), 7.44 (s, 1H), 7.37 (d, J = 2.4 Hz, 1H), 7.30-7.03 (m, 1H), 5.38- 5.18 (m, 1H), 4.85 (s, 1H), 4.79-4.70 (m, 1H), 4.22 (br s, 1H), 4.14 (br d, J = 10.4 Hz, 1H), 4.07-3.96 (m, 2H), 3.91 (s, 1H), 3.22 (s, 3H), 3.13-3.07 (m, 2H), 3.02 (br s, 1H), 2.88-2.78 (m, 1H), 2.16- 2.10 (m, 1H), 2.05 (br s, 2H), 1.90- 1.72 (m, 3H), 1.27 (br d, J = 6.4 Hz, 3H). |
| 21 | 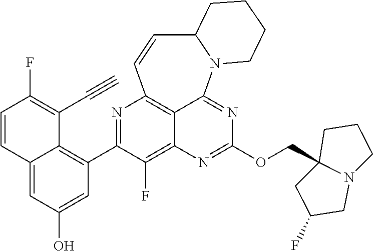 [SMILES: C#Cc1c(F)ccc2cc(O)cc(-c3nc4c5c(nc(OC[C@@]67CCCN6C[C@H](F)C7)nc5c3F)N3CCCCC3C=C4)c12] | B | 598.3 | δ ppm 10.69-10.91 (m, 1 H) 10.18 (br s, 1 H) 7.94-8.02 (m, 1 H) 7.42-7.53 (m, 1 H) 7.38-7.42 (m, 1 H) 7.10-7.26 (m, 1 H) 6.69-6.76 (m, 1 H) 6.32-6.46 (m, 1 H) 5.47-5.64 (m, 1 H) 4.49-4.69 (m, 2 H) 4.15-4.28 (m, 2 H) 4.01-4.08 (m, 1 H) 3.82-4.00 (m, 3 H) 3.76 (br dd, J = 11.50, 5.48 Hz, 2 H) 3.28-3.33 (m, 1 H) 2.51-2.54 (m, 1 H) 2.31 (br dd, J = 9.86, 7.67 Hz, 1 H) 2.12-2.24 (m, 2 H) 1.96-2.09 (m, 3 H) 1.68-1.94 (m, 4 H). |
| 22 | 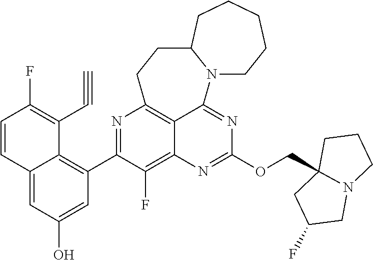 [SMILES: C#Cc1c(F)ccc2cc(O)cc(-c3nc4c5c(nc(OC[C@@]67CCCN6C[C@H](F)C7)nc5c3F)N3CCCCCC3CC4)c12] | A | 614.2 | δ = 8.50 (s, 1H), 7.91-7.81 (m, 1H), 7.38- 7.28 (m, 2H), 7.27-7.13 (m, 1H), 5.53- 5.30 (m, 1H), 4.65-4.55 (m, 1H), 4.53- 4.33 (m, 3H), 3.73-3.41 (m, 6H), 3.23- 3.16 (m, 1H), 3.06-2.93 (m, 1H), 2.55- 2.31 (m, 4H), 2.29-2.19 (m, 1H), 2.18- 2.08 (m, 2H), 2.04-1.89 (m, 5H), 1.87- 1.76 (m, 1H), 1.56-1.42 (m, 1H), 1.40- 1.19 (m, 2H) |
| 23 | 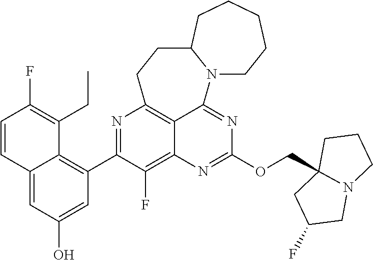 [SMILES: CCc1c(F)ccc2cc(O)cc(-c3nc4c5c(nc(OC[C@@]67CCCN6C[C@H](F)C7)nc5c3F)N3CCCCCC3CC4)c12] | A | 618.2 | δ = 8.59-8.49 (m, 1H), 7.82-7.59 (m, 1H), 7.45-7.19 (m, 2H), 7.16-6.95 (m, 1H), 5.50-5.22 (m, 1H), 4.68-4.55 (m, 1H), 4.52-4.20 (m, 3H), 3.79-3.48 (m, 3H), 3.29-3.24 (m, 1H), 3.19-2.90 (m, 3H), 2.61-2.13 (m, 8H), 2.12-1.75 (m, 8H), 1.60-1.19 (m, 2H), 1.09-0.76 (m, 3H) |
| 24 | 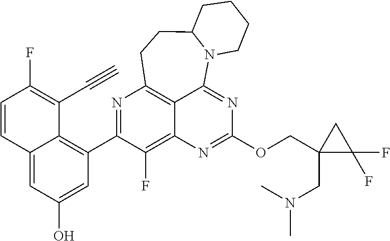 [SMILES: C#Cc1c(F)ccc2cc(O)cc(-c3nc4c5c(nc(OCC6(CN(C)C)CC6(F)F)nc5c3F)N3CCCCC3CC4)c12] | A | 606.2 | δ = 7.91-7.81 (m, 1H), 7.36-7.27 (m, 2H), 7.24-7.15 (m, 1H), 4.74-4.49 (m, 3H), 4.00-3.76 (m, 1H), 3.52-3.38 (m, 2H), 3.27-3.11 (m, 2H), 2.93-2.79 (m, 1H), 2.58-2.40 (m, 2H), 2.29 (s, 6H), 2.12-1.83 (m, 5H), 1.81-1.62 (m, 2H), 1.62-1.46 (m 1H), 1.46-1.35 (m, 1H), 1.35-1.24 (m, 1H). |
| 25 | 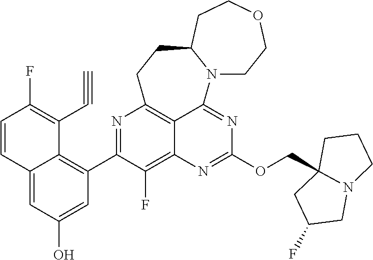 [SMILES: C#Cc1c(F)ccc2cc(O)cc(-c3nc4c5c(nc(OC[C@@]67CCCN6C[C@H](F)C7)nc5c3F)N3CCOCC[C@@H]3CC4)c12] | A | 616.4 | δ = 10.31-10.11 (m, 1H), 7.97 (t, J = 7.2 Hz, 1H), 7.52-7.42 (m, 1H), 7.41-7.34 (m, 1H), 7.30-7.14 (m, 1H), 5.45-5.18 (m, 1H), 4.51-4.34 (m, 1H), 4.23-4.09 (m, 2H), 4.07-3.97 (m, 2H), 3.89 (dd, J = 5.6, 11.2 Hz, 1H), 3.79-3.55 (m, 4H), 3.09 (d, J = 7.6 Hz, 2H), 3.05-2.99 (m, 2H), 2.89-2.79 (m, 2H), 2.68 (s, 1H), 2.34 (s, 2H), 2.12 (d, J = 8.0 Hz, 2H), 1.77 (s, 2H), 1.28-1.11 (m, 3H) |
| 26 | 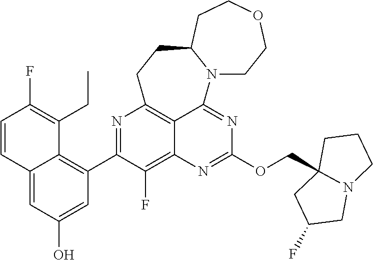 [SMILES: CCc1c(F)ccc2cc(O)cc(-c3nc4c5c(nc(OC[C@@]67CCCN6C[C@H](F)C7)nc5c3F)N3CCOCC[C@@H]3CC4)c12] | A | 620.5 | δ = 9.91 (d, J = 3.6 Hz, 1H), 7.82-7.70 (m, 1H), 7.42-7.28 (m, 2H), 7.14-6.90 (m, 1H), 5.41-5.16 (m, 1H), 4.50-4.34 (m, 1H), 4.15 (d, J = 9.6 Hz, 1H), 4.09- 3.87 (m, 3H), 3.81-3.54 (m, 3H), 3.14- 2.99 (m, 4H), 2.94-2.78 (m, 2H), 2.39- 2.19 (m, 5H), 2.18-2.10 (m, 2H), 2.10- 1.97 (m, 3H), 1.91-1.72 (m, 3H), 0.86- 0.73 (m, 3H) |
| 27 | 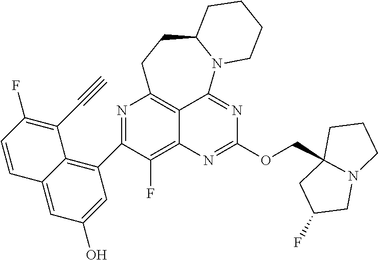 [SMILES: C#Cc1c(F)ccc2cc(O)cc(-c3nc4c5c(nc(OC[C@@]67CCCN6C[C@H](F)C7)nc5c3F)N3CCCC[C@@H]3CC4)c12] | A | 600.2 | δ = 7.88-7.81 (m, 1H), 7.36-7.28 (m, 2H), 7.22-7.16 (m, 1H), 5.46-5.28 (m, 1H), 4.72 (br d, J = 12.8 Hz, 1H), 4.45- 4.29 (m, 2H), 3.94-3.81 (m, 1H), 3.49- 3.34 (m, 4H), 3.28-3.08 (m, 4H), 2.55-2.28 (m, 3H), 2.23- 2.16 (m, 1H), 2.11-2.04 (m, 2H), 2.02- 1.83 (m, 6H), 1.79-1.69 (m, 1H), 1.58- 1.42 (m, 1H). |
| 28 | 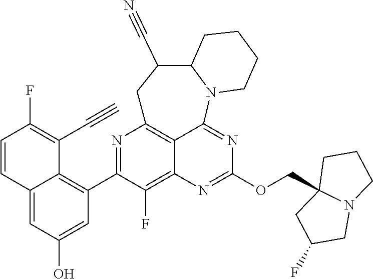 [SMILES: C#Cc1c(F)ccc2cc(O)cc(-c3nc4c5c(nc(OC[C@@]67CCCN6C[C@H](F)C7)nc5c3F)N3CCCCC3C(C#N)C4)c12] | B | 625.3 | δ ppm 10.68-10.87 (m, 1 H) 10.19 (br d, J = 10.40 Hz, 1 H) 7.99 (br dd, J = 9.31, 6.02 Hz, 1 H) 7.36-7.52 (m, 2 H) 7.08- 7.28 (m, 1 H) 5.48-5.66 (m, 1 H) 4.54- 4.71 (m, 2 H) 4.01-4.13 (m, 2 H) 3.72- 3.98 (m, 6 H) 3.27-3.35 (m, 2 H) 2.51- 2.57 (m, 2 H) 2.25-2.36 (m, 1 H) 2.13- 2.24 (m, 2 H) 1.79-2.12 (m, 5 H). |
| 29 | 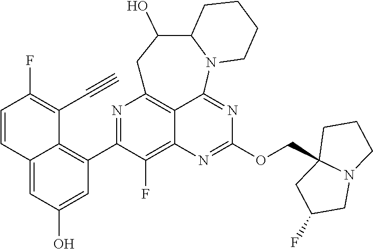 [SMILES: C#Cc1c(F)ccc2cc(O)cc(-c3nc4c5c(nc(OC[C@@]67CCCN6C[C@H](F)C7)nc5c3F)N3CCCCC3C(O)C4)c12] | B | 616.3 | δ ppm 10.68-10.97 (m, 1 H) 10.01- 10.41 (m, 1 H) 7.98 (dd, J = 9.17, 5.89 Hz, 1 H) 7.43-7.51 (m, 1 H) 7.39 (d, J = 2.19 Hz, 1 H) 7.07-7.23 (m, 1 H) 5.36-5.73 (m, 2 H) 4.50-4.77 (m, 3 H) 4.30-4.36 (m, 1 H) 4.16-4.29 (m, 2 H) 4.06-4.14 (m, 2 H) 3.41-3.53 (m, 2 H) 3.20-3.36 (m, 2 H) 2.98-3.15 (m, 1 H) 2.52-2.59 (m, 1 H) 2.24-2.34 (m, 1 H) 2.12-2.22 (m, 2 H) 1.98-2.08 (m, 2 H) 1.77-1.92 (m, 3 H) 1.36-1.75 (m, 3 H). |
| 30 | 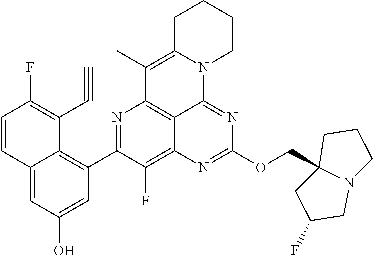 [SMILES: C#Cc1c(F)ccc2cc(O)cc(-c3nc4c5c(nc(OC[C@@]67CCCN6C[C@H](F)C7)nc5c3F)N3CCCCC3=C4C)c12] | B | 598.3 | ¹H NMR (400 MHz, MeOD-d₄) δ = 7.81 (s, 1H), 7.37-7.26 (m, 2H), 7.24-7.18 (m, 1H), 5.48-5.25 (m, 1H), 4.47-4.29 (m, 4H), 3.54-3.35 (m, 3H), 3.27-3.21 (m, 1H), 3.15-3.02 (m, 3H), 2.32 (s, 3H), 2.28 (br s, 2H), 2.12-2.04 (m, 4H), 2.03-1.89 (m, 4H). |
| 31 | 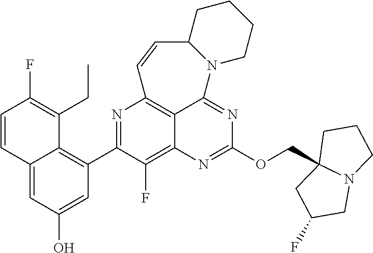 [SMILES: CCc1c(F)ccc2cc(O)cc(-c3nc4c5c(nc(OC[C@@]67CCCN6C[C@H](F)C7)nc5c3F)N3CCCCC3C=C4)c12] | B | 602.4 | 10.71 (br s, 1 H) 9.94 (br s, 1 H) 7.76 (dd, J = 9.31, 6.02 Hz, 1 H) 7.35 (t, J = 9.31 Hz, 1 H) 7.32 (d, J = 2.19 Hz, 1 H) 7.00 (s, 1 H) 5.74 (t, J = 7.39 Hz, 1 H), 5.52- 5.65 (m, 2 H) 4.59-4.68 (m, 2 H) 4.06- 4.23 (m, 3 H) 3.87 (br d, J = 18.62 Hz, 2 H) 3.78 (br dd, J = 12.05, 4.93 Hz, 2 H) 3.47 (br d, J = 7.12 Hz, 5 H) 3.28-3.34 (m, 2 H) 2.31-2.40 (m, 3 H) 2.11-2.28 (m, 4 H) 2.01-2.11 (m, 1 H) 1.81-1.88 (m, 2 H) 1.72-1.80 (m, 2 H) 0.75 (t, J = 7.39 Hz, 3 H). |
| 32 | 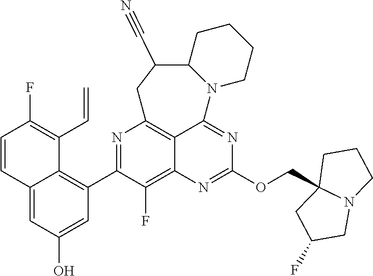 [SMILES: C=Cc1c(F)ccc2cc(O)cc(-c3nc4c5c(nc(OC[C@@]67CCCN6C[C@H](F)C7)nc5c3F)N3CCCCC3C(C#N)C4)c12] | B | 627.4 | δ ppm 10.80 (br s, 1 H) 9.98-10.23 (m, 1 H) 7.79-7.89 (m, 1 H) 7.31-7.45 (m, 2 H) 7.02-7.20 (m, 1 H) 5.88-6.13 (m, 1 H) 5.45-5.69 (m, 2 H) 4.76-5.06 (m, 3 H) 4.53-4.68 (m, 2 H) 3.73-3.90 (m, 6 H) 3.31 (br d, J = 6.02 Hz, 2 H) 2.52-2.54 (m, 1 H) 2.29-2.35 (m, 1 H) 2.11-2.23 (m, 2 H) 1.78-2.09 (m, 6 H) 1.65 (br d, J = 6.02 Hz, 1 H). |

### Screen Assays

### Cell Proliferation Assay:

Two thousand KRAS mutant cells per well are seeded in 384-well white plate and then treated with indicated compounds for 72 hours at 37° C. and 5% CO₂. Cell proliferation is measured using CellTiter-Glo 2.0 luciferase-based ATP detection assay (Promega, Madison, WI) following the manufacturer's protocol. IC₅₀ values are determined using Prism software (GraphPad Software, San Diego, CA).

Anti-proliferation IC₅₀ (nM)

| | GP2D (KRAS | SW620 (KRAS |
|---|---|---|
| Ex. # | G12D) | G12V) |
| 1 | 1.1 | 3.6 |
| 2 | 85 | 592 |
| 3 | 6.3 | 11 |
| 4 | 135 | 304 |
| 5 | 4.2 | 20 |
| 6 | 21 | 26 |
| 9 | 102 | 641 |
| 10 | 3000 | 3000 |
| 13 | 3.9 | 39 |
| 18 | 123 | 130 |
| 20 | 10 | 47 |
| 21 | 2.9 | 20 |
| 22 | 32 | 248 |
| 23 | 172 | 1446 |
| 24 | 23 | 91 |
| 25 | 276 | 638 |
| 26 | 321 | 557 |
| 27 | 10 | 32 |
| 28 | 61 | 787 |
| 29 | 14 | 142 |
| 30 | 819 | 1721 |
| 31 | 136 | 591 |
| 32 | 461 | 2045 |

What is claimed is:

1. A compound of the formula I, or a pharmaceutically acceptable salt thereof, 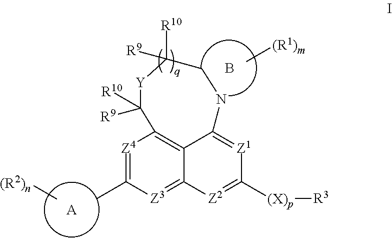
wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl, a 5- to 10-membered heteroaryl, or a 4- to 10-membered heterocycloalkyl;
ring B is a 4- to 9-membered heterocycloalkyl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}—, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R¹ and an R⁹ or an R¹⁰ on an adjacent carbon to ring B, taken together with the carbon atoms to which they are attached, combine form a carbon-carbon double bond;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{e}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

2. A compound of the formula I, or a pharmaceutically acceptable salt thereof, 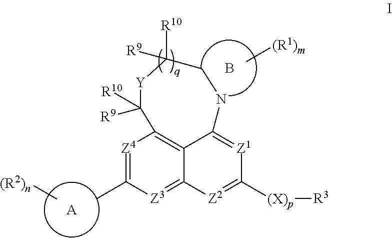
wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl or 5- to 10-membered heteroaryl;
ring B is a 4- to 9-membered heterocycloalkyl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NRC(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}—, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁹ and R¹⁰, taken together with the carbon atom to which they are attached, combine to form a C₂-C₆ alkenyl, C₃-C₆ cycloalkyl, a 4- to 10-membered heterocycloalkyl, or an oxo group, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC_{I}—C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C, —C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R¹ taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

3. A compound of the formula I, or a pharmaceutically acceptable salt thereof, 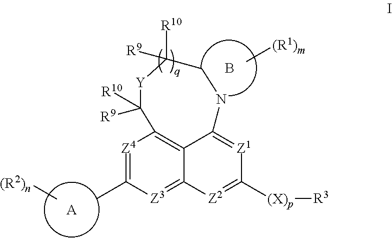
wherein
X is a —O—, —S—, or —NR⁴—;
Y is bond, —O—, —S—, —S(O)—, or —S(O)₂—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a C₆-C₁₀ aryl or 5- to 10-membered heteroaryl;
ring B is a 4- to 9-membered heterocycloalkyl ring having one nitrogen atom in the 4- to 9-membered heterocycloalkyl ring;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{e})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each of R⁹ and R¹⁰ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂₀R^{a}, —CN, or —NO₂, or R⁹ and R¹⁰, taken together with the carbon atom to which they are attached, form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or an R⁹ and an R¹⁰ on adjacent carbon atoms, taken together with the carbon atoms to which they are attached, combine form a carbon-carbon double bond substituted by an R⁹ and an R¹⁰;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂(H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or a C₂-C₆ alkenyl;
m is 0, 1, 2, 3, 4, 5, 6, or 7;
n is 0, 1, 2, 3, 4, 5, 6, or 7;
p is 0 or 1; and
q is 0, 1, or 2.

4. The compound of any one of the preceeding claims, or a pharmaceutically acceptable salt thereof, wherein the compound is of the formula II, III, IV, V, VI, VII, VIII, or IX, 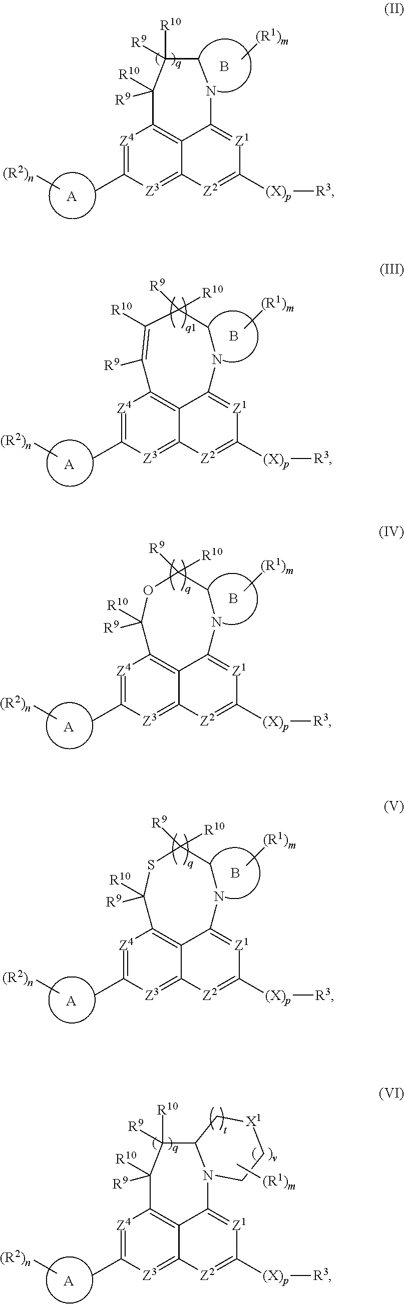 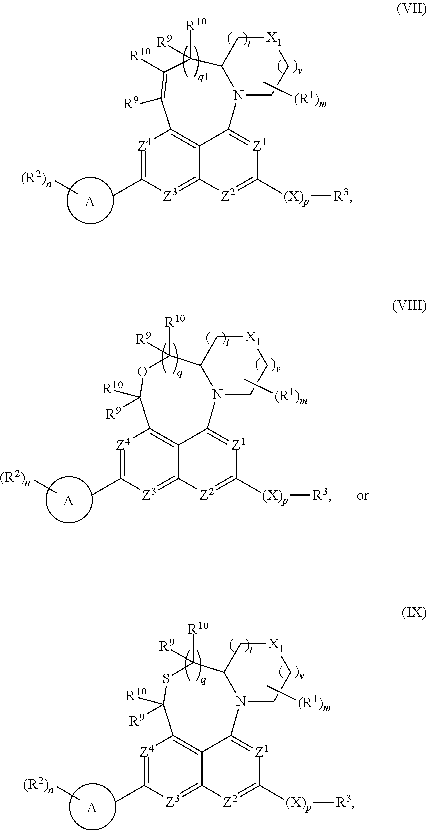
wherein
X¹ is —O—, —S—, —S(O)—, —S(O)₂—, or —C(R¹)₂—;
q1 is 0 or 1;
t is 0, 1, 2, or 3; and
v is 0, 1, or 2.

5. The compound of any one of the preceding claims, or a pharmaceutically acceptable salt thereof, wherein the compound is of the formula X, XI, XII, XIII, XIV, XV, XVI, or XVII, 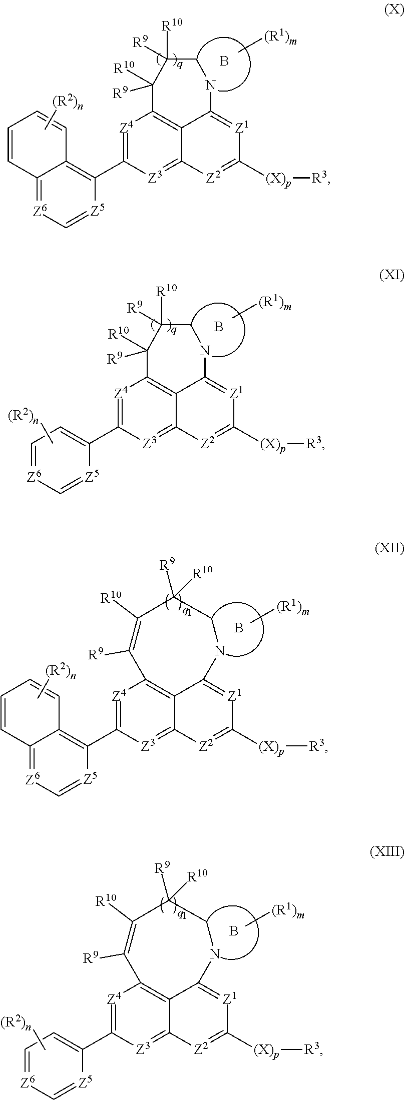 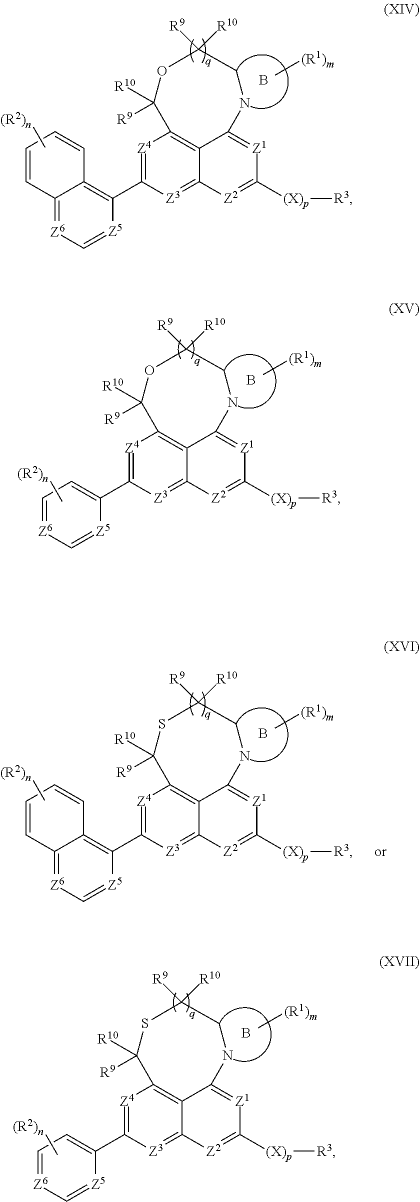
wherein
Z⁵ is N or C(R¹¹);
Z⁶ is N or C(R¹²);
each of R¹¹ and R¹², when present, is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂; and
q1 is 0 or 1.

6. The compound of any one of the preceding claims, or a pharmaceutically acceptable salt thereof, wherein the compound is of the formula XVIII, XIX, XX, XXI, XXII, XXIII, XXIV, or XXV, 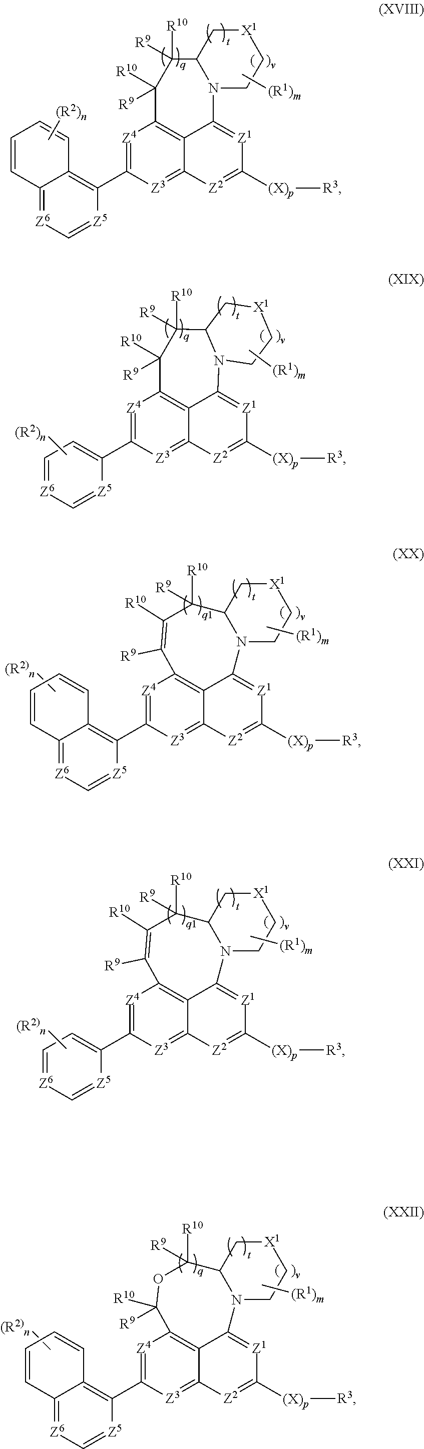 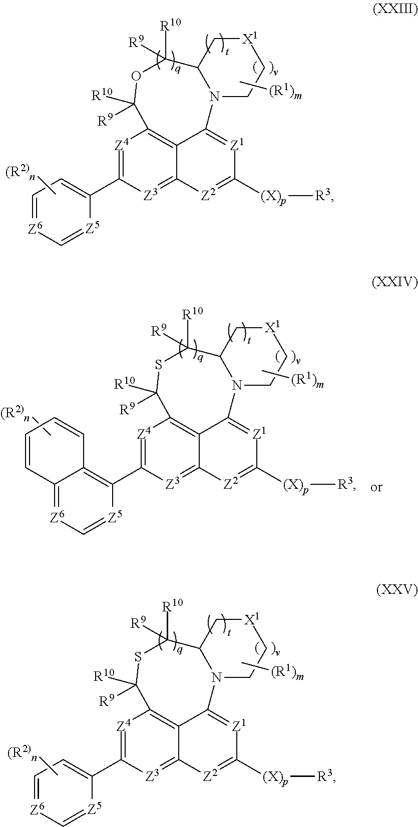
wherein
X¹ is —O—, —S—, —S(O)—, —S(O)₂—, —C(O)O—, —OC(O)—, or —C(R¹)₂—;
Z⁵ is N or C(R¹¹);
Z⁶ is N or C(R¹²);
each of R¹¹ and R¹², when present, is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R⁹, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
q1 is 0 or 1;
t is 0, 1, 2, or 3; and
v is 0, 1, or 2.

7. The compound of any one of claims 1 to 4, or a pharmaceutically acceptable salt thereof, wherein the compound is of the formula XXVI, XXVII, XXVIII, XXIX, XXX, XXXI, XXXII, or XXXIII, 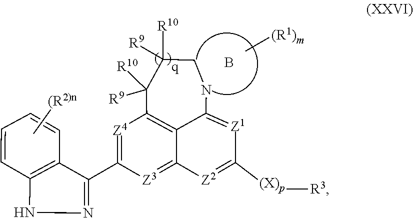 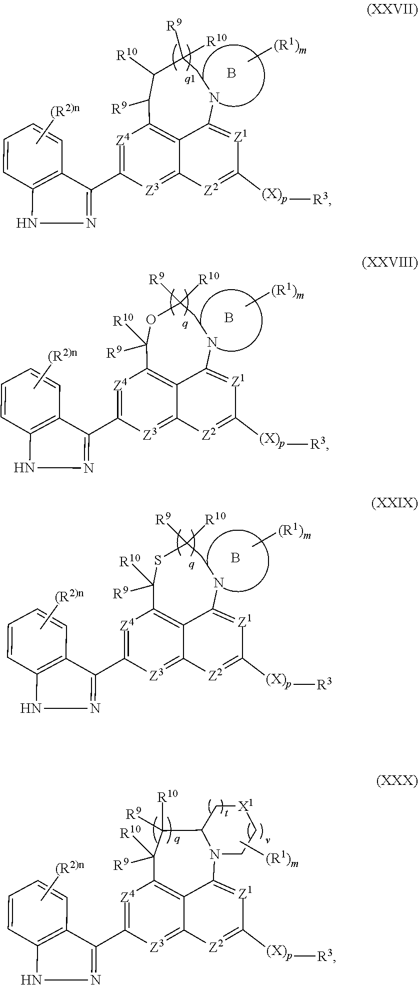 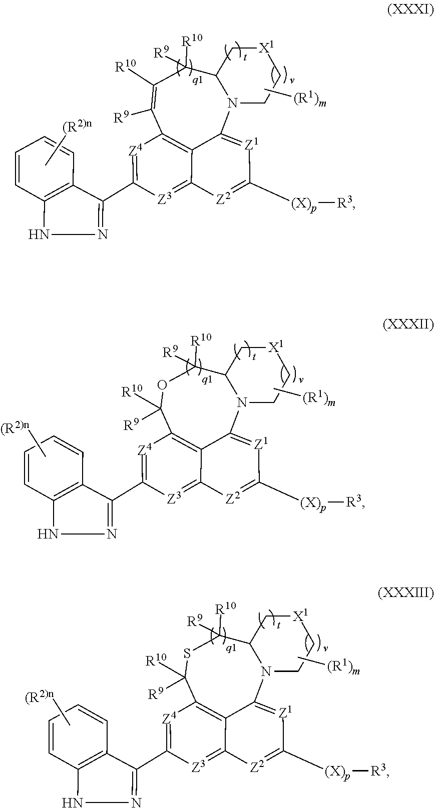
wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

8. The compound of claim 1 or 4, or a pharmaceutically acceptable salt thereof, wherein the compound is of the formula XXXIV, XXXV, XXXVI, XXXVII, XXXVIII, XXXIX, XXXX, or XXXXI, 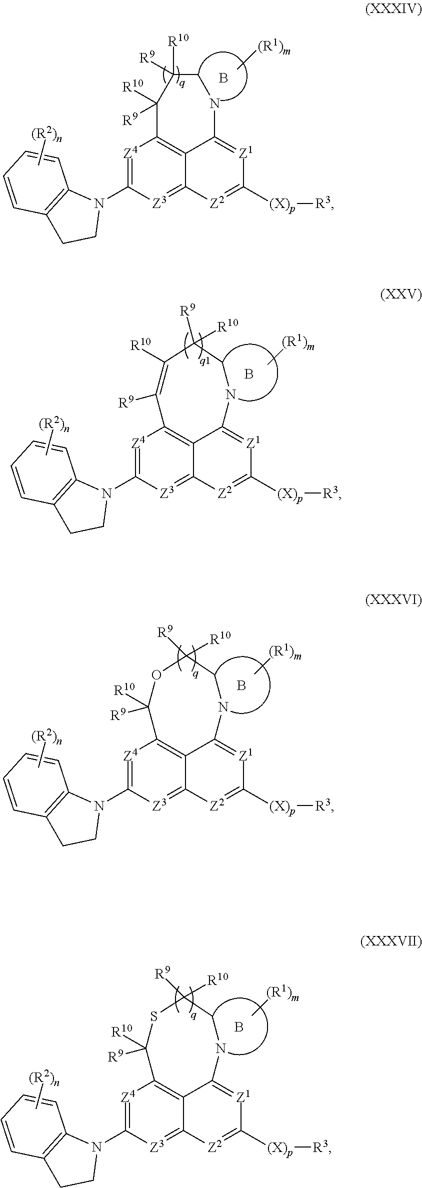 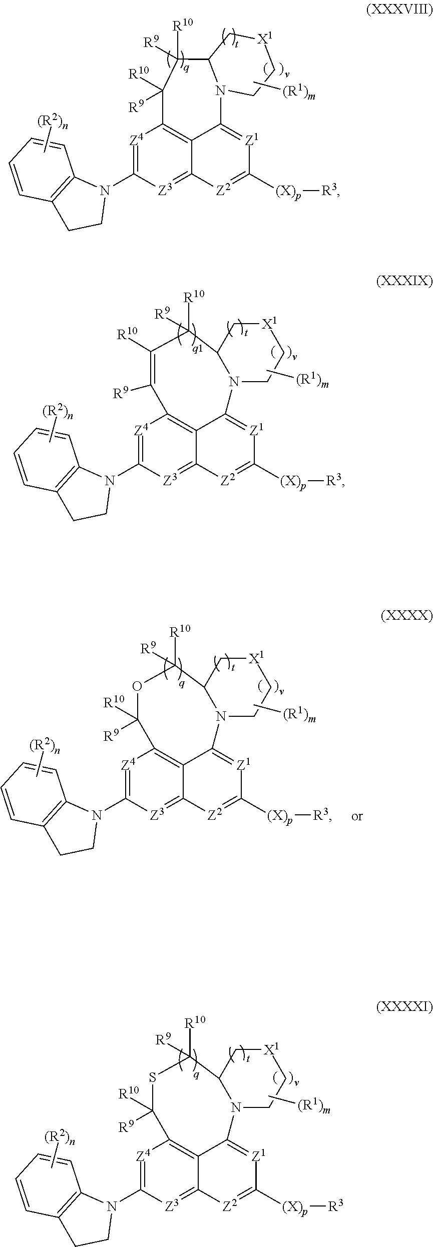
wherein
X¹ is —O—, —S(O)—, —S(O)₂—, —C(O)O—, —OC(O)—, or —C(R¹)₂—;
q1 is 0 or 1;
t is 0, 1, 2, or 3; and
v is 0, 1, or 2.

9. The compound of any one of claims 1 to 4, or a pharmaceutically acceptable salt thereof, wherein the compound is of the formula XXXXII, XXXXIII, XXXXIV, XXXV, XXXVI, XXXXVII, XXXXVIII, or XXXXIX, 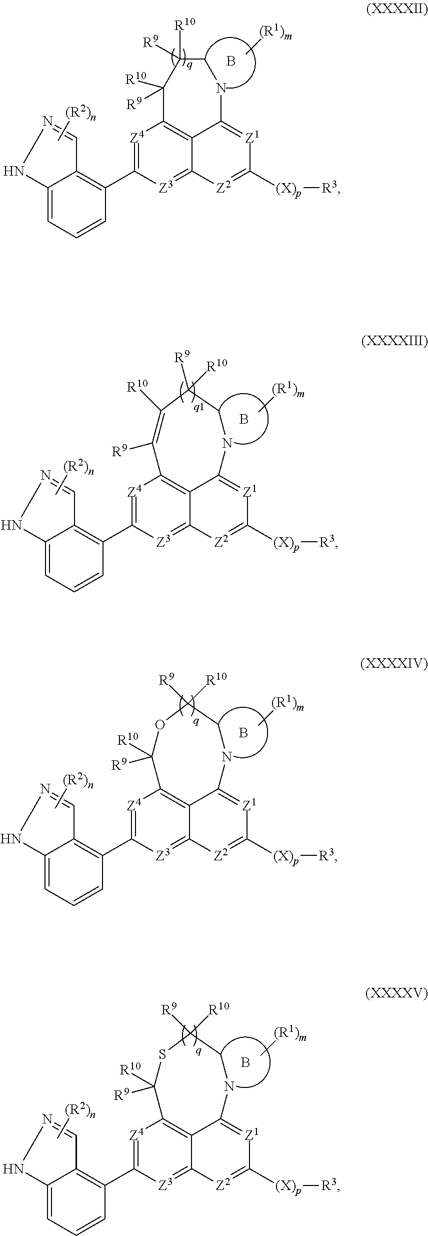 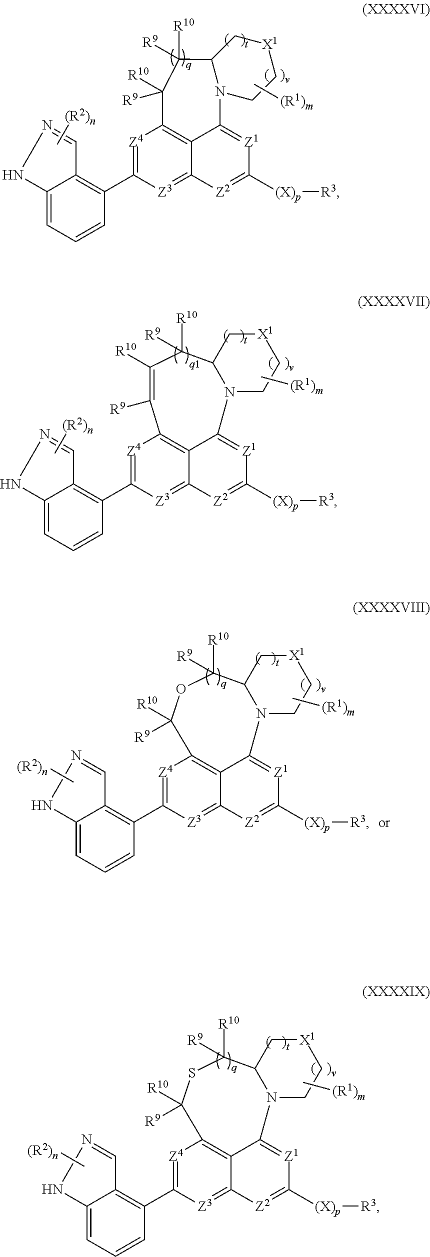
wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

10. The compound of any one of claims 1 to 4, or a pharmaceutically acceptable salt thereof, wherein the compound is of the formula L, LI, LII, LIII, LIV, LV, LVI, or LVII, 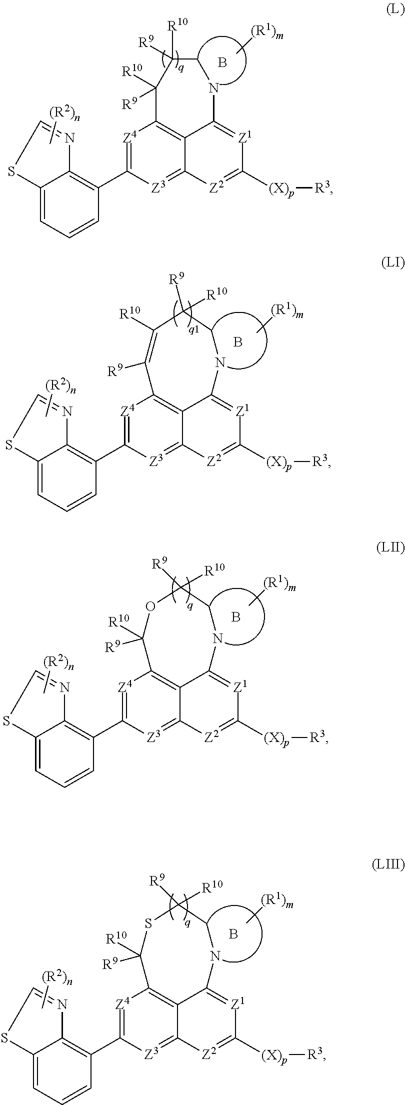 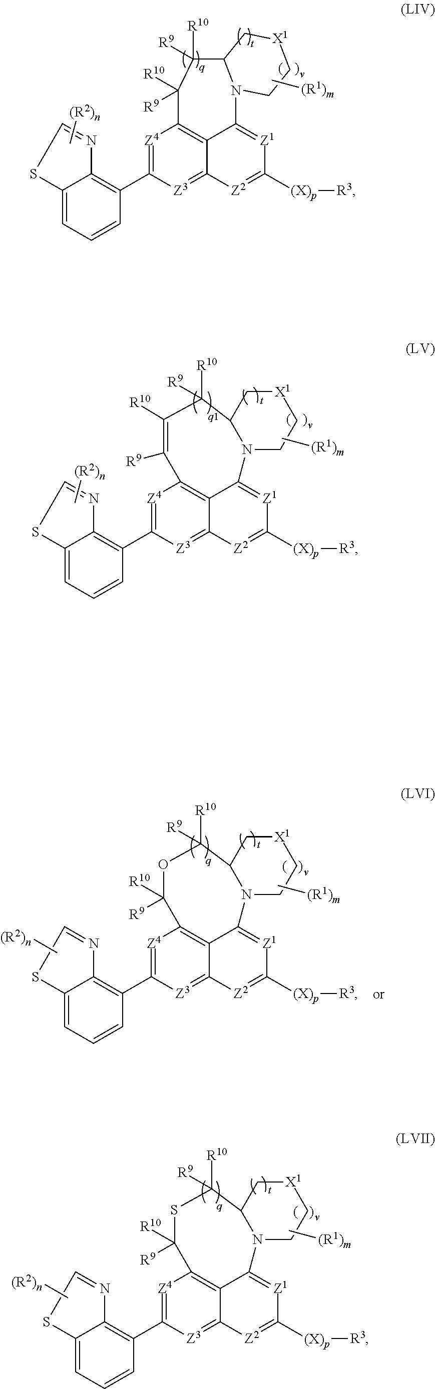
wherein
X¹ is —O—, —S—, —S(O)—, —S(O)₂—, —C(O)O—, —OC(O)—, or —C(R¹)₂—;
q1 is O or 1;
t is 0, 1, 2, or 3; and
v is 0, 1, or 2.

11. The compound of any one of claims 1 to 4, or a pharmaceutically acceptable salt thereof, wherein the compound is of the formula LVIII, LVIX, LX, LXI, LXII, LXIII, LXIV, or LXV, 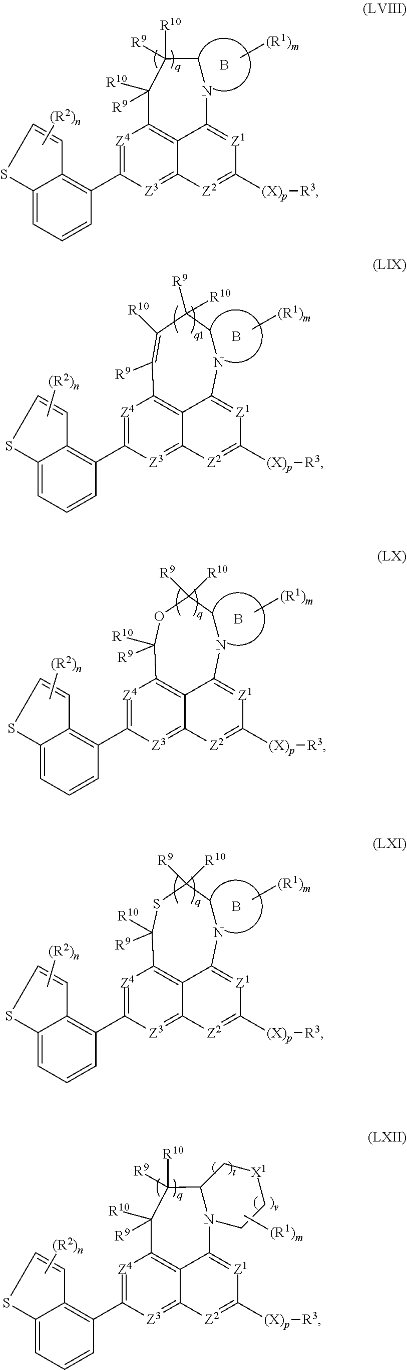 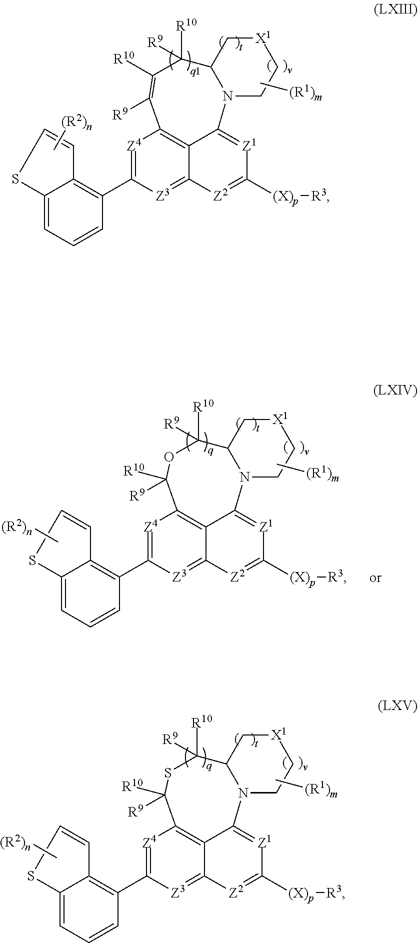
wherein
X¹ is —O—, —S—, —S(O)—, —S(O)₂—, —C(O)O—, —OC(O)—, or —C(R¹)₂—;
q1 is 0 or 1;
t is 0, 1, 2, or 3; and
v is 0, 1, or 2.

12. The compound of any one of the preceding claims, or a pharmaceutically acceptable salt thereof, wherein q, when present, is 0.

13. The compound of any one of the preceding claims, or a pharmaceutically acceptable salt thereof, wherein q, when present, is 1.

14. The compound of any one of the preceding claims, or a pharmaceutically acceptable salt thereof, wherein q1, when present, is 0.

15. The compound of any one of the preceding claims, or a pharmaceutically acceptable salt thereof, wherein q1, when present, is 1.

16. The compound of any one of the preceding claims, or a pharmaceutically acceptable salt thereof, wherein R¹ is deuterium, C₁-C₆ alkyl, C₂-C₆ alkenyl, or C₂-C₆ alkynyl, wherein each hydrogen atom in C₁-C₆ alkyl, C₂-C₆ alkenyl, or C₂-C₆ alkynyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂ or two hydrogen atoms on a single carbon atom of the 4- to 10-membered heterocycloalkyl combine to form an oxo group or an C₂-C₆ alkenyl group; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

17. The compound of any one of the preceding claims, or a pharmaceutically acceptable salt thereof, wherein the compound comprises a core having the formula 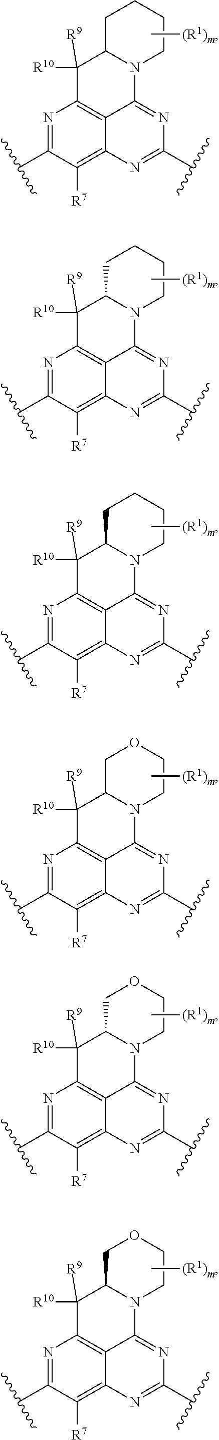 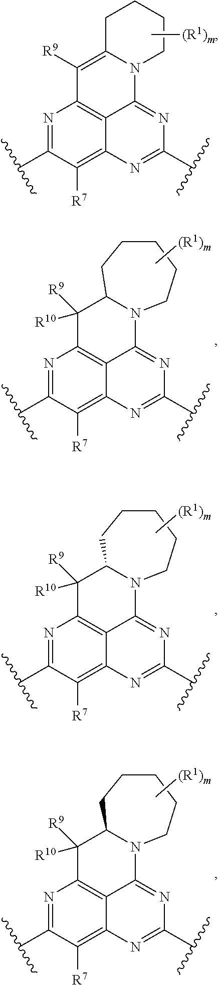 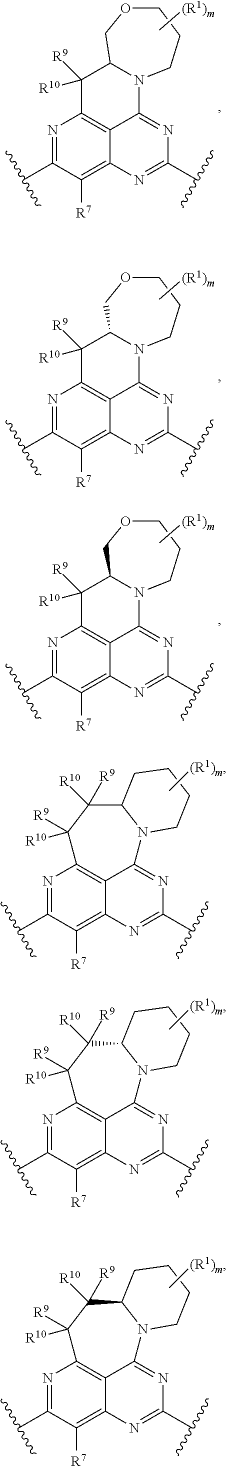 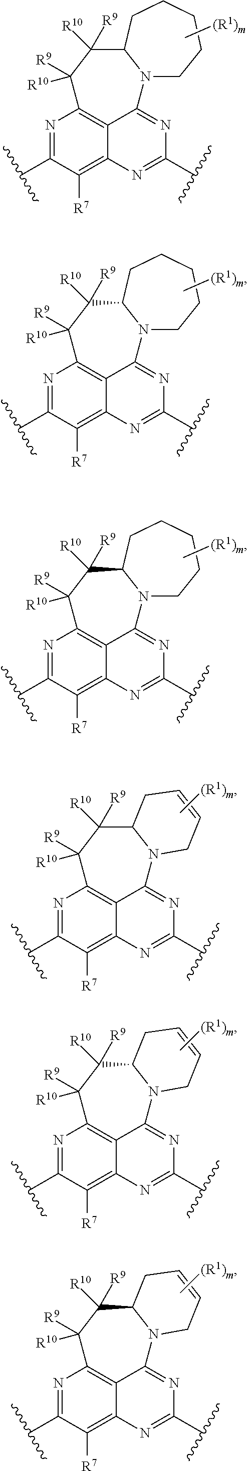 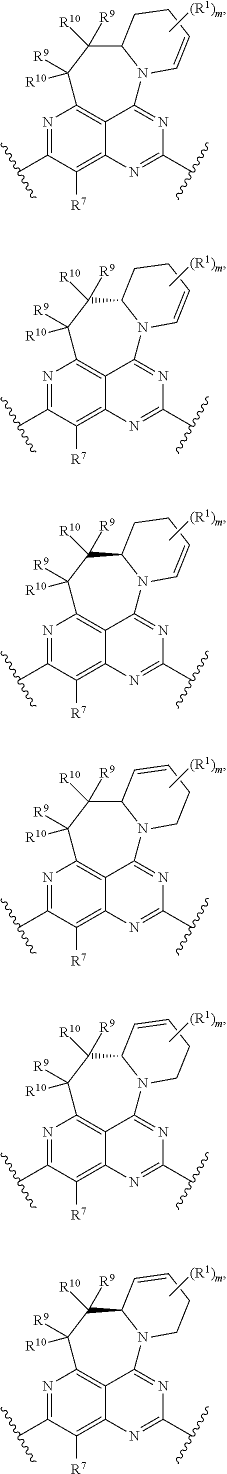 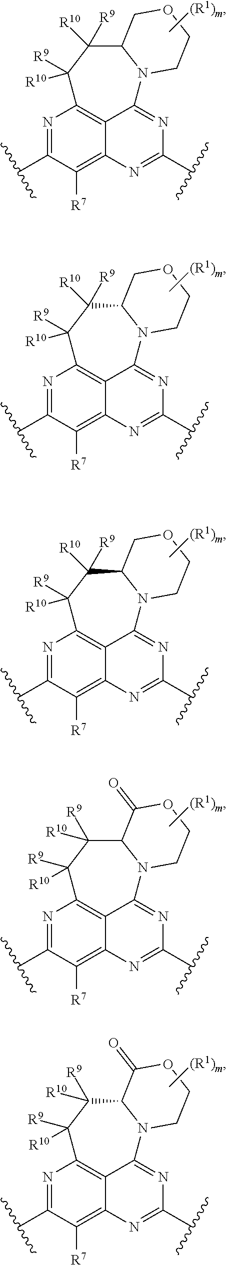 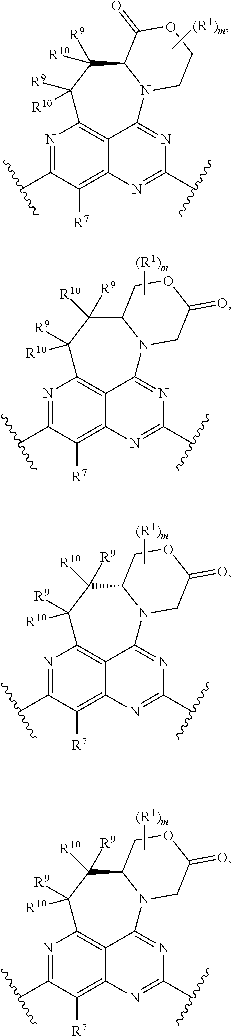 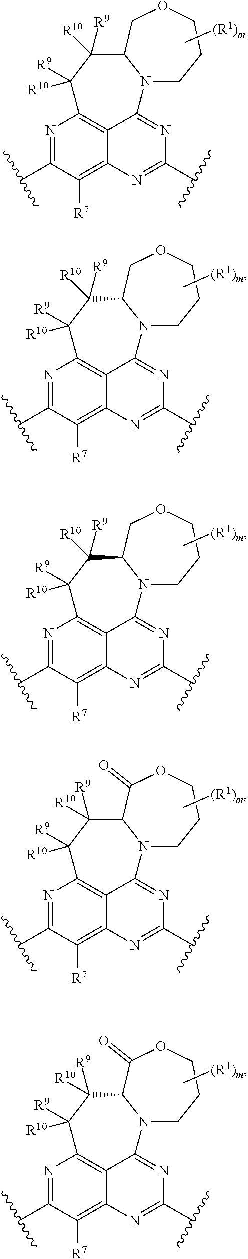 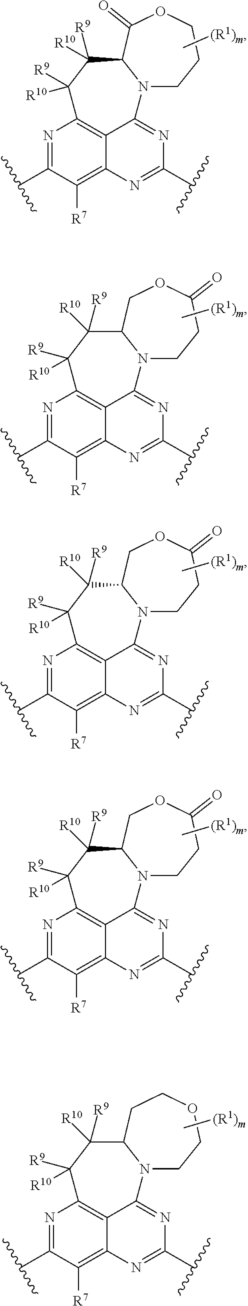 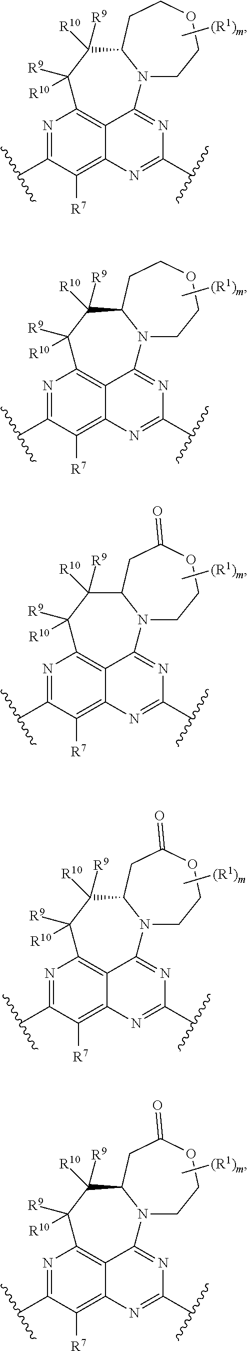 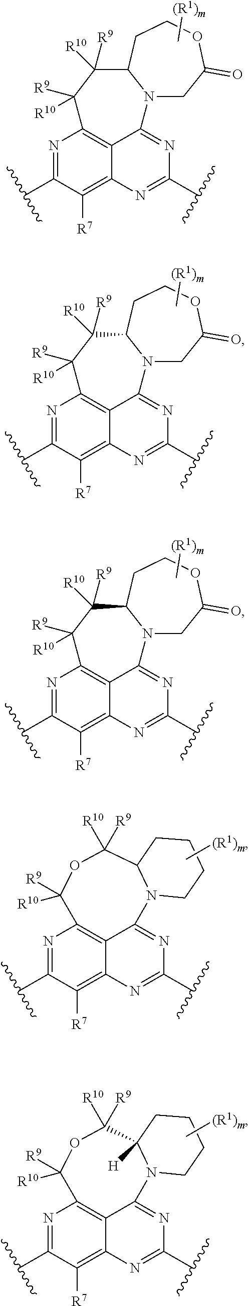 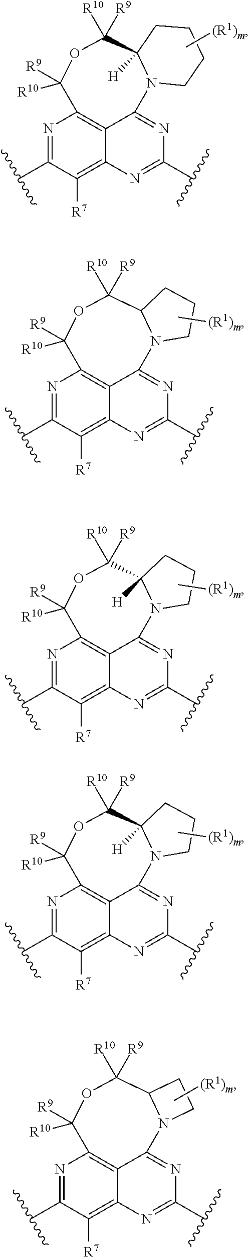 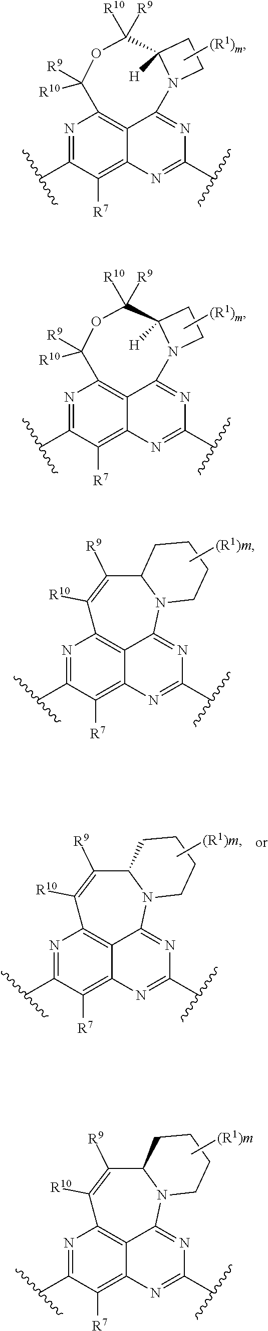
wherein each “ [symbol] ” is a point of covalent attachment to either ring A or —(X)ₚ—R³.

18. The compound of any one of the preceding claims, or a pharmaceutically acceptable salt thereof, wherein m is 0, 1, 2, or 3.

19. The compound of any one of the preceding claims, or a pharmaceutically acceptable salt thereof, wherein n is 0, 1, 2, or 3.

20. The compound of any one of the preceding claims, or a pharmaceutically acceptable salt thereof, wherein Z¹ is N.

21. The compound of any one of the preceding claims, or a pharmaceutically acceptable salt thereof, wherein Z² is N.

22. The compound of any one of the preceding claims, or a pharmaceutically acceptable salt thereof, wherein Z³ is CR⁷, and Z⁴ is N.

23. The compound of any one of claims 1 to 21, or a pharmaceutically acceptable salt thereof, wherein Z³ is CR⁷, and Z⁴ is CR⁸.

24. The compound of any one of claims 1 to 21, or a pharmaceutically acceptable salt thereof, wherein Z³ is N, and Z⁴ is CR⁸.

25. The compound of any one of claims 1 to 21, or a pharmaceutically acceptable salt thereof, wherein Z³ is N, and Z⁴ is N.

26. The compound of any one of claims 1 to 21, or a pharmaceutically acceptable salt thereof, wherein Z³ is N, Z⁴ is CR⁸, Z⁵ is CR¹¹, and Z⁶ is CR¹².

27. The compound of any one of claims 1 to 21, or a pharmaceutically acceptable salt thereof, wherein Z³ is CR⁷, Z⁴ is N, Z⁵ is CR¹¹, and Z⁶ is CR¹².

28. The compound of any one of claims 1 to 21, or a pharmaceutically acceptable salt thereof, wherein Z³ is N, Z⁴ is N, Z⁵ is CR¹¹, and Z⁵ is CR¹².

29. The compound of any one of claims 1 to 22, or a pharmaceutically acceptable salt thereof, wherein the compound comprises a core having the formula 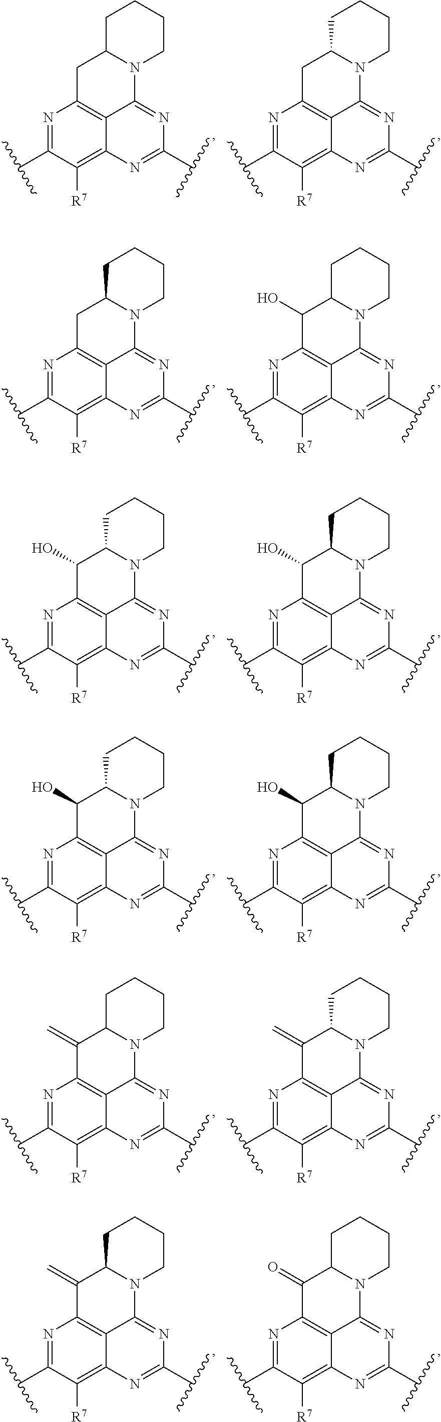 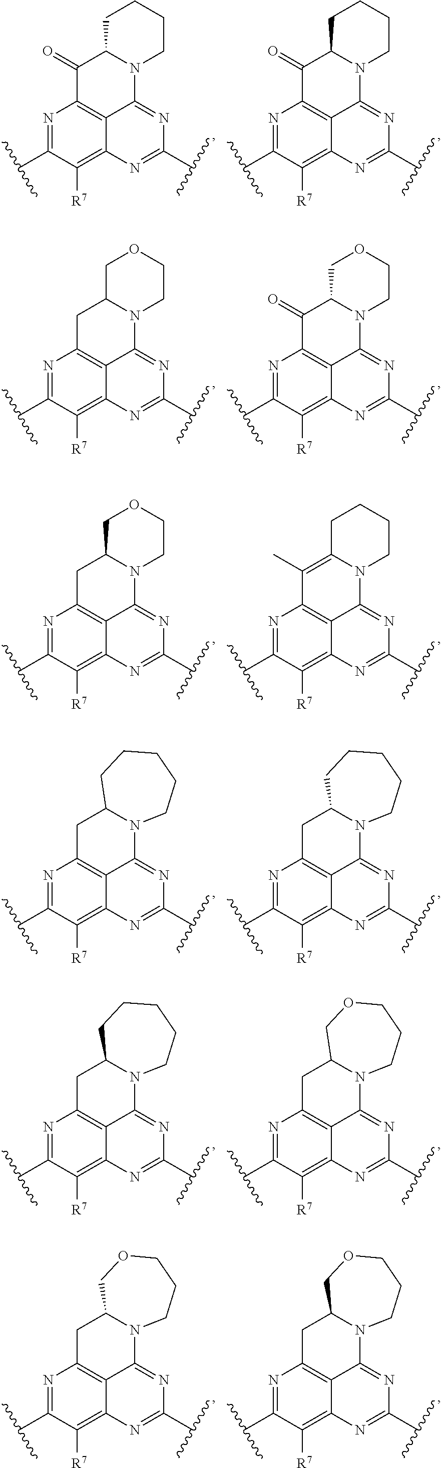 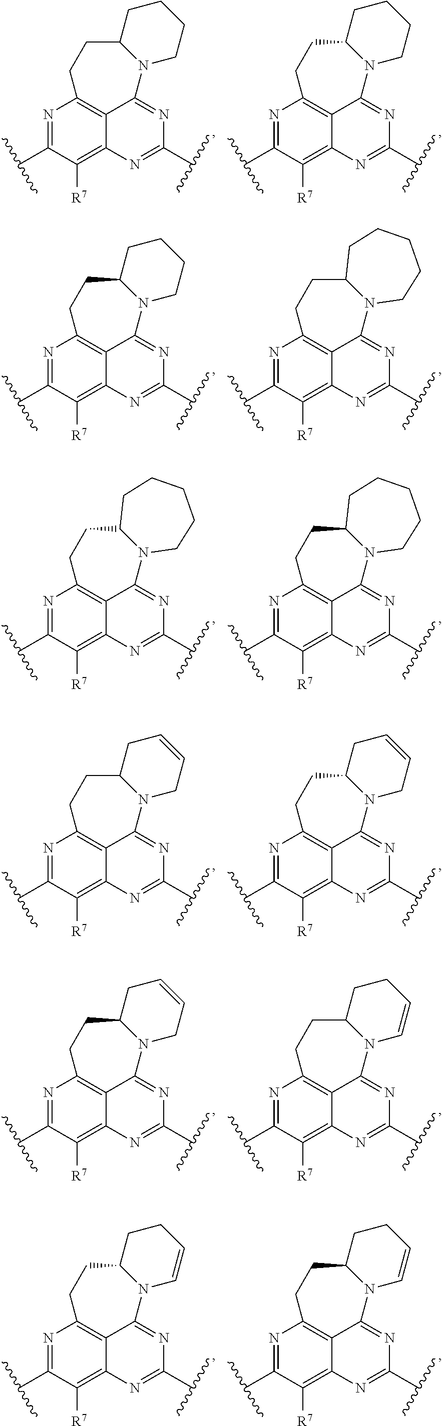 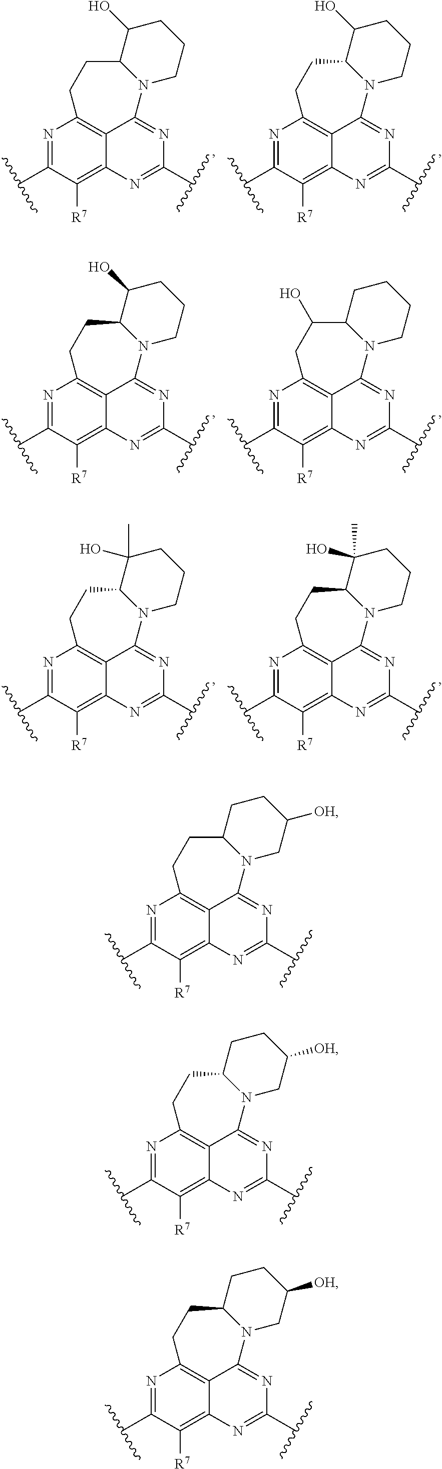 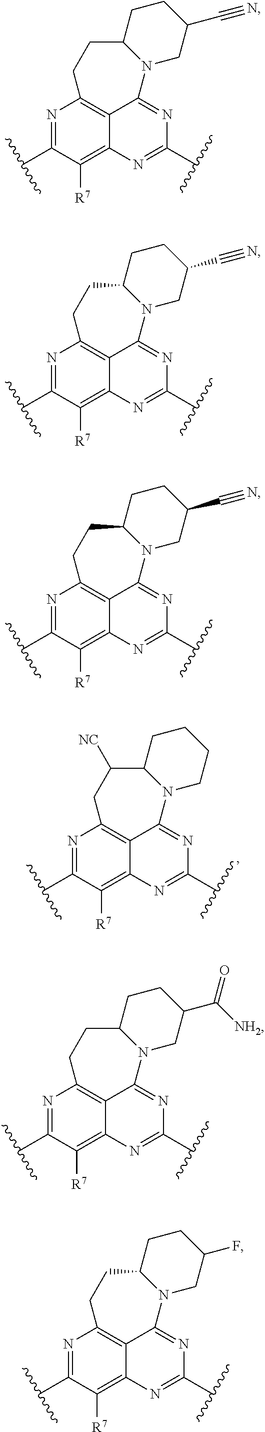 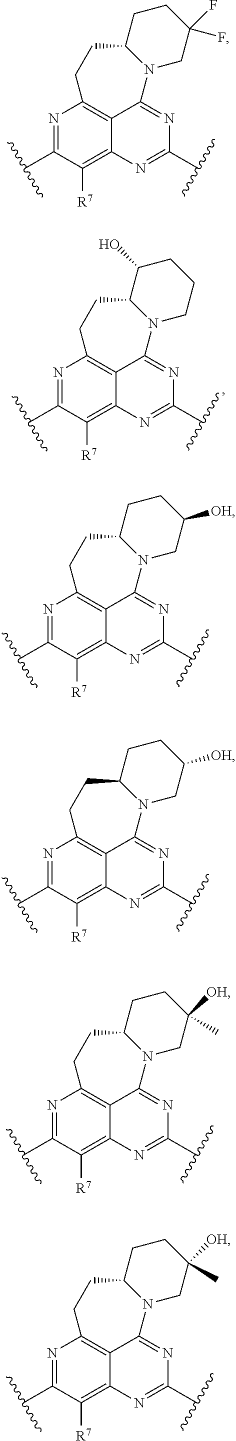 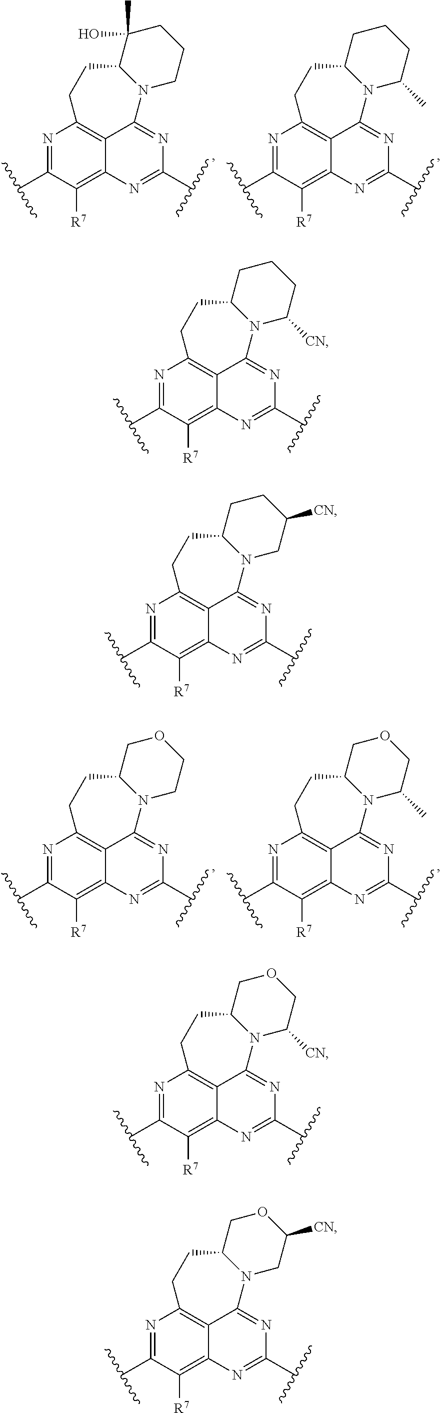 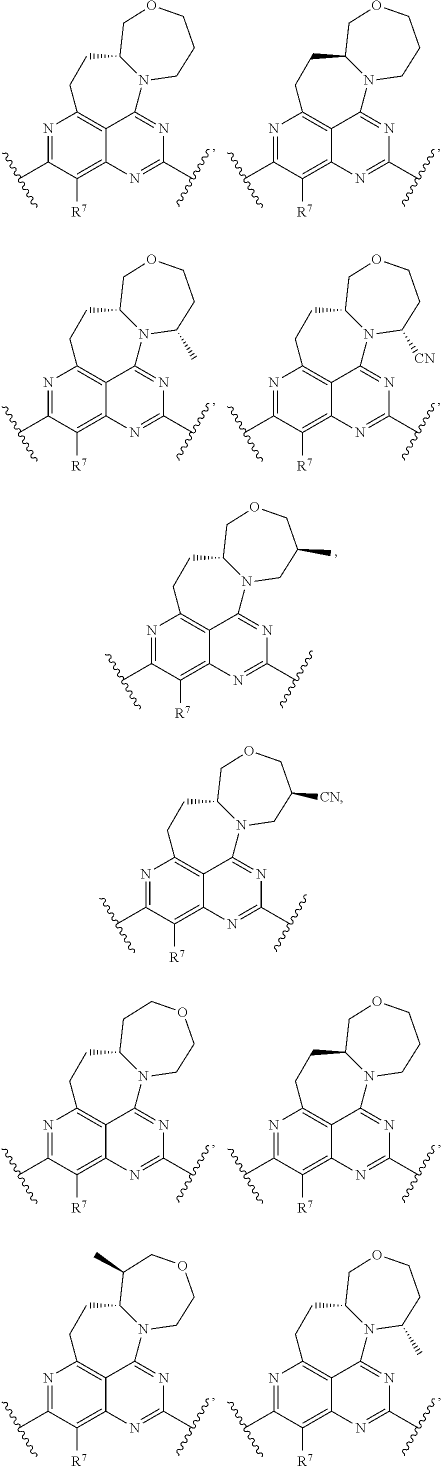 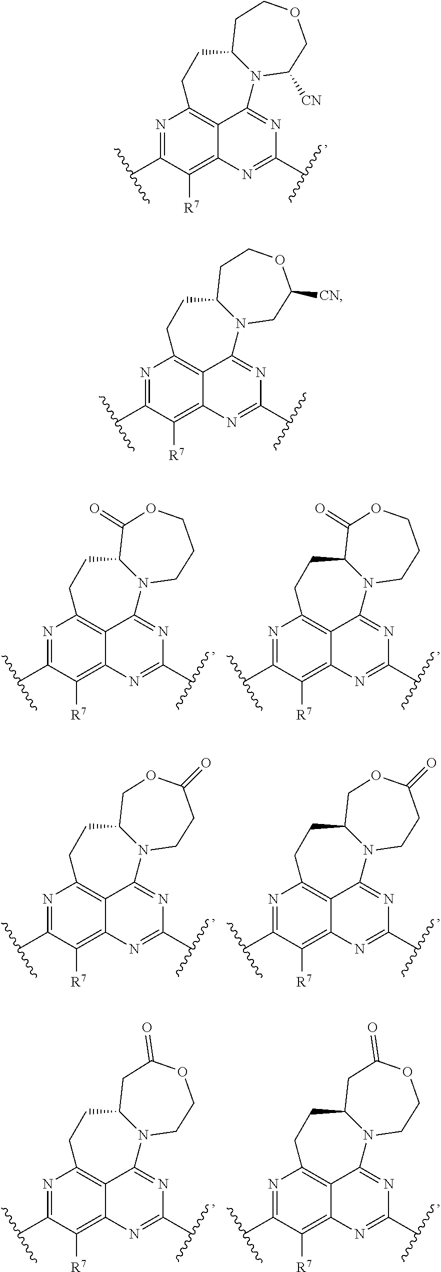 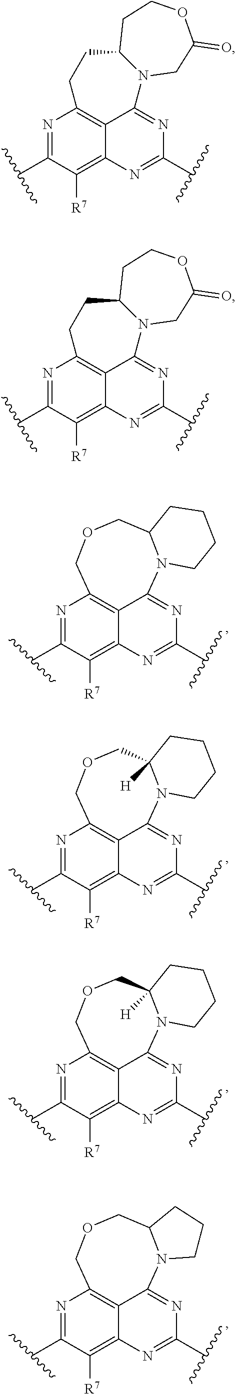 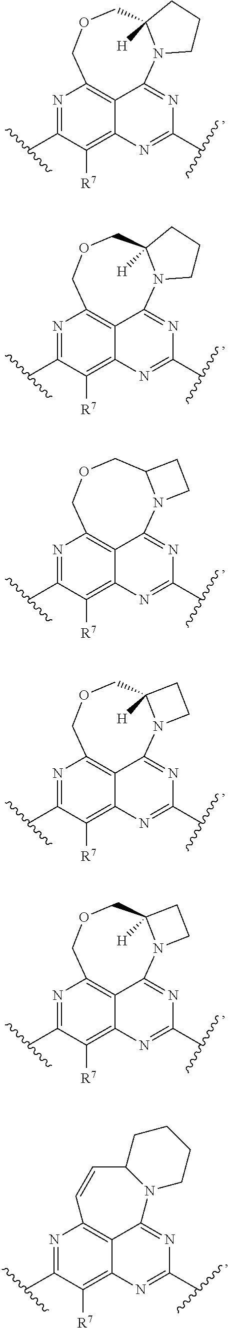 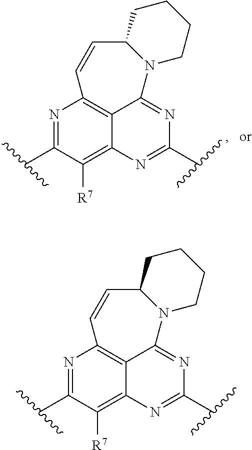
wherein each “ [symbol] ” is a point of covalent attachment to either ring A or —(X)ₚ—R³.

30. The compound of any one of the preceding claims, or a pharmaceutically acceptable salt thereof, wherein p is 0.

31. The compound of any one of claims 1 to 29, or a pharmaceutically acceptable salt thereof, wherein p is 1.

32. The compound of any one of claims 1 to 29, or 31, or a pharmaceutically acceptable salt thereof, wherein X is O.

33. The compound of any one of claims 1 to 29, or 31, or a pharmaceutically acceptable salt thereof, wherein X is —NR⁴—.

34. The compound of any one of claims 1 to 29, or 31, or a pharmaceutically acceptable salt thereof, wherein X is —S—.

35. The compound of any one of the preceding claims, or a pharmaceutically acceptable salt thereof, wherein R³ is —C₁-C₆ alkyl, 4- to 10-membered heterocycloalkyl, or —C₁-C₆ alkylene-(4- to 10-membered heterocycloalkyl), wherein each hydrogen atom in —C₁-C₆ alkyl, 4- to 10-membered heterocycloalkyl, or —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

36. The compound of any one of the preceding claims, or a pharmaceutically acceptable salt thereof, wherein R³ is —C₁-C₆ alkylene-(4- to 10-membered heterocycloalkyl), wherein each hydrogen atom in —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

37. The compound of any one of the preceding claims, or a pharmaceutically acceptable salt thereof, wherein R³ is 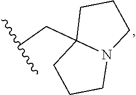 [SMILES: *CC12CCCN1CCC2]
wherein each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and “ [symbol] ” is a point of covalent attachment.

38. The compound of any one of the preceding claims, or a pharmaceutically acceptable salt thereof, wherein R³ is 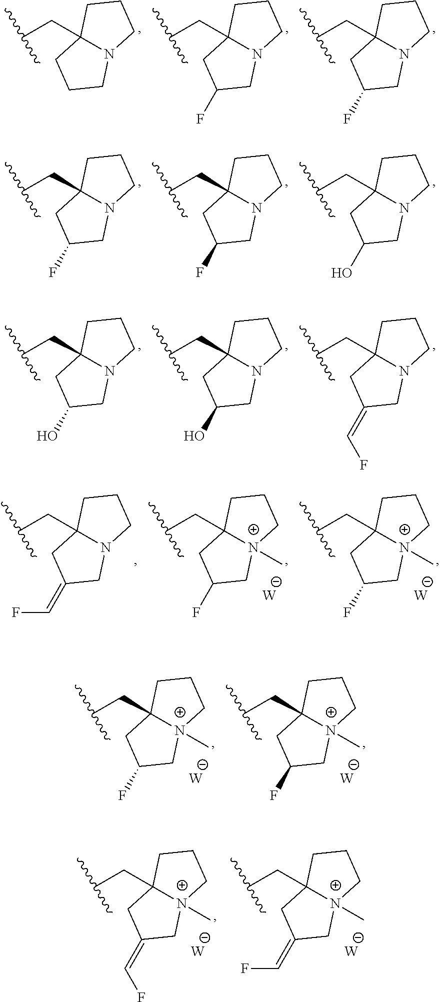
wherein W^{⊖} is an inorganic counter ion or an organic counter ion.

39. The compound of any one of claims 1 to 35, or a pharmaceutically acceptable salt thereof, wherein R³ is 4- to 10-membered heterocycloalkyl, wherein each hydrogen atom in 4- to 10-membered heterocycloalkyl is independently optionally substituted by deuterium, halogen, C₁-C₆alkyl, C₂-C₆alkenyl optionally substituted by halo —C₁-C₆alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

40. The compound of any one of claims 1 to 35, or 39, or a pharmaceutically acceptable salt thereof, wherein R³ is 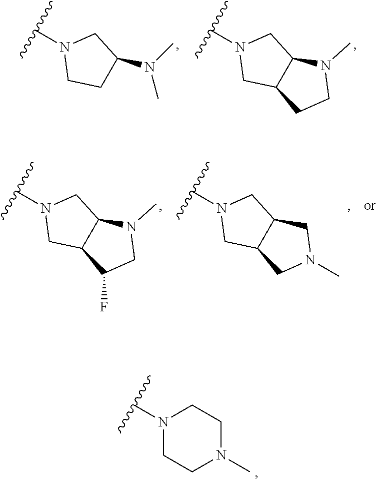
wherein “ [symbol] ” is a point of covalent attachment.

41. The compound of any one of the preceding claims, or a pharmaceutically acceptable salt thereof, wherein each R² is independently deuterium, halogen, C₁-C₆ alkyl, C₂-C₆ alkenyl, C₂-C₆ alkynyl, —OR^{c}, —C(O)R^{C}, —NR^{c}R^{d}, or —CN, wherein each hydrogen atom in C₁-C₆ alkyl, C₂-C₆ alkenyl, and C₂-C₆ alkynyl is independently optionally substituted with a deuterium or halogen.

42. The compound of any one of the preceding claims, or a pharmaceutically acceptable salt thereof, wherein ring A is 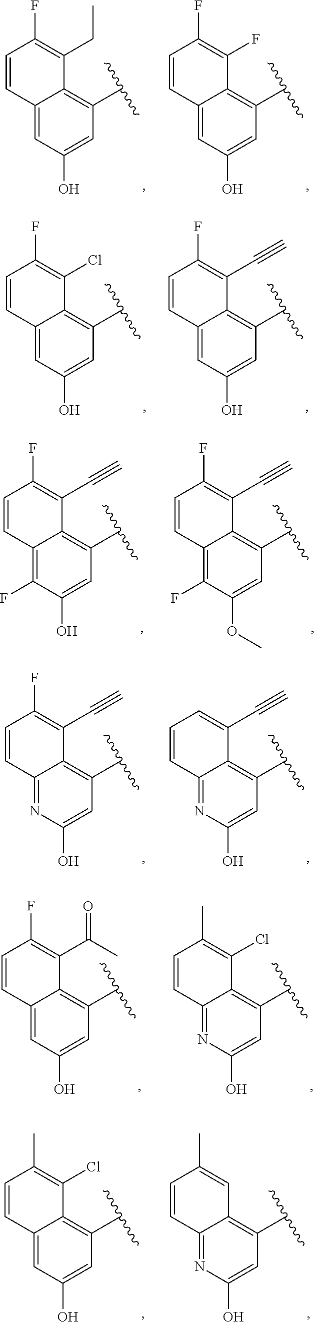 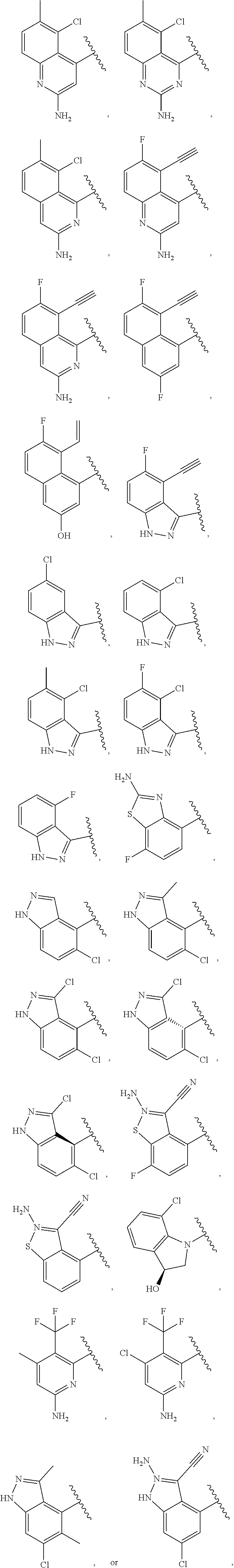
wherein “ [symbol] ” is a point of covalent attachment.

43. The compound of any one of the preceding claims, or a pharmaceutically acceptable salt thereof, wherein R⁴, when present, is H or methyl.

44. The compound of any one of the preceding claims, or a pharmaceutically acceptable salt thereof, wherein R⁵, when present, is H or deuterium.

45. The compound of any one of the preceding claims, or a pharmaceutically acceptable salt thereof, wherein R⁶, when present, is H or deuterium.

46. The compound of any one of the preceding claims, or a pharmaceutically acceptable salt thereof, wherein R⁷, when present, is H, deuterium, or F.

47. The compound of any one of the preceding claims, or a pharmaceutically acceptable salt thereof, wherein R⁸, when present, is H or deuterium.

48. The compound of any one of the preceding claims, or a pharmaceutically acceptable salt thereof, wherein Z⁵, when present, is N.

49. The compound of any one of the preceding claims, or a pharmaceutically acceptable salt thereof, wherein Z⁶, when present, is N.

50. The compound of any one of claims 1 to 42, or a pharmaceutically acceptable salt thereof, wherein Z⁵, when present, is CR¹¹.

51. The compound of claim 50, or a pharmaceutically acceptable salt thereof, wherein R¹¹, when present, is H or deuterium.

52. The compound of claim 50 or 51, or a pharmaceutically acceptable salt thereof, wherein Z⁶, when present, is N.

53. The compound of any one of claims 1 to 48, or a pharmaceutically acceptable salt thereof, wherein Z⁶, when present, is CR¹².

54. The compound of claim 53, or a pharmaceutically acceptable salt thereof, wherein R¹², when present, is H or deuterium.

55. The compound of claim 1, selected from the group consisting of 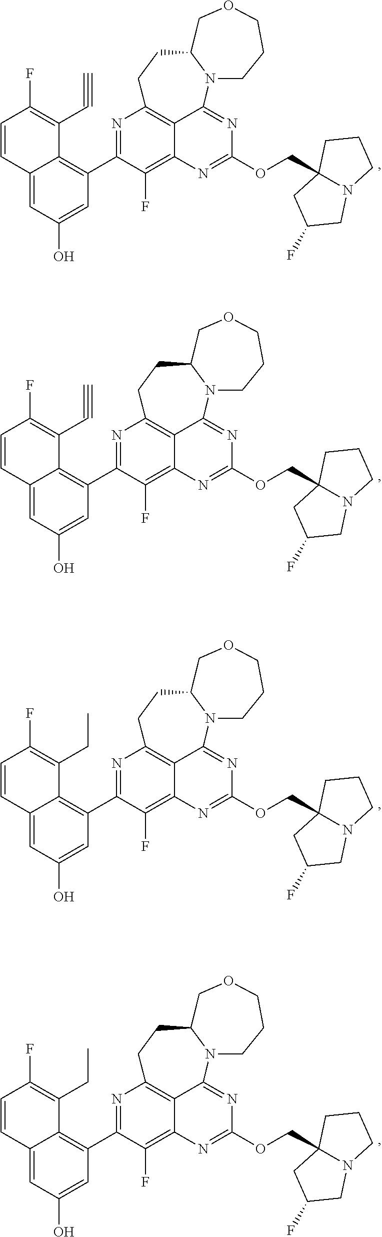 [SMILES: C#Cc1c(F)ccc2cc(O)cc(-c3nc4c5c(nc(OC[C@@]67CCCN6C[C@H](F)C7)nc5c3F)N3CCCOC[C@H]3CC4)c12 ; C#Cc1c(F)ccc2cc(O)cc(-c3nc4c5c(nc(OC[C@@]67CCCN6C[C@H](F)C7)nc5c3F)N3CCCOC[C@@H]3CC4)c12 ; CCc1c(F)ccc2cc(O)cc(-c3nc4c5c(nc(OC[C@@]67CCCN6C[C@H](F)C7)nc5c3F)N3CCCOC[C@H]3CC4)c12 ; CCc1c(F)ccc2cc(O)cc(-c3nc4c5c(nc(OC[C@@]67CCCN6C[C@H](F)C7)nc5c3F)N3CCCOC[C@@H]3CC4)c12] 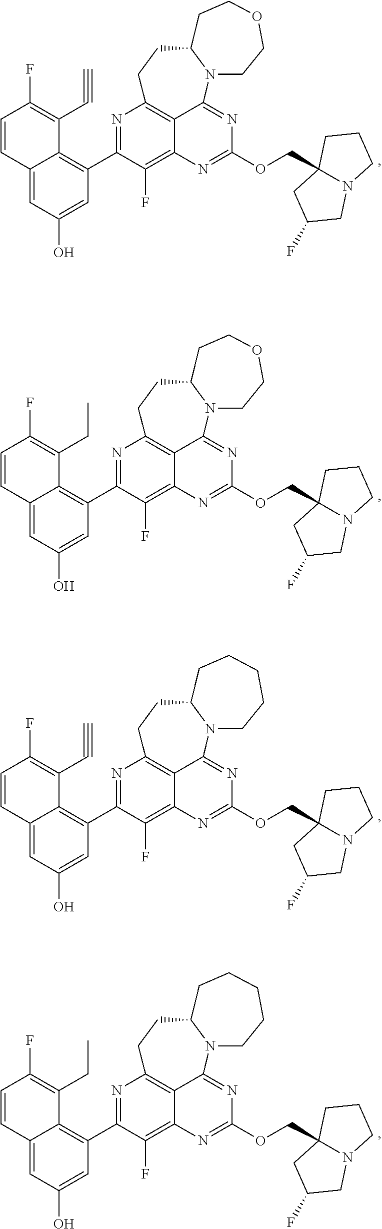 [SMILES: C#Cc1c(F)ccc2cc(O)cc(-c3nc4c5c(nc(OC[C@@]67CCCN6C[C@H](F)C7)nc5c3F)N3CCOCC[C@H]3CC4)c12 ; CCc1c(F)ccc2cc(O)cc(-c3nc4c5c(nc(OC[C@@]67CCCN6C[C@H](F)C7)nc5c3F)N3CCOCC[C@H]3CC4)c12 ; C#Cc1c(F)ccc2cc(O)cc(-c3nc4c5c(nc(OC[C@@]67CCCN6C[C@H](F)C7)nc5c3F)N3CCCCC[C@H]3CC4)c12 ; CCc1c(F)ccc2cc(O)cc(-c3nc4c5c(nc(OC[C@@]67CCCN6C[C@H](F)C7)nc5c3F)N3CCCCC[C@H]3CC4)c12] 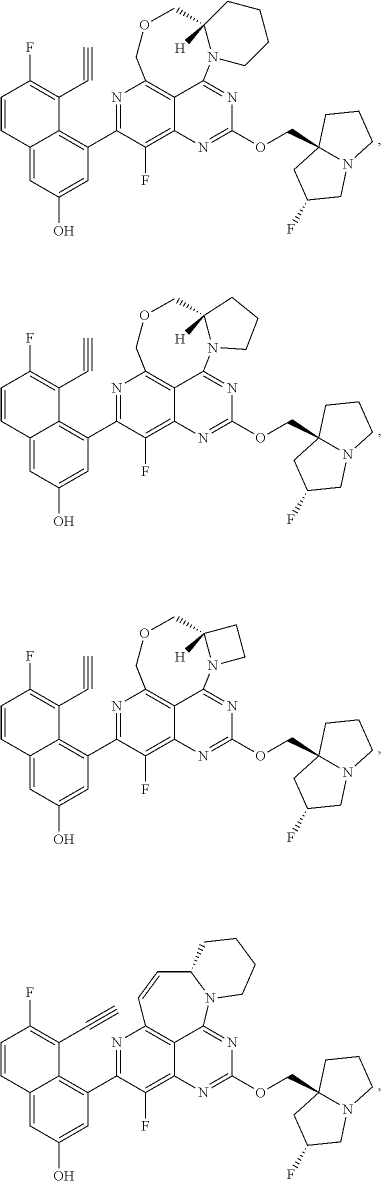 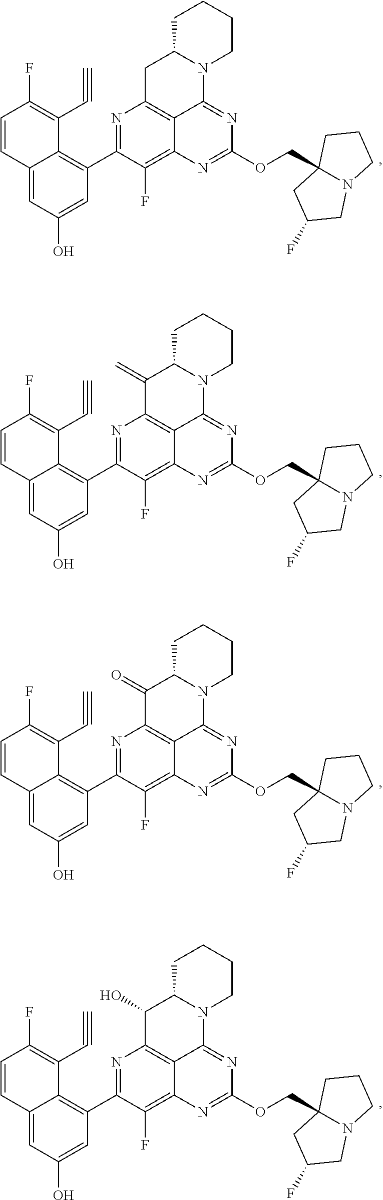 [SMILES: C#Cc1c(F)ccc2cc(O)cc(-c3nc4c5c(nc(OC[C@@]67CCCN6C[C@H](F)C7)nc5c3F)N3CCCC[C@H]3C4)c12 ; C#Cc1c(F)ccc2cc(O)cc(-c3nc4c5c(nc(OC[C@@]67CCCN6C[C@H](F)C7)nc5c3F)N3CCCC[C@H]3C4=C)c12 ; C#Cc1c(F)ccc2cc(O)cc(-c3nc4c5c(nc(OC[C@@]67CCCN6C[C@H](F)C7)nc5c3F)N3CCCC[C@H]3C4=O)c12 ; C#Cc1c(F)ccc2cc(O)cc(-c3nc4c5c(nc(OC[C@@]67CCCN6C[C@H](F)C7)nc5c3F)N3CCCC[C@H]3[C@H]4O)c12] 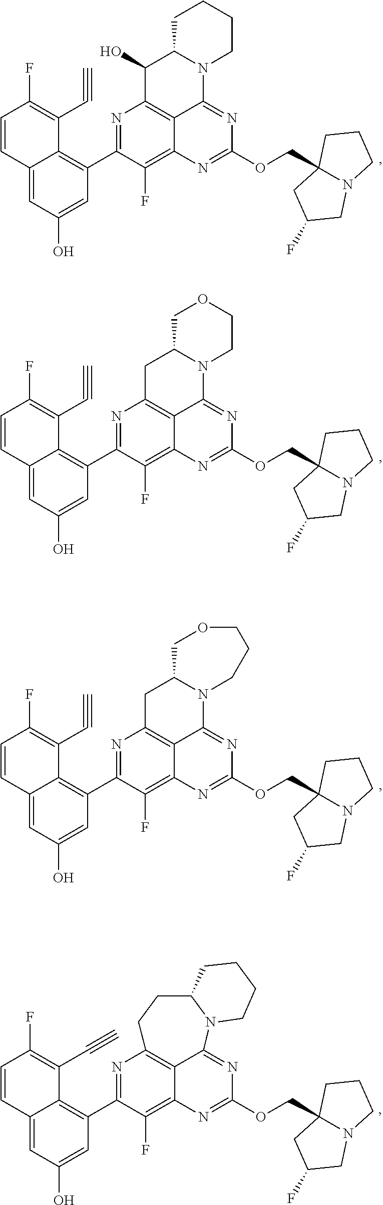 [SMILES: C#Cc1c(F)ccc2cc(O)cc(-c3nc4c5c(nc(OC[C@@]67CCCN6C[C@H](F)C7)nc5c3F)N3CCCC[C@H]3[C@@H]4O)c12 ; C#Cc1c(F)ccc2cc(O)cc(-c3nc4c5c(nc(OC[C@@]67CCCN6C[C@H](F)C7)nc5c3F)N3CCOC[C@H]3C4)c12 ; C#Cc1c(F)ccc2cc(O)cc(-c3nc4c5c(nc(OC[C@@]67CCCN6C[C@H](F)C7)nc5c3F)N3CCCOC[C@H]3C4)c12 ; C#Cc1c(F)ccc2cc(O)cc(-c3nc4c5c(nc(OC[C@@]67CCCN6C[C@H](F)C7)nc5c3F)N3CCCC[C@H]3CC4)c12] 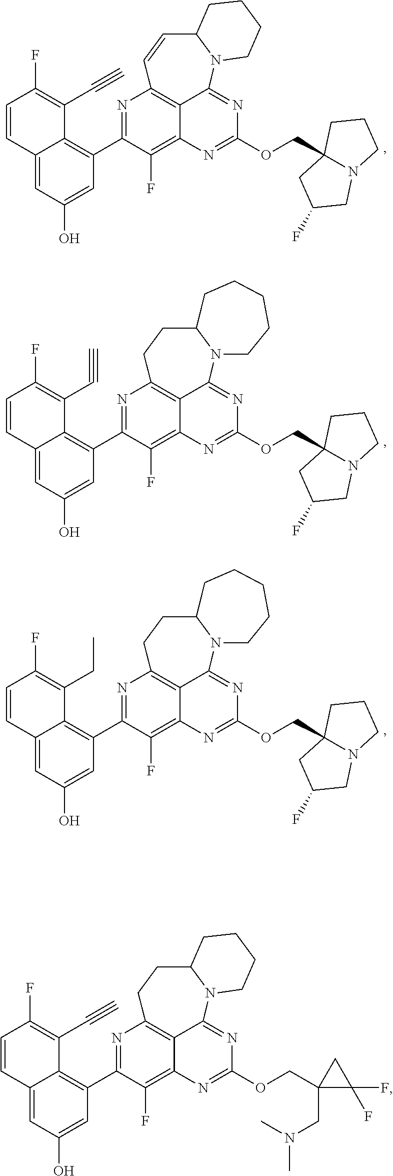 [SMILES: C#Cc1c(F)ccc2cc(O)cc(-c3nc4c5c(nc(OC[C@@]67CCCN6C[C@H](F)C7)nc5c3F)N3CCCCC3C=C4)c12 ; C#Cc1c(F)ccc2cc(O)cc(-c3nc4c5c(nc(OC[C@@]67CCCN6C[C@H](F)C7)nc5c3F)N3CCCCCC3CC4)c12 ; CCc1c(F)ccc2cc(O)cc(-c3nc4c5c(nc(OC[C@@]67CCCN6C[C@H](F)C7)nc5c3F)N3CCCCCC3CC4)c12 ; C#Cc1c(F)ccc2cc(O)cc(-c3nc4c5c(nc(OCC6(CN(C)C)CC6(F)F)nc5c3F)N3CCCCC3CC4)c12] 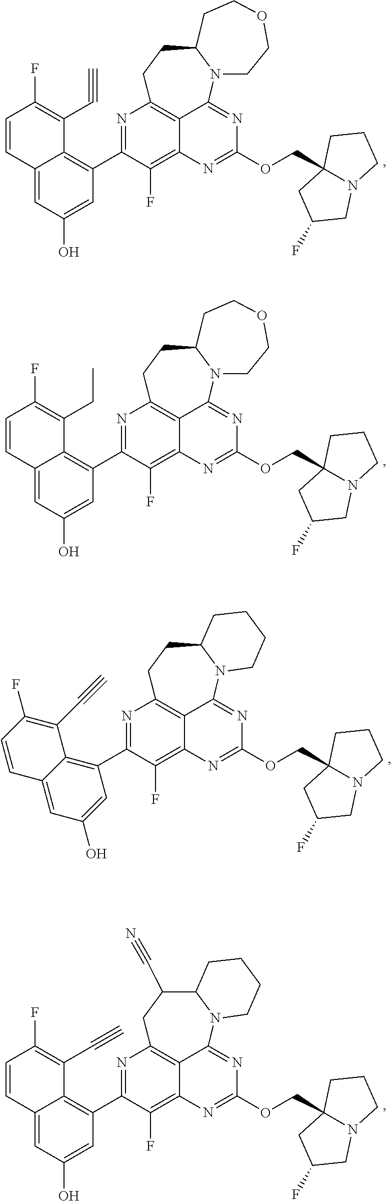 [SMILES: C#Cc1c(F)ccc2cc(O)cc(-c3nc4c5c(nc(OC[C@@]67CCCN6C[C@H](F)C7)nc5c3F)N3CCOCC[C@@H]3CC4)c12 ; CCc1c(F)ccc2cc(O)cc(-c3nc4c5c(nc(OC[C@@]67CCCN6C[C@H](F)C7)nc5c3F)N3CCOCC[C@@H]3CC4)c12 ; C#Cc1c(F)ccc2cc(O)cc(-c3nc4c5c(nc(OC[C@@]67CCCN6C[C@H](F)C7)nc5c3F)N3CCCC[C@@H]3CC4)c12 ; C#Cc1c(F)ccc2cc(O)cc(-c3nc4c5c(nc(OC[C@@]67CCCN6C[C@H](F)C7)nc5c3F)N3CCCCC3C(C#N)C4)c12] 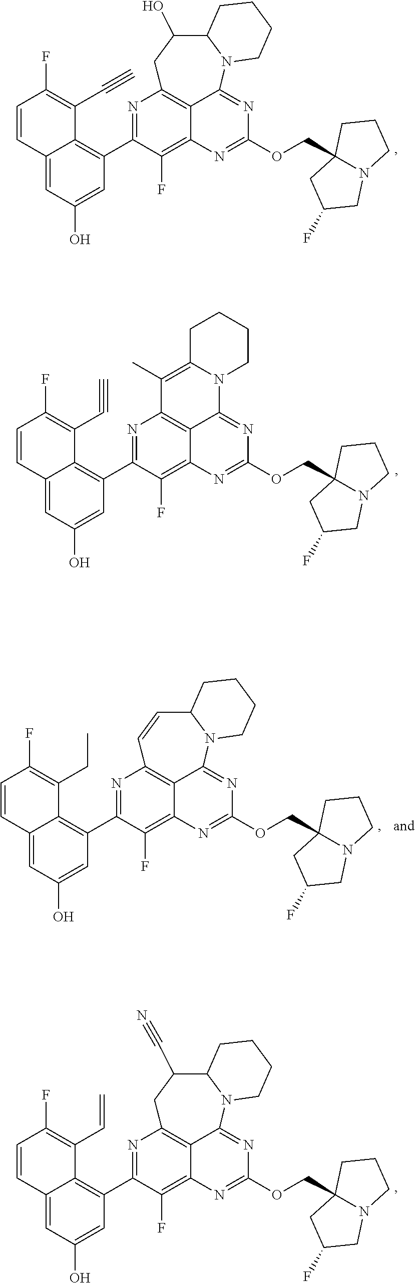 [SMILES: C#Cc1c(F)ccc2cc(O)cc(-c3nc4c5c(nc(OC[C@@]67CCCN6C[C@H](F)C7)nc5c3F)N3CCCCC3C(O)C4)c12 ; C#Cc1c(F)ccc2cc(O)cc(-c3nc4c5c(nc(OC[C@@]67CCCN6C[C@H](F)C7)nc5c3F)N3CCCCC3=C4C)c12 ; CCc1c(F)ccc2cc(O)cc(-c3nc4c5c(nc(OC[C@@]67CCCN6C[C@H](F)C7)nc5c3F)N3CCCCC3C=C4)c12 ; C=Cc1c(F)ccc2cc(O)cc(-c3nc4c5c(nc(OC[C@@]67CCCN6C[C@H](F)C7)nc5c3F)N3CCCCC3C(C#N)C4)c12]
or a pharmaceutically acceptable salt thereof.

56. A pharmaceutical composition comprising at least one compound of any one of claims 1 to 55, or a pharmaceutically acceptable salt thereof, and optionally one or more pharmaceutically acceptable excipients.

57. A method of treating disease, such as cancer, comprising administering to a subject in need of such treatment an effective amount of a compound of any one of claims 1 to 55, or a pharmaceutically acceptable salt thereof.

58. A compound of any one of claims 1 to 55, or a pharmaceutically acceptable salt thereof, for use in a method of treating cancer in a subject.

59. A compound of any one of claims 1 to 55, or a pharmaceutically acceptable salt thereof, for treating cancer in a subject.

60. Use of a compound of any one of claims 1 to 55, or a pharmaceutically acceptable salt thereof, in the manufacture of a medicament for treating cancer in a subject.
